(19) United States
(12) Patent Application Publication
(10) Pub. No.: US 2011/0092445 A1
(43) Pub. Date: Apr. 21, 2011

(54) Amyloid ß peptide analogues, oligomers thereof, processes for preparing and composi-tions comprising said analogues or oligomers, and their uses

(75) Inventors: Stefan Barghorn, Mannheim (DE); Heinz Hillen, Hassloch (DE); Rohinton Edalji, Wadsworth, IL (US); Leo Barrett, Lake Villa, IL (US); Paul Richardson, Libertyville, IL (US); Liping Yu, Iowa City, IA (US); Edward Olejniczak, Grayslake, IL (US); John E. Harlan, Lake Zurich, IL (US); Thomas Holzman, Libertyville, IL (US)

(73) Assignee: Abbott Laboratories, Abbott Park, IL (US)

(21) Appl. No.: 12/509,325

(22) Filed: Jul. 24, 2009

Related U.S. Application Data
(60) Provisional application No. 61/083,589, filed on Jul. 25, 2008.

(51) Int. Cl. A61K 38/12; C07K 4/00; C07K 14/00; G01N 33/68

(52) U.S. Cl. 514/21.1; 530/317; 436/501

(57) ABSTRACT

The present invention relates to an amyloid β peptide analogues comprising an amino acid sequence or a peptidomimetic thereof, wherein the sequence (i) forms a loop, (ii) has at least 66% identity to the amino acid sequence of native Aβ peptide or a portion thereof, (iii) comprises at least 6 contiguous amino acid residues and (iv) has at least 2 non-contiguous amino acid residues which are covalently linked with each other, oligomers comprising a plurality of said amyloid β peptide analogues, processes for preparing the amyloid β peptide analogues or oligomers, compositions comprising the amyloid β peptide analogues or oligomers, and uses of the amyloid β peptide analogues or oligomers such as their use for treating or preventing an amyloidosis (e.g. by active immunization), for diagnosing an amyloidosis, and for providing agents that are capable of binding to the amyloid β peptide analogues or oligomers. The subject invention also describes agents that are capable of binding to the amyloid β peptide analogues or oligomers, e.g. antibodies, compositions comprising the agents, and uses of the agents such as their use for treating or preventing an amyloidosis (e.g. by passive immunization) and for diagnosing an amyloidosis.

This application claims priority to the provisional application Ser. No. 61/083,589 filed Jul. 25, 2008, which is incorporated herein by reference.

### FIELD OF THE INVENTION

The subject invention relates to amyloid β peptide analogues, oligomers comprising a plurality of said amyloid β peptide analogues, processes for preparing the amyloid β peptide analogues or oligomers, compositions comprising the amyloid β peptide analogues or oligomers, and uses of the amyloid β peptide analogues or oligomers such as their use for treating or preventing an amyloidosis (e.g. by active immunization), for diagnosing an amyloidosis, and for providing agents that are capable of binding to the amyloid β peptide analogues or oligomers. The subject invention also describes agents that are capable of binding to the amyloid β peptide analogues or oligomers, e.g. antibodies, compositions comprising the agents, and uses of the agents such as their use for treating or preventing an amyloidosis (e.g. by passive immunization) and for diagnosing an amyloidosis.

### BACKGROUND OF THE INVENTION

In 1907, the physician Alois Alzheimer first described the neuropathological features of a form of dementia subsequently named in his honor as Alzheimer's disease (AD). In particular, AD is the most frequent cause for dementia among the aged, with an incidence of about 10% of the population in those above 65 years of age. With increasing age, the probability of disease also rises. Globally, there are about 15 million people affected with the disease and further increases in life expectancy are expected to increase the number of people affected with the disease to about three-fold over the next decades.

From a molecular point of view, Alzheimer's disease (AD) is characterized by a deposit of abnormally aggregated proteins. In the case of extracellular amyloid plaques, these deposits consist mostly of amyloid-β-peptide filaments, and in the case of the intracellular neurofibrillary tangles (NFTs), mostly of the tau protein. The amyloid β (Aβ) peptide arises from the β-amyloid precursor protein by proteolytic cleavage. This cleavage is effected by the cooperative activity of several proteases named α-, β- and γ-secretase. Cleavage leads to a number of specific fragments of differing length. The amyloid plaques consist mostly of peptides with a length of 40 or 42 amino acids (Aβ40, Aβ42). The dominant cleavage product is Aβ40; however, Aβ42 has a much stronger toxic effect. Cerebral amyloid deposits and cognitive impairments very similar to those observed in Alzheimer's disease are also hallmarks of Down's syndrome (trisomy 21), which occurs at a frequency of about 1 in 800 births.

The amyloid cascade hypothesis of Hardy and Higgins postulated that increased production of Aβ(1-42) would lead to the formation of protofibrils and fibrils (i.e., the principal components of Aβ plaques), these fibrils being responsible for the symptoms of Alzheimer's disease. Despite the poor correlation between severity of dementia and Aβ plaque burden deposited, this hypothesis was favored until recently.

U.S. Pat. No. 7,342,091 describes soluble cyclic analogues of amyloid β peptide having an intra-peptide bridge in the region between residues Gln15 and Val24 (686 and 695 in APP), with the two amino acids that participate in the bridge formation being arranged at relative separations of i+3, i+4, i+5, i+6 or i+7. Specifically, the side chains of Asp17 and Lys21 of the Aβ peptide (residues 688 and 692 in APP numbering) are connected via a covalent bridge. The soluble cyclic analogues of amyloid β peptide described in U.S. Pat. No. 7,342,091 are designed to inhibit amyloidogenesis or amyloid formation by endogenous Aβ peptide. That is, the soluble cyclic analogues are supposed to physically interact with the Aβ peptide and block amyloid from forming.

The discovery of soluble Aβ forms in AD brains, which correlates better with AD symptoms than plaque load does, has, however, led to a revised amyloid-cascade-hypothesis.

Under most conditions, amyloid β peptides rapidly convert into fibril forms. However, addition of detergent or fatty acid can result in long lived soluble forms (WO 2004/067561; WO 2006/094724; S. Barghorn et al., J. Neurochem. 95, 834 (2005)) that are potent antigens in mice and rabbits for eliciting specific antibodies. They have been shown to bind to dendritic processes of neurons in hippocamal cell cultures and completely block long-term potentiation in rat hippocampal slices. These data suggest that amyloid β-peptides with structural features similar to the soluble forms prepared in vitro are also present in vivo.

More specifically, WO 2004/067561 relates to globular oligomers (“globulomers”) of Aβ(1-42) peptide and a process for preparing them. The data suggest the existence of an amyloid fibril independent pathway of Aβ folding and assembly into Aβ oligomers which display one or more unique epitopes (hereinafter referred to as the globulomer epitopes). Since globulomer epitopes were detected in the brain of AD patients and APP transgenic mice and the globulomers specifically bind to neurons and blocks LTP, the globulomers represent a pathologically relevant Aβ conformer. WO 2004/067561 further describes that limited proteolysis of the globulomers yields truncated versions of said globulomers such as Aβ(20-42) or Aβ(12-42) globulomers. These Aβ(20-42) and Aβ(12-42) globulomers have been used to generate globulomer-specific antibodies. For instance, WO 2007/062852 describes several monoclonal antibodies which specifically recognize Aβ(20-42) globulomer.

WO 2006/094724 relates to non-diffusible globular Aβ(X-38 . . . 43) oligomers wherein X is selected from the group consisting of numbers 1 . . . 24. These globulomers are said to be obtainable by same processes as described in WO 2004/067561, i.e. SDS- or fatty acid-induced oligomerization of Aβ(1-38 . . . 43) peptide to generate Aβ(1-38 . . . 43) globulomers, and limited proteolysis of Aβ(1-38 . . . 43) globulomers to generate truncated versions thereof, i.e., Aβ(X-38 . . . 43) globulomers wherein X is selected from the group consisting of numbers 2 . . . 24.

Both, WO 2004/067561 and WO 2006/094724 also describe cross-linked globulomers obtained by reacting a globulomer with cross-linking agents such as glutardialdehyde. Cross-linking was observed to occur only between amino groups of the N-termini and Lys-16, while sparing Lys-28 which must therefore be hidden in the interior of the Aβ(1-42) globulomer. The resultant cross-link is primarily inter-molecular rather than intra-molecular.

WO 2007/064917 describes the cloning, expression and isolation of recombinant forms of amyloid β peptide. The peptide expressed in E. coli completely retains its N-terminal methionine residue and represents the native sequence of amyloid beta from positions 0 to position 42 (referred to hereafter as N-Met Aβ(1-42)).

Similar to Aβ-(1-42) peptide, adding either fatty acid or hydrocarbon detergent to preparations of N-Met Aβ-(1-42) peptides leads to the formation of stable soluble aggregates whose oligomeric state was observed to depend on the amount of residual detergent (SDS) or lipid-like additive. In the presence of 0.2% SDS, the amyloid β peptide forms a small soluble aggregate (referred to hereafter as N-Met Aβ(1-42) pre-globulomer) which can then be converted into a higher MW soluble aggregate (referred to hereafter as N-Met Aβ(1-42) globulomer) when the SDS concentration is diluted to 0.05%. Based on sedimentation studies, the N-Met Aβ(1-42) pre-globulomer has a MW of 16 kDa (corresponding to ˜4 peptides/soluble aggregate) while the N-Met Aβ(1-42) globulomer has a MW of ˜64 kDa (corresponding to ˜14-16 peptides/soluble aggregate).

Biophysical and structural characterization of N-Met Aβ(1-42) pre-globulomer revealed that it contains mixed intermolecular parallel/intramolecular anti-parallel β-sheets that are distinct from the all-parallel amyloid β-peptides found in structural studies of fibrils.

Said methods of globulomer formation represent a huge step in the ability to form homogeneous Aβ oligomer preparations in high yields. However, even this methodology can result in preparations showing some degree of heterogeneity, as sodium dodecyl sulfate (SDS) is removed, and over time. In addition, the truncations that have been made in order to best display the globulomer epitopes often further increase the heterogeneity and decrease the stability of the Aβ globulomers. These problems only increase as SDS is removed from the system. In addition, N-terminally truncated Aβ globulomers display very low solubility in the absence of detergents.

It was therefore an object of the present invention to provide amyloid β peptide analogues which display the relevant conformation or epitope, be it as a monomer or an oligomer. Preferably, such an amyloid β peptide analogue or oligomer thereof shows better physico/chemical properties than the known globulomers, e.g. a smaller size, enhanced homogeneity, enhanced stability, increased lifetime, and/or greater resistance to proteases in vivo. A better reproducibility would be a further advantage.

### SUMMARY OF THE INVENTION

The present invention provides a stabilized conformation of the Aβ peptide, or portions thereof, that displays an epitope important for: 1) the toxic response involved in progression of Alzheimer's disease (the “toxic principle” embodied in the Aβ misfolded peptide), 2) the generation of therapeutically relevant antibodies which are specific for this conformation and do not cross-react with endogenous physiological monomeric Aβ peptide as it is detectable in CSF and plasma, and/or 3) the engendering of an immune response by active immunization eliciting an antibody response which is polyclonal but mono-specific for this toxic conformation and does not cross-react with endogenous physiological monomer Aβ peptide as it is detectable in CSF and plasma. This stabilization is achieved by an intra-molecular covalent bond that locks the peptide or a peptidomimetic thereof into a conformation that both is more stable and displays the needed epitope. The desired potency of these stabilized peptides or peptidomimetics can easily be measured by cross-reaction with available globulomer selective antibodies as described in WO 2007/064972 and WO 2007/062852 in standard immunoassays (e.g., immunoprecipitation, ELISA, dot blot), or in standard cellular assays used to assess toxicity of Aβ peptides.

According to a first aspect, the present invention relates to amyloid β peptide analogues comprising an amino acid sequence, wherein the sequence (i) forms a loop, (ii) has at least 66% identity to the amino acid sequence of native Aβ peptide or a portion thereof, (iii) comprises at least 6 contiguous amino acid residues and (iv) has at least 2 non-contiguous amino acid residues which are covalently linked with each other.

According to a second aspect, the present invention also relates to amyloid β peptide analogues which comprise a peptidomimetic of said amino acid sequence as defined herein.

Particular embodiments of the amyloid β peptide analogues include the following:

the amyloid β peptide analogues, wherein the loop is a β-hairpin loop;
the amyloid β peptide analogues of any one of the preceding embodiments, wherein native Aβ human peptide or the portion thereof is Aβ(X . . . Y), X being selected from the group consisting of the numbers 1 . . . 23 and Y being selected from the group consisting of the numbers 28 . . . 43;
the amyloid β peptide analogues of the preceding embodiment, wherein X is selected from the group consisting of the numbers 15 . . . 23;
the amyloid β peptide analogues of the preceding embodiment, wherein X is selected from the group consisting of the numbers 18 . . . 22;
the amyloid β peptide analogues of the preceding embodiment, wherein Y is selected from the group consisting of the numbers 28 . . . 43;
the amyloid β peptide analogues of any one of the preceding embodiments, wherein native Aβ peptide or the portion thereof has a sequence selected from the group consisting of SEQ ID NO:1-368;
the amyloid β peptide analogues of any one of the preceding embodiments, wherein the 6 contiguous amino acid residues comprise the sequence VGSN or DVGSNK;
the amyloid β peptide analogues of any one of the preceding embodiments, wherein the 6 contiguous amino acid residues comprise the sequence AED;
the amyloid β peptide analogues of any one of the preceding embodiments, wherein the amino acid sequence of the amyloid β peptide analogue comprises the sequence X₁₉X₂₀X₂₁X₂₂X₂₃-VGSN-X₂₈X₂₉X₃₀X₃₁X₃₂, with each of X₁₉, X₂₀, X₂₁, X₂₂, X₂₃, X₂₈, X₂₉, X₃₀, X₃₁, X₃₂ independently representing an amino acid which may be covalently linked with another amino acid;
the amyloid β peptide analogues of the preceding embodiment, wherein the amino acid sequences X₁₉X₂₀X₂₁ and X₃₀X₃₁X₃₂ are in anti-parallel orientation;
the amyloid β peptide analogues of the preceding embodiments, wherein X₁₉ is an amino acid residue selected from the group consisting of phenylalanine, tyrosine, valine, leucine, isoleucine, and methionine;
the amyloid β peptide analogues of any one of the preceding embodiments, wherein X₂₀ is an amino acid residue selected from the group consisting of phenylalanine, tyrosine, valine, leucine, isoleucine, and methionine;
the amyloid β peptide analogues of any one of the preceding embodiments, wherein X₂₁ is an amino acid residue selected from the group consisting of alanine, valine, glycine, and serine;
the amyloid β peptide analogues of any one of the preceding embodiments, wherein X₂₂ is an amino acid residue selected from the group consisting of glutamic acid and aspartic acid;
the amyloid β peptide analogues of any one of the preceding embodiments, wherein X₂₃ is an amino acid residue selected from the group consisting of glutamic acid and aspartic acid;
the amyloid β peptide analogues of any one of the preceding embodiments, wherein X₂₈ is an amino acid residue selected from the group consisting of lysine and arginine;
the amyloid β peptide analogues of any one of the preceding embodiments, wherein X₂₉ is an amino acid residue selected from the group consisting of glycine, alanine, and serine;
the amyloid β peptide analogues of any one of the preceding embodiments, wherein X₃₀ is an amino acid residue selected from the group consisting of alanine, valine, glycine, and serine;
the amyloid β peptide analogues of any one of the preceding embodiments, wherein X₃₁ is an amino acid residue selected from the group consisting of isoleucine, leucine, valine, phenylalanine, and methionine;
the amyloid β peptide analogues of any one of the preceding embodiments, wherein X₃₂ is an amino acid residue selected from the group consisting of isoleucine, leucine, valine, phenylalanine, and methionine;
the amyloid β peptide analogues of any one of the preceding embodiments, wherein the amino acid sequence of the amyloid β peptide analogue comprises the sequence F₁₉X₂₀A₂₁-Q-A₃₀I₃₁I₃₂, with X₂₀ representing an amino acid and Q being an amino acid sequence comprising the sequence VGSN;
the amyloid β peptide analogues of the preceding embodiment, wherein at least part of the amino acid sequence Q forms the loop;
the amyloid β peptide analogues of the preceding embodiments, wherein the amino acid sequence Q consists of 5, 6, 7, or 8 amino acid residues;
the amyloid β peptide analogues of any one of the preceding embodiments, wherein the amino acid sequence of the amyloid β peptide analogues comprises the sequence F₁₉X₂₀A₂₁X₂₂D₂₃V₂₄G₂₅S₂₆N₂₇K₂₈X₂₉A₃₀I₃₁I₃₂ and each of X₂₀, X₂₂, X₂₉, independently represents an amino acid residue;
the amyloid β peptide analogues of the preceding embodiment, wherein the amino acid sequences F₁₀X₂₀A₂₁ and A₃₀I₃₁I₃₂ are in anti-parallel orientation;
the amyloid β peptide analogues of any one of the preceding embodiments, wherein the interproton distance for at least one atom pair selected from the group consisting of F₁₉(NH)—I₃₂(NH), F₁₉(NH)—I₃₂(HB), F₁₉(NH)—I₃₂(CG2), A₂₁(NH)-A₃₀(NH), A₂₁(NH)-A₃₀(CB), A₂₁(NH)—I₃₁(CD1), A₂₁(NH)—I₃₁(CG2), I₃₂(NH)—F₁₉(CD1), I₃₂(NH)—F₁₉(CD2), I₃₂(HN)—F₁₉(CB), and A₃₀(NH)-A₂₁(CB) is 1.8 to 6.5 Angstroms;
the amyloid β peptide analogues of any one of the preceding embodiments, wherein the atom pairs F₁₉(CO)—I₃₂(N), I₃₂(CO)—F₁₉(N), A₂₁(CO)-A₃₀(N), and A₃₀(CO)-A₂₁(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150;
the amyloid β peptide analogues of any one of the preceding embodiments, wherein the amino acid sequence of the amyloid β peptide analogue comprises a sequence selected from the group consisting of SEQ ID NO:1-368, at least two amino acid residues of said sequence being modified so as to form an intra-sequence covalent linkage;
the amyloid β peptide analogues of any one of the preceding embodiments, wherein the amino acid sequence of the amyloid β peptide analogue is a sequence selected from the group consisting of SEQ ID NO:369-698, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence;
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₁ is alanine, valine, glycine, serine, or an amino acid residue which is covalently linked to another amino acid residue of the sequence;
X₂₂ is glutamic acid, aspartic acid, or an amino acid residue which is covalently linked to another amino acid residue of the sequence;
X₂₉ is glycine, alanine, serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
at least one amino acid residue selected from the group consisting of X₁₂, X₁₃, X₁₄, X₁₅, X₁₆, X₁₇, X₁₈, X₁₉, X₂₀, X₂₁ and X₂₂ and at least one amino acid residue selected from the group consisting of X₂₉, X₃₀, X₃₁, X₃₂, X₃₃, X₃₄, X₃₅, X₃₆, X₃₇, X₃₈, X₃₀ being covalently linked with each other;
the amyloid β peptide analogues of the preceding embodiment, wherein at least one amino acid residue selected from the group consisting of X₁₂, X₁₃, X₁₄ and at least one amino acid residue selected from the group consisting of X₃₇, X₃₈, X₃₉ are covalently linked with each other, the amyloid β peptide analogues of the preceding embodiment, wherein at least one amino acid residue selected from the group consisting of X₁₃, X₁₄, X₁₅ and at least one amino acid residue selected from the group consisting of X₃₆, X₃₇, X₃₈ are covalently linked with each other, the amyloid β peptide analogues of the preceding embodiment, wherein at least one amino acid residue selected from the group consisting of X₁₄, X₁₅, X₁₆ and at least one amino acid residue selected from the group consisting of X₃₅, X₃₆, X₃₇ are covalently linked with each other, the amyloid β peptide analogues of the preceding embodiment, wherein at least one amino acid residue selected from the group consisting of X₁₅, X₁₆, X₁₇ and at least one amino acid residue selected from the group consisting of X₃₄, X₃₅, X₃₆ are covalently linked with each other, the amyloid β peptide analogues of the preceding embodiment, wherein at least one amino acid residue selected from the group consisting of X₁₆, X₁₇, X₁₈ and at least one amino acid residue selected from the group consisting of X₃₃, X₃₄, X₃₅ are covalently linked with each other, the amyloid β peptide analogues of the preceding embodiment, wherein at least one amino acid residue selected from the group consisting of X₁₇, X₁₈, X₁₉ and at least one amino acid residue selected from the group consisting of X₃₂, X₃₃, X₃₄ are covalently linked with each other, the amyloid β peptide analogues of the preceding embodiment, wherein at least one amino acid residue selected from the group consisting of X₁₈, X₁₉, X₂₀ and at least one amino acid residue selected from the group consisting of X₃₁, X₃₂, X₃₃ are covalently linked with each other, the amyloid β peptide analogues of the preceding embodiment, wherein at least one amino acid residue selected from the group consisting of X₁₉, X₂₀, X₂₁ and at least one amino acid residue selected from the group consisting of X₃₀, X₃₁, X₃₂ are covalently linked with each other, the amyloid β peptide analogues of the preceding embodiment, wherein at least one amino acid residue selected from the group consisting of X₂₀, X₂₁ and X₂₂ and at least one amino acid residue selected from the group consisting of X₂₉, X₃₀, X₃₁ are covalently linked with each other, or wherein the amino acid residues X₁₂ and X₃₉, X₁₃ and X₃₈, X₁₄ and X₃₇, X₁₅ and X₃₆, X₁₆ and X₃₅, X₁₇ and X₃₄, X₁₈ and X₃₃, X₁₉ and X₃₂, X₂₀ and X₃₁, X₂₁ and X₃₀, or X₂₂ and X₂₉ are covalently linked with each other;
the amyloid β peptide analogues of any one of the preceding embodiments, wherein the amino acid sequence of the amyloid β peptide analogue comprises the sequence X₂₀A₂₁E₂₂D₂₃-X₂₄X₂₅X₂₆X₂₇X₂₈X₂₉X₃₀X₃₁, with each of X₂₀, X₂₄, X₂₅, X₂₆, X₂₇, X₂₈, X₂₉, X₃₀, X₃₁ independently representing an amino acid which may be covalently linked with another amino acid;
the amyloid β peptide analogues of the preceding embodiment, wherein the amino acid sequences X₂₀X₂₁X₂₂X₂₃ and X₂₈X₂₉X₃₀X₃₁ are in anti-parallel orientation;
the amyloid β peptide analogues of the preceding embodiments, wherein X₂₀ is an amino acid residue selected from the group consisting of phenylalanine, tyrosine, valine, leucine, isoleucine, and methionine;
the amyloid β peptide analogues of any one of the preceding embodiments, wherein X₂₄ is an amino acid residue selected from the group consisting of valine, leucine, isoleucine, alanine, and methionine;
the amyloid β peptide analogues of any one of the preceding embodiments, wherein X₂₅ is an amino acid residue selected from the group consisting of glycine, alanine, and serine;
the amyloid β peptide analogues of any one of the preceding embodiments, wherein X₂₆ is an amino acid residue selected from the group consisting of serine, glycine, alanine, and threonine;
the amyloid β peptide analogues of any one of the preceding embodiments, wherein X₂₇ is an amino acid residue selected from the group consisting of asparagine, glutamine, and methionine;
the amyloid β peptide analogues of any one of the preceding embodiments, wherein X₂₈ is an amino acid residue selected from the group consisting of lysine and arginine;
the amyloid β peptide analogues of any one of the preceding embodiments, wherein X₂₉ is an amino acid residue selected from the group consisting of glycine, alanine, and serine;
the amyloid β peptide analogues of any one of the preceding embodiments, wherein X₃₀ is an amino acid residue selected from the group consisting of alanine, valine, glycine, and serine;
the amyloid β peptide analogues of any one of the preceding embodiments, wherein X₃₁ is an amino acid residue selected from the group consisting of isoleucine, leucine, valine, phenylalanine, and methionine;
the amyloid β peptide analogues of any one of the preceding embodiments, wherein the amino acid sequence of the amyloid β peptide analogue comprises the sequence X₂₀-Q-X₂₄X₂₅X₂₆X₂₇X₂₈X₂₉A₃₀I₃₁, with each of X₂₀, X₂₄ X₂₅, X₂₆, X₂₇, X₂₈, X₂₉ independently representing an amino acid and Q being an amino acid sequence comprising the sequence AED; the amyloid β peptide analogues of the preceding embodiment, wherein at least part of the amino acid sequence X₂₄X₂₅X₂₆X₂₇ forms the loop;
the amyloid β peptide analogues of the preceding embodiments, wherein the amino acid sequence Q consists of 3, 4, 5, or 6 amino acid residues;
the amyloid β peptide analogues of any one of the preceding embodiments, wherein the amino acid sequence of the amyloid β peptide analogue comprises the sequence X₂₀A₂₁E₂₂D₂₃X₂₄X₂₅X₂₆X₂₇X₂₈X₂₉A₃₀I₃₁ and each of X₂₀, X₂₄, X₂₅, X₂₆, X₂₇, X₂₈, X₂₉, independently represents an amino acid residue;
the amyloid β peptide analogues of the preceding embodiment, wherein the amino acid sequences X₂₀A₂₁E₂₂D₂₃ and X₂₈X₂₉A₃₀I₃₁ are in anti-parallel orientation;
the amyloid β peptide analogue of the preceding embodiments, wherein the interproton distance for at least one atom pair selected from the group consisting of A₂₁(NH)-A₃₀(NH),
A₂₁(NH)-A₃₀(CB), A₂₁(NH)—I₃₁(CD1), A₂₁(NH)—I₃₁(CG2), and A₃₀(NH)-A₂₁(CB) is 1.8 to 6.5 Angstroms;
the amyloid β peptide analogues of any one of the preceding embodiments, wherein the atom pairs A₂₁(CO)-A₃₀(N) and A₃₀(CO)-A₂₁(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150;
the amyloid β peptide analogues of any one of the preceding embodiments, wherein the amino acid sequence of the amyloid β peptide analogue is a sequence selected from the group consisting of SEQ ID NO:699-960, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence;
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₄ is valine, leucine, isoleucine, alanine, methionine, or an amino acid residue which is covalently linked to another amino acid residue of the sequence;
X₂₅ is glycine, alanine, serine, or an amino acid residue which is covalently linked to another amino acid residue of the sequence;
X₂₆ is serine, glycine, alanine, threonine, or an amino acid residue which is covalently linked to another amino acid residue of the sequence;
X₂₇ is asparagine, glutamine, methionine, or an amino acid residue which is covalently linked to another amino acid residue of the sequence;
X₂₈ is lysine, arginine, or an amino acid residue which is covalently linked to another amino acid residue of the sequence;
X₂₉ is glycine, alanine, serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
at least one amino acid residue selected from the group consisting of X₁₂, X₁₃, X₁₄, X₁₅, X₁₆, X₁₇, X₁₈, X₁₉, X₂₀, and at least one amino acid residue selected from the group consisting of X₂₉, X₃₀, X₃₁, X₃₂, X₃₃, X₃₄, X₃₅, X₃₆, X₃₇, X₃₈, X₃₉ being covalently linked with each other;
the amyloid β peptide analogues of the preceding embodiment, wherein at least one amino acid residue selected from the group consisting of X₁₂, X₁₃, X₁₄, and at least one amino acid residue selected from the group consisting of X₃₇, X₃₈, X₃₉ are covalently linked with each other, wherein at least one amino acid residue selected from the group consisting of X₁₃, X₁₄, X₁₅, and at least one amino acid residue selected from the group consisting of X₃₆, X₃₇, X₃₈ are covalently linked with each other, wherein at least one amino acid residue selected from the group consisting of X₁₄, X₁₅, X₁₆, and at least one amino acid residue selected from the group consisting of X₃₅, X₃₆, X₃₇ are covalently linked with each other, wherein at least one amino acid residue selected from the group consisting of X₁₅, X₁₆, X₁₇, and at least one amino acid residue selected from the group consisting of X₃₄, X₃₅, X₃₆ are covalently linked with each other, wherein at least one amino acid residue selected from the group consisting of X₁₆, X₁₇, X₁₈, and at least one amino acid residue selected from the group consisting of X₃₃, X₃₄, X₃₅ are covalently linked with each other; wherein at least one amino acid residue selected from the group consisting of X₁₇, X₁₈, X₁₉, and at least one amino acid residue selected from the group consisting of X₃₂, X₃₃, X₃₄ are covalently linked with each other, wherein at least one amino acid residue selected from the group consisting of X₁₈, X₁₉, X₂₀, and at least one amino acid residue selected from the group consisting of X₃₁, X₃₂, X₃₃ are covalently linked with each other, wherein at least one amino acid residue selected from the group consisting of X₁₉, X₂₀, and at least one amino acid residue selected from the group consisting of X₃₀, X₃₁, X₃₂ are covalently linked with each other, wherein amino acid residue X₂₀ and at least one amino acid residue selected from the group consisting of X₂₉, X₃₀, X₃₁ are covalently linked with each other, or wherein the amino acid residues X₁₂ and X₃₉, X₁₃ and X₃₈, X₁₄ and X₃₇, X₁₅ and X₃₆, X₁₆ and X₃₅, X₁₇ and X₃₄, X₁₈ and X₃₃, X₁₉ and X₃₂, or X₂₀ and X₃₁, are covalently linked with each other;
the amyloid β peptide analogues of any one of the preceding embodiments, wherein the amino acid residue is covalently linked via its side chain;
the amyloid β peptide analogues of the preceding embodiment, wherein the side chains of the amino acid residues has a functional group which is independently selected from the group consisting of thiol, amino, carboxyl and hydroxyl groups;
the amyloid β peptide analogues of any one of the preceding embodiments, wherein the amino acid residue that is covalently linked to the other amino acid residues is that of an amino acid residue selected from the group consisting of cysteine, lysine, aspartic acid and glutamic acid;
the amyloid β peptide analogues of the preceding embodiment, wherein the side chains of a cysteine and a cysteine, a cysteine and a lysine, an aspartic acid or a glutamic acid and a lysine, or alysine and a lysine are covalently linked with each other;
the amyloid β peptide analogues of any one of the preceding embodiments, wherein the side chains are covalently linked via a direct covalent bond;
the amyloid β peptide analogues of any one of the preceding embodiments, wherein the side chains are covalently linked via a linker;
the amyloid β peptide analogues of the preceding embodiment, wherein the linker is a homobifunctional or a heterobifunctional linker;
the amyloid β peptide analogues of the preceding embodiment, wherein the linker is a photo-reactive linker;
the amyloid β peptide analogues of any one of the preceding embodiments, wherein covalent linkage comprises a disulfide bond;
the amyloid β peptide analogues of any one of the preceding embodiments, wherein covalent linkage comprises an amide bond;
the amyloid β peptide analogues of any one of the preceding embodiments, wherein the amino acid sequence of the amyloid β peptide analogue comprises one covalent linkage between 2 non-contiguous amino acid residues.

According to a third aspect, the present invention relates to oligomers comprising a plurality of said amyloid β peptide analogues.

Particular embodiments of the oligomers include the following:

the oligomers, wherein the plurality is 2 to 28 amyloid β peptide analogues;
the oligomers of the preceding embodiments, wherein the amino acid sequence of each amyloid β peptide analogue comprises the sequence L₃₄M₃₅V₃₆G₃₇G₃₈, with the sequence L^{A}₃₄M^{A}₃₅V^{A}₃₆G^{A}₃₇G^{A}₃₈ of one amyloid β peptide analogue being in parallel orientation to the sequence L^{B}₃₄M^{B}₃₅V^{B}₃₆G^{B}₃₇G^{B}₃₈ of another amyloid β peptide analogue;
the oligomers of the preceding embodiment, wherein the interproton distance for at least one atom pair selected from the group consisting of M^{A}₃₅(NH)—V^{B}₃₆(NH), G^{A}₃₇(NH)-G^{B}₃₈(NH), L^{A}₃₄(NH)-L^{B}₃₄(C_{δ}H₃), M^{A}₃₅(NH)—V^{B}₃₆(CγH₃) is 1.8 to 6.5 Angstroms;
the oligomers of any one of the preceding embodiments, wherein the amino acid sequence of each amyloid β peptide analogue comprises the sequence G₃₃L₃₄M₃₅V₃₆G₃₇G₃₈V₃₉, with the sequence G^{A}₃₃L^{A}₃₄M^{A}₃₅V^{A}₃₆G^{A}₃₇G^{A}₃₈V^{A}₃₉ of one amyloid β peptide analogue being in parallel orientation to the sequence G^{B}₃₃L^{B}₃₄M^{B}₃₅V^{B}₃₆G^{B}₃₇G^{B}₃₈V^{B}₃₉ of another amyloid β peptide analogue;
the oligomers of the preceding embodiment, wherein the interproton distance for at least one atom pair selected from the group consisting of G^{A}₃₃(NH)-G^{B}₃₄(NH), M^{A}₃₅(NH)—V^{B}₃₆(NH), G^{A}₃₇(NH)-G^{B}₃₈(NH), L^{A}₃₄(NH)-L^{B}₃₄(C_{δ}H₃), M^{A}₃₅(NH)—V^{B}₃₆(CyH₃), G^{A}₃₈(NH)—V^{B}₃₉(CγH₃) and V^{A}₃₉(NH)—V^{B}₃₉(CγH₃) is 1.8 to 6.5 Angstroms;
the oligomers of any one of the preceding embodiments, wherein the oligomers comprise an inter-molecular parallel β-sheet;
the oligomers of the preceding embodiment, wherein the β-sheet comprises the amino acid sequence G^{A}₃₃L^{A}₃₄M^{A}₃₅V^{A}₃₆G^{A}₃₇G^{A}₃₈V^{A}₃₉ of one amyloid β peptide analogue and the amino acid sequence G^{A}₃₃L^{A}₃₄M^{A}₃₅V^{A}₃₆G^{A}₃₇G^{A}₃₈V^{A}₃₉ of another amyloid β peptide analogue; the oligomers of the preceding embodiment, wherein the atom pairs G^{A}33(CO)-L^{B}34(N), L^{B}34(CO)-M^{A}35(N), M^{A}35(CO)—V^{B}36(N), V^{B}36(CO)-G^{A}37(N), and G^{B}37(CO)-G^{A}38(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150.

A further particular embodiment includes the amyloid β peptide analogues or oligomers of any one of the preceding embodiments, which comprise an epitope recognized by a monoclonal antibody selected from the group consisting of the monoclonal antibody 5F7 obtainable from a hybridoma designated by American Type Culture Collection deposit number PTA-7241, the monoclonal antibody 7C6 obtainable from a hybridoma designated by American Type Culture Collection deposit number PTA-7240, the monoclonal antibody 4D10 obtainable from a hybridoma designated by American Type Culture Collection deposit number PTA-7405, or the monoclonal antibody 7E5 obtainable from a hybridoma designated by American Type Culture Collection deposit number PTA-7809.

The present invention also relates to a process for preparing an amyloid β peptide analogue as defined herein, which process comprises
(i) providing a peptide or peptidomimetic thereof;
(ii) subjecting the peptide or peptidomimetic to conditions sufficient for the formation of the linkage.

The present invention also relates to a process for preparing an oligomer as defined herein, which process comprises
(i) providing a peptide or peptidomimetic thereof;
(ii) subjecting the peptide or peptidomimetic to conditions sufficient for the formation of the oligomer and linkage.

Particular embodiments of the processes include processes, wherein the oligomer formation precedes that linkage formation.

Further, the present invention relates to compositions comprising an amyloid β peptide analogue or oligomer as defined herein.

Particular embodiments of the processes include composition, wherein the composition is a vaccine and further comprises a pharmaceutical acceptable carrier.

The present invention also relates to the use of an amyloid β peptide analogue or oligomer as defined herein for preparing a pharmaceutical composition for treating or preventing an amyloidosis and to corresponding methods of treating or preventing an amyloidosis in a subject in need thereof, which comprises administering an amyloid β peptide analogue or oligomer as defined herein to the subject.

Particular embodiments of the use and methods include the following: the use and methods, wherein the pharmaceutical composition is for active immunization; the use and methods of the preceding embodiments, wherein the amyloidosis is Alzheimer's disease or wherein the amyloidosis is the amyloidosis of Down's syndrome.

The present invention also relates to the use of an amyloid β peptide analogue or oligomer as defined herein for preparing a composition for diagnosing an amyloidosis and to corresponding methods of diagnosing an amyloidosis which comprises providing a sample from the subject suspected of having the amyloidosis, contacting the sample with an amyloid β peptide analogue or oligomer as defined herein for a time and under conditions sufficient for the formation of a complex comprising the amyloid β peptide analogue or oligomer and an antibody, the presence of the complex indicating the subject has the amyloidosis.

Particular embodiments of the use and methods include the use and methods, wherein the amyloidosis is Alzheimer's disease or wherein the amyloidosis is the amyloidosis of Down's syndrome.

Further, the present invention relates to a method of enriching an agent capable of binding to an amyloid β peptide analogue or oligomer as defined herein in a preparation comprising said agent, which method comprises the steps of: a) exposing to the amyloid β peptide analogue or oligomer the preparation comprising the agent for a time and under conditions sufficient for the agent to bind to amyloid β peptide analogue or oligomer; and b) obtaining the agent in enriched form.

Particular embodiments of the use and methods include the use and methods, wherein the agent is an antibody, an aptamer or a small molecular weight compound.

Also, the present invention relates to the use of an amyloid β peptide analogue or oligomer as defined herein for providing an agent that is capable of binding to the amyloid β peptide analogue or oligomer and to corresponding methods, e.g. a method of providing an antibody capable of binding to an amyloid β peptide analogue or oligomer as defined herein, which comprises
i) providing an antigen comprising the amyloid β peptide analogue or oligomer;
ii) exposing an antibody repertoire to said antigen; and
iii) selecting from said repertoire an antibody which binds to the amyloid β peptide analogue or oligomer.

Particular embodiments of the use and methods include the use and methods, wherein the agent is an antibody, a non-antibody binding molecule, an aptamer or a small molecular weight compound.

Antibodies which are obtainable by said process are also described as well as agents capable of binding to an amyloid β peptide analogue or oligomer of the invention.

Further, the present invention describes compositions comprising an agent capable of binding to an amyloid β peptide analogue or oligomer of the invention; the use of an agent capable of binding to an amyloid β peptide analogue or oligomer of the invention for preparing a pharmaceutical composition for treating or preventing an amyloidosis and corresponding methods of treating or preventing an amyloidosis in a subject in need thereof, which comprises administering an agent capable of binding to an amyloid β peptide analogue or oligomer of the invention to the subject; the use of an agent capable of binding to an amyloid β peptide analogue or oligomer of the invention for preparing a composition for diagnosing an amyloidosis and corresponding methods of diagnosing an amyloidosis which comprises providing a sample from the subject suspected of having the amyloidosis, contacting the sample with an agent capable of binding to an amyloid β peptide analogue or oligomer of the invention for a time and under conditions sufficient for the formation of a complex comprising the agent and an antigen, the presence of the complex indicating the subject has the amyloidosis.

### BRIEF DESCRIPTION OF DRAWINGS

FIG. 1 shows (A) a diagram of the NMR derived structure of Aβ pre-globulomer, depicting the inter-residue NOE's used to define the three-dimensional fold; dashed lines indicate observed NOEs and circles the backbone amides that exhibit slow exchange in the NH/ND exchange experiments; (B) a ribbon diagram depicting NMR derived structure of Aβ pre-globulomer in SDS; residues with defined structure are high-lighted in bold text; (C) a diagram of one monomer observed in the NMR Aβ pre-globulomer structure, showing the Leu to Cys mutations; (D) a diagram of one monomer observed in the NMR Aβ pre-globulomer structure, showing the Leu to Cys mutations and the resulting disulfide cross-link structure.

FIG. 2 shows (A) an SDS-PAGE gel with typical Aβ globulomer banding pattern formed from globulomers made with S#6046: wt N-Met Aβ(1-42) peptide, Mut Pre: (17C, 34C) N-Met Aβ(1-42) mutant peptide in 0.2% SDS, Mut Post: (L17C, L34C) N-Met Aβ(1-42) mutant peptide in 0.05% SDS; (B) an SDS-PAGE of 1) marker proteins, 2) (14C, 37C) N-Met Aβ(1-42) oligomer, 3) (14C, 37C) N-Met Aβ(1-42) oligomer after thermolysin digestion, 4) (15C, 36C) N-Met Aβ(1-42) oligomer, 5) (15C, 36C) N-Met Aβ(1-42) oligomer after thermolysin digestion, 6) (16C, 35C) N-Met Aβ(1-42) oligomer, 7) (16C, 35C) N-Met Aβ(1-42) oligomer after thermolysin digestion, 8) (17C, 34C) N-Met Aβ(1-42) oligomer, 9) (17C, 34C) N-Met Aβ(1-42) oligomer after thermolysin digestion, 10) (18C, 33C) N-Met Aβ(1-42) oligomer, 11) (18C, 33C) N-Met Aβ(1-42) oligomer after thermolysin digestion; (C) an SDS-PAGE of 1) marker proteins, 2) (19C, 32C) N-Met Aβ(1-42) oligomer, 3) (19C, 32C) N-Met Aβ(1-42) oligomer after thermolysin digestion, 4) (20C, 31C) N-Met Aβ(1-42) oligomer, 5) (20C, 31C) N-Met Aβ(1-42) oligomer after thermolysin digestion, 6) (21C, 30C) N-Met Aβ(1-42) oligomer, 7) (21C, 30C) N-Met Aβ(1-42) oligomer after thermolysin digestion, 8) (22C, 29C) N-Met Aβ(1-42) oligomer, 9) (22C, 29C) N-Met Aβ(1-42) oligomer after thermolysin digestion, 10) Aβ(1-42) globulomer, 11) Aβ(1-42) globulomer after thermolysin digestion; (D) an SDS-PAGE of 1) (17K, 34E) N-Met Aβ(1-42) oligomer (0.2% SDS), 2) (17K, 34E) N-Met Aβ(1-42) oligomer (0.05% SDS), 3) (17C(ACM), 34C(ACM)) Aβ(16-35) oligomer (0.2% SDS), 4) (17C(ACM), 34C(ACM)) Aβ(16-35) oligomer (0.05% SDS), 5) (17K, 34C) N-Met Aβ(1-42) oligomer (0.2% SDS), 6) (17K, 34C) N-Met Aβ(1-42) oligomer (0.05% SDS), 7) (17C, 34C) Aβ(16-42) oligomer (0.2% SDS), 8) (17C, 34C) Aβ(16-42) oligomer (0.05% SDS), 9) (17KC, 34C) Aβ(13-42) oligomer (0.2% SDS), 10) (17KC, 34C) Aβ(13-42) oligomer (0.05% SDS), 11) N-Met Aβ(1-42) oligomer. Standards (lanes 1 and 5 of (A) and marker proteins of (B) and (C)) are: myosin (210 kDa), phosphorylase (98 kDa), BSA (78 kDa), glutamic dehydrogenase (55 kDa), alcohol dehydrogenase (45 kDa), carbonic anhydrase (34 kDa), myoglobin red (17 kDa), lysozyme (16 kDa), aprotinin (7 kDa) and insulin (4 kDa). The attributes of the banding pattern are: a cluster of bands-40-50 kDa, a cluster of bands ˜15 kDa, and a band at ˜5 kDa (monomer).

FIG. 3 shows (A) a comparison of direct Elisa response of N-Met Aβ(1-42) globulomer and (L17C, L34C) N-Met Aβ(1-42) mutant globulomer to the globulomer-specific monoclonal antibody 5F7;

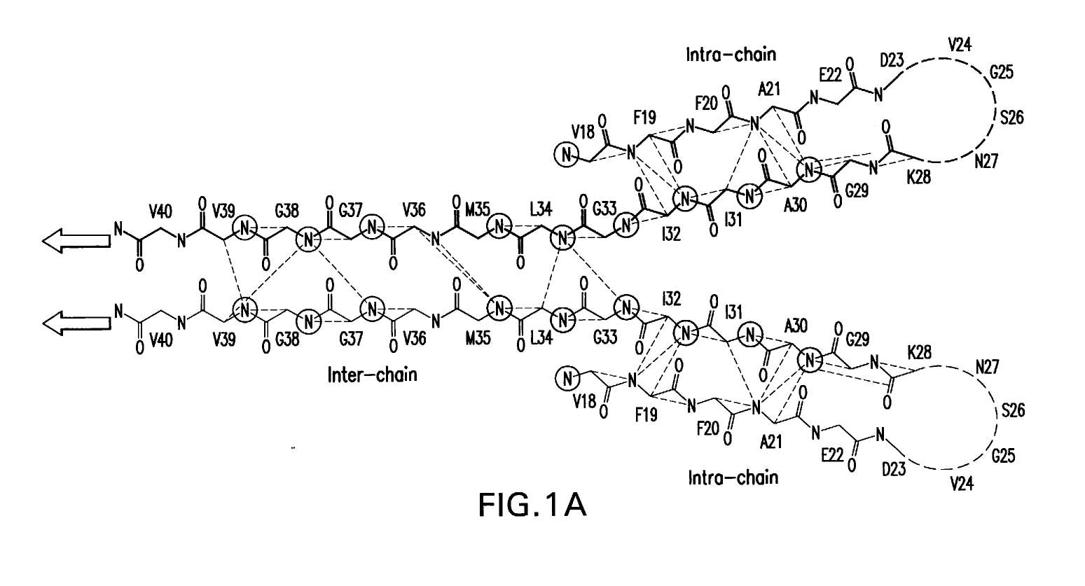

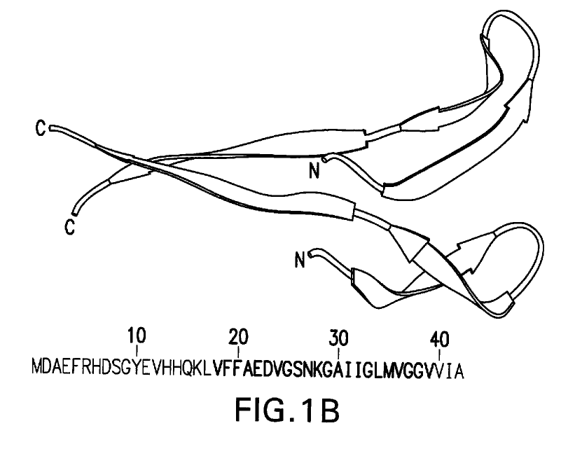

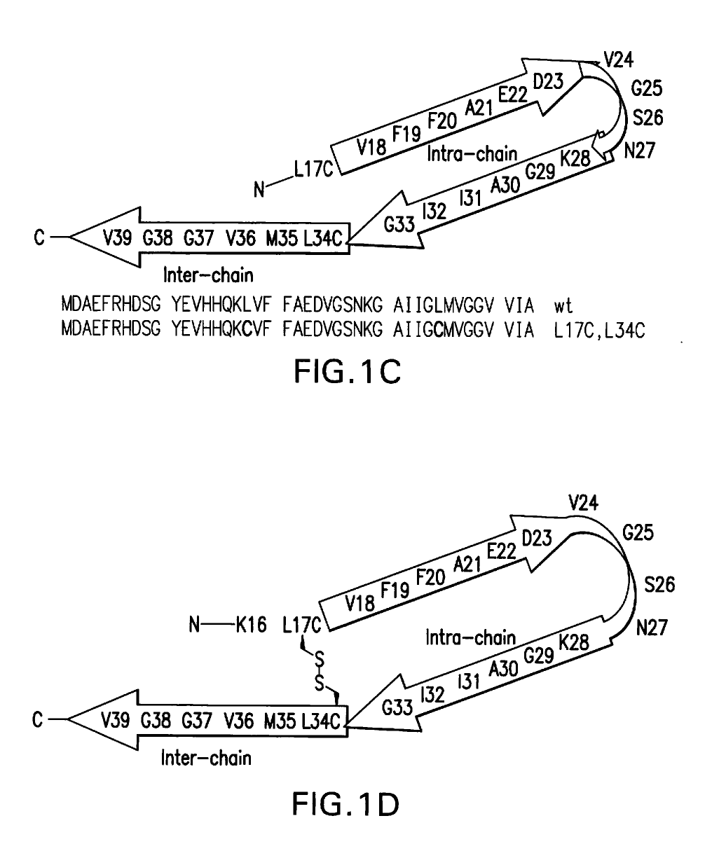

(B) a comparison of direct ELISA results comparing the apparent binding affinity of the globulomer-specific mAb 5F7 to disulfide stabilized (17C, 34C) N-Met Aβ(1-42) mutant globulomer vs. the same mutant globulomer truncated at residue 20 by enzymatic cleavage with thermolysin;
(C) a comparison of direct ELISA results comparing the apparent binding affinity of the globulomer-specific mAb 7C6 to disulfide stabilized (17C, 34C) N-Met Aβ(1-42) mutant globulomer vs. the same mutant globulomer truncated at residue 20 by enzymatic cleavage with thermolysin;
(D) a comparison of direct ELISA results comparing the apparent binding affinity of the globulomer-specific rabbit polyclonal antiserum 5599 to disulfide stabilized (17C, 34C) N-Met Aβ(1-42) mutant globulomer vs. the same mutant globulomer truncated at residue 20 by enzymatic cleavage with thermolysin;
(E) a comparison of direct ELISA results comparing the apparent binding affinity of the globulomer-specific mAb 5F7 to disulfide stabilized (17C, 34C) Aβ(16-35) oligomer vs. the same oligomer truncated at residue 20 by enzymatic cleavage with thermolysin;
(F) a comparison of direct ELISA results comparing the apparent binding affinity of the globulomer-specific mAb 7C6 to disulfide stabilized (17C, 34C) Aβ(16-35) oligomer vs. the same oligomer truncated at residue 20 by enzymatic cleavage with thermolysin;
(G) a comparison of direct ELISA results comparing the apparent binding affinity of the globulomer-specific rabbit polyclonal antiserum 5599 to disulfide stabilized (17C, 34C) Aβ(16-35) oligomer vs. the same oligomer truncated at residue 20 by enzymatic cleavage with thermolysin;
(H) a comparison of direct ELISA results comparing the apparent binding affinity of the globulomer-specific mAb 5F7 to Aβ(16-35) oligomer vs. the same oligomer truncated at residue 20 by enzymatic cleavage with thermolysin;
(I) a comparison of direct ELISA results comparing the apparent binding affinity of the globulomer-specific mAb 7C6 to Aβ(16-35) oligomer vs. the same oligomer truncated at residue 20 by enzymatic cleavage with thermolysin;
(J) a comparison of direct ELISA results comparing the apparent binding affinity of the globulomer-specific rabbit polyclonal antiserum 5599 to Aβ(16-35) oligomer vs. the same oligomer truncated at residue 20 by enzymatic cleavage with thermolysin;
(K) a comparison of direct ELISA results comparing the apparent binding affinity of the globulomer-specific mAb 5F7 to (17C(ACM), 34C(ACM)) Aβ(16-35) oligomer vs. the same oligomer truncated at residue 20 by enzymatic cleavage with thermolysin;
(L) a comparison of direct ELISA results comparing the apparent binding affinity of the globulomer-specific mAb 7C6 to (17C(ACM), 34C(ACM)) Aβ(16-35) oligomer vs. the same oligomer truncated at residue 20 by enzymatic cleavage with thermolysin;
(M) a comparison of direct ELISA results comparing the apparent binding affinity of the globulomer-specific rabbit polyclonal antiserum 5599 to (17C(ACM), 34C(ACM)) Aβ(16-35) oligomer vs. the same oligomer truncated at residue 20 by enzymatic cleavage with thermolysin;
(N) a comparison of direct ELISA results comparing the apparent binding affinity of the globulomer-specific mAb 5F7 to disulfide stabilized (17C, 34C) Aβ(16-35) oligomer (cyclised before oligomer formation) vs. the same oligomer truncated at residue 20 by enzymatic cleavage with thermolysin;
(O) a comparison of direct ELISA results comparing the apparent binding affinity of the globulomer-specific mAb 7C6 to disulfide stabilized (17C, 34C) Aβ(16-35) oligomer (cyclised before oligomer formation) vs. the same oligomer truncated at residue 20 by enzymatic cleavage with thermolysin;
(P) a comparison of direct ELISA results comparing the apparent binding affinity of the globulomer-specific rabbit polyclonal antiserum 5599 to disulfide stabilized (17C, 34C) Aβ(16-35) oligomer (cyclised before oligomer formation) vs. the same oligomer truncated at residue 20 by enzymatic cleavage with thermolysin;
(Q) a comparison of direct ELISA results comparing the apparent binding affinity of the globulomer-specific mAb 5F7 to disulfide stabilized (17C, 34C) Aβ(16-35) oligomer (cyclised before oligomer formation) vs. disulfide stabilized (17C, 34C) Aβ(16-42), (17C, 34C) Aβ(16-35) and (17C, 34C) N-Met Aβ(1-42) oligomer (all cyclised after oligomer formation);
(R) a comparison of direct ELISA results comparing the apparent binding affinity of the globulomer-specific mAb 5F7 to thermolysin truncated disulfide stabilized (17C, 34C) Aβ(16-35) oligomer (cyclised before oligomer formation) vs. thermolysin truncated disulfide stabilized (17C, 34C) Aβ(16-42), (17C, 34C) Aβ(16-35) and (17C, 34C) N-Met Aβ(1-42) oligomer (all cyclised after oligomer formation);
(S) a comparison of direct ELISA results comparing the apparent binding affinity of the globulomer-specific mAb 5F7 to disulfide stabilized (17C, 34C) Aβ(16-42) oligomer vs. the same oligomer truncated at residue 20 by enzymatic cleavage with thermolysin;
(T) a comparison of direct ELISA results comparing the apparent binding affinity of the globulomer-specific mAb 7C6 to disulfide stabilized (17C, 34C) Aβ(16-42) oligomer vs. the same oligomer truncated at residue 20 by enzymatic cleavage with thermolysin;
(U) a comparison of direct ELISA results comparing the apparent binding affinity of the globulomer-specific rabbit polyclonal antiserum 5599 to disulfide stabilized (17C, 34C) Aβ(16-42) oligomer vs. the same oligomer truncated at residue 20 by enzymatic cleavage with thermolysin;
(V) a comparison of direct ELISA results comparing the apparent binding affinity of the globulomer-specific mAb 5F7 to disulfide stabilized (17C, 34C) Aβ(12-42) oligomer vs. the same oligomer truncated at residue 20 by enzymatic cleavage with thermolysin;
(W) a comparison of direct ELISA results comparing the apparent binding affinity of the globulomer-specific mAb 7C6 to disulfide stabilized (17C, 34C) Aβ(12-42) oligomer vs. the same oligomer truncated at residue 20 by enzymatic cleavage with thermolysin;
(X) a comparison of direct ELISA results comparing the apparent binding affinity of the globulomer-specific rabbit polyclonal antiserum 5599 to disulfide stabilized (17C, 34C) Aβ(12-42) oligomer vs. the same oligomer truncated at residue 20 by enzymatic cleavage with thermolysin;
(Y) direct ELISA results comparing the apparent binding affinity of the globulomer-specific mAb 5F7 to disulfide stabilized (17C, 34C) (K insertion) Aβ(13-42) oligomer;
(Z) direct ELISA results comparing the apparent binding affinity of the globulomer-specific mAb 7C6 to disulfide stabilized (17C, 34C) (K insertion) Aβ(13-42) oligomer;
(AA) direct ELISA results comparing the apparent binding affinity of the globulomer-specific rabbit polyclonal antiserum 5599 to disulfide stabilized (17C, 34C) (K insertion) Aβ(13-42) oligomer.

FIG. 4 shows mass spectra obtained from Aβ globulomers formed with the (L17C, L34C) N-Met Aβ(1-42) mutant peptide under (A) disulfide bond forming conditions and (B) after reduction with DTT; mass spectra obtained from (17C, 34C) Aβ(16-35) oligomer under (C) disulfide bond forming conditions and (D) after reduction with DTT; mass spectra obtained from (17C, 34C) Aβ(16-42) oligomer under (E) disulfide bond forming conditions and (F) after reduction with DTT; mass spectra obtained from (17C, 42C) Aβ(12-42) oligomer under (G) disulfide bond forming conditions and (H) after reduction with DTT; mass spectra obtained from (17KC, 42C) Aβ(13-42) oligomer under (I) disulfide bond forming conditions and (J) after reduction with DTT; complete isotopic deconvolution is shown.

FIG. 5 shows (A) a sedimentation velocity analysis of heterogeneity of N-Met Aβ(1-42) globulomer (dashed line) and disulfide stabilized (L17C, L34C) N-Met Aβ(1-42) mutant globulomer (solid line) in 5 mM NaPO₄, 35 mM NaCl, pH 7.4; (B) a sedimentation velocity analysis of heterogeneity N-Met Aβ(1-42) globulomer (dashed line) and disulfide stabilized (L17C, L34C) N-Met Aβ(1-42) mutant globulomer truncated at residue 20 by enzymatic cleavage with thermolysin (solid line) in 5 mM NaPO₄, 35 mM NaCl, pH 7.4 supplemented with 0.05% SDS; (C) a sedimentation velocity analysis of heterogeneity of disulfide stabilized (L17C, L34C) N-Met Aβ(1-42) mutant globulomer truncated at residue 20 by enzymatic cleavage with thermolysin (solid line) in 5 mM NaPO₄, 35 mM NaCl, pH 7.4.

FIG. 6 is a table indicating the peptide masses of the (xC, yC) N-Met Aβ(1-42) oligomers before and after thermolysin digestion detected by SELDI-MS.

FIG. 7 is a table indicating peptide mass peaks of iodoacetamide treated thermolysin truncated (xC, yC) N-Met Aβ(1-42) oligomers or thermolysin truncated Aβ(1-42) globulomer detected by SELDI-MS.

FIG. 8 shows dot blot analyses of the reactivity with

1. (14C, 37C) N-Met Aβ(1-42) oligomer;
2. thermolysin truncated (14C, 37C) N-Met Aβ(1-42) oligomer;
3. (15C, 36C) N-Met Aβ(1-42) oligomer;
4. thermolysin truncated (15C, 36C) N-Met Aβ(1-42) oligomer;
5. (16C, 35C) N-Met Aβ(1-42) oligomer;
6. thermolysin truncated (16C, 35C) N-Met Aβ(1-42) oligomer;
7. (17C, 34C) N-Met Aβ(1-42) oligomer;
8. thermolysin truncated (17C, 34C) N-Met Aβ(1-42) oligomer;
9. (18C, 33C) N-Met Aβ(1-42) oligomer;
10. thermolysin truncated (18C, 33C) N-Met Aβ(1-42) oligomer;
11. (19C, 32C) N-Met Aβ(1-42) oligomer;
12. thermolysin truncated (19C, 32C) N-Met Aβ(1-42) oligomer;
13. (20C, 31C) N-Met Aβ(1-42) oligomer;
14. thermolysin truncated (20C, 31C) N-Met Aβ(1-42) oligomer;
15. (21C, 30C) N-Met Aβ(1-42) oligomer;
16. thermolysin truncated (21C, 30C) N-Met Aβ(1-42) oligomer;
17. (22C, 29C) N-Met Aβ(1-42) oligomer;
18. thermolysin truncated (22C, 29C) N-Met Aβ(1-42); oligomer
19. Aβ(1-42) globulomer; and
20. Aβ(1-42) thermolysin truncated globulomer
of A) monoclonal antibody 7C6 and B) rabbit polyclonal antibody 5599.

FIG. 9 is a table indicating peptide mass peaks of iodoacetamide treated thermolysin truncated (17C, 34C) N-Met Aβ(16-35) oligomers detected by SELDI-MS.

FIG. 10 is a table indicating amounts of immunoprecipitated (17C, 34C) Aβ(16-35) oligomer (A) without prior iodoacetamide alkylation and (B) after iodoacetamide alkylation in the presence of DTT.

FIG. 11 shows a schematic diagram indicating the position of cross-links of (17K, 34C) N-Met Aβ(1-42) upon treatment with (A) sulfo-SMCC, (B) sulfo-MBS or (C) sulfo-SIAB at either K16 or K17; and (D) a schematic diagram indicating the position of potential cross links of (17K, 34E) N-Met Aβ(1-42) upon treatment with EDAC/NHS.

FIG. 12 shows (A) the mass spectrum (ESI) of oligomers made with (17K, 34C) N-Met Aβ(1-42) peptide after cross-linking reaction with the heterobifunctional cross-linking reagent sulfo-SMCC; (B) the mass spectrum (MALDI) of globulomers made with (17K, 34C) N-Met Aβ(1-42) peptide after cross-linking reaction with the heterobifunctional cross-linking reagent sulfo-MBS; (C) the mass spectrum (ESI) of globulomers made with (17K, 34C) N-Met Aβ(1-42) peptide after cross-linking reaction with the heterobifunctional cross-linking reagent sulfo-SIAB; and (D) the mass spectrum (MALDI) of globulomers made with (17K, 34E) N-Met Aβ(1-42) peptide after cross-linking reaction with the heterobifunctional cross-linking reagent EDC and NHS. The arrows indicates the expected mass after the desired cross-link forms.

FIG. 13 shows (A) a comparison of direct Elisa response of (17K, 34C) N-Met Aβ(1-42) oligomer without cross-link with disulfide-stabilized (17C, 34C) N-Met Aβ(1-42) oligomer to (A) the globulomer-specific monoclonal antibody 5F7; (B) the globulomer-specific monoclonal antibody 7C6; and (C) the globulomer-specific rabbit polyclonal antiserum 5599.

FIG. 14 shows (A) a comparison of direct Elisa response of (17K, 34C) N-Met Aβ(1-42) oligomer without cross-link with (17K, 34C) N-Met Aβ(1-42) oligomer cross-linked with SMCC before and after thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 15 shows (A) a comparison of direct Elisa response of (17K, 34C) N-Met Aβ(1-42) oligomer without cross-link with (17K, 34C) N-Met Aβ(1-42) oligomer cross-linked with MBS before and after thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 16 shows (A) a comparison of direct Elisa response of (17K, 34C) N-Met Aβ(1-42) oligomer without cross-link with (17K, 34C) N-Met Aβ(1-42) oligomer cross-linked with SIAB before and after thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 17 shows (A) a comparison of direct Elisa response of (17C, 34E) N-Met Aβ(1-42) oligomer without cross-link with disulfide-stabilized (17C, 34C) N-Met Aβ(1-42) oligomer to (A) the globulomer-specific monoclonal antibody 5F7; (B) the globulomer-specific monoclonal antibody 7C6; and (C) the globulomer-specific rabbit polyclonal antiserum 5599.

FIG. 18 shows (A) a comparison of direct Elisa response of (17K, 34C) N-Met Aβ(1-42) oligomer without cross-link with (17K, 34C) N-Met Aβ(1-42) oligomer cross-linked with EDC/NHS before and after thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 19 shows a schematic diagram indicating strategies for (A) forming a methylenedithioether linkage; (B) performing a ring closing metathesis reaction between allylglycines; (C) performing a ring closing metathesis reaction between the modified amino acids X═(S)-Fmoc-α(2′ pentenyl)alanine; and (D) perfoming click chemistry of Lys(N3) and propargylglycine amino acids.

### DETAILED DESCRIPTION OF THE INVENTION

The amyloid β peptide analogues of the present invention comprise an amino acid sequence (peptide) or a peptidomimetic of an amino acid sequence.

An embodiment is an amyloid β peptide analogue comprising an amino acid sequence, wherein the sequence (i) forms a loop, (ii) has at least 66% identity to native human Aβ peptide or a portion thereof, (iii) comprises at least 6 contiguous amino acid residues and (iv) has at least 2 non-contiguous amino acid residues which are covalently linked with each other; or a derivative thereof.

According to a particular embodiment, the amyloid β peptide analogues of the present invention do not comprise any further amino acid or any further peptidomimetic of an amino acid than said amino acid sequence or said peptidomimetic of the amino acid sequence (but the amyloid β peptide analogues of the present invention may comprise further chemical groups or moieties which are attached said amino acid sequence or peptidomimetic). According to one aspect, the amino acid sequence consists of up to 45, 44, 43, 42, 41, 40, 39, 34, 33, 32, 31, 30, 29, 28, 27, 26, 25, 24, 23, 20, 16 amino acids (or a corresponding peptidomimetic). According to another aspect, the amino acid sequence consists of at least 10, 11, 12, 13, 14, 15, 16 amino acids (or a corresponding peptidomimetic).

Unless otherwise indicated, the term “amino acid” as employed herein, alone or as part of another group, includes, without limitation, an amino group and a carboxyl group linked to the same carbon, referred to as “α” carbon —CRR′—, where R and/or R′ can be a natural or an un-natural side chain, including hydrogen. The absolute “S” configuration at the “α” carbon is commonly referred to as the “L” or “natural” configuration. In the case where both the “R” and the “R′” (prime) substituents equal hydrogen, the amino acid is glycine and is not chiral. Amino acids include the genetically encoded L-enantiomeric amino acids (such as alanine (A Ala), arginine (R; Arg), asparagine (N; Asn), aspartic acid (D; Asp). cysteine (C; Cys), glutamine (Q; Gln), glutamic acid (E; Glu), glycine (G; Gly), histidine (H; His), isoleucine (I; Ile), leucine (L; Leu), lysine (K; Lys), phenylalanine (F; Phe), proline (P; Pro), serine (S; Ser), threonine (T; Thr), tryptophan (W; Trp), tyrosine (Y; Tyr), valine (V; Val)), the corresponding D-amino acids, as well as a number of genetically non-encoded amino acids which include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); naphthylalanine (NaI); 4-phenylphenylalanine, 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

For purposes of determining conservative amino acid substitutions, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into nonpolar and aromatic amino acids.

The term “hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), H is (H), Glu (E), Asn (N), Gln (O), Asp (D), Lys (K) and Arg (R).

The term “hydrophobic amino acid” refers to an amino acid exhibiting a hydrophobicity of greater than zero according to the normalized consensus hydrophobicity scale of Eisenberg, 1984, J. Mol. Biol. 179: 1.25-142. Genetically encoded hydrophobic amino acids include Pro (P), Ile (I), Phe (F), Val (V), Leu (L), Trp (W), Met (M), Ala (A), Gly (G) and Tyr (Y).

The term “acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

The term “basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20-membered heteroaryl, substituted 5-20-membered heteroaryl, 6-26-membered alkheteroaryl or substituted 6-26-membered alkheteroaryl. Genetically encoded aromatic amino acids include Phe (F), Tyr (Y) and Trp (W).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. The appropriate categorization of any amino acid will be apparent to those of skill in the art, especially in light of the detailed disclosure provided herein.

Instead of a sequence of amino acids (peptide) the amyloid β peptide analogues of the invention may comprise an analogue of said sequence having properties analogous to those of the template amino acid sequence (peptide). These types of non-peptide sequences are termed “peptide mimetics” or “peptidomimetics” (Fauchere, J. (1986) Adv. Drug Res. 15: 29; Veber and Freidinger (1985) TINS p. 392; and Evans et al. (1987) J. Med. Chem. 30: 1229) and are usually developed with the aid of computerized molecular modeling.

The peptidomimetics are referred to as being “derivable from” a certain amino acid sequence. By this it is meant that the peptidomimetic is designed with reference to a defined amino acid sequence, such that it retains the structural features of the amino acid sequence which are essential for its function. This may be the particular side chains of the amino acid sequence, or hydrogen bonding potential of the structure. Such features may be provided by non-peptide components or one or more of the amino acid residues or the bonds linking said amino acid residues of the amino acid sequence may be modified so as to improve certain functions of the amino acid sequence such as stability or protease resistance, while retaining the structural features of the amino acid sequence which are essential for its function. In other words an amyloid β peptide analogue comprising a peptidomimetic of an amino acid sequence and the amyloid β peptide analogue comprising the amino acid sequence from which the peptidomimetic is derived have the same functional characteristics with respect to their ability to form the loop and, if applicable, to display the further structural and functional properties of the amyloid β peptide analogues as defined herein.

Peptidomimetics are usually structurally similar to the paradigm peptide (i.e., the amino acid sequence comprised by the amyloid β peptide analogues of the invention), but have one or more peptide linkages optionally replaced by a linkage similar to an amide linkage (e.g. an amide isostere such as an N-methyl amide, thioamide, thioester, phosphonate, ketomethylene, hydroxymethylene, fluorovinyl, (E)-vinyl, methyleneamino, methylenethio or alkane linkage). Such linkages may, in particular, be selected from the group consisting of: —CH₂—NH—, —CH₂—S—, —CH₂—CH₂—, —CH═CH— (cis and trans), —COCH₂—, —CH(OH)CH₂—, and —CH₂SO—. These linkages are well-known in the art and further described in the following references: Spatola, A. F. in “Chemistry and Biochemistry of Amino Acids, Peptides, and Proteins,” B. Weinstein, eds., Marcel Dekker, New York, p. 267 (1983); Szatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pent Prot Res (1979) 14: 177-185; Spatola, A. F. et al., Life Sci (1986) 38: 1243-1249; Hann, M. M., J Chem Soc Perkin Trans I (1982) 307-314; Almquist, R. G. et al., J Med Chem (1980) 23: 1392-1398; Jennings-White, C. et al., Tetrahedron Lett (1982) 23: 2533; Szelke, M. et al., EP 45665 (1982) CA: 97: 39405 (1982); Holladay, M. W. et al., Tetrahedron Lett (1983) 24: 4401-4404; and Hruby, V. J., Life Sci (1982) 31: 189-199. A particularly preferred non-peptide linkage is —CH₂NH—. Such peptide mimetics may have significant advantages over peptide embodiments, including, for example: more economical production, greater chemical stability, enhanced pharmacological properties (half-life, absorption, potency, efficacy, etc.), and others.

Peptidomimetics also include “reversed” or “retro” amino acid sequences. Reversed or retro amino acid sequences comprise covalently-bonded amino acid residues wherein the normal carboxyl-to-amino direction of peptide bond formation in the amino acid backbone is reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed amino acid sequences of the invention can be formed by utilizing an appropriate amino acid mimetic moiety to link the two adjacent portions of the sequences utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed amino acid. The configuration of amino acids in the reversed portion of the peptides is preferably (D), and the configuration of the non-reversed portion is preferably (L). Opposite or mixed configurations are acceptable when appropriate to optimize a binding activity.

The amino acid sequence or peptidomimetic comprised by the amyloid β peptide analogues of the invention is characterized by a particular secondary structure comprising a loop (synonym: turn). A loop (or turn) as used herein is meant to define the close approach (usually <7 Å) of at least two Cα atoms.

Suitable loops include α-, β-, γ-, and π-loops. According to a particular embodiment of the invention, the loop is a β-loop. A β-loop as used herein is meant to define a loop which is characterized by hydrogen bond(s) in which the donor and acceptor residues are separated by three residues i→i+/−3H-bonding).

According to a particular embodiment of the invention, the loop is a β-hairpin loop. A β-hairpin loop as used herein is meant to define a loop, in which the direction of the peptide or peptidomimetic backbone reverses and the flanking secondary structure elements interact.

According to a further particular embodiment of the invention, the amyloid β peptide analogues comprise an amino acid sequence which forms an intra-molecular antiparallel β-sheet. An antiparallel β-sheet as used herein is meant to define an assembly of at least two β-strands connected laterally by three or more hydrogen bonds, forming a generally twisted, pleated sheet. A β-strand is a stretch of amino acids comprising typically 3-10 amino acids whose peptide backbones are almost fully extended, or a peptidomimetic thereof.

According to a particular embodiment, the amyloid β peptide analogues of the invention comprise an amino acid sequence in which the β-strands forming the antiparallel β-sheet are connected via the loop, preferably the β-hairpin loop as defined herein.

The amino acid sequence of the amyloid β peptide analogues of the invention has at least 66% identity to the amino acid sequence of native human Aβ peptide or a portion thereof.

The term “native human Aβ peptide” as used herein refers to a naturally-occurring Aβ(X-Y) peptide of human origin, such as Aβ(1-40) or Aβ(1-42) peptide.

The term “naturally-occurring Aβ(X-Y) peptide” here refers to the amino acid sequence from amino acid position X to amino acid position Y of the human amyloid β protein including both X and Y, in particular to the amino acid sequence from amino acid position X to amino acid position Y of the amino acid sequence DAEFRHDSGY EVHHQKLVFF AEDVGSNKGA IIGLMVGGVV IAT (corresponding to amino acid positions 1 to 43; the human query sequence) or any of its naturally occurring variants, in particular those with at least one mutation selected from the group consisting of A2T, H6R, D7N, A21G (“Flemish”), E22G (“Arctic”), E22Q (“Dutch”), E22K (“Italian”), D23N (“Iowa”), A42T and A42V wherein the numbers are relative to the start of the Aβ peptide, including both position X and position Y.

For instance, the term “naturally-occurring Aβ(1-42) peptide” here refers to the amino acid sequence from amino acid position 1 to amino acid position 42 of the human amyloid β protein including both 1 and 42, in particular to the amino acid sequence DAEFRHDSGY EVHHQKLVFF AEDVGSNKGA IIGLMVGGVV IA or any of its naturally occurring variants, in particular those with at least one mutation selected from the group consisting of A21 G (“Flemish”), E22G (“Arctic”), E22Q (“Dutch”), E22K (“Italian”), D23N (“Iowa”), A42T and A42V wherein the numbers are relative to the start of the Aβ peptide, including both 1 and 42.

Thus the amyloid β peptide analogues of the invention comprise an amino acid sequence which corresponds to naturally occurring Aβ peptides, functional fragments and variant sequences thereof that are at least about 66%, 70%, 75%, 80%, 85%, 90%, 95%, 97%, 99% or more identical to the human query sequence described above or a portion thereof.

The term “corresponds to” is used herein to mean that an amino acid sequence is homologous (i.e., is identical, not strictly evolutionarily related) to all or a portion of a reference amino acid sequence.

The following terms are used to describe the sequence relationships between two or more polynucleotide or amino acid sequences: “reference sequence”, “comparison window”, “sequence identity”, and “percentage of sequence identity”. A “reference sequence” or “query sequence” is a defined sequence used as a basis for a sequence comparison; a reference sequence may be a subset of a larger sequence, for example, as a portion of a full-length cDNA, gene sequence or polypeptide sequence, or may comprise a complete cDNA, gene sequence or polypeptide sequence, such as a human Aβ peptide sequence described above. Since two sequences may each (1) comprise a sequence (i.e., a portion of the complete sequence) that is similar between the two sequences, and (2) may further comprise a sequence that is divergent between the two sequences, sequence comparisons between two (or more) polynucleotides are typically performed by comparing sequences of the two sequences over a “comparison window” to identify and compare local regions of sequence similarity. A “comparison window”, as used herein, refers to a conceptual segment of at least 6 contiguous amino acid or 24 nucleotide positions wherein a sequence may be compared to a reference sequence of at least 6, 8, 10, 12, 14, 16, 18, 20, 22, 24, 26, 28, 30, 32, 34, 36, 38, 40, or 42 contiguous amino acids or 18, 24, 30, 36, 42, 48, 54, 60, 66, 72, 78, 84, 90, 96, 102, 108, 114, 120, or 126 nucleotides and wherein the portion of the sequence in the comparison window may comprise additions or deletions (i.e., gaps) of 20 percent or less as compared to the reference sequence (which does not comprise additions or deletions) for optimal alignment of the two sequences. In the context of optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.) or by inspection, and the best alignment (i.e., resulting in the highest percentage of homology over the comparison window) generated by the various methods is selected. The term “sequence identity” means that two sequences are identical (i.e., on a nucleotide-by-nucleotide or amino acid-by-amino acid basis) over the window of comparison. The term “percentage of sequence identity” is calculated by comparing two optimally aligned sequences over the window of comparison, determining the number of positions at which the identical nucleic acid base or amino acid occurs in both sequences to yield the number of matched positions, dividing the number of matched positions by the total number of positions in the window of comparison (i.e., the window size), and multiplying the result by 100 to yield the percentage of sequence identity.

If the identity of the amino acid sequence of the amyloid β peptide analogue to the amino acid sequence of native human Aβ peptide is to be determined, the window of comparison may include the entire amino acid sequence comprised by the amyloid β peptide analogue or a portion thereof. If the amino acid sequence comprised by the amyloid β peptide analogue is shorter than the amino acid sequence of native human Aβ peptide, the window of comparison will include only a portion of the native human Aβ peptide so that the percentage of sequence identity refers to that portion only. If the amino acid sequence comprised by the amyloid β peptide analogue is longer than the amino acid sequence of native human Aβ peptide, the window of comparison will include only a portion of the amino acid sequence comprised by the amyloid β peptide analogue so that the percentage of sequence identity refers to that portion only.

According to a particular embodiment, the invention relates to amyloid β peptide analogues wherein the amino acid sequence of the amyloid β peptide analogues has at least 66% identity to a native human Aβ(X-Y) sequence, X being selected from the group consisting of the numbers 1 . . . 23, e.g. 15, 18, 19, 20, 21, 22, or 23, and Y being selected from the group consisting of the numbers 28 . . . 43, e.g. 28, 29, 30, 31, 34, 37, 40, 42, or 43.

As used herein, the ellipsis A . . . B denotes the set comprising all natural numbers from A to B, including both, e.g. “17 . . . 20” thus denotes the group of the numbers 17, 18, 19 and 20. The hyphen denotes a contiguous sequence of amino acids, i.e., “X-Y” comprises the sequence from amino acid X to amino acid Y, including both. Thus, “A . . . B-C . . . D” comprises all possible combinations between members of these two sets, e.g. “17 . . . 20-40 . . . 42” comprises all of the following: 17-40, 17-41, 17-42, 18-40, 18-41, 18-42, 19-40, 19-41, 19-42, 20-40, 20-41 and 20-42. Unless stated otherwise, all numbers refer to the beginning of the mature peptide, 1 indicating the N-terminal amino acid.

In particular, the amino acid sequence of the amyloid β peptide analogues has at least 66% identity to a sequence selected from the group consisting of SEQ ID NO:1-368:

| | SEQ |
|---|---|
| | ID |
| Sequence | NO |
| DAEFRHDSGYEVHHQKLVFFAEDVGSNKGAIIGLMVGGVVIAT- | 1 |
| -AEFRHDSGYEVHHQKLVFFAEDVGSNKGAIIGLMVGGVVIAT- | 2 |
| --EFRHDSGYEVHHQKLVFFAEDVGSNKGAIIGLMVGGVVIAT- | 3 |
| ---FRHDSGYEVHHQKLVFFAEDVGSNKGAIIGLMVGGVVIAT- | 4 |
| ----RHDSGYEVHHQKLVFFAEDVGSNKGAIIGLMVGGVVIAT- | 5 |
| -----HDSGYEVHHQKLVFFAEDVGSNKGAIIGLMVGGVVIAT- | 6 |
| ------DSGYEVHHQKLVFFAEDVGSNKGAIIGLMVGGVVIAT- | 7 |
| -------SGYEVHHQKLVFFAEDVGSNKGAIIGLMVGGVVIAT- | 8 |
| --------GYEVHHQKLVFFAEDVGSNKGAIIGLMVGGVVIAT- | 9 |
| ---------YEVHHQKLVFFAEDVGSNKGAIIGLMVGGVVIAT- | 10 |
| ----------EVHHQKLVFFAEDVGSNKGAIIGLMVGGVVIAT- | 11 |
| -----------VHHQKLVFFAEDVGSNKGAIIGLMVGGVVIAT- | 12 |
| ------------HHQKLVFFAEDVGSNKGAIIGLMVGGVVIAT- | 13 |
| -------------HQKLVFFAEDVGSNKGAIIGLMVGGVVIAT- | 14 |
| --------------QKLVFFAEDVGSNKGAIIGLMVGGVVIAT- | 15 |
| ---------------KLVFFAEDVGSNKGAIIGLMVGGVVIAT- | 16 |
| ----------------LVFFAEDVGSNKGAIIGLMVGGVVIAT- | 17 |
| -----------------VFFAEDVGSNKGAIIGLMVGGVVIAT- | 18 |
| ------------------FFAEDVGSNKGAIIGLMVGGVVIAT- | 19 |
| -------------------FAEDVGSNKGAIIGLMVGGVVIAT- | 20 |
| --------------------AEDVGSNKGAIIGLMVGGVVIAT- | 21 |
| ---------------------EDVGSNKGAIIGLMVGGVVIAT- | 22 |
| ----------------------DVGSNKGAIIGLMVGGVVIAT- | 23 |
| DAEFRHDSGYEVHHQKLVFFAEDVGSNKGAIIGLMVGGVVIA-- | 24 |
| -AEFRHDSGYEVHHQKLVFFAEDVGSNKGAIIGLMVGGVVIA-- | 25 |
| --EFRHDSGYEVHHQKLVFFAEDVGSNKGAIIGLMVGGVVIA-- | 26 |
| ---FRHDSGYEVHHQKLVFFAEDVGSNKGAIIGLMVGGVVIA-- | 27 |
| ----RHDSGYEVHHQKLVFFAEDVGSNKGAIIGLMVGGVVIA-- | 28 |
| -----HDSGYEVHHQKLVFFAEDVGSNKGAIIGLMVGGVVIA-- | 29 |
| ------DSGYEVHHQKLVFFAEDVGSNKGAIIGLMVGGVVIA-- | 30 |
| -------SGYEVHHQKLVFFAEDVGSNKGAIIGLMVGGVVIA-- | 31 |
| --------GYEVHHQKLVFFAEDVGSNKGAIIGLMVGGVVIA-- | 32 |
| ---------YEVHHQKLVFFAEDVGSNKGAIIGLMVGGVVIA-- | 33 |
| ----------EVHHQKLVFFAEDVGSNKGAIIGLMVGGVVIA-- | 34 |
| -----------VHHQKLVFFAEDVGSNKGAIIGLMVGGVVIA-- | 35 |
| ------------HHQKLVFFAEDVGSNKGAIIGLMVGGVVIA-- | 36 |
| -------------HQKLVFFAEDVGSNKGAIIGLMVGGVVIA-- | 37 |
| --------------QKLVFFAEDVGSNKGAIIGLMVGGVVIA-- | 38 |
| ---------------KLVFFAEDVGSNKGAIIGLMVGGVVIA-- | 39 |
| ----------------LVFFAEDVGSNKGAIIGLMVGGVVIA-- | 40 |
| -----------------VFFAEDVGSNKGAIIGLMVGGVVIA-- | 41 |
| ------------------FFAEDVGSNKGAIIGLMVGGVVIA-- | 42 |
| -------------------FAEDVGSNKGAIIGLMVGGVVIA-- | 43 |
| --------------------AEDVGSNKGAIIGLMVGGVVIA-- | 44 |
| ---------------------EDVGSNKGAIIGLMVGGVVIA-- | 45 |
| ----------------------DVGSNKGAIIGLMVGGVVIA-- | 46 |
| DAEFRHDSGYEVHHQKLVFFAEDVGSNKGAIIGLMVGGVVI--- | 47 |
| -AEFRHDSGYEVHHQKLVFFAEDVGSNKGAIIGLMVGGVVI--- | 48 |
| --EFRHDSGYEVHHQKLVFFAEDVGSNKGAIIGLMVGGVVI--- | 49 |
| ---FRHDSGYEVHHQKLVFFAEDVGSNKGAIIGLMVGGVVI--- | 50 |
| ----RHDSGYEVHHQKLVFFAEDVGSNKGAIIGLMVGGVVI--- | 51 |
| -----HDSGYEVHHQKLVFFAEDVGSNKGAIIGLMVGGVVI--- | 52 |
| ------DSGYEVHHQKLVFFAEDVGSNKGAIIGLMVGGVVI--- | 53 |
| -------SGYEVHHQKLVFFAEDVGSNKGAIIGLMVGGVVI--- | 54 |
| --------GYEVHHQKLVFFAEDVGSNKGAIIGLMVGGVVI--- | 55 |
| ---------YEVHHQKLVFFAEDVGSNKGAIIGLMVGGVVI--- | 56 |
| ----------EVHHQKLVFFAEDVGSNKGAIIGLMVGGVVI--- | 57 |
| -----------VHHQKLVFFAEDVGSNKGAIIGLMVGGVVI--- | 58 |
| ------------HHQKLVFFAEDVGSNKGAIIGLMVGGVVI--- | 59 |
| -------------HQKLVFFAEDVGSNKGAIIGLMVGGVVI--- | 60 |
| --------------QKLVFFAEDVGSNKGAIIGLMVGGVVI--- | 61 |
| ---------------KLVFFAEDVGSNKGAIIGLMVGGVVI--- | 62 |
| ----------------LVFFAEDVGSNKGAIIGLMVGGVVI--- | 63 |
| -----------------VFFAEDVGSNKGAIIGLMVGGVVI--- | 64 |
| ------------------FFAEDVGSNKGAIIGLMVGGVVI--- | 65 |
| -------------------FAEDVGSNKGAIIGLMVGGVVI--- | 66 |
| --------------------AEDVGSNKGAIIGLMVGGVVI--- | 67 |
| ---------------------EDVGSNKGAIIGLMVGGVVI--- | 68 |
| ----------------------DVGSNKGAIIGLMVGGVVI--- | 69 |
| DAEFRHDSGYEVHHQKLVFFAEDVGSNKGAIIGLMVGGVV---- | 70 |
| -AEFRHDSGYEVHHQKLVFFAEDVGSNKGAIIGLMVGGVV---- | 71 |
| --EFRHDSGYEVHHQKLVFFAEDVGSNKGAIIGLMVGGVV---- | 72 |
| ---FRHDSGYEVHHQKLVFFAEDVGSNKGAIIGLMVGGVV---- | 73 |
| ----RHDSGYEVHHQKLVFFAEDVGSNKGAIIGLMVGGVV---- | 74 |
| -----HDSGYEVHHQKLVFFAEDVGSNKGAIIGLMVGGVV---- | 75 |
| ------DSGYEVHHQKLVFFAEDVGSNKGAIIGLMVGGVV---- | 76 |
| -------SGYEVHHQKLVFFAEDVGSNKGAIIGLMVGGVV---- | 77 |
| --------GYEVHHQKLVFFAEDVGSNKGAIIGLMVGGVV---- | 78 |
| ---------YEVHHQKLVFFAEDVGSNKGAIIGLMVGGVV---- | 79 |
| ----------EVHHQKLVFFAEDVGSNKGAIIGLMVGGVV---- | 80 |
| -----------VHHQKLVFFAEDVGSNKGAIIGLMVGGVV---- | 81 |
| ------------HHQKLVFFAEDVGSNKGAIIGLMVGGVV---- | 82 |
| -------------HQKLVFFAEDVGSNKGAIIGLMVGGVV---- | 83 |
| --------------QKLVFFAEDVGSNKGAIIGLMVGGVV---- | 84 |
| ---------------KLVFFAEDVGSNKGAIIGLMVGGVV---- | 85 |
| ----------------LVFFAEDVGSNKGAIIGLMVGGVV---- | 86 |
| -----------------VFFAEDVGSNKGAIIGLMVGGVV---- | 87 |
| ------------------FFAEDVGSNKGAIIGLMVGGVV---- | 88 |
| -------------------FAEDVGSNKGAIIGLMVGGVV---- | 89 |
| --------------------AEDVGSNKGAIIGLMVGGVV---- | 90 |
| ---------------------EDVGSNKGAIIGLMVGGVV---- | 91 |
| ----------------------DVGSNKGAIIGLMVGGVV---- | 92 |
| DAEFRHDSGYEVHHQKLVFFAEDVGSNKGAIIGLMVGGV----- | 93 |
| -AEFRHDSGYEVHHQKLVFFAEDVGSNKGAIIGLMVGGV----- | 94 |
| --EFRHDSGYEVHHQKLVFFAEDVGSNKGAIIGLMVGGV----- | 95 |
| ---FRHDSGYEVHHQKLVFFAEDVGSNKGAIIGLMVGGV----- | 96 |
| ----RHDSGYEVHHQKLVFFAEDVGSNKGAIIGLMVGGV----- | 97 |
| -----HDSGYEVHHQKLVFFAEDVGSNKGAIIGLMVGGV----- | 98 |
| ------DSGYEVHHQKLVFFAEDVGSNKGAIIGLMVGGV----- | 99 |
| -------SGYEVHHQKLVFFAEDVGSNKGAIIGLMVGGV----- | 100 |
| --------GYEVHHQKLVFFAEDVGSNKGAIIGLMVGGV----- | 101 |
| ---------YEVHHQKLVFFAEDVGSNKGAIIGLMVGGV----- | 102 |
| ----------EVHHQKLVFFAEDVGSNKGAIIGLMVGGV----- | 103 |
| -----------VHHQKLVFFAEDVGSNKGAIIGLMVGGV----- | 104 |
| ------------HHQKLVFFAEDVGSNKGAIIGLMVGGV----- | 105 |
| -------------HQKLVFFAEDVGSNKGAIIGLMVGGV----- | 106 |
| --------------QKLVFFAEDVGSNKGAIIGLMVGGV----- | 107 |
| ---------------KLVFFAEDVGSNKGAIIGLMVGGV----- | 108 |
| ----------------LVFFAEDVGSNKGAIIGLMVGGV----- | 109 |
| -----------------VFFAEDVGSNKGAIIGLMVGGV----- | 110 |
| ------------------FFAEDVGSNKGAIIGLMVGGV----- | 111 |
| -------------------FAEDVGSNKGAIIGLMVGGV----- | 112 |
| --------------------AEDVGSNKGAIIGLMVGGV----- | 113 |
| ---------------------EDVGSNKGAIIGLMVGGV----- | 114 |
| ----------------------DVGSNKGAIIGLMVGGV----- | 115 |
| DAEFRHDSGYEVHHQKLVFFAEDVGSNKGAIIGLMVGG------ | 116 |
| -AEFRHDSGYEVHHQKLVFFAEDVGSNKGAIIGLMVGG------ | 117 |
| --EFRHDSGYEVHHQKLVFFAEDVGSNKGAIIGLMVGG------ | 118 |
| ---FRHDSGYEVHHQKLVFFAEDVGSNKGAIIGLMVGG------ | 119 |
| ----RHDSGYEVHHQKLVFFAEDVGSNKGAIIGLMVGG------ | 120 |
| -----HDSGYEVHHQKLVFFAEDVGSNKGAIIGLMVGG------ | 121 |
| ------DSGYEVHHQKLVFFAEDVGSNKGAIIGLMVGG------ | 122 |
| -------SGYEVHHQKLVFFAEDVGSNKGAIIGLMVGG------ | 123 |
| --------GYEVHHQKLVFFAEDVGSNKGAIIGLMVGG------ | 124 |
| ---------YEVHHQKLVFFAEDVGSNKGAIIGLMVGG------ | 125 |
| ----------EVHHQKLVFFAEDVGSNKGAIIGLMVGG------ | 126 |
| -----------VHHQKLVFFAEDVGSNKGAIIGLMVGG------ | 127 |
| ------------HHQKLVFFAEDVGSNKGAIIGLMVGG------ | 128 |
| -------------HQKLVFFAEDVGSNKGAIIGLMVGG------ | 129 |
| --------------QKLVFFAEDVGSNKGAIIGLMVGG------ | 130 |
| ---------------KLVFFAEDVGSNKGAIIGLMVGG------ | 131 |
| ----------------LVFFAEDVGSNKGAIIGLMVGG------ | 132 |
| -----------------VFFAEDVGSNKGAIIGLMVGG------ | 133 |
| ------------------FFAEDVGSNKGAIIGLMVGG------ | 134 |
| -------------------FAEDVGSNKGAIIGLMVGG------ | 135 |
| --------------------AEDVGSNKGAIIGLMVGG------ | 136 |
| ---------------------EDVGSNKGAIIGLMVGG------ | 137 |
| ----------------------DVGSNKGAIIGLMVGG------ | 138 |
| DAEFRHDSGYEVHHQKLVFFAEDVGSNKGAIIGLMVG------- | 139 |
| -AEFRHDSGYEVHHQKLVFFAEDVGSNKGAIIGLMVG------- | 140 |
| --EFRHDSGYEVHHQKLVFFAEDVGSNKGAIIGLMVG------- | 141 |
| ---FRHDSGYEVHHQKLVFFAEDVGSNKGAIIGLMVG------- | 142 |
| ----RHDSGYEVHHQKLVFFAEDVGSNKGAIIGLMVG------- | 143 |
| -----HDSGYEVHHQKLVFFAEDVGSNKGAIIGLMVG------- | 144 |
| ------DSGYEVHHQKLVFFAEDVGSNKGAIIGLMVG------- | 145 |
| -------SGYEVHHQKLVFFAEDVGSNKGAIIGLMVG------- | 146 |
| --------GYEVHHQKLVFFAEDVGSNKGAIIGLMVG------- | 147 |
| ---------YEVHHQKLVFFAEDVGSNKGAIIGLMVG------- | 148 |
| ----------EVHHQKLVFFAEDVGSNKGAIIGLMVG------- | 149 |
| -----------VHHQKLVFFAEDVGSNKGAIIGLMVG------- | 150 |
| ------------HHQKLVFFAEDVGSNKGAIIGLMVG------- | 151 |
| -------------HQKLVFFAEDVGSNKGAIIGLMVG------- | 152 |
| --------------QKLVFFAEDVGSNKGAIIGLMVG------- | 153 |
| ---------------KLVFFAEDVGSNKGAIIGLMVG------- | 154 |
| ----------------LVFFAEDVGSNKGAIIGLMVG------- | 155 |
| -----------------VFFAEDVGSNKGAIIGLMVG------- | 156 |
| ------------------FFAEDVGSNKGAIIGLMVG------- | 157 |
| -------------------FAEDVGSNKGAIIGLMVG------- | 158 |
| --------------------AEDVGSNKGAIIGLMVG------- | 159 |
| ---------------------EDVGSNKGAIIGLMVG------- | 160 |
| ----------------------DVGSNKGAIIGLMVG------- | 161 |
| DAEFRHDSGYEVHHQKLVFFAEDVGSNKGAIIGLMV-------- | 162 |
| -AEFRHDSGYEVHHQKLVFFAEDVGSNKGAIIGLMV-------- | 163 |
| --EFRHDSGYEVHHQKLVFFAEDVGSNKGAIIGLMV-------- | 164 |
| ---FRHDSGYEVHHQKLVFFAEDVGSNKGAIIGLMV-------- | 165 |
| ----RHDSGYEVHHQKLVFFAEDVGSNKGAIIGLMV-------- | 166 |
| -----HDSGYEVHHQKLVFFAEDVGSNKGAIIGLMV-------- | 167 |
| ------DSGYEVHHQKLVFFAEDVGSNKGAIIGLMV-------- | 168 |
| -------SGYEVHHQKLVFFAEDVGSNKGAIIGLMV-------- | 169 |
| --------GYEVHHQKLVFFAEDVGSNKGAIIGLMV-------- | 170 |
| ---------YEVHHQKLVFFAEDVGSNKGAIIGLMV-------- | 171 |
| ----------EVHHQKLVFFAEDVGSNKGAIIGLMV-------- | 172 |
| -----------VHHQKLVFFAEDVGSNKGAIIGLMV-------- | 173 |
| ------------HHQKLVFFAEDVGSNKGAIIGLMV-------- | 174 |
| -------------HQKLVFFAEDVGSNKGAIIGLMV-------- | 175 |
| --------------QKLVFFAEDVGSNKGAIIGLMV-------- | 176 |
| ---------------KLVFFAEDVGSNKGAIIGLMV-------- | 177 |
| ----------------LVFFAEDVGSNKGAIIGLMV-------- | 178 |
| -----------------VFFAEDVGSNKGAIIGLMV-------- | 179 |
| ------------------FFAEDVGSNKGAIIGLMV-------- | 180 |
| -------------------FAEDVGSNKGAIIGLMV-------- | 181 |
| --------------------AEDVGSNKGAIIGLMV-------- | 182 |
| ---------------------EDVGSNKGAIIGLMV-------- | 183 |
| ----------------------DVGSNKGAIIGLMV-------- | 184 |
| DAEFRHDSGYEVHHQKLVFFAEDVGSNKGAIIGLM--------- | 185 |
| -AEFRHDSGYEVHHQKLVFFAEDVGSNKGAIIGLM--------- | 186 |
| --EFRHDSGYEVHHQKLVFFAEDVGSNKGAIIGLM--------- | 187 |
| ---FRHDSGYEVHHQKLVFFAEDVGSNKGAIIGLM--------- | 188 |
| ----RHDSGYEVHHQKLVFFAEDVGSNKGAIIGLM--------- | 189 |
| -----HDSGYEVHHQKLVFFAEDVGSNKGAIIGLM--------- | 190 |
| ------DSGYEVHHQKLVFFAEDVGSNKGAIIGLM--------- | 191 |
| -------SGYEVHHQKLVFFAEDVGSNKGAIIGLM--------- | 192 |
| --------GYEVHHQKLVFFAEDVGSNKGAIIGLM--------- | 193 |
| ---------YEVHHQKLVFFAEDVGSNKGAIIGLM--------- | 194 |
| ----------EVHHQKLVFFAEDVGSNKGAIIGLM--------- | 195 |
| -----------VHHQKLVFFAEDVGSNKGAIIGLM--------- | 196 |
| ------------HHQKLVFFAEDVGSNKGAIIGLM--------- | 197 |
| -------------HQKLVFFAEDVGSNKGAIIGLM--------- | 198 |
| --------------QKLVFFAEDVGSNKGAIIGLM--------- | 199 |
| ---------------KLVFFAEDVGSNKGAIIGLM--------- | 200 |
| ----------------LVFFAEDVGSNKGAIIGLM--------- | 201 |
| -----------------VFFAEDVGSNKGAIIGLM--------- | 202 |
| ------------------FFAEDVGSNKGAIIGLM--------- | 203 |
| -------------------FAEDVGSNKGAIIGLM--------- | 204 |
| --------------------AEDVGSNKGAIIGLM--------- | 205 |
| ---------------------EDVGSNKGAIIGLM--------- | 206 |
| ----------------------DVGSNKGAIIGLM--------- | 207 |
| DAEFRHDSGYEVHHQKLVFFAEDVGSNKGAIIGL---------- | 208 |
| -AEFRHDSGYEVHHQKLVFFAEDVGSNKGAIIGL---------- | 209 |
| --EFRHDSGYEVHHQKLVFFAEDVGSNKGAIIGL---------- | 210 |
| ---FRHDSGYEVHHQKLVFFAEDVGSNKGAIIGL---------- | 211 |
| ----RHDSGYEVHHQKLVFFAEDVGSNKGAIIGL---------- | 212 |
| -----HDSGYEVHHQKLVFFAEDVGSNKGAIIGL---------- | 213 |
| ------DSGYEVHHQKLVFFAEDVGSNKGAIIGL---------- | 214 |
| -------SGYEVHHQKLVFFAEDVGSNKGAIIGL---------- | 215 |
| --------GYEVHHQKLVFFAEDVGSNKGAIIGL---------- | 216 |
| ---------YEVHHQKLVFFAEDVGSNKGAIIGL---------- | 217 |
| ----------EVHHQKLVFFAEDVGSNKGAIIGL---------- | 218 |
| -----------VHHQKLVFFAEDVGSNKGAIIGL---------- | 219 |
| ------------HHQKLVFFAEDVGSNKGAIIGL---------- | 220 |
| -------------HQKLVFFAEDVGSNKGAIIGL---------- | 221 |
| --------------QKLVFFAEDVGSNKGAIIGL---------- | 222 |
| ---------------KLVFFAEDVGSNKGAIIGL---------- | 223 |
| ----------------LVFFAEDVGSNKGAIIGL---------- | 224 |
| -----------------VFFAEDVGSNKGAIIGL---------- | 225 |
| ------------------FFAEDVGSNKGAIIGL---------- | 226 |
| -------------------FAEDVGSNKGAIIGL---------- | 227 |
| --------------------AEDVGSNKGAIIGL---------- | 228 |
| ---------------------EDVGSNKGAIIGL---------- | 229 |
| ----------------------DVGSNKGAIIGL---------- | 230 |
| DAEFRHDSGYEVHHQKLVFFAEDVGSNKGAIIG----------- | 231 |
| -AEFRHDSGYEVHHQKLVFFAEDVGSNKGAIIG----------- | 232 |
| --EFRHDSGYEVHHQKLVFFAEDVGSNKGAIIG----------- | 233 |
| ---FRHDSGYEVHHQKLVFFAEDVGSNKGAIIG----------- | 234 |
| ----RHDSGYEVHHQKLVFFAEDVGSNKGAIIG----------- | 235 |
| -----HDSGYEVHHQKLVFFAEDVGSNKGAIIG----------- | 236 |
| ------DSGYEVHHQKLVFFAEDVGSNKGAIIG----------- | 237 |
| -------SGYEVHHQKLVFFAEDVGSNKGAIIG----------- | 238 |
| --------GYEVHHQKLVFFAEDVGSNKGAIIG----------- | 239 |
| ---------YEVHHQKLVFFAEDVGSNKGAIIG----------- | 240 |
| ----------EVHHQKLVFFAEDVGSNKGAIIG----------- | 241 |
| -----------VHHQKLVFFAEDVGSNKGAIIG----------- | 242 |
| ------------HHQKLVFFAEDVGSNKGAIIG----------- | 243 |
| -------------HQKLVFFAEDVGSNKGAIIG----------- | 244 |
| --------------QKLVFFAEDVGSNKGAIIG----------- | 245 |
| ---------------KLVFFAEDVGSNKGAIIG----------- | 246 |
| ----------------LVFFAEDVGSNKGAIIG----------- | 247 |
| -----------------VFFAEDVGSNKGAIIG----------- | 248 |
| ------------------FFAEDVGSNKGAIIG----------- | 249 |
| -------------------FAEDVGSNKGAIIG----------- | 250 |
| --------------------AEDVGSNKGAIIG----------- | 251 |
| ---------------------EDVGSNKGAIIG----------- | 252 |
| ----------------------DVGSNKGAIIG----------- | 253 |
| DAEFRHDSGYEVHHQKLVFFAEDVGSNKGAII------------ | 254 |
| -AEFRHDSGYEVHHQKLVFFAEDVGSNKGAII------------ | 255 |
| --EFRHDSGYEVHHQKLVFFAEDVGSNKGAII------------ | 256 |
| ---FRHDSGYEVHHQKLVFFAEDVGSNKGAII------------ | 257 |
| ----RHDSGYEVHHQKLVFFAEDVGSNKGAII------------ | 258 |
| -----HDSGYEVHHQKLVFFAEDVGSNKGAII------------ | 259 |
| ------DSGYEVHHQKLVFFAEDVGSNKGAII------------ | 260 |
| -------SGYEVHHQKLVFFAEDVGSNKGAII------------ | 261 |
| --------GYEVHHQKLVFFAEDVGSNKGAII------------ | 262 |
| ---------YEVHHQKLVFFAEDVGSNKGAII------------ | 263 |
| ----------EVHHQKLVFFAEDVGSNKGAII------------ | 264 |
| -----------VHHQKLVFFAEDVGSNKGAII------------ | 265 |
| ------------HHQKLVFFAEDVGSNKGAII------------ | 266 |
| -------------HQKLVFFAEDVGSNKGAII------------ | 267 |
| --------------QKLVFFAEDVGSNKGAII------------ | 268 |
| ---------------KLVFFAEDVGSNKGAII------------ | 269 |
| ----------------LVFFAEDVGSNKGAII------------ | 270 |
| -----------------VFFAEDVGSNKGAII------------ | 271 |
| ------------------FFAEDVGSNKGAII------------ | 272 |
| -------------------FAEDVGSNKGAII------------ | 273 |
| --------------------AEDVGSNKGAII------------ | 274 |
| ---------------------EDVGSNKGAII------------ | 275 |
| ----------------------DVGSNKGAII------------ | 276 |
| DAEFRHDSGYEVHHQKLVFFAEDVGSNKGAI------------- | 277 |
| -AEFRHDSGYEVHHQKLVFFAEDVGSNKGAI------------- | 278 |
| --EFRHDSGYEVHHQKLVFFAEDVGSNKGAI------------- | 279 |
| ---FRHDSGYEVHHQKLVFFAEDVGSNKGAI------------- | 280 |
| ----RHDSGYEVHHQKLVFFAEDVGSNKGAI------------- | 281 |
| -----HDSGYEVHHQKLVFFAEDVGSNKGAI------------- | 282 |
| ------DSGYEVHHQKLVFFAEDVGSNKGAI------------- | 283 |
| -------SGYEVHHQKLVFFAEDVGSNKGAI------------- | 284 |
| --------GYEVHHQKLVFFAEDVGSNKGAI------------- | 285 |
| ---------YEVHHQKLVFFAEDVGSNKGAI------------- | 286 |
| ----------EVHHQKLVFFAEDVGSNKGAI------------- | 287 |
| -----------VHHQKLVFFAEDVGSNKGAI------------- | 288 |
| ------------HHQKLVFFAEDVGSNKGAI------------- | 289 |
| -------------HQKLVFFAEDVGSNKGAI------------- | 290 |
| --------------QKLVFFAEDVGSNKGAI------------- | 291 |
| ---------------KLVFFAEDVGSNKGAI------------- | 292 |
| ----------------LVFFAEDVGSNKGAI------------- | 293 |
| -----------------VFFAEDVGSNKGAI------------- | 294 |
| ------------------FFAEDVGSNKGAI------------- | 295 |
| -------------------FAEDVGSNKGAI------------- | 296 |
| --------------------AEDVGSNKGAI------------- | 297 |
| ---------------------EDVGSNKGAI------------- | 298 |
| ----------------------DVGSNKGAI------------- | 299 |
| DAEFRHDSGYEVHHQKLVFFAEDVGSNKGA-------------- | 300 |
| -AEFRHDSGYEVHHQKLVFFAEDVGSNKGA-------------- | 301 |
| --EFRHDSGYEVHHQKLVFFAEDVGSNKGA-------------- | 302 |
| ---FRHDSGYEVHHQKLVFFAEDVGSNKGA-------------- | 303 |
| ----RHDSGYEVHHQKLVFFAEDVGSNKGA-------------- | 304 |
| -----HDSGYEVHHQKLVFFAEDVGSNKGA-------------- | 305 |
| ------DSGYEVHHQKLVFFAEDVGSNKGA-------------- | 306 |
| -------SGYEVHHQKLVFFAEDVGSNKGA-------------- | 307 |
| --------GYEVHHQKLVFFAEDVGSNKGA-------------- | 308 |
| ---------YEVHHQKLVFFAEDVGSNKGA-------------- | 309 |
| ----------EVHHQKLVFFAEDVGSNKGA-------------- | 310 |
| -----------VHHQKLVFFAEDVGSNKGA-------------- | 311 |
| ------------HHQKLVFFAEDVGSNKGA-------------- | 312 |
| -------------HQKLVFFAEDVGSNKGA-------------- | 313 |
| --------------QKLVFFAEDVGSNKGA-------------- | 314 |
| ---------------KLVFFAEDVGSNKGA-------------- | 315 |
| ----------------LVFFAEDVGSNKGA-------------- | 316 |
| -----------------VFFAEDVGSNKGA-------------- | 317 |
| ------------------FFAEDVGSNKGA-------------- | 318 |
| -------------------FAEDVGSNKGA-------------- | 319 |
| --------------------AEDVGSNKGA-------------- | 320 |
| ---------------------EDVGSNKGA-------------- | 321 |
| ----------------------DVGSNKGA-------------- | 322 |
| DAEFRHDSGYEVHHQKLVFFAEDVGSNKG--------------- | 323 |
| -AEFRHDSGYEVHHQKLVFFAEDVGSNKG--------------- | 324 |
| --EFRHDSGYEVHHQKLVFFAEDVGSNKG--------------- | 325 |
| ---FRHDSGYEVHHQKLVFFAEDVGSNKG--------------- | 326 |
| ----RHDSGYEVHHQKLVFFAEDVGSNKG--------------- | 327 |
| -----HDSGYEVHHQKLVFFAEDVGSNKG--------------- | 328 |
| ------DSGYEVHHQKLVFFAEDVGSNKG--------------- | 329 |
| -------SGYEVHHQKLVFFAEDVGSNKG--------------- | 330 |
| --------GYEVHHQKLVFFAEDVGSNKG--------------- | 331 |
| ---------YEVHHQKLVFFAEDVGSNKG--------------- | 332 |
| ----------EVHHQKLVFFAEDVGSNKG--------------- | 333 |
| -----------VHHQKLVFFAEDVGSNKG--------------- | 334 |
| ------------HHQKLVFFAEDVGSNKG--------------- | 335 |
| -------------HQKLVFFAEDVGSNKG--------------- | 336 |
| --------------QKLVFFAEDVGSNKG--------------- | 337 |
| ---------------KLVFFAEDVGSNKG--------------- | 338 |
| ----------------LVFFAEDVGSNKG--------------- | 339 |
| -----------------VFFAEDVGSNKG--------------- | 340 |
| ------------------FFAEDVGSNKG--------------- | 341 |
| -------------------FAEDVGSNKG--------------- | 342 |
| --------------------AEDVGSNKG--------------- | 343 |
| ---------------------EDVGSNKG--------------- | 344 |
| ----------------------DVGSNKG--------------- | 345 |
| DAEFRHDSGYEVHHQKLVFFAEDVGSNK---------------- | 346 |
| -AEFRHDSGYEVHHQKLVFFAEDVGSNK---------------- | 347 |
| --EFRHDSGYEVHHQKLVFFAEDVGSNK---------------- | 348 |
| ---FRHDSGYEVHHQKLVFFAEDVGSNK---------------- | 349 |
| ----RHDSGYEVHHQKLVFFAEDVGSNK---------------- | 350 |
| -----HDSGYEVHHQKLVFFAEDVGSNK---------------- | 351 |
| ------DSGYEVHHQKLVFFAEDVGSNK---------------- | 352 |
| -------SGYEVHHQKLVFFAEDVGSNK---------------- | 353 |
| --------GYEVHHQKLVFFAEDVGSNK---------------- | 354 |
| ---------YEVHHQKLVFFAEDVGSNK---------------- | 355 |
| ----------EVHHQKLVFFAEDVGSNK---------------- | 356 |
| -----------VHHQKLVFFAEDVGSNK---------------- | 357 |
| ------------HHQKLVFFAEDVGSNK---------------- | 358 |
| -------------HQKLVFFAEDVGSNK---------------- | 359 |
| --------------QKLVFFAEDVGSNK---------------- | 360 |
| ---------------KLVFFAEDVGSNK---------------- | 361 |
| ----------------LVFFAEDVGSNK---------------- | 362 |
| -----------------VFFAEDVGSNK---------------- | 363 |
| ------------------FFAEDVGSNK---------------- | 364 |
| -------------------FAEDVGSNK---------------- | 365 |
| --------------------AEDVGSNK---------------- | 366 |
| ---------------------EDVGSNK---------------- | 367 |
| ----------------------DVGSNK---------------- | 368 |

According to a particular embodiment, the amino acid sequence of the amyloid β peptide analogue of the invention corresponds to a sequence selected from the group consisting of SEQ ID NO:1-368 wherein at least one amino acid of the selected sequence is substituted by another amino acid.

It will be recognized that in certain embodiments of the invention, the amino acid substitutions are conservative, i.e., the replacing amino acid residue has physical and chemical properties that are similar to the amino acid residue being replaced. Especially preferred conservative amino acids substitution groups are: valine-leucine-isoleucine, phenylalanine-tyrosine, lysine-arginine, alanine-valine, and asparagine-glutamine.

Moreover, systematic substitution of one or more amino acids of the above sequences with a D-amino acid of the same type (e.g., D-lysine in place of L-lysine) may be used to enhance stability.

It will further be recognized that in certain embodiments of the invention, the amino acid substitutions are non-conservative, i.e., the replacing amino acid residue has physical and chemical properties that differ from those of the amino acid residue being replaced. This embodiment in particular relates to replacing amino acid residues which are capable of forming linkages. That is, such an amino acid substitution enables the formation of a covalent linkage between the replacing amino acid and another amino acid which, in turn, may or may not be a replacing amino acid, too. This allows the introduction of covalent linkages between amino acids at defined positions within the amino acid sequence.

According to a further particular embodiment, the amino acid sequence of the amyloid β peptide analogues of the invention comprises a sequence selected from the group consisting of SEQ ID NO:1-368, at least two, e.g. two, amino acid residues of said sequence being modified so as to form the required intra-sequence covalent linkage. For instance, each of two amino acids my be replaced by a cysteine. This will allow the formation of a variety of linkages. Particular positions for such amino acid replacements and a variety of appropriate linkages are described herein. Further, one amino acid may be replaced by a cysteine and another amino acid may be replaced by a lysine. This will allow the formation of a variety of linkages. Particular positions for such amino acid replacements and a variety of appropriate linkages are described herein. Further, one amino acid may be replaced by a glutamic acid or aspartic acid and another amino acid may be replaced by a lysine. This will allow the formation of a variety of linkages. Particular positions for such amino acid replacements and a variety of appropriate linkages are described herein.

The amino acid sequence of the amyloid β peptide analogues of the invention comprises at least 6, preferably at least 8, 10, 12, 14, 16 or 18, contiguous amino acid residues. Further, in a typical embodiment, the amino acid sequence of the amyloid β peptide analogues of the invention will comprise less that 45, 43, 41, 39, 37, or 36 contiguous amino acid residues. According to a preferred embodiment, the contiguous amino acid residues comprise the sequence VGSN, DVGSN, or VGSNK. According to another preferred embodiment, the contiguous amino acid residues comprise the sequence AED. According to still another preferred embodiment, the contiguous amino acid residues comprise both the sequence AED and one of the sequences VGSN, DVGSN, or VGSNK, in particular the sequence AEDVGSN or AEDVGSNK.

According to a further embodiment of the invention, the amino acid sequence of the amyloid β peptide analogues of the invention comprises the sequence X₁₉X₂₀X₂₁X₂₂X₂₃-VGSN-X₂₈X₂₉X₃₀X₃₁X₃₂, wherein each of X₁₉, X₂₀, X₂₁, X₂₂ X₂₃, X₂₈, X₂₉, X₃₀, X₃₁, X₃₂ independently represents an amino acid, in particular as defined herein. Each of said amino acids may be covalently linked with another amino acid, with the other amino acid being selected among X₁₉, X₂₀ X₂₁, X₂₂ X₂₃, X₂₈, X₂₉, X₃₀, X₃₁, X₃₂, or representing a different amino acid. Preferably, the amino acid sequences X₁₉X₂₀X₂₁ and X₃₀X₃₁X₃₂ are in anti-parallel orientation.

Particular amyloid β peptide analogues of the invention include those wherein

X₁₉ is an amino acid residue selected from the group consisting of phenylalanine, tyrosine, valine, leucine, isoleucine, and methionine, with phenylalanine being preferred;
X₂₀ is an amino acid residue selected from the group consisting of phenylalanine, tyrosine, valine, leucine, isoleucine, and methionine, with phenylalanine being preferred;
X₂₁ is an amino acid residue selected from the group consisting of alanine, valine, glycine, and serine, with alanine being preferred;
X₂₂ is an amino acid residue selected from the group consisting of glutamic acid and aspartic acid, with glutamic acid being preferred;
X₂₃ is an amino acid residue selected from the group consisting of glutamic acid and aspartic acid, with aspartic acid being preferred;
X₂₈ is an amino acid residue selected from the group consisting of lysine and arginine, with lysine being preferred;
X₂₉ is an amino acid residue selected from the group consisting of glycine, alanine, and serine, with glycine being preferred;
X₃₀ is an amino acid residue selected from the group consisting of alanine, valine, glycine, and serine, with alanine being preferred;
X₃₁ is an amino acid residue selected from the group consisting of isoleucine, leucine, valine, phenylalanine, and methionine, with isoleucine being preferred;
X₃₂ is an amino acid residue selected from the group consisting of isoleucine, leucine, valine, phenylalanine, and methionine, with isoleucine being preferred; or any combination thereof.

Further particular amyloid β peptide analogues of the invention include those wherein the amino acid sequence of the amyloid β peptide analogue comprises the sequence F₁₉X₂₀A₂₁-Q-A₃₀I₃₁I₃₂, with X₂₀ representing an amino acid, in particular as defined herein, and Q being an amino acid sequence comprising the sequence VGSN.

The amino acid sequence Q usually consists of 5, 6, 7, or 8 amino acid residues. According to a particular embodiment, it forms the loop. i.e., some or all amino acids of Q are arranged so as to form the loop.

Amyloid β peptide analogues comprising the sequence

F₁₉X₂₀A₂₁X₂₂D₂₃V₂₄G₂₅S₂₆N₂₇K₂₈X₂₉A₃₀I₃₁I₃₂, wherein each of X₂₀, X₂₂, X₂₉ independently represents an amino acid residue, in particular as defined herein, represent a preferred embodiment of the invention. In these amyloid β peptide analogues, the amino acid sequences F₁₉X₂₀A₂₁ and A₃₀I₃₁I₃₂ are preferably in anti-parallel orientation. More particularly, it is preferred if the interproton distance for at least one atom pair selected from the group consisting of F₁₉(NH)—I₃₂(NH), F₁₉(NH)—I₃₂(HB), F₁₉(NH)—I₃₂(CG2), A₂₁(NH)-A₃₀(NH), A₂₁(NH)-A₃₀(CB), A₂₁(NH)—I₃₁(CD1), A₂₁(NH)—I₃₁(CG2), I₃₂(NH)—F₁₉(CD1), I₃₂(NH)—F₁₉(CD2), I₃₂(HN)—F₁₉(CB), and A₃₀(NH)-A₂₁(CB) is 1.8 to 6.5 Angstroms. It is also preferred if the atom pairs F₁₉(CO)—I₃₂(N), I₃₂(CO)—F₁₉(N), A₂₁(CO)-A₃₀(N), and A₃₀(CO)-A₂₁(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150.

According to a further particular embodiment, the amino acid sequence of the amyloid β peptide analogue is a sequence selected from the group consisting of SEQ ID NO: 369-698, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid residue which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₁ is alanine, valine, glycine, serine, or an amino acid residue which is covalently linked to another amino acid residue of the sequence;
X₂₂ is glutamic acid, aspartic acid, or an amino acid residue which is covalently linked to another amino acid residue of the sequence;
X₂₉ is glycine, alanine, and serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
   at least one amino acid residue selected from the group consisting of X₁₂, X₁₃, X₁₄, X₁₅, X₁₆, X₁₇, X₁₈, X₁₉, X₂₀, X₂₁ and X₂₂ and at least one amino acid residue selected from the group consisting of X₂₉, X₃₀, X₃₁, X₃₂, X₃₃, X₃₄, X₃₅, X₃₆, X₃₇, X₃₈ and X₃₉ being covalently linked with each other.

| Sequence | |
|---|---|
| DAEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 369 |
| AEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 370 |
| EFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 371 |
| FRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 372 |
| RHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 373 |
| HDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 374 |
| DSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 375 |
| SGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 376 |
| GYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 377 |
| YEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 378 |
| EX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 379 |
| X₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIAT | 380 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈VVIAT | 381 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇GVVIAT | 382 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GGVVIAT | 383 |
| X₁₆X₁₇X₁₈X₁₉X₂₀X₂₁X₂₂DVGSNKX₂₉X₃₀X₃₁X₃₂X₃₃X₃₄X₃₅VGGVVIAT | 384 |
| X₁₇X₁₈X₁₉X₂₀X₂₁X₂₂DVGSNKX₂₉X₃₀X₃₁X₃₂X₃₃X₃₄MVGGVVIAT | 385 |
| X₁₈X₁₉X₂₀X₂₁X₂₂DVGSNKX₂₉X₃₀X₃₁X₃₂X₃₃LMVGGVVIAT | 386 |
| X₁₉X₂₀X₂₁X₂₂DVGSNKX₂₉X₃₀X₃₁X₃₂GLMVGGVVIAT | 387 |
| X₂₀X₂₁X₂₂DVGSNKX₂₉X₃₀X₃₁IGLMVGGVVIAT | 388 |
| X₂₁X₂₂DVGSNKX₂₉X₃₀IIGLMVGGVVIAT | 389 |
| X₂₂DVGSNKX₂₉AIIGLMVGGVVIAT | 390 |
| DAEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 391 |
| AEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 392 |
| EFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 393 |
| FRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 394 |
| RHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 395 |
| HDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 396 |
| DSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 397 |
| SGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 398 |
| GYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 399 |
| YEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 400 |
| EX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 401 |
| X₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VIA | 402 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈VVIA | 403 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇GVVIA | 404 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GGVVIA | 405 |
| X₁₆X₁₇X₁₈X₁₉X₂₀X₂₁X₂₂DVGSNKX₂₉X₃₀X₃₁X₃₂X₃₃X₃₄X₃₅VGGVVIA | 406 |
| X₁₇X₁₈X₁₉X₂₀X₂₁X₂₂DVGSNKX₂₉X₃₀X₃₁X₃₂X₃₃X₃₄MVGGVVIA | 407 |
| X₁₈X₁₉X₂₀X₂₁X₂₂DVGSNKX₂₉X₃₀X₃₁X₃₂X₃₃LMVGGVVIA | 408 |
| X₁₉X₂₀X₂₁X₂₂DVGSNKX₂₉X₃₀X₃₁X₃₂GLMVGGVVIA | 409 |
| X₂₀X₂₁X₂₂DVGSNKX₂₉X₃₀X₃₁IGLMVGGVVIA | 410 |
| X₂₁X₂₂DVGSNKX₂₉X₃₀IIGLMVGGVVIA | 411 |
| X₂₂DVGSNKX₂₉AIIGLMVGGVVIA | 412 |
| DAEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 413 |
| AEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 414 |
| EFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 415 |
| FRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 416 |
| RHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 417 |
| HDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 418 |
| DSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 419 |
| SGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 420 |
| GYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 421 |
| YEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 422 |
| EX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 423 |
| X₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉VI | 424 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈VVI | 425 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇GVVI | 426 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GGVVI | 427 |
| X₁₆X₁₇X₁₈X₁₉X₂₀X₂₁X₂₂DVGSNKX₂₉X₃₀X₃₁X₃₂X₃₃X₃₄X₃₅VGGVVI | 428 |
| X₁₇X₁₈X₁₉X₂₀X₂₁X₂₂DVGSNKX₂₉X₃₀X₃₁X₃₂X₃₃X₃₄MVGGVVI | 429 |
| X₁₈X₁₉X₂₀X₂₁X₂₂DVGSNKX₂₉X₃₀X₃₁X₃₂X₃₃LMVGGVVI | 430 |
| X₁₉X₂₀X₂₁X₂₂DVGSNKX₂₉X₃₀X₃₁X₃₂GLMVGGVVI | 431 |
| X₂₀X₂₁X₂₂DVGSNKX₂₉X₃₀X₃₁IGLMVGGVVI | 432 |
| X₂₁X₂₂DVGSNKX₂₉X₃₀IIGLMVGGVVI | 433 |
| X₂₂DVGSNKX₂₉AIIGLMVGGVVI | 434 |
| DAEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 435 |
| AEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 436 |
| EFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 437 |
| FRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 438 |
| RHDSGYX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 439 |
| HDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 440 |
| DSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 441 |
| SGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 442 |
| GYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 443 |
| YEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 444 |
| EX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 445 |
| X₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉V | 446 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈VV | 447 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇GVV | 448 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GGVV | 449 |
| X₁₆X₁₇X₁₈X₁₉X₂₀X₂₁X₂₂DVGSNKX₂₉X₃₀X₃₁X₃₂X₃₃X₃₄X₃₅VGGVV | 450 |
| X₁₇X₁₈X₁₉X₂₀X₂₁X₂₂DVGSNKX₂₉X₃₀X₃₁X₃₂X₃₃X₃₄MVGGVV | 451 |
| X₁₈X₁₉X₂₀X₂₁X₂₂DVGSNKX₂₉X₃₀X₃₁X₃₂X₃₃LMVGGVV | 452 |
| X₁₉X₂₀X₂₁X₂₂DVGSNKX₂₉X₃₀X₃₁X₃₂GLMVGGVV | 453 |
| X₂₀X₂₁X₂₂DVGSNKX₂₉X₃₀X₃₁IGLMVGGVV | 454 |
| ₂₁X₂₂DVGSNKX₂₉X₃₀IIGLMVGGVV | 455 |
| X₂₂DVGSNKX₂₉AIIGLMVGGVV | 456 |
| DAEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 457 |
| AEFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 458 |
| EFRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 459 |
| FRHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 460 |
| RHDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 461 |
| HDSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 462 |
| DSGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 463 |
| SGYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 464 |
| GYEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 465 |
| YEX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 466 |
| EX₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 467 |
| X₁₂X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈X₃₉ | 468 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈V | 469 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇GV | 470 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GGV | 471 |
| X₁₆X₁₇X₁₈X₁₉X₂₀X₂₁X₂₂DVGSNKX₂₉X₃₀X₃₁X₃₂X₃₃X₃₄X₃₅VGGV | 472 |
| X₁₇X₁₈X₁₉X₂₀X₂₁X₂₂DVGSNKX₂₉X₃₀X₃₁X₃₂X₃₃X₃₄MVGGV | 473 |
| X₁₈X₁₉X₂₀X₂₁X₂₂DVGSNKX₂₉X₃₀X₃₁X₃₂X₃₃LMVGGV | 474 |
| X₁₉X₂₀X₂₁X₂₂DVGSNKX₂₉X₃₀X₃₁X₃₂GLMVGGV | 475 |
| X₂₀X₂₁X₂₂DVGSNKX₂₉X₃₀X₃₁IGLMVGGV | 476 |
| X₂₁X₂₂DVGSNKX₂₉X₃₀IIGLMVGGV | 477 |
| X₂₂DVGSNKX₂₉AIIGLMVGGV | 478 |
| DAEFRHDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 479 |
| AEFRHDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 480 |
| EFRHDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 481 |
| FRHDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 482 |
| RHDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 483 |
| HDSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 484 |
| DSGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 485 |
| SGYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 486 |
| GYEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 487 |
| YEVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 488 |
| EVX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 489 |
| VX₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 490 |
| X₁₃X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇X₃₈ | 491 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇G | 492 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆GG | 493 |
| X₁₆X₁₇X₁₈X₁₉X₂₀X₂₁X₂₂DVGSNKX₂₉X₃₀X₃₁X₃₂X₃₃X₃₄X₃₅VGG | 494 |
| X₁₇X₁₈X₁₉X₂₀X₂₁X₂₂DVGSNKX₂₉X₃₀X₃₁X₃₂X₃₃X₃₄MVGG | 495 |
| X₁₈X₁₉X₂₀X₂₁X₂₂DVGSNKX₂₉X₃₀X₃₁X₃₂X₃₃LMVGG | 496 |
| X₁₉X₂₀X₂₁X₂₂DVGSNKX₂₉X₃₀X₃₁X₃₂GLMVGG | 497 |
| X₂₀X₂₁X₂₂DVGSNKX₂₉X₃₀X₃₁IGLMVGG | 498 |
| X₂₁X₂₂DVGSNKX₂₉X₃₀IIGLMVGG | 499 |
| X₂₂DVGSNKX₂₉AIIGLMVGG | 500 |

| | |
|---|---|
| DAEFRHDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 501 |

| | |
|---|---|
| AEFRHDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 502 |
| EFRHDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 503 |
| FRHDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 504 |
| RHDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 505 |
| HDSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 506 |
| DSGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 507 |
| SGYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 508 |
| GYEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 509 |
| YEVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 510 |
| EVHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 511 |
| VHX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 512 |
| HX₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 513 |
| X₁₄X₁₅X₁₆X₁₇X₁₈X₁₉X₂₀X₂₁X₂₂DVGSNKX₂₉X₃₀X₃₁X₃₂X₃₃X₃₄X₃₅X₃₆X₃₇ | 514 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆G | 515 |
| X₁₆X₁₇X₁₈X₁₉X₂₀X₂₁X₂₂DVGSNKX₂₉X₃₀X₃₁X₃₂X₃₃X₃₄X₃₅VG | 516 |
| X₁₇X₁₈X₁₉X₂₀X₂₁X₂₂DVGSNKX₂₉X₃₀X₃₁X₃₂X₃₃X₃₄MVG | 517 |
| X₁₈X₁₉X₂₀X₂₁X₂₂DVGSNKX₂₉X₃₀X₃₁X₃₂X₃₃LMVG | 518 |
| X₁₉X₂₀X₂₁X₂₂DVGSNKX₂₉X₃₀X₃₁X₃₂GLMVG | 519 |
| X₂₀X₂₁X₂₂DVGSNKX₂₉X₃₀X₃₁IGLMVG | 520 |
| X₂₁X₂₂DVGSNKX₂₉X₃₀IGLMVGIIGLMVG | 521 |
| X₂₂DVGSNKX₂₉IGLMVGAIIGLMVG | 522 |
| DAEFRHDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 523 |
| AEFRHDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 524 |
| EFRHDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 525 |
| FRHDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 526 |
| RHDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 527 |
| HDSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 528 |
| DSGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 529 |
| SGYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 530 |
| GYEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 531 |
| YEVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 532 |
| EVHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 533 |
| VHHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 534 |
| VHX₁₅X₁₆X₁₇X₁₈X₁₉X₂₀X₂₁X₂₂DVGSNKX₂₉X₃₀X₃₁X₃₂X₃₃X₃₄X₃₅X₃₆ | 535 |
| HX₁₅X₁₆X₁₇X₁₈X₁₉X₂₀X₂₁X₂₂DVGSNKX₂₉X₃₀X₃₁X₃₂X₃₃X₃₄X₃₅X₃₆ | 536 |
| X₁₅X₁₆X₁₇X₁₈X₁₉X₂₀X₂₁X₂₂DVGSNKX₂₉X₃₀X₃₁X₃₂X₃₃X₃₄X₃₅X₃₆ | 537 |
| X₁₆X₁₇X₁₈X₁₉X₂₀X₂₁X₂₂DVGSNKX₂₉X₃₀X₃₁X₃₂X₃₃X₃₄X₃₅V | 538 |
| X₁₇X₁₈X₁₉X₂₀X₂₁X₂₂DVGSNKX₂₉X₃₀X₃₁X₃₂X₃₃X₃₄₆MV | 539 |
| X₁₈X₁₉X₂₀X₂₁X₂₂DVGSNKX₂₉X₃₀X₃₁X₃₂X₃₃LMV | 540 |
| X₁₉X₂₀X₂₁X₂₂DVGSNKX₂₉X₃₀X₃₁X₃₂GLMV | 541 |
| X₂₀X₂₁X₂₂DVGSNKX₂₉X₃₀X₃₁IGLMV | 542 |
| X₂₁X₂₂DVGSNKX₂₉X₃₀IIGLMV | 543 |
| X₂₂DVGSNKX₂₉AIIGLMV | 544 |
| DAEFRHDSGYEVHHQX₁₆X₁₇X₁₈X₁₉X₂₀X₂₁X₂₂DVGSNKX₂₉X₃₀X₃₁X₃₂X₃₃X₃₄X₃₅ | 545 |
| AEFRHDSGYEVHHQX₁₆X₁₇X₁₈X₁₉X₂₀X₂₁X₂₂DVGSNKX₂₉X₃₀X₃₁X₃₂X₃₃X₃₄X₃₅ | 546 |
| EFRHDSGYEVHHQX₁₆X₁₇X₁₈X₁₉X₂₀X₂₁X₂₂DVGSNKX₂₉X₃₀X₃₁X₃₂X₃₃X₃₄X₃₅ | 547 |
| FRHDSGYEVHHQX₁₆X₁₇X₁₈X₁₉X₂₀X₂₁X₂₂DVGSNKX₂₉X₃₀X₃₁X₃₂X₃₃X₃₄X₃₅ | 548 |
| RHDSGYEVHHQX₁₆X₁₇X₁₈X₁₉X₂₀X₂₁X₂₂DVGSNKX₂₉X₃₀X₃₁X₃₂X₃₃X₃₄X₃₅ | 549 |
| HDSGYEVHHQX₁₆X₁₇X₁₈X₁₉X₂₀X₂₁X₂₂DVGSNKX₂₉X₃₀X₃₁X₃₂X₃₃X₃₄X₃₅ | 550 |
| DSGYEVHHQX₁₆X₁₇X₁₈X₁₉X₂₀X₂₁X₂₂DVGSNKX₂₉X₃₀X₃₁X₃₂X₃₃X₃₄X₃₅ | 551 |
| SGYEVHHQX₁₆X₁₇X₁₈X₁₉X₂₀X₂₁X₂₂DVGSNKX₂₉X₃₀X₃₁X₃₂X₃₃X₃₄X₃₅ | 552 |
| GYEVHHQX₁₆X₁₇X₁₈X₁₉X₂₀X₂₁X₂₂DVGSNKX₂₉X₃₀X₃₁X₃₂X₃₃X₃₄X₃₅ | 553 |
| YEVHHQX₁₆X₁₇X₁₈X₁₉X₂₀X₂₁X₂₂DVGSNKX₂₉X₃₀X₃₁X₃₂X₃₃X₃₄X₃₅ | 554 |
| EVHHQX₁₆X₁₇X₁₈X₁₉X₂₀X₂₁X₂₂DVGSNKX₂₉X₃₀X₃₁X₃₂X₃₃X₃₄X₃₅ | 555 |
| VHHQX₁₆X₁₇X₁₈X₁₉X₂₀X₂₁X₂₂DVGSNKX₂₉X₃₀X₃₁X₃₂X₃₃X₃₄X₃₅ | 556 |
| HHQX₁₆X₁₇X₁₈X₁₉X₂₀X₂₁X₂₂DVGSNKX₂₉X₃₀X₃₁X₃₂X₃₃X₃₄X₃₅ | 557 |
| HQX₁₆X₁₇X₁₈X₁₉X₂₀X₂₁X₂₂DVGSNKX₂₉X₃₀X₃₁X₃₂X₃₃X₃₄X₃₅ | 558 |
| QX₁₆X₁₇X₁₈X₁₉X₂₀X₂₁X₂₂DVGSNKX₂₉X₃₀X₃₁X₃₂X₃₃X₃₄X₃₅ | 559 |
| X₁₆X₁₇X₁₈X₁₉X₂₀X₂₁X₂₂DVGSNKX₂₉X₃₀X₃₁X₃₂X₃₃X₃₄X₃₅ | 560 |
| X₁₇X₁₈X₁₉X₂₀X₂₁X₂₂DVGSNKX₂₉X₃₀X₃₁X₃₂X₃₃X₃₄M | 561 |
| X₁₈X₁₉X₂₀X₂₁X₂₂DVGSNKX₂₉X₃₀X₃₁X₃₂X₃₃LM | 562 |
| X₁₉X₂₀X₂₁X₂₂DVGSNKX₂₉X₃₀X₃₁X₃₂GLM | 563 |
| X₂₀X₂₁X₂₂DVGSNKX₂₉X₃₀X₃₁IGLM | 564 |
| X₂₁X₂₂DVGSNKX₂₉X₃₀IIGLM | 565 |
| X₂₂DVGSNKX₂₉AIIGLM | 566 |
| DAEFRHDSGYEVHHQKX₁₇X₁₈X₁₉X₂₀X₂₁X₂₂DVGSNKX₂₉X₃₀X₃₁X₃₂X₃₃X₃₄ | 567 |
| AEFRHDSGYEVHHQKX₁₇X₁₈X₁₉X₂₀X₂₁X₂₂DVGSNKX₂₉X₃₀X₃₁X₃₂X₃₃X₃₄ | 568 |
| EFRHDSGYEVHHQKX₁₇X₁₈X₁₉X₂₀X₂₁X₂₂DVGSNKX₂₉X₃₀X₃₁X₃₂X₃₃X₃₄ | 569 |
| FRHDSGYEVHHQKX₁₇X₁₈X₁₉X₂₀X₂₁X₂₂DVGSNKX₂₉X₃₀X₃₁X₃₂X₃₃X₃₄ | 570 |
| RHDSGYEVHHQKX₁₇X₁₈X₁₉X₂₀X₂₁X₂₂DVGSNKX₂₉X₃₀X₃₁X₃₂X₃₃X₃₄ | 571 |
| HDSGYEVHHQKX₁₇X₁₈X₁₉X₂₀X₂₁X₂₂DVGSNKX₂₉X₃₀X₃₁X₃₂X₃₃X₃₄ | 572 |
| DSGYEVHHQKX₁₇X₁₈X₁₉X₂₀X₂₁X₂₂DVGSNKX₂₉X₃₀X₃₁X₃₂X₃₃X₃₄ | 573 |
| SGYEVHHQKX₁₇X₁₈X₁₉X₂₀X₂₁X₂₂DVGSNKX₂₉X₃₀X₃₁X₃₂X₃₃X₃₄ | 574 |
| GYEVHHQKX₁₇X₁₈X₁₉X₂₀X₂₁X₂₂DVGSNKX₂₉X₃₀X₃₁X₃₂X₃₃X₃₄ | 575 |
| YEVHHQKX₁₇X₁₈X₁₉X₂₀X₂₁X₂₂DVGSNKX₂₉X₃₀X₃₁X₃₂X₃₃X₃₄ | 576 |
| EVHHQKX₁₇X₁₈X₁₉X₂₀X₂₁X₂₂DVGSNKX₂₉X₃₀X₃₁X₃₂X₃₃X₃₄ | 577 |
| VHHQKX₁₇X₁₈X₁₉X₂₀X₂₁X₂₂DVGSNKX₂₉X₃₀X₃₁X₃₂X₃₃X₃₄ | 578 |
| HHQKX₁₇X₁₈X₁₉X₂₀X₂₁X₂₂DVGSNKX₂₉X₃₀X₃₁X₃₂X₃₃X₃₄ | 579 |
| HQKX₁₇X₁₈X₁₉X₂₀X₂₁X₂₂DVGSNKX₂₉X₃₀X₃₁X₃₂X₃₃X₃₄ | 580 |
| QKX₁₇X₁₈X₁₉X₂₀X₂₁X₂₂DVGSNKX₂₉X₃₀X₃₁X₃₂X₃₃X₃₄ | 581 |
| KX₁₇X₁₈X₁₉X₂₀X₂₁X₂₂DVGSNKX₂₉X₃₀X₃₁X₃₂X₃₃X₃₄ | 582 |
| X₁₇X₁₈X₁₉X₂₀X₂₁X₂₂DVGSNKX₂₉X₃₀X₃₁X₃₂X₃₃X₃₄ | 583 |
| X₁₈X₁₉X₂₀X₂₁X₂₂DVGSNKX₂₉X₃₀X₃₁X₃₂X₃₃L | 584 |
| X₁₉X₂₀X₂₁X₂₂DVGSNKX₂₉X₃₀X₃₁X₃₂GL | 585 |
| X₂₀X₂₁X₂₂DVGSNKX₂₉X₃₀X₃₁IGL | 586 |
| X₂₁X₂₂DVGSNKX₂₉X₃₀IIGL | 587 |
| X₂₂DVGSNKX₂₉AIIGL | 588 |
| DAEFRHDSGYEVHHQKLX₁₈X₁₉X₂₀X₂₁X₂₂DVGSNKX₂₉X₃₀X₃₁X₃₂X₃₃ | 589 |
| AEFRHDSGYEVHHQKLX₁₈X₁₉X₂₀X₂₁X₂₂DVGSNKX₂₉X₃₀X₃₁X₃₂X₃₃ | 590 |
| EFRHDSGYEVHHQKLX₁₈X₁₉X₂₀X₂₁X₂₂DVGSNKX₂₉X₃₀X₃₁X₃₂X₃₃ | 591 |
| FRHDSGYEVHHQKLX₁₈X₁₉X₂₀X₂₁X₂₂DVGSNKX₂₉X₃₀X₃₁X₃₂X₃₃ | 592 |
| RHDSGYEVHHQKLX₁₈X₁₉X₂₀X₂₁X₂₂DVGSNKX₂₉X₃₀X₃₁X₃₂X₃₃ | 593 |
| HDSGYEVHHQKLX₁₈X₁₉X₂₀X₂₁X₂₂DVGSNKX₂₉X₃₀X₃₁X₃₂X₃₃ | 594 |
| DSGYEVHHQKLX₁₈X₁₉X₂₀X₂₁X₂₂DVGSNKX₂₉X₃₀X₃₁X₃₂X₃₃ | 595 |
| SGYEVHHQKLX₁₈X₁₉X₂₀X₂₁X₂₂DVGSNKX₂₉X₃₀X₃₁X₃₂X₃₃ | 596 |
| GYEVHHQKLX₁₈X₁₉X₂₀X₂₁X₂₂DVGSNKX₂₉X₃₀X₃₁X₃₂X₃₃ | 597 |
| YEVHHQKLX₁₈X₁₉X₂₀X₂₁X₂₂DVGSNKX₂₉X₃₀X₃₁X₃₂X₃₃ | 598 |
| EVHHQKLX₁₈X₁₉X₂₀X₂₁X₂₂DVGSNKX₂₉X₃₀X₃₁X₃₂X₃₃ | 599 |
| VHHQKLX₁₈X₁₉X₂₀X₂₁X₂₂DVGSNKX₂₉X₃₀X₃₁X₃₂X₃₃ | 600 |
| HHQKLX₁₈X₁₉X₂₀X₂₁X₂₂DVGSNKX₂₉X₃₀X₃₁X₃₂X₃₃ | 601 |
| HQKLX₁₈X₁₉X₂₀X₂₁X₂₂DVGSNKX₂₉X₃₀X₃₁X₃₂X₃₃ | 602 |
| QKLX₁₈X₁₉X₂₀X₂₁X₂₂DVGSNKX₂₉X₃₀X₃₁X₃₂X₃₃ | 603 |
| KLX₁₈X₁₉X₂₀X₂₁X₂₂DVGSNKX₂₉X₃₀X₃₁X₃₂X₃₃ | 604 |
| LX₁₈X₁₉X₂₀X₂₁X₂₂DVGSNKX₂₉X₃₀X₃₁X₃₂X₃₃ | 605 |
| X₁₈X₁₉X₂₀X₂₁X₂₂DVGSNKX₂₉X₃₀X₃₁X₃₂X₃₃ | 606 |
| X₁₉X₂₀X₂₁X₂₂DVGSNKX₂₉X₃₀X₃₁X₃₂G | 607 |
| X₂₀X₂₁X₂₂DVGSNKX₂₉X₃₀X₃₁IG | 608 |
| X₂₁X₂₂DVGSNKX₂₉X₃₀IIG | 609 |
| X₂₂DVGSNKX₂₉AIIG | 610 |
| DAEFRHDSGYEVHHQKLVX₁₉X₂₀X₂₁X₂₂DVGSNKX₂₉X₃₀X₃₁X₃₂ | 611 |
| AEFRHDSGYEVHHQKLVX₁₉X₂₀X₂₁X₂₂DVGSNKX₂₉X₃₀X₃₁X₃₂ | 612 |
| EFRHDSGYEVHHQKLVX₁₉X₂₀X₂₁X₂₂DVGSNKX₂₉X₃₀X₃₁X₃₂ | 613 |
| FRHDSGYEVHHQKLVX₁₉X₂₀X₂₁X₂₂DVGSNKX₂₉X₃₀X₃₁X₃₂ | 614 |
| RHDSGYEVHHQKLVX₁₉X₂₀X₂₁X₂₂DVGSNKX₂₉X₃₀X₃₁X₃₂ | 615 |
| HDSGYEVHHQKLVX₁₉X₂₀X₂₁X₂₂DVGSNKX₂₉X₃₀X₃₁X₃₂ | 616 |
| DSGYEVHHQKLVX₁₉X₂₀X₂₁X₂₂DVGSNKX₂₉X₃₀X₃₁X₃₂ | 617 |
| SGYEVHHQKLVX₁₉X₂₀X₂₁X₂₂DVGSNKX₂₉X₃₀X₃₁X₃₂ | 618 |
| GYEVHHQKLVX₁₉X₂₀X₂₁X₂₂DVGSNKX₂₉X₃₀X₃₁X₃₂ | 619 |
| YEVHHQKLVX₁₉X₂₀X₂₁X₂₂DVGSNKX₂₉X₃₀X₃₁X₃₂ | 620 |
| EVHHQKLVX₁₉X₂₀X₂₁X₂₂DVGSNKX₂₉X₃₀X₃₁X₃₂ | 621 |
| VHHQKLVX₁₉X₂₀X₂₁X₂₂DVGSNKX₂₉X₃₀X₃₁X₃₂ | 622 |
| HHQKLVX₁₉X₂₀X₂₁X₂₂DVGSNKX₂₉X₃₀X₃₁X₃₂ | 623 |
| HQKLVX₁₉X₂₀X₂₁X₂₂DVGSNKX₂₉X₃₀X₃₁X₃₂ | 624 |
| QKLVX₁₉X₂₀X₂₁X₂₂DVGSNKX₂₉X₃₀X₃₁X₃₂ | 625 |
| KLVX₁₉X₂₀X₂₁X₂₂DVGSNKX₂₉X₃₀X₃₁X₃₂ | 626 |
| LVX₁₉X₂₀X₂₁X₂₂DVGSNKX₂₉X₃₀X₃₁X₃₂ | 627 |
| VX₁₉X₂₀X₂₁X₂₂DVGSNKX₂₉X₃₀X₃₁X₃₂ | 628 |
| X₁₉X₂₀X₂₁X₂₂DVGSNKX₂₉X₃₀X₃₁X₃₂ | 629 |
| X₂₀X₂₁X₂₂DVGSNKX₂₉X₃₀X₃₁I | 630 |
| X₂₁X₂₂DVGSNKX₂₉X₃₀II | 631 |
| X₂₂DVGSNKX₂₉AII | 632 |
| DAEFRHDSGYEVHHQKLVFX₂₀X₂₁X₂₂DVGSNKX₂₉X₃₀X₃₁ | 633 |
| AEFRHDSGYEVHHQKLVFX₂₀X₂₁X₂₂DVGSNKX₂₉X₃₀X₃₁ | 634 |
| EFRHDSGYEVHHQKLVFX₂₀X₂₁X₂₂DVGSNKX₂₉X₃₀X₃₁ | 635 |
| FRHDSGYEVHHQKLVFX₂₀X₂₁X₂₂DVGSNKX₂₉X₃₀X₃₁ | 636 |
| RHDSGYEVHHQKLVFX₂₀X₂₁X₂₂DVGSNKX₂₉X₃₀X₃₁ | 637 |
| HDSGYEVHHQKLVFX₂₀X₂₁X₂₂DVGSNKX₂₉X₃₀X₃₁ | 638 |
| DSGYEVHHQKLVFX₂₀X₂₁X₂₂DVGSNKX₂₉X₃₀X₃₁ | 639 |
| SGYEVHHQKLVFX₂₀X₂₁X₂₂DVGSNKX₂₉X₃₀X₃₁ | 640 |
| GYEVHHQKLVFX₂₀X₂₁X₂₂DVGSNKX₂₉X₃₀X₃₁ | 641 |
| YEVHHQKLVFX₂₀X₂₁X₂₂DVGSNKX₂₉X₃₀X₃₁ | 642 |
| EVHHQKLVFX₂₀X₂₁X₂₂DVGSNKX₂₉X₃₀X₃₁ | 643 |
| VHHQKLVFX₂₀X₂₁X₂₂DVGSNKX₂₉X₃₀X₃₁ | 644 |
| HHQKLVFX₂₀X₂₁X₂₂DVGSNKX₂₉X₃₀X₃₁ | 645 |
| HQKLVFX₂₀X₂₁X₂₂DVGSNKX₂₉X₃₀X₃₁ | 646 |
| QKLVFX₂₀X₂₁X₂₂DVGSNKX₂₉X₃₀X₃₁ | 647 |
| KLVFX₂₀X₂₁X₂₂DVGSNKX₂₉X₃₀X₃₁ | 648 |
| LVFX₂₀X₂₁X₂₂DVGSNKX₂₉X₃₀X₃₁ | 649 |
| VFX₂₀X₂₁X₂₂DVGSNKX₂₉X₃₀X₃₁ | 650 |
| FX₂₀X₂₁X₂₂DVGSNKX₂₉X₃₀X₃₁ | 651 |
| X₂₀X₂₁X₂₂DVGSNKX₂₉X₃₀X₃₁ | 652 |
| X₂₁X₂₂DVGSNKX₂₉X₃₀I | 653 |
| X₂₂DVGSNKX₂₉AI | 654 |
| DAEFRHDSGYEVHHQKLVFFX₂₁X₂₂DVGSNKX₂₉X₃₀ | 655 |
| AEFRHDSGYEVHHQKLVFFX₂₁X₂₂DVGSNKX₂₉X₃₀ | 656 |
| EFRHDSGYEVHHQKLVFFX₂₁X₂₂DVGSNKX₂₉X₃₀ | 657 |
| FRHDSGYEVHHQKLVFFX₂₁X₂₂DVGSNKX₂₉X₃₀ | 658 |
| RHDSGYEVHHQKLVFFX₂₁X₂₂DVGSNKX₂₉X₃₀ | 659 |
| HDSGYEVHHQKLVFFX₂₁X₂₂DVGSNKX₂₉X₃₀ | 660 |
| DSGYEVHHQKLVFFX₂₁X₂₂DVGSNKX₂₉X₃₀ | 661 |
| SGYEVHHQKLVFFX₂₁X₂₂DVGSNKX₂₉X₃₀ | 662 |
| GYEVHHQKLVFFX₂₁X₂₂DVGSNKX₂₉X₃₀ | 663 |
| YEVHHQKLVFFX₂₁X₂₂DVGSNKX₂₉X₃₀ | 664 |
| EVHHQKLVFFX₂₁X₂₂DVGSNKX₂₉X₃₀ | 665 |
| VHHQKLVFFX₂₁X₂₂DVGSNKX₂₉X₃₀ | 666 |
| HHQKLVFFX₂₁X₂₂DVGSNKX₂₉X₃₀ | 667 |
| HQKLVFFX₂₁X₂₂DVGSNKX₂₉X₃₀ | 668 |
| QKLVFFX₂₁X₂₂DVGSNKX₂₉X₃₀ | 669 |
| KLVFFX₂₁X₂₂DVGSNKX₂₉X₃₀ | 670 |
| LVFFX₂₁X₂₂DVGSNKX₂₉X₃₀ | 671 |
| VFFX₂₁X₂₂DVGSNKX₂₉X₃₀ | 672 |
| FFX₂₁X₂₂DVGSNKX₂₉X₃₀ | 673 |
| FX₂₁X₂₂DVGSNKX₂₉X₃₀ | 674 |
| X₂₁X₂₂DVGSNKX₂₉X₃₀ | 675 |
| X₂₂DVGSNKX₂₉A | 676 |
| DAEFRHDSGYEVHHQKLVFFAX₂₂DVGSNKX₂₉ | 677 |
| AEFRHDSGYEVHHQKLVFFAX₂₂DVGSNKX₂₉ | 678 |
| EFRHDSGYEVHHQKLVFFAX₂₂DVGSNKX₂₉ | 679 |
| FRHDSGYEVHHQKLVFFAX₂₂DVGSNKX₂₉ | 680 |
| RHDSGYEVHHQKLVFFAX₂₂DVGSNKX₂₉ | 681 |
| HDSGYEVHHQKLVFFAX₂₂DVGSNKX₂₉ | 682 |
| DSGYEVHHQKLVFFAX₂₂DVGSNKX₂₉ | 683 |
| SGYEVHHQKLVFFAX₂₂DVGSNKX₂₉ | 684 |
| GYEVHHQKLVFFAX₂₂DVGSNKX₂₉ | 685 |
| YEVHHQKLVFFAX₂₂DVGSNKX₂₉ | 686 |
| EVHHQKLVFFAX₂₂DVGSNKX₂₉ | 687 |
| VHHQKLVFFAX₂₂DVGSNKX₂₉ | 688 |
| HHQKLVFFAX₂₂DVGSNKX₂₉ | 689 |
| HQKLVFFAX₂₂DVGSNKX₂₉ | 690 |
| QKLVFFAX₂₂DVGSNKX₂₉ | 691 |
| KLVFFAX₂₂DVGSNKX₂₉ | 692 |
| LVFFAX₂₂DVGSNKX₂₉ | 693 |
| VFFAX₂₂DVGSNKX₂₉ | 694 |
| FFAX₂₂DVGSNKX₂₉ | 695 |
| FAX₂₂DVGSNKX₂₉ | 696 |
| AX₂₂DVGSNKX₂₉ | 697 |
| X₂₂DVGSNKX₂₉ | 698 |

Said intra-sequence covalent linkage(s) is (are) intended to stabilize the loop and optionally further secondary structure elements of the amyloid β peptide analogues of the invention, as described. Accordingly, the covalently linked amino acid residues are expediently separated by at least the loop-forming amino acids, for instance the sequence VGSN, DVGSN, VGSNK, or Q as described herein. Particularly preferred positions of the intra-sequence covalent linkages in SEQ ID NO: 369-698 may be described in the form of the following embodiments:

At least one amino acid residue selected from the group consisting of X₁₂, X₁₃, X₁₄ and at least one amino acid residue selected from the group consisting of X₃₇, X₃₈, X₃₉ are covalently linked with each other.

At least one amino acid residue selected from the group consisting of X₁₃, X₁₄, X₁₅ and at least one amino acid residue selected from the group consisting of X₃₆, X₃₇, X₃₈ are covalently linked with each other.

At least one amino acid residue selected from the group consisting of X₁₄, X₁₅, X₁₆ and at least one amino acid residue selected from the group consisting of X₃₅, X₃₆, X₃₇ are covalently linked with each other.

At least one amino acid residue selected from the group consisting of X₁₅, X₁₆, X₁₇ and at least one amino acid residue selected from the group consisting of X₃₄, X₃₅, X₃₆ are covalently linked with each other.

At least one amino acid residue selected from the group consisting of X₁₆, X₁₇, X₁₈ and at least one amino acid residue selected from the group consisting of X₃₃, X₃₄, X₃₅ are covalently linked with each other.

At least one amino acid residue selected from the group consisting of X₁₇, X₁₈, X₁₉ and at least one amino acid residue selected from the group consisting of X₃₂, X₃₃, X₃₄ are covalently linked with each other.

At least one amino acid residue selected from the group consisting of X₁₈, X₁₉, X₂₀ and at least one amino acid residue selected from the group consisting of X₃₁, X₃₂, X₃₃ are covalently linked with each other.

At least one amino acid residue selected from the group consisting of X₁₉, X₂₀, X₂₁ and at least one amino acid residue selected from the group consisting of X₃₀, X₃₁, X₃₂ are covalently linked with each other.

At least one amino acid residue selected from the group consisting of X₂₀, X₂₁ and X₂₂ and at least one amino acid residue selected from the group consisting of X₂₉, X₃₀, X₃₁ are covalently linked with each other.

More particularly, the amino acid residues X₁₂ and X₃₉, X₁₃ and X₃₈, X₁₄ and X₃₇, X₁₅ and X₃₆, X₁₆ and X₃₅, X₁₇ and X₃₄, X₁₈ and X₃₃, X₁₉ and X₃₂, X₂₀ and X₃₁, X₂₁ and X₃₀, or X₂₂ and X₂₉ may expediently covalently linked with each other.

According to a further embodiment of the invention, the amino acid sequence of the amyloid β peptide analogues of the invention comprises the sequence X₂₀A₂₁E₂₂D₂₃-X₂₄X₂₅X₂₆X₂₇X₂₈X₂₀X₃₀X₃₁, wherein each of X₂₀, X₂₄, X₂₅, X₂₆, X₂₇, X₂₈, X₂₉, X₃₀, X₃₁ independently represents an amino acid, in particular as defined herein. Each of said amino acids may be covalently linked with another amino acid, with the other amino acid being selected among X₂₀, X₂₄, X₂₅, X₂₆, X₂₇, X₂₈, X₂₉, X₃₀, X₃₁, or representing a different amino acid. Preferably, the amino acid sequences X₂₀A₂₁E₂₂D₂₃ and X₂₈X₂₉X₃₀X₃₁ are in anti-parallel orientation.

Particular amyloid β peptide analogues of the invention include those wherein

X₂₀ is an amino acid residue selected from the group consisting of phenylalanine, tyrosine, valine, leucine, isoleucine, and methionine, with phenylalanine being preferred;
X₂₄ is an amino acid residue selected from the group consisting of valine, leucine, isoleucine, alanine, and methionine, with valine being preferred;
X₂₅ is an amino acid residue selected from the group consisting of glycine, alanine, and serine, with glycine being preferred;
X₂₆ is an amino acid residue selected from the group consisting of serine, glycine, alanine, and threonine, with serine being preferred;
X₂₇ is an amino acid residue selected from the group consisting of asparagine, glutamine, and methionine, with asparagine being preferred;
X₂₈ is an amino acid residue selected from the group consisting of lysine and arginine, with lysine being preferred;
X₂₉ is an amino acid residue selected from the group consisting of glycine, alanine, and serine, with glycine being preferred;
X₃₀ is an amino acid residue selected from the group consisting of alanine, valine, glycine, and serine, with alanine being preferred;
X₃₁ is an amino acid residue selected from the group consisting of isoleucine, leucine, valine, phenylalanine, and methionine, with isoleucine being preferred;
or any combination thereof.

Further particular amyloid β peptide analogues of the invention include those wherein the amino acid sequence of the amyloid β peptide analogue comprises the sequence X₂₀-Q-X₂₄X₂₅X₂₆X₂₇X₂₈X₂₉A₃₀I₃₁, with each of X₂₀, X₂₄, X₂₅, X₂₆, X₂₇, X₂₈, X₂₉, independently representing an amino acid, in particular as defined herein, and Q being an amino acid sequence comprising the sequence AED.

The amino acid sequence Q usually consists of 3, 4, 5, or 6 amino acid residues. According to a particular embodiment, at least part of the amino acid sequence X₂₄X₂₅X₂₆X₂₇ forms the loop.

Amyloid β peptide analogues comprising the sequence X₂₀A₂₁E₂₂D₂₃X₂₄X₂₅X₂₆X₂₇X₂₈X₂₉A₃₀I₃₁, wherein each of X₂₀, X₂₄, X₂₅, X₂₆, X₂₇, X₂₈, X₂₉, independently represents an amino acid residue, in particular as defined herein, represent a preferred embodiment of the invention. In these amyloid β peptide analogues, at least three contiguous amino acids of the amino acid sequences X₂₀A₂₁E₂₂D₂₃ and X₂₈X₂₉A₃₀I₃₁ are preferably in anti-parallel orientation. More particularly, it is preferred if the interproton distance for at least one atom pair selected from the group consisting of A₂₁ (NH)-A₃₀(NH), A₂₁(NH)-A₃₀(CB), A₂₁(NH)—I₃₁(CD1), A₂₁(NH)—I₃₁(CG2), and A₃₀(NH)-A₂₁(CB) is 1.8 to 6.5 Angstroms. It is also preferred if the atom pairs A₂₁(CO)-A₃₀(N) and A₃₀(CO)-A₂₁(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150.

According to a further particular embodiment, the amino acid sequence of the amyloid β peptide analogue is a sequence selected from the group consisting of SEQ ID NO: 699-960, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid residue which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₄ is valine, leucine, isoleucine, alanine, methionine, or an amino acid residue which is covalently linked to another amino acid residue of the sequence;
X₂₅ is glycine, alanine, serine, or an amino acid residue which is covalently linked to another amino acid residue of the sequence;
X₂₆ is serine, glycine, alanine, threonine, or an amino acid residue which is covalently linked to another amino acid residue of the sequence;
X₂₇ is asparagine, glutamine, methionine, or an amino acid residue which is covalently linked to another amino acid residue of the sequence;
X₂₈ is lysine, arginine, or an amino acid residue which is covalently linked to another amino acid residue of the sequence;
X₂₉ is glycine, alanine, serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
   at least one amino acid residue selected from the group consisting of X₁₂, X₁₃, X₁₄, X₁₅, X₁₆, X₁₇, X₁₈, X₁₉, X₂₀, and at least one amino acid residue selected from the group consisting of X₂₉, X₃₀, X₃₁, X₃₂, X₃₃, X₃₄, X₃₅, X₃₆, X₃₇, X₃₈ and X₃₉ being covalently linked with other.

| Sequence | |
|---|---|
| DAEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 699 |
| AEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 701 |
| EFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 702 |
| FRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 703 |
| RHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 704 |
| HDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 705 |
| DSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 706 |
| SGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 707 |
| GYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 708 |
| YEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 709 |
| EX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 710 |
| X₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIAT | 711 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈VVIAT | 712 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇GVVIAT | 713 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆GGVVIAT | 714 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGGVV1AT | 715 |
| X₁₇X₁₈X₁₉X₂₀AEDX₂₄X₂₅X₂₆X₂₇X₂₈X₂₉X₃₀X₃₁X₃₂X₃₃X₃₄MVGGVV1AT | 716 |
| X₁₈X₁₉X₂₀AEDX₂₄X₂₅X₂₆X₂₇X₂₈X₂₉X₃₀X₃₁X₃₂X₃₃LMVGGVVIAT | 717 |
| X₁₉X₂₀AEDX₂₄X₂₅X₂₆X₂₇X₂₈X₂₉X₃₀X₃₁X₃₂GLMVGGVVIAT | 718 |
| X₂₀AEDX₂₄X₂₅X₂₆X₂₇X₂₈X₂₉X₃₀X₃₁IGLMVGGVVIAT | 719 |
| DAEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 720 |
| AEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 721 |
| EFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 722 |
| FRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 723 |
| RHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 724 |
| HDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 725 |
| DSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 726 |
| SGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 727 |
| GYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 728 |
| YEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 729 |
| EX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 730 |
| X₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VIA | 731 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈VVIA | 732 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇GVVIA | 733 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆GGVVIA | 734 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGGVVIA | 735 |
| X₁₇X₁₈X₁₉X₂₀AEDX₂₄X₂₅X₂₆X₂₇X₂₈X₂₉X₃₀X₃₁X₃₂X₃₃X₃₄MVGGVVIA | 736 |
| X₁₈X₁₉X₂₀AEDX₂₄X₂₅X₂₆X₂₇X₂₈X₂₉X₃₀X₃₁X₃₂X₃₃LMVGGVVIA | 737 |
| X₁₉X₂₀AEDX₂₄X₂₅X₂₆X₂₇X₂₈X₂₉X₃₀X₃₁X₃₂GLMVGGVVIA | 738 |
| X₂₀AEDX₂₄X₂₅X₂₆X₂₇X₂₈X₂₉X₃₀X₃₁IGLMVGGVVIA | 739 |
| DAEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 740 |
| AEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 741 |
| EFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 742 |
| FRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 743 |
| RHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 744 |
| HDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 745 |
| DSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 746 |
| SGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 747 |
| GYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 748 |
| YEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 749 |
| EX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 750 |
| X₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉VI | 751 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈VVI | 752 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇GVVI | 753 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆GGVVI | 754 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGGVVI | 755 |
| X₁₇X₁₈X₁₉X₂₀AEDX₂₄X₂₅X₂₆X₂₇X₂₈X₂₉X₃₀X₃₁X₃₂X₃₃X₃₄MVGGVVI | 756 |
| X₁₈X₁₉X₂₀AEDX₂₄X₂₅X₂₆X₂₇X₂₈X₂₉X₃₀X₃₁X₃₂X₃₃LMVGGVVI | 757 |
| X₁₉X₂₀AEDX₂₄X₂₅X₂₆X₂₇X₂₈X₂₉X₃₀X₃₁X₃₂GLMVGGVVI | 758 |
| X₂₀AEDX₂₄X₂₅X₂₆X₂₇X₂₈X₂₉X₃₀X₃₁IGLMVGGVVI | 759 |
| DAEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 760 |
| AEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 761 |
| EFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 762 |
| FRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 763 |
| RHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 764 |
| HDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 765 |
| DSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 766 |
| SGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 767 |
| GYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 768 |
| YEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 769 |
| EX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 770 |
| X₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉V | 771 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈VV | 772 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇GVV | 773 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆GGVV | 774 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGGVV | 775 |
| X₁₇X₁₈X₁₉X₂₀AEDX₂₄X₂₅X₂₆X₂₇X₂₈X₂₉X₃₀X₃₁X₃₂X₃₃X₃₄MVGGVV | 776 |
| X₁₈X₁₉X₂₀AEDX₂₄X₂₅X₂₆X₂₇X₂₈X₂₉X₃₀X₃₁X₃₂X₃₃LMVGGVV | 777 |
| X₁₉X₂₀AEDX₂₄X₂₅X₂₆X₂₇X₂₈X₂₉X₃₀X₃₁X₃₂GLMVGGVV | 778 |
| X₂₀AEDX₂₄X₂₅X₂₆X₂₇X₂₈X₂₉X₃₀X₃₁IGLMVGGVV | 779 |
| DAEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 780 |
| AEFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 781 |
| EFRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 782 |
| FRHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 783 |
| RHDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 784 |
| HDSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 785 |
| DSGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 786 |
| SGYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 787 |
| GYEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 788 |
| YEX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 789 |
| EX₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 790 |
| X₁₂X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈X₃₉ | 791 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈V | 792 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇GV | 793 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆GGV | 794 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGGV | 795 |
| X₁₇X₁₈X₁₉X₂₀AEDX₂₄X₂₅X₂₆X₂₇X₂₈X₂₉X₃₀X₃₁X₃₂X₃₃X₃₄MVGGV | 796 |
| X₁₈X₁₉X₂₀AEDX₂₄X₂₅X₂₆X₂₇X₂₈X₂₉X₃₀X₃₁X₃₂X₃₃LMVGGV | 797 |
| X₁₉X₂₀AEDX₂₄X₂₅X₂₆X₂₇X₂₈X₂₉X₃₀X₃₁X₃₂GLMVGGV | 798 |
| X₂₀AEDX₂₄X₂₅X₂₆X₂₇X₂₈X₂₉X₃₀X₃₁IGLMVGGV | 799 |
| DAEFRHDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 800 |
| AEFRHDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 801 |
| EFRHDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 802 |
| FRHDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 803 |
| RHDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 804 |
| HDSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 805 |
| DSGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 806 |
| SGYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 807 |
| GYEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 808 |
| YEVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 809 |
| EVX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 810 |
| VX₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 811 |
| X₁₃X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇X₃₈ | 813 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇G | 814 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆₃₈GG | 815 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VGG | 816 |
| X₁₇X₁₈X₁₉X₂₀AEDX₂₄X₂₅X₂₆X₂₇X₂₈X₂₉X₃₀X₃₁X₃₂X₃₃X₃₄MVGG | 817 |
| X₁₈X₁₉X₂₀AEDX₂₄X₂₅X₂₆X₂₇X₂₈X₂₉X₃₀X₃₁X₃₂X₃₃LMVGG | 818 |
| X₁₉X₂₀AEDX₂₄X₂₅X₂₆X₂₇X₂₈X₂₉X₃₀X₃₁X₃₂GLMVGG | 819 |
| X₂₀AEDX₂₄X₂₅X₂₆X₂₇X₂₈X₂₉X₃₀X₃₁IGLMVGG | 820 |
| DAEFRHDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 821 |
| AEFRHDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 822 |
| EFRHDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 823 |
| FRHDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 824 |
| RHDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 825 |
| HDSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 826 |
| DSGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 827 |
| SGYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 828 |
| GYEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 829 |
| YEVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 830 |
| EVHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 831 |
| VHX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 832 |
| HX₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 833 |
| X₁₄X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆X₃₇ | 834 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆G | 835 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅VG | 836 |
| X₁₇X₁₈X₁₉X₂₀AEDX₂₄X₂₅X₂₆X₂₇X₂₈X₂₉X₃₀X₃₁X₃₂X₃₃X₃₄MVG | 837 |
| X₁₈X₁₉X₂₀AEDX₂₄X₂₅X₂₆X₂₇X₂₈X₂₉X₃₀X₃₁X₃₂X₃₃LMVG | 838 |
| X₁₉X₂₀AEDX₂₄X₂₅X₂₆X₂₇X₂₈X₂₉X₃₀X₃₁X₃₂GLMVG | 839 |
| X₂₀AEDX₂₄X₂₅X₂₆X₂₇X₂₈X₂₉X₃₀X₃₁IGLMVG | 840 |
| DAEFRHDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 841 |
| AEFRHDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 842 |
| EFRHDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 843 |
| FRHDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 844 |
| RHDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 845 |
| HDSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 846 |
| DSGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 847 |
| SGYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 848 |
| GYEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 849 |
| YEVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 850 |
| EVHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 851 |
| VHHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 852 |
| VHX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 853 |
| HX₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 854 |
| X₁₅X₁₆X₁₇X₁₈X₁₉X₂₀AEDX₂₄X₂₅X₂₆X₂₇X₂₈X₂₉X₃₀X₃₁X₃₂X₃₃X₃₄X₃₅X₃₆ | 855 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅V | 856 |
| X₁₇X₁₈X₁₉X₂₀AEDX₂₄X₂₅X₂₆X₂₇X₂₈X₂₉X₃₀X₃₁X₃₂X₃₃X₃₄MV | 857 |
| X₁₈X₁₉X₂₀AEDX₂₄X₂₅X₂₆X₂₇X₂₈X₂₉X₃₀X₃₁X₃₂X₃₃LMV | 858 |
| X₁₉X₂₀AEDX₂₄X₂₅X₂₆X₂₇X₂₈X₂₉X₃₀X₃₁X₃₂GLMV | 859 |
| X₂₀AEDX₂₄X₂₅X₂₆X₂₇X₂₈X₂₉X₃₀X₃₁IGLMV | 860 |
| DAEFRHDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 861 |
| AEFRHDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 862 |
| EFRHDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 863 |
| FRHDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 864 |
| RHDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 865 |
| HDSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 866 |
| DSGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 867 |
| SGYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 868 |
| GYEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 869 |
| YEVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 870 |
| EVHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 871 |
| VHHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 872 |
| HHQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 873 |
| HQX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 874 |
| QX₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 875 |
| X₁₆X₁₇X₁₈X₁₉X₂₀AEDX₂₄X₂₅X₂₆X₂₇X₂₈X₂₉X₃₀X₃₁X₃₂X₃₃X₃₄X₃₅ | 876 |
| X₁₇X₁₈X₁₉X₂₀AEDX₂₄X₂₅X₂₆X₂₇X₂₈X₂₉X₃₀X₃₁X₃₂X₃₃X₃₄M | 877 |
| X₁₈X₁₉X₂₀AEDX₂₄X₂₅X₂₆X₂₇X₂₈X₂₉X₃₀X₃₁X₃₂X₃₃LM | 878 |
| X₁₉X₂₀AEDX₂₄X₂₅X₂₆X₂₇X₂₈X₂₉X₃₀X₃₁X₃₂GLM | 879 |
| X₂₀AEDX₂₄X₂₅X₂₆X₂₇X₂₈X₂₉X₃₀X₃₁IGLM | 880 |
| DAEFRHDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 881 |
| AEFRHDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 882 |
| EFRHDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 883 |
| FRHDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 884 |
| RHDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 885 |
| HDSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 886 |
| DSGYEVHHQKX₁₇X₁₈X₁₉X₂₀AEDX₂₄X₂₅X₂₆X₂₇X₂₈X₂₉X₃₀X₃₁X₃₂X₃₃X₃₄ | 887 |
| SGYEVHHQKX₁₇X₁₈X₁₉X₂₀AEDX₂₄X₂₅X₂₆X₂₇X₂₈X₂₉X₃₀X₃₁X₃₂X₃₃X₃₄ | 888 |
| GYEVHHQKX₁₇X₁₈X₁₉X₂₀AEDX₂₄X₂₅X₂₆X₂₇X₂₈X₂₉X₃₀X₃₁X₃₂X₃₃X₃₄ | 889 |
| YEVHHQKX₁₇X₁₈X₁₉X₂₀AEDX₂₄X₂₅X₂₆X₂₇X₂₈X₂₉X₃₀X₃₁X₃₂X₃₃X₃₄ | 890 |
| EVHHQKX₁₇X₁₈X₁₉X₂₀AEDX₂₄X₂₅X₂₆X₂₇X₂₈X₂₉X₃₀X₃₁X₃₂X₃₃X₃₄ | 891 |
| VHHQKX₁₇X₁₈X₁₉X₂₀AEDX₂₄X₂₅X₂₆X₂₇X₂₈X₂₉X₃₀X₃₁X₃₂X₃₃X₃₄ | 892 |
| HHQKX₁₇X₁₈X₁₉X₂₀AEDX₂₄X₂₅X₂₆X₂₇X₂₈X₂₉X₃₀X₃₁X₃₂X₃₃X₃₄ | 893 |
| HQKX₁₇X₁₈X₁₉X₂₀AEDX₂₄X₂₅X₂₆X₂₇X₂₈X₂₉X₃₀X₃₁X₃₂X₃₃X₃₄ | 894 |
| QKX₁₇X₁₈X₁₉X₂₀AEDX₂₄X₂₅X₂₆X₂₇X₂₈X₂₉X₃₀X₃₁X₃₂X₃₃X₃₄ | 895 |
| KX₁₇X₁₈X₁₉X₂₀AEDX₂₄X₂₅X₂₆X₂₇X₂₈X₂₉X₃₀X₃₁X₃₂X₃₃X₃₄ | 896 |
| X₁₇X₁₈X₁₉X₂₀AEDX₂₄X₂₅X₂₆X₂₇X₂₈X₂₉X₃₀X₃₁X₃₂X₃₃X₃₄ | 897 |
| X₁₈X₁₉X₂₀AEDX₂₄X₂₅X₂₆X₂₇X₂₈X₂₉X₃₀X₃₁X₃₂X₃₃L | 898 |
| X₁₉X₂₀AEDX₂₄X₂₅X₂₆X₂₇X₂₈X₂₉X₃₀X₃₁X₃₂GL | 899 |
| X₂₀AEDX₂₄X₂₅X₂₆X₂₇X₂₈X₂₉X₃₀X₃₁IGL | 900 |
| DAEFRHDSGYEVHHQKLX₁₈X₁₉X₂₀AEDX₂₄X₂₅X₂₆X₂₇X₂₈X₂₉X₃₀X₃₁X₃₂X₃₃ | 901 |
| AEFRHDSGYEVHHQKLX₁₈X₁₉X₂₀AEDX₂₄X₂₅X₂₆X₂₇X₂₈X₂₉X₃₀X₃₁X₃₂X₃₃ | 902 |
| EFRHDSGYEVHHQKLX₁₈X₁₉X₂₀AEDX₂₄X₂₅X₂₆X₂₇X₂₈X₂₉X₃₀X₃₁X₃₂X₃₃ | 903 |
| FRHDSGYEVHHQKLX₁₈X₁₉X₂₀AEDX₂₄X₂₅X₂₆X₂₇X₂₈X₂₉X₃₀X₃₁X₃₂X₃₃ | 904 |
| RHDSGYEVHHQKLX₁₈X₁₉X₂₀AEDX₂₄X₂₅X₂₆X₂₇X₂₈X₂₉X₃₀X₃₁X₃₂X₃₃ | 905 |
| HDSGYEVHHQKLX₁₈X₁₉X₂₀AEDX₂₄X₂₅X₂₆X₂₇X₂₈X₂₉X₃₀X₃₁X₃₂X₃₃ | 906 |
| DSGYEVHHQKLX₁₈X₁₉X₂₀AEDX₂₄X₂₅X₂₆X₂₇X₂₈X₂₉X₃₀X₃₁X₃₂X₃₃ | 907 |
| SGYEVHHQKLX₁₈X₁₉X₂₀AEDX₂₄X₂₅X₂₆X₂₇X₂₈X₂₉X₃₀X₃₁X₃₂X₃₃ | 908 |
| GYEVHHQKLX₁₈X₁₉X₂₀AEDX₂₄X₂₅X₂₆X₂₇X₂₈X₂₉X₃₀X₃₁X₃₂X₃₃ | 909 |
| YEVHHQKLX₁₈X₁₉X₂₀AEDX₂₄X₂₅X₂₆X₂₇X₂₈X₂₉X₃₀X₃₁X₃₂X₃₃ | 910 |
| EVHHQKLX₁₈X₁₉X₂₀AEDX₂₄X₂₅X₂₆X₂₇X₂₈X₂₉X₃₀X₃₁X₃₂X₃₃ | 911 |
| VHHQKLX₁₈X₁₉X₂₀AEDX₂₄X₂₅X₂₆X₂₇X₂₈X₂₉X₃₀X₃₁X₃₂X₃₃ | 912 |
| HHQKLX₁₈X₁₉X₂₀AEDX₂₄X₂₅X₂₆X₂₇X₂₈X₂₉X₃₀X₃₁X₃₂X₃₃ | 913 |
| HQKLX₁₈X₁₉X₂₀AEDX₂₄X₂₅X₂₆X₂₇X₂₈X₂₉X₃₀X₃₁X₃₂X₃₃ | 914 |
| QKLX₁₈X₁₉X₂₀AEDX₂₄X₂₅X₂₆X₂₇X₂₈X₂₉X₃₀X₃₁X₃₂X₃₃ | 915 |
| KLX₁₈X₁₉X₂₀AEDX₂₄X₂₅X₂₆X₂₇X₂₈X₂₉X₃₀X₃₁X₃₂X₃₃ | 916 |
| LX₁₈X₁₉X₂₀AEDX₂₄X₂₅X₂₆X₂₇X₂₈X₂₉X₃₀X₃₁X₃₂X₃₃ | 917 |
| X₁₈X₁₉X₂₀AEDX₂₄X₂₅X₂₆X₂₇X₂₈X₂₉X₃₀X₃₁X₃₂X₃₃ | 918 |
| X₁₉X₂₀AEDX₂₄X₂₅X₂₆X₂₇X₂₈X₂₉X₃₀X₃₁X₃₂G | 919 |
| X₂₀AEDX₂₄X₂₅X₂₆X₂₇X₂₈X₂₉X₃₀X₃₁IG | 920 |
| DAEFRHDSGYEVHHQKLVX₁₉X₂₀AEDX₂₄X₂₅X₂₆X₂₇X₂₈X₂₉X₃₀X₃₁X₃₂ | 921 |
| AEFRHDSGYEVHHQKLVX₁₉X₂₀AEDX₂₄X₂₅X₂₆X₂₇X₂₈X₂₉X₃₀X₃₁X₃₂ | 922 |
| EFRHDSGYEVHHQKLVX₁₉X₂₀AEDX₂₄X₂₅X₂₆X₂₇X₂₈X₂₉X₃₀X₃₁X₃₂ | 923 |
| FRHDSGYEVHHQKLVX₁₉X₂₀AEDX₂₄X₂₅X₂₆X₂₇X₂₈X₂₉X₃₀X₃₁X₃₂ | 924 |
| RHDSGYEVHHQKLVX₁₉X₂₀AEDX₂₄X₂₅X₂₆X₂₇X₂₈X₂₉X₃₀X₃₁X₃₂ | 925 |
| HDSGYEVHHQKLVX₁₉X₂₀AEDX₂₄X₂₅X₂₆X₂₇X₂₈X₂₉X₃₀X₃₁X₃₂ | 926 |
| DSGYEVHHQKLVX₁₉X₂₀AEDX₂₄X₂₅X₂₆X₂₇X₂₈X₂₉X₃₀X₃₁X₃₂ | 927 |
| SGYEVHHQKLVX₁₉X₂₀AEDX₂₄X₂₅X₂₆X₂₇X₂₈X₂₉X₃₀X₃₁X₃₂ | 928 |
| GYEVHHQKLVX₁₉X₂₀AEDX₂₄X₂₅X₂₆X₂₇X₂₈X₂₉X₃₀X₃₁X₃₂ | 929 |
| YEVHHQKLVX₁₉X₂₀AEDX₂₄X₂₅X₂₆X₂₇X₂₈X₂₉X₃₀X₃₁X₃₂ | 930 |
| EVHHQKLVX₁₉X₂₀AEDX₂₄X₂₅X₂₆X₂₇X₂₈X₂₉X₃₀X₃₁X₃₂ | 931 |
| VHHQKLVX₁₉X₂₀AEDX₂₄X₂₅X₂₆X₂₇X₂₈X₂₉X₃₀X₃₁X₃₂ | 932 |
| HHQKLVX₁₉X₂₀AEDX₂₄X₂₅X₂₆X₂₇X₂₈X₂₉X₃₀X₃₁X₃₂ | 933 |
| HQKLVX₁₉X₂₀AEDX₂₄X₂₅X₂₆X₂₇X₂₈X₂₉X₃₀X₃₁X₃₂ | 934 |
| QKLVX₁₉X₂₀AEDX₂₄X₂₅X₂₆X₂₇X₂₈X₂₉X₃₀X₃₁X₃₂ | 935 |
| KLVX₁₉X₂₀AEDX₂₄X₂₅X₂₆X₂₇X₂₈X₂₉X₃₀X₃₁X₃₂ | 936 |
| LVX₁₉X₂₀AEDX₂₄X₂₅X₂₆X₂₇X₂₈X₂₉X₃₀X₃₁X₃₂ | 937 |
| VX₁₉X₂₀AEDX₂₄X₂₅X₂₆X₂₇X₂₈X₂₉X₃₀X₃₁X₃₂ | 938 |
| X₁₉X₂₀AEDX₂₄X₂₅X₂₆X₂₇X₂₈X₂₉X₃₀X₃₁X₃₂ | 939 |
| X₂₀AEDX₂₄X₂₅X₂₆X₂₇X₂₈X₂₉X₃₀X₃₁I | 940 |
| DAEFRHDSGYEVHHQKLVFX₂₀AEDX₂₄X₂₅X₂₆X₂₇X₂₈X₂₉X₃₀X₃₁ | 941 |
| AEFRHDSGYEVHHQKLVFX₂₀AEDX₂₄X₂₅X₂₆X₂₇X₂₈X₂₉X₃₀X₃₁ | 942 |
| EFRHDSGYEVHHQKLVFX₂₀AEDX₂₄X₂₅X₂₆X₂₇X₂₈X₂₉X₃₀X₃₁ | 943 |
| FRHDSGYEVHHQKLVFX₂₀AEDX₂₄X₂₅X₂₆X₂₇X₂₈X₂₉X₃₀X₃₁ | 944 |
| RHDSGYEVHHQKLVFX₂₀AEDX₂₄X₂₅X₂₆X₂₇X₂₈X₂₉X₃₀X₃₁ | 945 |
| HDSGYEVHHQKLVFX₂₀AEDX₂₄X₂₅X₂₆X₂₇X₂₈X₂₉X₃₀X₃₁ | 946 |
| DSGYEVHHQKLVFX₂₀AEDX₂₄X₂₅X₂₆X₂₇X₂₈X₂₉X₃₀X₃₁ | 947 |
| SGYEVHHQKLVFX₂₀AEDX₂₄X₂₅X₂₆X₂₇X₂₈X₂₉X₃₀X₃₁ | 948 |
| GYEVHHQKLVFX₂₀AEDX₂₄X₂₅X₂₆X₂₇X₂₈X₂₉X₃₀X₃₁ | 949 |
| YEVHHQKLVFX₂₀AEDX₂₄X₂₅X₂₆X₂₇X₂₈X₂₉X₃₀X₃₁ | 950 |
| EVHHQKLVFX₂₀AEDX₂₄X₂₅X₂₆X₂₇X₂₈X₂₉X₃₀X₃₁ | 951 |
| VHHQKLVFX₂₀AEDX₂₄X₂₅X₂₆X₂₇X₂₈X₂₉X₃₀X₃₁ | 952 |
| HHQKLVFX₂₀AEDX₂₄X₂₅X₂₆X₂₇X₂₈X₂₉X₃₀X₃₁ | 953 |
| HQKLVFX₂₀AEDX₂₄X₂₅X₂₆X₂₇X₂₈X₂₉X₃₀X₃₁ | 954 |
| QKLVFX₂₀AEDX₂₄X₂₅X₂₆X₂₇X₂₈X₂₉X₃₀X₃₁ | 955 |
| KLVFX₂₀AEDX₂₄X₂₅X₂₆X₂₇X₂₈X₂₉X₃₀X₃₁ | 956 |
| LVFX₂₀AEDX₂₄X₂₅X₂₆X₂₇X₂₈X₂₉X₃₀X₃₁ | 957 |
| VFX₂₀AEDX₂₄X₂₅X₂₆X₂₇X₂₈X₂₉X₃₀X₃₁ | 958 |
| FX₂₀AEDX₂₄X₂₅X₂₆X₂₇X₂₈X₂₉X₃₀X₃₁ | 959 |
| X₂₀AEDX₂₄X₂₅X₂₆X₂₇X₂₈X₂₉X₃₀X₃₁ | 960 |

Said intra-sequence covalent linkage(s) is (are) intended to stabilize the loop and optionally further secondary structure elements of the amyloid β peptide analogues of the invention, as described. Accordingly, the covalenty linked amino acid residues are expediently separated by at least the loop-forming amino acids, for instance the sequence X₂₄X₂₅X₂₆X₂₇, as described herein. Particularly preferred positions of the intra-sequence covalent linkages in SEQ ID NO: 699-960 may be described in the form of the following embodiments:

At least one amino acid residue selected from the group consisting of X₁₂, X₁₃, X₁₄, and at least one amino acid residue selected from the group consisting of X₃₇, X₃₈, X₃₉ are covalently linked with each other.

At least one amino acid residue selected from the group consisting of X₁₃, X₁₄, X₁₅, and at least one amino acid residue selected from the group consisting of X₃₆, X₃₇, X₃₈ are covalently linked with each other.

At least one amino acid residue selected from the group consisting of X₁₄, X₁₅, X₁₆, and at least one amino acid residue selected from the group consisting of X₃₅, X₃₆, X₃₇ are covalently linked with each other.

At least one amino acid residue selected from the group consisting of X₁₅, X₁₆, X₁₇, and at least one amino acid residue selected from the group consisting of X₃₄, X₃₅, X₃₆ are covalently linked with each other.

At least one amino acid residue selected from the group consisting of X₁₆, X₁₇, X₁₈, and at least one amino acid residue selected from the group consisting of X₃₃, X₃₄, X₃₅ are covalently linked with each other.

At least one amino acid residue selected from the group consisting of X₁₇, X₁₈, X₁₉, and at least one amino acid residue selected from the group consisting of X₃₂, X₃₃, X₃₄ are covalently linked with each other.

At least one amino acid residue selected from the group consisting of X₁₈, X₁₉, X₂₀, and at least one amino acid residue selected from the group consisting of X₃₁, X₃₂, X₃₃ are covalently linked with each other.

At least one amino acid residue selected from the group consisting of X₁₉, X₂₀, and at least one amino acid residue selected from the group consisting of X₃₀, X₃₁, X₃₂ are covalently linked with each other.

Amino acid residue X₂₀ and at least one amino acid residue selected from the group consisting of X₂₉, X₃₀, X₃₁ are covalently linked with each other.

More particularly, the amino acid residues X₁₂ and X₃₉, X₁₃ and X₃₈, X₁₄ and X₃₇, X₁₅ and X₃₆, X₁₆ and X₃₅, X₁₇ and X₃₄, X₁₈ and X₃₃, X₁₉ and X₃₂, or X₂₀ and X₃₁ may expediently covalently linked with each other.

It is noted that the amino acid sequence of the amyloid β peptide analogues of the invention may comprise further amino acid residues than those specifically described herein. In particular, there may be one or more than one additional amino acid residue at the N-terminal and/or C-terminal position of the sequences selected from the group consisting of SEQ ID NO:1-960. For instance, the amino acid sequence of the amyloid β peptide analogues of the invention may comprise a methionine at the N-terminal position of one of the sequences set forth in SEQ ID NO:1-960, especially if the peptide corresponding to the amino acid sequence is produced recombinantly in a prokaryotic host. Further, one or more than one amino acids may be inserted into the amino acid sequences described herein.

It is further noted that those sequences among SEQ ID NO:1-960 which lack 3, 11, 15 or 19 N-terminal amino acids from either SEQ ID NO:1, SEQ ID NO:369 or SEQ ID NO:699 represent particular embodiments of the present invention.

Likewise, it is noted that those sequences among SEQ ID NO:1-960 which lack 1, 2, 3, 4, 5, 6, 7 or 8 C-terminal amino acids from either SEQ ID NO:1, SEQ ID NO:369 or SEQ ID NO:699 represent particular embodiments of the present invention.

Particular embodiments of the present invention include amyloid β peptide analogues as defined herein, wherein the amino acid sequence is Aβ(1-42), Aβ(12-42), Aβ(16-42), Aβ(20-42), or Aβ(16-35), with the amino acid at position 14, 15, 16, 17, 18, 19, 20, 21, or 22 being selected from the group consisting cysteine, lysine, glutamic acid or aspartic acid, in particular cysteine or lysine, and the amino acid at position 37, 36, 35, 34, 33, 32, 31, 30, or 29 being selected from the group consisting cysteine, lysine, glutamic acid or aspartic acid, in particular cysteine, lysine, or glutamic acid, and the amino acid at position 14 is covalently linked with the amino acid at position 37, the amino acid at position 15 is covalently linked with the amino acid at position 36, the amino acid at position 16 is covalently linked with the amino acid at position 35, the amino acid at position 17 is covalently linked with the amino acid at position 34, the amino acid at position 18 is covalently linked with the amino acid at position 33, the amino acid at position 19 is covalently linked with the amino acid at position 32, the amino acid at position 120 is covalently linked with the amino acid at position 31, the amino acid at position 21 is covalently linked with the amino acid at position 30, or the amino acid at position 22 is covalently linked with the amino acid at position 29.

A covalent linkage between two amino acid residues may be established by a variety of means well known in the art, for instance by disulfide bridge formation or cross-linking techniques. In particular, the side chains of amino acid residues may be linked with each other. Especially side chains with a functional group, e.g., a thiol, amino, carboxyl or hydroxyl group, may linked with each other directly, such as two cysteine residues which form a disulfide bridge, or indirectly via a linker. Accordingly, the amino acid residue that is covalently linked to the other amino acid residues may in particular be that of an amino acid residue selected from the group consisting of cysteine, lysine, aspartic acid and glutamic acid.

The crosslinking of proteins has a long and exhaustive history, with large amounts of literature precedent. Any methodology known to those familiar with the art that allows for specific covalent cross-links to be made between natural or non-natural amino acid side chains can be used to form the position specific cross-links envisioned in this invention. Some examples of this methodology are listed below.

There are a large number of chemical cross-linking agents that are known to those skilled in the art. For the present invention, the preferred cross-linking agents include homobifunctional and heterobifunctional cross-linkers, with heterobifunctional cross-linkers being preferred due to their suitability to link amino acids in a stepwise manner.

Also, heterobifunctional cross-linkers provide the ability to establish more specific linkages, thereby reducing the occurrences of unwanted side reactions.

A wide variety of heterobifunctional cross-linkers are known in the art.

These include heterobifunctional cross-linkers for forming linkages between two amino (—NH₂) groups, one amino and one thiol (or sufhydryl, i.e., —SH) group, or two thiol groups.

One reactive group useful as part of a heterobifunctional cross-linker is an amine-reactive group. Common amine-reactive groups include N-hydroxysuccinimide (NHS) esters. NHS esters react specifically with free amines (e.g., lysine residues) in minutes, under slightly acidic to neutral (pH 6.5-7.5) conditions.

It is noted that the cross-linking agents having N-hydroxysuccinimide moieties can also be used in the form of their N-hydroxysulfosuccinimide analogs, which generally have greater water solubility.

Another reactive group useful as part of a heterobifunctional cross-linker is a thiol reactive group. Common thiol reactive groups include maleimides, halogens, and pyridyl disulfides. Maleimides react specifically with free thiol groups (e.g., in cysteine residues) in minutes, preferably under slightly acidic to neutral (pH 6.5-7.5) conditions. Halogens (iodoacetyl functions) react with —SH groups at physiological pH's. Both of these reactive groups result in the formation of stable thioether bonds.

For instance, succinimidyl-4-(N-maleimidomethyl)-cyclohexane-1-carboxylate (SMCC) or sulfo-SMCC can be used to form a cross-link between the amine of, e.g., a Lys side chain and the free —SH of, e.g., a Cys side chain. The amine-reactive N-hydroxysuccinimide (NHS) ester will react with an amino group (e.g., that of a Lys residue) to form a stable amide bond. The resulting maleimide-activated peptide will then react with a sulfhydryl group of the same peptide (e.g., that of a Cys residue) to form a disulfide bond, thereby establishing the covalent linkage. This chemistry is well described in the literature; see for instance: Uto, I., et al. (1991). J. Immunol. Methods 138, 87-94; Bieniarz, C., et al. (1996). Extended Length Heterobifunctional Coupling Agents for Protein Conjugations. Bioconjug. Chem. 7, 88-95; Chrisey, L. A., et al. (1996). Nucleic Acids Res. 24(15), 3031-3039; Kuijpers, W. H., et al. (1993). Bioconjug. Chem. 4(1), 94-102; Brinkley, M. A. (1992). A survey of methods for preparing protein conjugates with dyes, haptens and crosslinking reagents. Bioconjugate Chem. 3, 2-13; Hashida, S., et al. (1984). More useful maleimide compounds for the conjugation of Fab to horseradish peroxidase through thiol groups in the hinge. J. Appl. Biochem. 6, 56-63; Mattson, G., et al. (1993). A practical approach to crosslinking. Molecular Biology Reports 17, 167-183; Partis, M. D., et al. (1983). Crosslinking of proteins by omega-maleimido alkanoyl N-hydroxysuccinimide esters. J. Protein. Chem. 2, 263-277; Samoszuk, M. K., et al. (1989). A peroxide-generating immunoconjugate directed to eosinophil peroxidase is cytotoxic to Hodgkin's disease cells in vitro. Antibody, Immunoconjugates and Radiopharmaceuticals 2, 37-45; Yoshitake, S., et al. (1982). Mild and efficient conjugation of rabbit Fab and horseradish peroxidase using a maleimide compound and its use for enzyme immunoassay. J. Biochem. 92, 1413-1424.

Further heterobifunctional cross-linkers can be used in a similar fashion, e.g., ([N-ε-maleimidocaproyloxy]succinimide ester, N-[γ-maleimidobutyryloxy]succinimide ester, N-[κ-maleimidoundecanoyloxy]sulfosuccinimide ester, m-maleimidobenzoyl-N-hydroxysuccinimide ester (MBS), or their sulfosuccinimide analogs (e.g. sulfo-MBS).

A further example of a heterobifunctional cross-linker that can be used to form a cross-link between the amine of, e.g., a Lys side chain and the free —SH of, e.g., a Cys side chain, is succinimidyl-6-[(3-(2-pyridyldithio)-propionate]-hexanoate (LC-SPDP) or sulfo-LC-SPDP. The amine-reactive N-hydroxysuccinimide (NHS) ester will react with an amino group (e.g., that of a Lys residue) to form a stable amide bond. The resulting peptide has a pyridyldisulfide group that will then react with a sulfhydryl group of the same peptide (e.g., that of a Cys residue) to form a disulfide bond, thereby establishing the covalent linkage. This chemistry is well described in the literature; see for instance: Carlsson, J., et al. (1978) Biochem. J. 173, 723-737; Stan, R. V. (2004) Am. J. Physiol. Heart Circ. Physiol. 286, H1347-H1353; Mader, C., et al. (2004) J. Bacteriol. 186, 1758-1768.

Further heterobifunctional cross-linkers can be used in a similar fashion, e.g., 4-succinimidyloxycarbonyl-α-methyl-α-(2-pyridyldithio)-toluene (SMPT) or sulfo-SMPT, N-succinimidyl-3-(2-pyridyldithio)-propionate (SPDP) or sulfo-SPDP.

A further example of a heterobifunctional cross-linker that can be used to form a cross-link between the amine of, e.g., a Lys side chain and the free —SH of, e.g., a Cys side chain is N-succinimidyl-S-acetylthioacetate (SATA) or sulfo-SATA. The amine-reactive N-hydroxysuccinimide (NHS) ester will react with an amino group (e.g., that of a Lys residue) to form a stable amide bond. The protected —SH group of the resulting peptide will then be deprotected by treatment with hydroxylamine, and the resulting free —SH will then react with a sulfhydryl group of the same peptide (e.g., that of a Cys residue) to form a disulfide bond, thereby establishing the covalent linkage.

Further heterobifunctional cross-linkers can be used in a similar fashion, e.g., N-succinimidyl-S-acetylthiopropionate or its sulfosuccinimide analog.

Further suitable heterobifunctional cross-linkers include N-succinimidyl-(4-iodoacetyl)-aminobenzoate (SIAB) or sulfo-SIAB.

Specific, stepwise cross-linkages can also be formed between amino (—NH₂) and carboxy (—COOH) groups.

For instance, 1-ethyl-3-(3-dimethylaminopropyl)-carbodiimide hydrochloride (EDC) can be used to form a cross-link between the amine of, e.g., a Lys side chain and the free —COOH of an acidic side chain. The carboxy-reactive carbodiimide will react with an carboxy group (e.g., that of an Asp, Glu, Dab (2,4-diaminobutyric acid), Dap (2,4-diaminopropionic acid), or ornithine residue) to form an unstable o-acylisourea ester. The reactive o-acylisourea ester will then react with an amino group of the same peptide (e.g., that of a Lys residue) to form an amide bond, thereby establishing the covalent linkage. Alternatively, the reactive o-acylisourea ester may be reacted with N-hydroxysuccinimide, N-hydroxysulfosuccinimide or sulfo-N-hydroxysulfosuccinimide to give the semi-stable amine-reactive NHS ester which will then react with an amino group of the same peptide (e.g., that of a Lys residue) to form an amide bond, thereby establishing the covalent linkage. This chemistry is well described in the literature; see for instance: DeSilva, N. S. (2003) Interactions of Surfactant Protein D with Fatty Acids. Am. J. Respir. Cell Mol. Biol. 29, 757-770; Grabarek, Z. and Gergely, J. (1990) Zero-length crosslinking procedure with the use of active esters. Anal. Biochem. 185, 131-135; Sinz, A. (2003). J. Mass Spectrom. 38, 1225-1237. Staros, J. V., Wright, R. W. and Swingle, D. M. (1986) Enhancement by N-hydroxysulfosuccinimide of water-soluble carbodiimide-mediated coupling reactions. Anal. Biochem. 156, 220-222; Taniuchi, M., et al. (1986). Induction of nerve growth factor receptor in Schwann cells after axotomy. Proc. Natl. Acad. Sci. USA 83, 4094-4098.

Heterobifunctional cross-linkers also include the reaction of Lys(N3) and propargyl glycine amino acids. This reaction can be performed in solution or on resin (as described, for instance, in Jiang, S., (2008) Curr. Org. Chem. 12, 1502-1542 and references therein).

A particular class of cross-linkers, in particular heterobifunctional cross-linkers, includes photoreactive cross-linkers.

For instance, (SDA) can be used to form a cross-link between the amine of, e.g., a Lys side chain and the amine of, e.g., another Lys side chain. The amine-reactive N-hydroxysuccinimide (NHS) ester will react with an amino group (e.g., that of a Lys residue) to form a stable amide bond. The resulting peptide has a photo-labile diazirine moiety that, upon exposure to UV light, will react with an amino group (e.g., that of a Lys residue) of the same peptide to form a stable bond, thereby establishing the covalent linkage.

Further suitable photoreactive cross-linkers include bis-[β-(4-azidosalicylamido)-ethyl]-disulfide (BASED) and N-succinimidyl-6-(4′-azido-2′-nitrophenyl-amino)-hexanoate (SAN-PAH).

In addition to the heterobifunctional cross-linkers, there exist a number of other cross-linking agents including homobifunctional cross-linkers.

These include homobifunctional cross-linkers for forming linkages between two amino (—NH₂) groups.

For instance, disuccinimidyl suberate (DSS) can be used to form a cross-link between the amine of, e.g., a Lys side chain and the amine of, e.g., another Lys side chain. The amine-reactive N-hydroxysuccinimide (NHS) ester will react with an amino group (e.g., that of a Lys residue) to form a stable amide bond. The resulting peptide will then react with another amine group of the same peptide (e.g., that of a Lys residue) to form a further stable amide bond, thereby establishing the covalent linkage.

Further suitable homobifunctional cross-linkers include bismaleimidohexane (BMH) and dimethylpimelimidate (DMP).

Further suitable homobifunctional cross-linkers include a methylenedithioether linkage between two cysteines. The reaction of the peptide with TBAF (tetrabutylammonium fluoride) can be performed on resin containing the partially deprotected peptide followed by cleavage (see, for instance, Ueki et al., (1999) Bioorg. Med. Chem. Lett., 9, 1767-1772, and Ueki et al. in Peptide Science, 1999, 539-541).

Further suitable homobifunctional cross-linking systems include ring closing metathesis reactions between allylglycines (see, for instance, Wels, B. et al., (2005) Bioorg. Med. Chem. 13, 4221-4227) or modified amino acids, e.g. (S)-Fmoc-α(2′ pentenyl)alanine (see, for instance, Walensky, L. D., et al., (2004) Science 305, 1466-1470; Schafineister, C. E., et al., (2000) J. Am. Chem. Soc. 122, 5891-5892; Qiu, W., et al., (2000) Tetrahedron 56, 2577-2582; Belokon, Y. N., et al., (1998) Tetrahedron: Asymmetry, 9, 4249-4252); Qiu W., (2008) Anaspec poster at 20th American Peptide Society Annual Meeting). These reactions can be performed in solution on the protected peptide fragment or on the resin, respectively.

Homo- and heterobifunctional cross-linker may comprise a spacer arm or bridge. The bridge is the structure that connects the two reactive ends. The most apparent attribute of the bridge is its effect on steric hindrance. In some instances, a longer bridge can more easily span the distance necessary to link two amino acid residues.

While one covalent linkage between 2 non-contiguous amino acid residues may provide sufficient stabilization, the amyloid β peptide analogues of the invention may comprise more than one covalent linkage.

The present invention also relates to oligomers comprising a plurality of amyloid β peptide analogues as defined herein.

The term “oligomer” here refers to a non-covalent association of two or more amyloid β peptide analogues as defined herein, possessing homogeneity and distinct physical characteristics. According to one aspect, oligomers are stable, non-fibrillar, oligomeric assemblies of amyloid β peptide analogues. According to one embodiment, said assemblies comprise 2 to 28 amyloid β peptide analogues.

Such oligomers may be further characterized by particular interactions between two or amyloid β peptide analogues.

For instance, it is preferred if the amino acid sequence of each amyloid β peptide analogue comprises the sequence L₃₄M₃₅V₃₆G₃₇G₃₈, with the sequence L^{A}₃₄M^{A}₃₅V^{A}₃₆G^{A}₃₇G^{A}₃₈ of one amyloid β peptide analogue (A) being in parallel orientation to the sequence L^{B}₃₄M^{B}₃₅V^{B}₃₆G^{B}₃₇G^{B}₃₈ of another amyloid β peptide analogue (B). More particularly, it is preferred if the interproton distance for at least one atom pair selected from the group consisting of M^{A}₃₅(NH)—V^{B}₃₆(NH), G^{A}₃₇(NH)-G^{B}₃₈(NH), L^{A}₃₄(NH)-L^{B}₃₄(C_{δ}H₃), M^{A}₃₅(NH)—V^{B}₃₆(CγH₃) is 1.8 to 6.5 Angstroms.

According to a further particular embodiment, the invention relates to oligomers wherein the amino acid sequence of each amyloid β peptide analogue comprises the sequence G₃₃L₃₄M₃₅V₃₆G₃₇G₃₈V₃₉, with the sequence G^{A}₃₃L^{A}₃₄M^{A}₃₅V^{A}₃₆G^{A}₃₇G^{A}₃₈V^{A}₃₉ of one amyloid β peptide analogue (A) being in parallel orientation to the sequence G^{B}₃₃L^{B}₃₄M^{B}₃₅V^{B}₃₆G^{B}₃₇G^{B}₃₈V^{B}₃₉ of another amyloid β peptide analogue (B). More particularly, it is preferred if the interproton distance for at least one atom pair selected from the group consisting of G^{A}₃₃(NH)-G^{B}₃₄(NH), M^{A}₃₅(NH)—V^{B}₃₆(NH), G^{A}₃₇(NH)-G^{B}₃₈(NH), L^{A}₃₄(NH)-L^{B}₃₄(C_{δ}H₃), M^{A}₃₅(NH)—V^{B}₃₆(CγH₃), G^{A}₃₈(NH)—V^{B}₃₉(CγH₃) and V^{A}₃₉(NH)—V^{B}₃₉(CγH₃) is 1.8 to 6.5 Angstroms.

Further, it is preferred if the oligomer comprises an inter-molecular parallel β-sheet formed by β-strands of different amyloid β peptide analogues. According to a further particular embodiment, the β-sheet comprises the amino acid sequence G^{A}₃₃L^{A}₃₄M^{A}₃₅V^{A}₃₆G^{A}₃₇G^{A}₃₈V^{A}₃₉ of one amyloid β peptide analogue (A) and the amino acid sequence G^{A}₃₃L^{A}₃₄M^{A}₃₅V^{A}₃₆G^{A}₃₇G^{A}₃₈V^{A}₃₉ of another amyloid β peptide analogue (B). More particularly, it is preferred if the atom pairs G^{A}33(CO)-L^{B}34(N), L^{B}34(CO)-M^{A}35(N), M^{A}35(CO)—V^{B}36(N), V^{B}36(CO)-G^{A}37(N), and G^{B}37(CO)-G^{A}38(N) are at a distance of 3.3±0.5 Å, wherein CO indicates the backbone oxygen atom, and the phi (φ) angles of the residues range from −180 to −30 and psi (ψ) angles of the residues range from approximately 60 to 180 or from approximately −180 to −150.

Interproton distances defining the structure of the antiparallel β-sheet can be determined by the intra-molecular nuclear Overhauser effects (NOEs) between the backbone amides and between the backbone amides and side chains.

Interproton distances defining the structure of the parallel β-sheet can be determined by inter-molecular NOEs between backbone NH—NH and between backbone NH and methyl groups of the side chains.

The intra- vs. inter-molecular NOEs can be distinguished using different isotope-labeled samples, as described, for instance in WO2007/064917, in particular Example V, part G, NMR Features, which is incorporated herein by reference.

Using the NOE-derived distance restraints from the analysis of the NMR data, structures can be calculated, e.g. using the program CNX [A. T. Brunger, et al., Acta Crystallogr. D54 (Pt 5), 905-21, (1998)] by using a simulated annealing protocol [M. Nilges, et al., FEBS Lett. 229, 317-324, (1988)], thereby providing further intra-molecular and/or inter-molecular distances between two atoms.

According to a further particular embodiment, the amyloid β peptide analogues or oligomers of the invention are characterized by their reactivity with particular antibodies. Such antibodies include in particular antibodies having a binding affinity to an Aβ(20-42) truncated globulomer that is greater than the binding affinity of the antibody to an Aβ(1-42) globulomer.

The term “Aβ(X-Y) globulomer” (Aβ(X-Y) globular oligomer) here refers to a soluble, globular, non-covalent association of Aβ(X-Y) peptides as defined herein, possessing homogeneity and distinct physical characteristics. According to one aspect, Aβ(X-Y) globulomers are stable, non-fibrillar, oligomeric assemblies of Aβ(X-Y) peptides. In contrast to monomer and fibrils, these globulomers are characterized by defined assembly numbers of subunits (e.g. early assembly forms, n=4-6, “oligomers A”, and late assembly forms, n=12-14, “oligomers B”, as described in WO2004/067561). The globulomers have a 3-dimensional globular type structure (“molten globule”, see Barghorn et al., 2005, J Neurochem, 95, 834-847). They may be further characterized by one or more of the following features:
cleavability of N-terminal amino acids X-23 with promiscuous proteases (such as thermolysin or endoproteinase GluC) yielding truncated forms of globulomers;
non-accessibility of C-terminal amino acids 24-Y with promiscuous proteases and antibodies;
truncated forms of these globulomers maintain the 3-dimensional core structure of said globulomers with a better accessibility of the core epitope Aβ(20-Y) in its globulomer conformation.

The term “Aβ(X-Y) globulomer” also includes the N-Met Aβ(X-Y) globulomers described in WO2007/064917.

The term “Aβ(X-Y) truncated globulomer” here refers to a truncated form of Aβ(X-Y) globulomer which can be obtained by subjecting Aβ(X-Y) globulomer to limited proteolytic digestion. More specifically, Aβ(X-Y) truncated globulomers include N-terminally truncated forms wherein X is selected from the group consisting of the numbers 2 . . . 24, with X preferably being 20 or 12, and Y is as defined herein, which are obtainable by truncating Aβ(1-Y) globulomers by treatment with appropriate proteases. For instance, an Aβ(20-42) globulomer can be obtained by subjecting an Aβ(1-42) globulomer to thermolysin proteolysis, and an Aβ(12-42) globulomer can be obtained by subjecting an Aβ(1-42) globulomer to endoproteinase GIuC proteolysis. When the desired degree of proteolysis is reached, the protease is inactivated in a generally known manner. The resulting globulomers may then be isolated following the procedures already described herein and, if required, processed further by further work-up and purification steps. A detailed description of said processes is disclosed in WO 2004/067561, which is incorporated herein by reference.

For the purposes of the present invention, an Aβ(1-42) globulomer is in particular the Aβ(1-42) globulomer as described in reference example 1 herein; an N-Met Aβ(1-42) globulomer is in particular the N-Met Aβ(1-42) globulomer as described in reference example 2; an Aβ(20-42) truncated globulomer is in particular the Aβ(20-42) truncated globulomer as described in reference example 3 herein, and an Aβ(12-42) truncated globulomer is in particular the Aβ(12-42) truncated globulomer as described in reference example 4 herein.

Antibodies having a binding affinity to an Aβ(20-42) truncated globulomer that is greater than the binding affinity of the antibody to an Aβ(1-42) globulomer are described in WO 2007/062852 and include, for instance, a monoclonal antibody selected from the group consisting of 7C6, 7E5, 4D10 and 5F7.

According to a particular embodiment, the said antibody binds to the amyloid β peptide analogues or oligomers of the invention with a K_{D} in the range of at least 1×10⁻⁶ M. Preferably, the antibodies bind to the amyloid β peptide analogues or oligomers of the present invention with high affinity, for instance with a K_{D} of 1×10⁻⁷ M or greater affinity, e.g. with a K_{D} of 3×10⁻⁸ M or greater affinity, with a K_{D} of 1×10⁻⁸ M or greater affinity, e.g. with a K_{D} of 3×10⁻⁹ M or greater affinity, with a K_{D} of 1×10⁻⁹ M or greater affinity, e.g. with a K_{D} of 3×10⁻¹⁰ M or greater affinity, with a K_{D} of 1×10⁻¹⁰ M or greater affinity, e.g. with a K_{D} of 3×10⁻¹¹ M or greater affinity, or with a K_{D} of 1×10⁻¹¹ M or greater affinity.

The term “greater affinity” here refers to a degree of interaction where the equilibrium between unbound antibody and unbound amyloid β peptide analogue or oligomer on the one hand and antibody-amyloid β peptide analogue/oligomer complex on the other is further in favour of the antibody-amyloid β peptide analogue/oligomer complex. Likewise, the term “smaller affinity” here refers to a degree of interaction where the equilibrium between unbound antibody and unbound amyloid β peptide analogue or oligomer on the one hand and antibody-amyloid β peptide analogue/oligomer complex on the other is further in favour of the unbound antibody and unbound amyloid β peptide analogue or oligomer. The term “greater affinity” is synonymous with the term “higher affinity” and term “smaller affinity” is synonymous with the term “lower affinity”.

The binding affinities of antibodies (monoclonal or polyclonal) to a given antigen (such as amyloid β peptide analogues or oligomers of the present invention) may be evaluated by using standardized in-vitro immunoassays such as ELISA, dot blot or BIAcore analyses (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnsson, B., et al. (1991) Anal. Biochem. 198:268-277.

According to a particular embodiment, the affinities defined herein refer to the values obtained by performing a dot blot as described in example 12 and evaluating it by densitometry. According to a particular embodiment of the invention, determining the binding affinity by dot blot comprises the following: a certain amount of the antigen or, expediently, an appropriate dilution thereof, for instance in 20 mM NaH₂PO₄, 140 mM NaCl, pH 7.4, 0.2 mg/ml BSA to an antigen concentration of, for example, 100 pmol/μl, 10 pmol/μl, 1 pmol/μl, 0.1 pmol/μl and 0.01 pmol/μl, is dotted onto a nitrocellulose membrane, the membrane is then blocked with milk to prevent unspecific binding and washed, then contacted with the antibody of interest followed by detection of the latter by means of an enzyme-conjugated secondary antibody and a colorimetric reaction; at defined antibody concentrations, the amount of antibody bound allows affinity determination. Thus the relative affinity of two different antibodies to one target, or of one antibody to two different targets, is here defined as the relation of the respective amounts of target-bound antibody observed with the two antibody-target combinations under otherwise identical dot blot conditions. Unlike a similar approach based on Western blotting, the dot blot approach will determine an antibody's affinity to a given target in the latter's natural conformation.

The term “K_{d}”, as used herein, is intended to refer to the dissociation constant of a particular antibody-antigen interaction as is known in the art.

Amyloid β peptide analogues or oligomers of the present invention that react with globulomer-specific antibodies are believed to display at least one globulomer epitope. Therefore, the amyloid β peptide analogues or oligomers of the present invention are capable of eliciting an immune response having a similar profil as the immune response elicited when Aβ(20-42) truncated globulomers or other truncated globulomers are used as immunogen.

The term “epitope” includes any polypeptide determinant capable of specific binding to an immunoglobulin. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. An epitope may also be recognized by other binding agents than immunoglobulins.

According to a further particular embodiment, the amyloid β peptide analogues or oligomers of the invention are characterized by their ability of eliciting such a particular immune response, for instance if a mammal, e.g. a rabbit or a mouse, is immunized with an oligomer or derivative of the invention.

An immune response may be regarded as a mixture of antibodies resulting from challenging (immunizing) a host with an antigen (the immunogen). Said mixture of antibodies can be obtained from the host and is hereinafter referred to as the polyclonal antiserum.

In one aspect, such a particular immune response, i.e., the corresponding polyclonal antiserum, is characterized by comprising an antibody having a binding affinity to an amyloid β peptide analogue or oligomer of the invention or to an Aβ(20-42) truncated globulomer that is greater than the binding affinity of the antibody to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer.

According to a particular embodiment, the immune response, i.e., the corresponding polyclonal antiserum, is characterized by having an affinity to an amyloid β peptide analogue or oligomer of the invention or to an Aβ(20-42) truncated globulomer which is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antiserum to at least one Aβ form selected from the group consisting of monomeric Aβ(1-42), monomeric Aβ(1-40), monomeric Aβ(20-42), fibrillomeric Aβ(1-42), fibrillomeric Aβ(1-40) and Aβ(1-42) globulomer.

The term “Aβ(X-Y) monomer” or “monomeric Aβ(X-Y)” here refers to the isolated form of the Aβ(X-Y) peptide, preferably a form of the Aβ(X-Y) peptide which is not engaged in essentially non-covalent interactions with other Aβ peptides. It represents the essentially unfolded peptide which does not display a globulomer epitope. Practically, the Aβ(X-Y) monomer is usually provided in the form of an aqueous solution. In a particularly preferred embodiment of the invention, the aqueous monomer solution contains 0.05% to 0.2%, more preferably about 0.1% NH₄OH. In another particularly preferred embodiment of the invention, the aqueous monomer solution contains 0.05% to 0.2%, more preferably about 0.1% NaOH. When used (for instance for determining the binding affinities of the antibodies of the present invention), it may be expedient to dilute said solution in an appropriate manner. Further, it is usually expedient to use said solution within 2 hours, in particular within 1 hour, and especially within 30 minutes after its preparation.

More specifically, the term “Aβ(1-40) monomer” here refers to an Aβ(1-40) monomer preparation as described in reference example 5 herein, and the term “Aβ(1-42) monomer” here refers to an Aβ(1-42) preparation as described in reference example 6 herein.

In another aspect, such an immune response is characterized by comprising an antibody having a binding affinity to an amyloid β peptide analogue or oligomer of the invention or to an Aβ(20-42) truncated globulomer that is greater than the binding affinity of the antibody to an Aβ(1-42) globulomer.

The term “fibril” here refers to a molecular structure that comprises assemblies of non-covalently associated, individual Aβ(X-Y) peptides, which show fibrillary structure in the electron microscope, which bind Congo red and then exhibit birefringence under polarized light and whose X-ray diffraction pattern is a cross-G3 structure.

In another aspect of the invention, a fibril is a molecular structure obtainable by a process that comprises the self-induced polymeric aggregation of a suitable Aβ peptide in the absence of detergents, e.g. in 0.1 M HCl, leading to the formation of aggregates of more than 24, preferably more than 100 units. This process is well known in the art. Expediently, Aβ(X-Y) fibrils are used in the form of an aqueous solution. In a particularly preferred embodiment of the invention, the aqueous fibril solution is made by dissolving the Aβ peptide in 0.1% NH₄OH, diluting it 1:4 with 20 mM NaH₂PO₄, 140 mM NaCl, pH 7.4, followed by readjusting the pH to 7.4, incubating the solution at 37° C. for 20 h, followed by centrifugation at 10,000 g for 10 min and resuspension in 20 mM NaH₂PO₄, 140 mM NaCl, pH 7.4.

The term “Aβ(X-Y) fibril” here refers to a fibril consisting essentially of Aβ(X-Y) subunits, where it is preferred if on average at least 90% of the subunits are of the Aβ(X-Y) type, more preferred if at least 98% of the subunits are of the Aβ(X-Y) type, and most preferred if the content of non-Aβ(X-Y) peptides is below the detection threshold.

More specifically, the term “Aβ(1-42) fibril” here refers to a Aβ(1-42) fibril preparation as described in reference example 7 herein.

The present invention also relates to a purified amyloid β peptide analogue or oligomer of the invention. According to one embodiment of the present invention, a purified amyloid β peptide analogue or oligomer is one which has a purity of more than 80% by weight of total Aβ peptide, preferably of more than 90% by weight of total Aβ peptide, preferably of more than 95% by weight of total Aβ peptide.

It may be expedient that the amyloid β peptide analogues or oligomers of the invention comprise, in addition to the amyloid β-derived amino acid sequence or peptidomimetic thereof, one or more further moieties. For instance, diagnostic applications may require labelling the amyloid β peptide analogues or oligomers. Also, in active immunization it may be of advantage to attach moieties which prove expedient in active immunization applications.

Thus, the present invention also relates to amyloid β peptide analogues or oligomers, as defined herein, which comprise a covalently linked group that facilitates detection, preferably a fluorophore, e.g. fluorescein isothiocyanate, phycoerythrin, Alexa-488, Aequorea victoria fluorescent protein, Dictyosoma fluorescent protein or any combination or fluorescence-active derivative thereof; a chromophore; a chemoluminophore, e.g. luciferase, preferably Photinus pyralis luciferase, Vibrio fischeri luciferase, or any combination or chemoluminescence-active derivative thereof; an enzymatically active group, e.g. peroxidase, e.g. horseradish peroxidase, or any enzymatically active derivative thereof; an electron-dense group, e.g. a heavy metal containing group, e.g. a gold containing group; a hapten, e.g. a phenol derived hapten; a strongly antigenic structure, e.g. peptide sequence predicted to be anti-genic, e.g. predicted to be antigenic by the algorithm of Kolaskar and Tongaonkar; a molecule which helps elicit an immune response to the amyloid β peptide analogue or oligomer, e.g., serum albumin, ovalbumin, keyhole limpet hemocyanin, thyroglobulin, a toxoid from bacteria such as tetanus toxoid and diphtheria toxoid, a naturally occurring T cell epitope, a naturally occurring T helper cell epitope; an artificial T-cell epitope such as the pan DR epitope (“PADRE”; WO 95/07707), or another immunostimulatory agent, e.g., mannan, tripalmitoyl-5-glycerine cysteine, and the like; an aptamer for another molecule; a chelating group, e.g. hexahistidinyl; a natural or nature-derived protein structure mediating further specific protein-protein interactions, e.g. a member of the fos/jun pair; a magnetic group, e.g. a ferromagnetic group; or a radioactive group, e.g. a group comprising 1H, 14C, 32P, 35S or 125I or any combination thereof. With a view to avoiding the unfavored pro-inflammatory immune response Th1-pathway, amyloid β peptide analogues or oligomers comprising a molecule which is capable of directing the immune response to the anti-inflammatory pathway (Th2-pathway), e.g. molecules comprising a B cell epitope such as PADRE are expected to provide particular advantages in active immunization (see also, Petrushina I., et al., The Journal of Neuroscience 2007, 27(46): 12721-12731; Woodhouse A., et al., Drugs Aging 2007; 24(2): 107-119).

Such groups and methods for linking them to peptides or peptidomimetics are known in the art.

The amyloid β peptide analogues and oligomers disclosed herein can be produced in a manner which known per se in the art.

In a first step, a peptide comprising the amino acid sequence of the desired amyloid β peptide analogue or a peptidomimetic thereof is provided.

Said peptide or peptidomimetic may be produced by chemical synthesis using various solid-phase techniques such as those described in G. Barany and R. B. Merrifield, “The Peptides: Analysis, Synthesis, Biology”; Volume 2—“Special Methods in Peptide Synthesis, Part A”, pp. 3-284, E. Gross and J. Meienhofer, Eds., Academic Press, New York, 1980; and in J. M. Stewart and J. D. Young, “Solid-Phase Peptide Synthesis”, 2nd Ed., Pierce Chemical Co., Rockford, Ill., 1984. This strategy is based on the Fmoc (9-Fluorenylmethyl methyloxycarbonyl) group for temporary protection of the α-amino group, in combination with the tert-butyl group for temporary protection of the amino acid side chains (see for example E. Atherton and R. C. Sheppard, “The Fluorenylmethoxycarbonyl Amino Protecting Group”, in “The Peptides: Analysis, Synthesis, Biology”; Volume 9-“Special Methods in Peptide Synthesis, Part C”, pp. 1-38, S. Undenfriend and J. Meienhofer, Eds., Academic Press, San Diego, 1987.

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively. Alternatively, in cases where a C-terminal amino alcohol is present, the C-terminal residue may be attached to 2-Methoxy-4-alkoxybenzyl alcohol resin (SASRIN™, Bachem Bioscience, Inc., King of Prussia, Pa.) as described herein and, after completion of the peptide sequence assembly, the resulting peptide alcohol is released with LiBH₄ in THF (see J. M. Stewart and J. D. Young, supra, p. 92).

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, Calif.; Applied Biosystems, Foster City, Calif.). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valeryl-aminomethyl-Merrifield resin (PAL resin), for C-terminal carboxamides. Coupling of first and subsequent amino acids can be accomplished using HOBT or HOAT active esters produced from DIC/HOBT, HBTU/HOBT, BOP, PyBOP, or from DIC/HOAT, HATU/HOAT, respectively. Preferred solid supports are: 2-Chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to facilitate dissolution of the amino acid.

The syntheses can be carried out by using a peptide synthesizer, such as an Advanced Chemtech Multiple Peptide Synthesizer (MPS396) or an Applied Biosystems Inc. peptide synthesizer (ABI 433a).

Alternatively, any other appropriate methodology known to those familiar with the art could be used, including: 1) synthesis of multiple copies of the desired peptide or peptidomimetic separated by the appropriate cleavage sites for enzymatic or chemical cleavage of peptide bonds, resulting in the desired peptide peptidomimetic, 2) recombinant expression of APP in any system known to those familiar with the art, and containing the amino acid sequence, followed by either enzymatic or chemical processing to yield the desired peptide, 3) recombinant expression of the desired peptide as a fusion protein in any system known to those familiar with the art, 4) recombinant expression of the desired peptide directly in any system known to those familiar with the art.

The recombinant expression of amyloid β peptides is described in WO2007/064917. Moreover, general methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91: 501; Chaiken 1. M. (1981) CRC Crit. Rev. Biochem. 11: 255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing).

In a second step, the peptide or peptidomimetic obtained is subjected to conditions that allow the linkage to form. The conditions will, of course, depend on the type of linkage to form and can easily be determined by the skilled artisan. Reference is made to description of linkages and their chemistry provided herein.

Oligomer synthesis additionally involves oligomer formation. Thus the second step will not only comprise linkage but also oligomer formation.

Conditions suitable for oligomer formation are described in, for instance, WO 2004/067561; WO 2006/094724; S. Barghorn et al., J. Neurochem. 95, 834 (2005) and WO2007/064917, which are incorporated herein by reference.

In principle, oligomer formation may precede linkage formation. This is advantageous if the pre-formed oligomer directs or promotes the linkages to be formed. Alternatively, linkage formation may precede oligomer formation. This is advantageous if pre-formed linkages direct or promote oligomer formation. Oligomer formation and linkage formation may also take place concomitantly.

Both the amyloid β peptide analogue and the oligomer may be prepared using a peptide or peptidomimetic which differs from the amino acid sequence or the peptidomimetic comprised by the final amyloid β peptide analogue or oligomer. For instance, the starting peptide may comprise additional amino acids at its C- and/or N-terminus which will then be removed during the synthesis, e.g., by proteolytic cleavage.

In one embodiment of the invention, oligomers are formed with a peptide or peptidomimetic thereof and subsequently stabilized by one or more intra-peptide or intra-peptidomimetic covalent bond(s). In this embodiment, it may be expedient to use a peptide rather than a peptidomimetic.

In another embodiment of the invention, oligomers are formed with a peptide or peptidomimetic thereof, stabilized by one or more intra-peptide or intra-peptidomimetic covalent bond(s), and subsequently processed by chemical or enzymatic means to a truncated form that better displays the relevant structural elements. Alternatively, oligomers are formed with a peptide or peptidomimetic thereof, processed by chemical or enzymatic means to a truncated form that better displays relevant structural elements, and subsequently stabilized by one or more intra-peptide or intra-peptidomimetic covalent bond(s). In these embodiments, it may be expedient to use a peptide rather than a peptidomimetic.

In still another embodiment of the invention, a peptide or peptidomimetic thereof is used to form the relevant structural elements, wherein the peptide or peptidomimetic would be held in the proper conformation by one or more intra-peptide or intra-peptidomimetic covalent bond(s), rather than by interaction with adjacent peptides or peptidomimetic in an oligomer. It is envisioned that these amyloid β peptide analogues, stabilized with the appropriate intra-peptide or intra-peptidomimetic covalent bond(s), will present the relevant structural elements as a monomer. In this embodiment, it may be expedient to use a peptidomimetic rather than a peptide.

The amyloid β peptide analogues or oligomers of the invention have many utilities. For instance, they can be used in: 1) immunization-based interventional therapies (e.g., amyloid β peptide analogues or oligomers may be used in active immunization to treat or prevent an amyloidosis); 2) diagnostic testing (e.g., the amyloid β peptide analogues or oligomers may be used to diagnose an amyloidosis; 3) providing agents such as antibodies and aptamers that bind to the amyloid β peptide analogues or oligomers; and 4) crystallographic or NMR-based structure-based design research for developing agents such as antibodies and aptamers that bind to the amyloid β peptide analogues or oligomers.

The term “amyloidosis” here denotes a number of disorders characterized by abnormal folding, clumping, aggregation and/or accumulation of particular proteins (amyloids, fibrous proteins and their precursors) in various tissues of the body. In Alzheimer's disease and Down's syndrome, nerve tissue is affected, and in cerebral amyloid angiopathy (CAA) blood vessels are affected. According to a particular embodiment of the present invention, an amyloidosis is selected from the group consisting of Alzheimer's disease (AD) and the amyloidosis of Down's syndrome.

In active immunization, Aβ(20-42) truncated globulomer was shown to be effective in reversing cognitive defects in Alzheimer Disease transgenic mice. The amyloid β peptide analogues or oligomers of the present invention are able to elicit an immune response whose profil is similar to the profil of the immune response elicited by Aβ(20-42) truncated globulomer.

Thus, the invention also relates to the amyloid β peptide analogue or oligomer as defined herein for therapeutic uses.

In one aspect, the invention relates to therapeutic compositions comprising an amyloid β peptide analogue or oligomer as defined herein. According to a particular embodiment, said compositions are pharmaceutical compositions which further comprise a pharmaceutical acceptable carrier.

In a further aspect, the invention relates to the use of the amyloid β peptide analogue or oligomer as defined herein for preparing a pharmaceutical composition for treating or preventing an amyloidosis.

In a preferred embodiment of the invention, the pharmaceutical composition is a vaccine for active immunization.

Accordingly, the invention also relates to a method of treating or preventing an amyloidosis in a subject in need thereof, which comprises administering the amyloid β peptide analogue or oligomer as defined herein to the subject.

In a preferred embodiment of the invention, administering the amyloid β peptide analogue or oligomer is for actively immunizing the subject against an amyloidosis.

In the context of active immunization, it is particularly preferred if the amyloid β peptide analogue or oligomer is not able to enter the patient's CNS in significant amounts.

It is also particularly preferred if the pharmaceutical composition comprising the amyloid β peptide analogue or oligomer is capable of inducing a strong immune response against Aβ oligomers, preferably a strong immune response directed against Aβ oligomers only, more preferably a strong non-inflammatory antibody-based immune response against Aβ oligomers only. Thus, in one embodiment of the invention the pharmaceutical composition comprises an immunological adjuvant, preferably an adjuvant and a signalling molecule, e.g. a cytokine, that directs the immune response towards the non-inflammatory, antibody-based type. Such adjuvants and signalling molecules are well known to those skilled in the art.

It is particularly preferred if the pharmaceutical composition for active immunization is administered via a route selected from the group consisting of the intravenous route, the intramuscular route, the subcutaneous route, the intranasal route, and by inhalation. It is also particularly preferred if the composition is administered by a method selected from injection, bolus infusion and continuous infusion, each of which may be performed once, repeatedly or in regular intervals.

In a particular embodiment of the invention, long-term continuous infusion is achieved by employing an implantable device. In a further particular embodiment of the invention, the composition is applied as an implantable sustained release or controlled release depot formulation. Suitable formulations and devices are known to those skilled in the art. The details of the method to be used for any given route will depend on the stage and severity of the disease and the overall medical parameters of the subject and are preferably decided upon individually at the treating physician's or veterinary's discretion.

In an especially preferred embodiment of the invention, the pharmaceutical composition for active immunization comprises one or more substances selected from the group consisting of pharmaceutically acceptable preservatives, pharmaceutically acceptable colorants, pharmaceutically acceptable protective colloids, pharmaceutically acceptable pH regulators and pharmaceutically acceptable osmotic pressure regulators. Such substances are described in the art.

In line with globulomer hypothesis, it is believed that subjects suffering from an amyloidosis develop an immune response against endogenous globulomer epitopes. As the amyloid β peptide analogues or oligomers of the present invention react with antibodies that are specifically reactive with said epitopes the oligomers are believed to display the same or a very similar epitope.

The invention thus also relates to the amyloid β peptide analogue or oligomer as defined herein for diagnostic uses.

In one aspect, the invention relates to diagnostic compositions comprising an amyloid β peptide analogue or oligomer as defined herein. According to a particular embodiment, said compositions are pharmaceutical compositions which further comprise a pharmaceutical acceptable carrier.

In a further aspect, the invention relates to the use of an amyloid β peptide analogue or oligomer as defined herein for preparing a composition for diagnosing an amyloidosis.

Accordingly, the invention also relates to a method of diagnosing an amyloidosis which comprises providing a sample from the subject suspected of having the amyloidosis, contacting the sample with an amyloid β peptide analogue or oligomer as defined herein for a time and under conditions sufficient for the formation of a complex comprising the amyloid β peptide analogue or oligomer and an antibody, the presence of the complex indicating the subject has the amyloidosis. According to a particular embodiment, at least the step of contacting the sample is carried out ex vivo and in particular in vitro.

Thus, the amyloid β peptide analogue or oligomer of the present invention may be used in a variety of diagnostic methods and assays.

According to one embodiment, the method of diagnosing an amyloidosis in a patient suspected of having this disease comprises the steps of: a) isolating a biological sample from the patient; b) contacting the biological sample with an amyloid β peptide analogue or oligomer for a time and under conditions sufficient for the formation of antibody/amyloid β peptide analogue or oligomer complexes; c) adding a conjugate to the resulting antibody/amyloid β peptide analogue or oligomer complexes for a time and under conditions sufficient to allow the conjugate to bind to the bound antibody, wherein the conjugate comprises an antibody attached to a signal generating compound capable of generating a detectable signal; and d) detecting the presence of antibodies which may be present in the biological sample by detecting a signal generated by the signal generating compound, the signal indicating a diagnosis of an amyloidosis in the patient. According to a particular embodiment, at least one of steps b), c) and d) is carried out ex vivo and in particular in vitro. According to a further particular embodiment, the method does not comprise step a).

According to a further embodiment, the method of diagnosing an amyloidosis in a patient suspected of having this disease comprises the steps of: a) isolating a biological sample from the patient; b) contacting the biological sample with anti-antibody specific for antibodies in the sample for a time and under conditions sufficient to allow for formation of anti-antibody/antibody complexes; b) adding a conjugate to resulting anti-antibody/antibody complexes for a time and under conditions sufficient to allow the conjugate to bind to bound antibody, wherein the conjugate comprises an amyloid β peptide analogue or oligomer of the present invention attached to a signal generating compound capable of generating a detectable signal; and c) detecting a signal generated by the signal generating compound, the signal indicating a diagnosis of an amyloidosis in the patient. According to a particular embodiment, at least one of said steps b) and c) is carried out ex vivo and in particular in vitro. According to a further particular embodiment, the method does not comprise step a).

More specifically, as the amyloid β peptide analogues or oligomers of the present invention display the globulomer epitope and the globulomer epitope is believed to be an endogenous antigen which gives rise to an endogenous immune response, diagnosis of amyloidoses can be related to the determination of the presence of auto-antibodies which specifically bind to the amyloid β peptide analogues or oligomers of the invention.

The invention thus also relates to the use of the amyloid β peptide analogue or oligomer as defined herein for preparing a composition for detecting in a subject auto-antibodies that bind to the oligomer or derivative. Accordingly, the invention also relates to a method of detecting auto-antibodies that bind to the amyloid β peptide analogue or oligomer in a subject, which method comprises administering to the subject amyloid β peptide analogue or oligomer as defined herein and detecting a complex formed by the antibody and the amyloid β peptide analogue or oligomer, the presence of the complex indicating the presence of the auto-antibodies. In a particular embodiment of the invention, the subject is suspected of having any form of amyloidosis, e.g. Alzheimer's disease, and detecting auto-antibodies is for diagnosing the presence or absence of any form of amyloidosis, e.g. Alzheimer's disease, in the subject.

The invention also relates to the use of the amyloid β peptide analogue or oligomer as defined herein for detecting auto-antibodies that bind to the oligomer or derivative in a sample. Accordingly, the invention also refers to a method of detecting auto-antibodies that bind to the Aβ amyloid β peptide analogue or oligomer in a sample, which method comprises contacting the sample with the amyloid β peptide analogue or oligomer as defined herein and detecting a complex formed by the antibody and the amyloid β peptide analogue or oligomer, the presence of the complex indicating the presence of the auto-antibodies. According to a particular embodiment, at least the step of contacting the sample is carried out ex vivo and in particular in vitro. In a preferred embodiment of the invention, the sample is derived from a subject suspected of having an amyloidosis, e.g. Alzheimer's disease, and detecting the auto-antibodies is for diagnosing the presence or absence of the amyloidosis, e.g. Alzheimer's disease in the subject. Suitable samples include biological fluids which may be tested by the aforesaid method. These include plasma, whole blood, dried whole blood, serum, cerebrospinal fluid or aqueous or organo-aqueous extracts of tissues and cells.

It is particularly preferred if the subject suspected of having an amyloidosis is a subject having the amyloidosis or having an increased risk of getting the amyloidosis.

According to a particular embodiment of the invention, detecting auto-antibodies as described herein further comprises a pre-treatment of the preparation (sample) which causes dissociation of auto-antibody/antigen complexes. A method comprising such a pre-treatment may therefore be used in order to determine the total amount of auto-antibodies present in the preparation (sample) while a method not comprising said pre-treatment may be used in order to determine the amount of auto-antibodies which can still bind to the antigen. Further, both methods will allow to indirectly determine the amount of complexed auto-antibodies.

Conditions suitable for inducing dissociation of auto-antibody/antigen complexes are known to the skilled person. For instance, treating the preparation (sample) with acid, e.g., using a buffer such that the pH of the resulting preparation (sample) is in the range of 1 to 5, preferably in the range of 2 to 4 and in particular in the range of 2 to 3, may be expedient. Suitable buffers include salts in a physiological concentration, e.g. NaCl and acetic acid. A method for separation of antibody/antigen complexes has been described in WO2005/037209, which is incorporated herein in its entirety.

Briefly, dissociating the antibody from the antigen in the antibody/antigen complex comprises the steps of: contacting the sample containing an antibody/antigen complex with a dissociation buffer; incubating the sample; and optionally concentrating the sample.

The dissociation buffer may be a PBS buffer which has a pH in the range as indicated herein. For instance a PBS buffer containing about 1.5% BSA and 0.2 M glycine-acetate pH 2.5, or 140 mM NaCl and 0.58% acetic acid is suitable.

Incubation for several minutes, for instance such as 10 to 30, e.g., 20 minutes at a temperature in the range of 20 to 40° C. has proven sufficient.

Concentration can be achieved in a manner known per se, for instance by passing the sample over a Centriprep YM30 (Amincon Inc.).

In one embodiment of the present invention, the amyloid β peptide analogue or oligomer, or a portion thereof is coated on a solid phase. The sample (e.g., whole blood, cerebrospinal fluid, serum, etc.) is then contacted with the solid phase. If the antibodies, e.g. the auto-antibodies, are present in the sample, such antibodies bind to the amyloid β peptide analogue or oligomer on the solid phase and are then detected by either a direct or indirect method. The direct method comprises simply detecting presence of the complex itself and thus presence of the antibodies. In the indirect method, a conjugate is added to the bound antibody. The conjugate comprises a second antibody, which binds to the first bound antibodies, attached to a signal-generating compound or label. Should the second antibody bind to a bound first antibody, the signal-generating compound generates a measurable signal. Such a signal then indicates presence of the first antibodies in the sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of the solid phase material and the method of labeling the antigen or antibodies present in the conjugate, if desired, are determined based upon desired assay format performance characteristics.

As noted herein, the conjugate (or indicator reagent) will comprise an antibody (or perhaps anti-antibodies, depending upon the assay), attached to a signal-generating compound or “label”. This signal-generating compound or label is itself detectable or may be reacted with one or more additional compounds to generate a detectable product. Examples of signal-generating compounds are described herein and in particular include chromophores, radio-isotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Kits are also included within the scope of the present invention. More specifically, the present invention includes kits for determining the presence of antibodies such as auto-antibodies in a subject. In particular, a kit for determining the presence of said antibodies in a sample comprises a) a amyloid β peptide analogue or oligomer as defined herein; and optionally b) a conjugate comprising an antibody attached to a signal generating compound capable of generating a detectable signal. The kit may also contain a control or calibrator which comprises a reagent which binds to the antigen.

The present invention also includes another type of kit for detecting antibodies such as auto-antibodies in a sample. The kit may comprise a) an anti-antibody specific for the antibody of interest, and b) amyloid β peptide analogue or oligomer as defined herein. A control or calibrator comprising a reagent which binds to the amyloid β peptide analogue or oligomer may also be included. More specifically, the kit may comprise a) an anti-antibody specific for the auto-antibody and b) a conjugate comprising the amyloid β peptide analogue or oligomer, the conjugate being attached to a signal generating compound capable of generating a detectable signal. Again, the kit may also comprise a control or calibrator comprising a reagent which binds to the antigen.

The kit may also comprise one container such as a vial, bottle or strip, with each container with a pre-set solid phase, and other containers containing the respective conjugates. These kits may also contain vials or containers of other reagents needed for performing the assay, such as washing, processing and indicator reagents.

The amyloid β peptide analogues or oligomers of the invention are useful for providing agents that are capable of binding to the amyloid β peptide analogue or oligomer. Such agents include, e.g., antibodies (hereinafter also referred to as anti-oligomer antibody), non-antibody binding molecules (such as affibodies, affilin molecules, AdNectins, Anticalins, DARPins, domain antibodies, evibodies, knotins, Kunitz-type domains, maxibodies, tetranectins, trans-bodies, and V(NAR)s, as described, for instance, in the Handbook of Therapeutic Antibodies, ed. by Stefan Dübel, Volume II, Chapter 7, Wiley-VCH Verlag GmbH & Co. KGaA, Weinheim, 2007), aptamers or small-molecular weight compounds. In one aspect, the invention thus relates to the use of the amyloid β peptide analogue or oligomer for screening an agent that is capable of binding to the amyloid β peptide analogue or oligomer. Accordingly, the invention also relates to a method of identifying such agents, which method comprises the steps of: a) exposing one or more agents of interest to the amyloid β peptide analogue or oligomer described herein for a time and under conditions sufficient for the one or more agents to bind to the amyloid β peptide analogue or oligomer; and b) identifying those agents which bind to the amyloid β peptide analogue or oligomer.

In another aspect, the invention relates to the use of the amyloid β peptide analogue or oligomer for enriching an agent that is capable of binding to the amyloid β peptide analogue or oligomer in a preparation comprising said agent. Accordingly, the invention also relates to a method of enriching such an agent in a preparation comprising said agent, which method comprises the steps of: a) exposing to the amyloid β peptide analogue or oligomer the preparation comprising the agent that is capable of binding to the amyloid β peptide analogue or oligomer for a time and under conditions sufficient for the agent to bind to the amyloid β peptide analogue or oligomer; and b) obtaining the agent in enriched form. More particularly, the amyloid β peptide analogue or oligomer can be immobilized (for instance on a resin), which allows the agent to be captured. Obtaining the agent in enriched form may then comprise desorbing the captured agent, preferably in such a way that desorbing the captured agent comprises contacting the captured agent with a high salt buffer or an acidic solution. This method can, for instance, be used for enriching auto-antibodies as described herein by subjecting commercial immunoglobulin preparations like IVIG or Octagam® (Octa-pharma Inc. Vienna, Austria) to this method. It is believed that these immunoglobulin preparations contain auto-antibodies to AR, and by treating subjects one raises the level of anti-Aβ antibodies in their body. A preparation that is enriched for said auto-antibodies would be expected to be more efficacious.

In a further aspect, the invention thus relates to the use of the amyloid β peptide analogue or oligomer for providing an antibody that binds to the amyloid β peptide analogue or oligomer. Accordingly, the invention also relates to a method for providing an antibody that binds to the amyloid β peptide analogue or oligomer as defined herein, which method comprises:

a) providing an antigen comprising the amyloid β peptide analogue or oligomer;
b) exposing an antibody repertoire or potential antibody repertoire to said antigen; and
c) selecting from said repertoire an antibody which binds to said amyloid β peptide analogue or oligomer.

Here it is to be understood that a “potential antibody repertoire” refers to any library, collection, assembly or set of amino acid or corresponding nucleic acid sequences or to any generator of such a library, collection, assembly or set of amino acid sequences that can be used for producing an antibody repertoire in vivo or in vitro. In a preferred embodiment of the invention, the generator is the adaptive immune system of an animal, in particular the antigen-producing part of the immune system of a mammal which generates antibody diversity by a recombination process well known to those skilled in the art. In another preferred embodiment of the invention, the generator is a system for the spawning of random nucleic acid sequences which can then, by insertion into a suitable antibody framework, be used to produce an antibody repertoire in vitro.

In a preferred embodiment of the invention, the antibody repertoire or potential antibody repertoire is exposed to the antigen in vivo by immunizing an organism with said antigen. In another preferred embodiment of the invention, the potential antibody repertoire is a library of suitable nucleic acids which is exposed to the antibody by in vitro affinity screening as described in the art, e.g. a phage display and panning system.

In another aspect, the invention also provides antibodies that bind to the Aβ(X-38 . . . 43) amyloid β peptide analogue or oligomer as defined herein.

In a preferred embodiment of the invention, the antibody is obtainable by a method comprising selecting the antibody from a repertoire or potential repertoire as described herein.

According to a particularly preferred embodiment, the present invention provides amyloid β peptide analogue- or oligomer-specific antibodies. These include in particular antibodies having a comparatively smaller affinity for both the monomeric and fibrillomeric forms of Aβ peptide than for the amyloid β peptide analogue or oligomer of the invention. In certain embodiments, an antibody is said to specifically bind an antigen when it preferentially recognizes its target antigen in a complex mixture of proteins and/or macromolecules.

In a preferred embodiment of the invention, the affinity of the antibody to the amyloid β peptide analogue or oligomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a monomeric Aβ(1-42).

In a preferred embodiment of the invention, the affinity of the antibody to the amyloid β peptide analogue or oligomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a monomeric Aβ(1-40).

Expediently, the antibody of the present invention binds to one or, more preferably, both monomers with low affinity, most preferably with a K_{D} of 1×10⁻⁸ M or smaller affinity, e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁶ M or smaller affinity, e.g. with a K_{D} of 3×10⁻⁵ M or smaller affinity, or with a K_{D} of 1×10⁻⁵ M or smaller affinity.

In a preferred embodiment of the invention, the affinity of the antibody to the amyloid β peptide analogue or oligomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a fibrillomeric Aβ(1-42).

In a preferred embodiment of the invention, the affinity of the antibody to the amyloid β peptide analogue or oligomer is at least 2 times, e.g. at least 3 times or at least 5 times, preferably at least 10 times, e.g. at least 20 times, at least 30 times or at least 50 times, more preferably at least 100 times, e.g. at least 200 times, at least 300 times or at least 500 times, and even more preferably at least 1000 times, e.g. at least 2000 times, at least 3000 times or at least 5000 times, even more preferably at least 10000 times, e.g. at least 20000 times, at least 30000 or at least 50000 times, and most preferably at least 100000 times greater than the binding affinity of the antibody to a fibrillomeric Aβ(1-40).

Expediently, the antibody of the present invention binds to one or, more preferably, both fibrils with low affinity, most preferably with a K_{D} of 1×10⁻⁸ M or smaller affinity, e.g. with a K_{D} of 3×10⁻⁸ M or smaller affinity, with a K_{D} of 1×10⁻⁷ M or smaller affinity, e.g. with a K_{D} of 3×10⁻⁷ M or smaller affinity, or with a K_{D} of 1×10⁻⁶ M or smaller affinity, e.g. with a K_{D} of 3×10⁻⁵ M or smaller affinity, or with a K_{D} of 1×10⁻⁵ M or smaller affinity.

The antibodies of the present invention are preferably isolated antibodies. An “isolated antibody” means an antibody having the binding affinities as described above and which is essentially free of other antibodies having different binding affinities. The term “essentially free” here refers to an antibody preparation in which at least 95% of the antibodies, preferably at least 98% of the antibodies and more preferably at least 99% of the antibodies have the desired binding affinity. Moreover, an isolated antibody may be substantially free of other cellular material and/or chemicals.

The isolated antibodies of the present invention include monoclonal antibodies. A “monoclonal antibody” as used herein is intended to refer to a preparation of antibody molecules, antibodies which share a common heavy chain and common light chain amino acid sequence, in contrast with “polyclonal” antibody preparations which contain a mixture of antibodies of different amino acid sequence. Monoclonal antibodies can be generated by several novel technologies like phage, bacteria, yeast or ribosomal display, as well as by classical methods exemplified by hybridoma-derived antibodies (e.g., an antibody secreted by a hybridoma prepared by hybridoma technology, such as the standard Kohler and Milstein hybridoma methodology ((1975) Nature 256:495-497). Thus, a non-hybridoma-derived antibody with uniform sequence is still referred to as a monoclonal antibody herein although it may have been obtained by non-classical methodologies, and the term “monoclonal” is not restricted to hybridoma-derived antibodies but used to refer to all antibodies derived from one nucleic acid clone.

Thus, the monoclonal antibodies of the present invention include recombinant antibodies. The term “recombinant” herein refers to any artificial combination of two otherwise separated segments of sequence, e.g., by chemical synthesis or by the manipulation of isolated segments of nucleic acids by genetic engineering techniques. In particular, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g. a mouse) which is transgenic due to human immunoglobulin genes (see, for example, Taylor, L. D., et al. (1992) Nucl. Acids Res. 20:6287-6295); or antibodies which are produced, expressed, generated or isolated in any other way in which particular immunoglobulin gene sequences (such as human immunoglobulin gene sequences) are assembled with other DNA sequences. Recombinant antibodies include, for example, chimeric, CDR graft and humanized antibodies. The person skilled in the art will be aware that expression of a conventional hybridoma-derived monoclonal antibody in a heterologous system will require the generation of a recombinant antibody even if the amino acid sequence of the resulting antibody protein is not changed or intended to be changed.

In a particular embodiment of the invention, the antibody is a humanized antibody.

According to a multiplicity of embodiments, the antibody may comprise an amino acid sequence derived entirely from a single species, such as a human antibody or a mouse antibody. According to other embodiments, the antibody may be a chimeric antibody or a CDR graft antibody or another form of a humanized antibody.

The term “antibody” is intended to refer to immunoglobulin molecules consisting of 4 polypeptide chains, two heavy (H) chains and two light (L) chains. The chains are usually linked to one another via disulfide bonds. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

The term “antigen-binding moiety” of an antibody (or simply “antibody moiety”) refers to one or more fragments of an antibody of the invention, said fragment(s) still having the binding affinities as defined above. Fragments of a complete antibody have been shown to be able to carry out the antigen-binding function of an antibody. In accordance with the term “antigen-binding moiety” of an antibody, examples of binding fragments include (i) an Fab fragment, i.e. a monovalent fragment composed of the VL, VH, CL and CH1 domains; (ii) an F(ab′)₂ fragment, i.e. a bivalent fragment comprising two Fab fragments linked to one another in the hinge region via a disulfide bridge; (iii) an Fd fragment composed of the VH and CH1 domains; (iv) an Fv fragment composed of the FL and VH domains of a single arm of an antibody; (v) a dAb fragment (Ward et al., (1989) Nature 341:544-546) consisting of a VH domain or of VH, CH1, CH2, DH3, or VH, CH2, CH3; and (vi) an isolated complementarity-determining region (CDR). Although the two domains of the Fv fragment, namely VL and VH, are encoded by separate genes, they may further be linked to one another using a synthetic linker, e.g. a poly-G₄S amino acid sequence, and recombinant methods, making it possible to prepare them as a single protein chain in which the VL and VH regions combine in order to form monovalent molecules (known as single chain Fv (ScFv); see, for example, Bird et al. (1988) Science 242:423-426; and Huston et al. (1988) Proc. Natl. Acad. Sci. USA 85:5879-5883). The term “antigen-binding moiety” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites (see, for example, Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art.

Furthermore, an antibody of the present invention or antigen-binding moiety thereof may be part of a larger immunoadhesion molecule formed by covalent or noncovalent association of said antibody or antibody-binding moiety with one or more further proteins or peptides. Relevant to such immunoadhesion molecules are the use of the streptavidin core region in order to prepare a tetrameric scFv molecule (Kipriyanov, S. M., et al. (1995) Human Antibodies and Hybridomas 6:93-101) and the use of a cysteine residue, a marker peptide and a C-terminal polyhistidinyl, e.g. hexahistidinyl, tag in order to produce bivalent and biotinylated scFv molecules (Kipriyanov, S. M., et al. (1994) Mol. Immunol. 31:1047-1058).

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies of the invention may contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies of the invention have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody.

The term “chimeric antibody” refers to antibodies which contain sequences for the variable region of the heavy and light chains from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which contain sequences of the variable region of heavy and light chains from a nonhuman species (e.g. mouse, rat, rabbit, chicken, camelid, sheep or goat) but in which at least one part of the VH and/or VL sequence has been altered in order to be more “human-like”, i.e. to be more similar to variable sequences of the human germ line. One type of a humanized antibody is a CDR graft antibody in which human CDR sequences have been inserted into nonhuman VH and VL sequences to replace the corresponding nonhuman CDR sequences.

Methods of producing antibodies of the invention are described below. A distinction is made here between in-vivo approaches, in-vitro approaches or a combination of both.

Some methods of producing antibodies of the invention are described below. A distinction is made here between in-vivo approaches, in-vitro approaches or a combination of both.

### In-Vivo Approaches

Depending on the type of the desired antibody, various host animals may be used for in-vivo immunization. A host expressing itself an endogenous version of the antigen of interest may be used. Alternatively, it is possible to use a host which has been made deficient in an endogenous version of the antigen of interest. For example, mice which had been made deficient in a particular endogenous protein via homologous recombination at the corresponding endogenous gene (i.e. knockout mice) have been shown to generate a humoral response to the protein with which they have been immunized and therefore to be able to be used for production of high-affinity monoclonal antibodies to the protein (see, for example, Roes, J. et al. (1995) J. Immunol. Methods 183:231-237; Lunn, M. P. et al. (2000) J. Neurochem. 75:404-412).

A multiplicity of nonhuman mammals are suitable hosts for antibody production in order to produce nonhuman antibodies of the invention. They include mice, rats, chickens, camelids, rabbits, sheep and goats (and knockout versions thereof), although preference is given to mice for the production of hybridomas. Furthermore, a nonhuman host animal expressing a human antibody repertoire may be used for producing essentially human antibodies to a human antigen with dual specificity. Nonhuman animals of this kind include transgenic animals (e.g. mice) bearing human immunoglobulin transgenes (chimeric hu-PBMC SCID mice) and human/mouse irradiation chimeras which are described in more detail below.

According to one embodiment, the animal immunized with amyloid β peptide analogue or oligomer is a nonhuman mammal, preferably a mouse, which is transgenic due to human immunoglobulin genes so that said nonhuman mammal makes human antibodies upon anti-genic stimulation. Typically, immunoglobulin transgenes for heavy and light chains with human germ line configuration are introduced into such animals which have been altered such that their endogenous heavy and light chain loci are inactive. If such animals are stimulated with antigen (e.g. with a human antigen), antibodies derived from the human immunoglobulin sequences (human antibodies) are produced. It is possible to make from the lymphocytes of such animals human monoclonal antibodies by means of standardized hybridoma technology. For a further description of transgenic mice with human immunoglobulins and their use in the production of human antibodies, see, for example, U.S. Pat. No. 5,939,598, WO 96/33735, WO 96/34096, WO 98/24893 and WO 99/53049 (Abgenix Inc.), and U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, U.S. Pat. No. 5,814,318, U.S. Pat. No. 5,877,397 and WO 99/45962 (Genpharm Inc.); see also MacQuitty, J. J. and Kay, R. M. (1992) Science 257:1188; Taylor, L. D. et al. (1992) Nucleic Acids Res. 20:6287-6295; Lonberg, N. et al. (1994) Nature 368:856-859; Lonberg, N. and Huszar, D. (1995) Int. Rev. Immunol. 13:65-93; Harding, F. A. and Lonberg, N. (1995) Ann. N.Y. Acad. Sci. 764:536-546; Fishwild, D. M. et al. (1996) Nature Biotechnology 14:845-851; Mendez, M. J. et al. (1997) Nature Genetics 15:146-156; Green, L. L. and Jakobovits, A. (1998) J. Exp. Med. 188:483-495; Green, L. L. (1999) J. Immunol. Methods 231:11-23; Yang, X. D. et al. (1999) J. Leukoc. Biol. 66:401-410; Gallo, M. L. et al. (2000) Eur. J. Immunol. 30:534-540.

According to another embodiment, the animal which is immunized with the amyloid β peptide analogue or oligomer may be a mouse with severe combined immunodeficiency (SCID), which has been reconstituted with human peripheral mononuclear blood cells or lymphoid cells or precursors thereof. Such mice which are referred to as chimeric hu-PBMC SCID mice produce human immunoglobulin responses upon antigenic stimulation, as has been proved. For a further description of these mice and of their use for generating antibodies, see, for example, Leader, K. A. et al. (1992) Immunology 76:229-234; Bombil, F. et al. (1996) Immunobiol. 195:360-375; Murphy, W. J. et al. (1996) Semin. Immunol. 8:233-241; Herz, U. et al. (1997) Int. Arch. Allergy Immunol. 113:150-152; Albert, S. E. et al. (1997) J. Immunol. 159:1393-1403; Nguyen, H. et al. (1997) Microbiol. Immunol. 41:901-907; Arai, K. et al. (1998) J. Immunol. Methods 217:79-85; Yoshinari, K. and Arai, K. (1998) Hybridoma 17:41-45; Hutchins, W. A. et al. (1999) Hybridoma 18:121-129; Murphy, W. J. et al. (1999) Clin. Immunol. 90:22-27; Smithson, S. L. et al. (1999) Mol. Immunol. 36:113-124; Chamat, S. et al. (1999) J. Infect. Diseases 180:268-277; and Heard, C. et al. (1999) Molec. Med. 5:35-45.

According to another embodiment, the animal which is immunized with the amyloid β peptide analogue or oligomer is a mouse which has been treated with a lethal dose of total body irradiation, then protected from radiation with bone marrow cells from mice with severe combined immunodeficiency (SCID) and subsequently transplanted with functional human lymphocytes. This type of chimera, referred to as the Trimera system, is used in order to produce human monoclonal antibodies by immunizing said mice with the antigen of interest and then producing monoclonal antibodies by using standardized hybridoma technology. For a further description of these mice and of their use for generating antibodies, see, for example, Eren, R. et al. (1998) Immunology 93:154-161; Reisner, Y. and Dagan, S. (1998) Trends Biotechnol. 16:242-246; Ilan, E. et al. (1999) Hepatology 29:553-562; and Bocher, W. O. et al. (1999) Immunology 96:634-641.

Starting from the in-vivo generated antibody-producing cells, monoclonal antibodies may be produced by means of standardized techniques such as the hybridoma technique originally described by Kohler and Milstein (1975, Nature 256:495-497) (see also Brown et al. (1981) J. Immunol. 127:539-46; Brown et al. (1980) J Biol Chem 255:4980-83; Yeh et al. (1976) PNAS 76:2927-31; and Yeh et al. (1982) Int. J. Cancer 29:269-75). The technology of producing monoclonal antibody hybridomas is sufficiently known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36). Briefly, an immortalized cell line (typically a myeloma) is fused with lymphocytes (typically splenocytes or lymph node cells or peripheral blood lymphocytes) of a mammal immunized with the amyloid β peptide analogue or oligomer of the invention, and the culture supernatants of the resulting hybridoma cells are screened in order to identify a hybridoma which produces a monoclonal antibody of the present invention. Any of the many well known protocols for fusing lymphocytes and immortalized cell lines can be applied for this purpose (see also G. Galfre et al. (1977) Nature 266:550-52; Gefter et al. Somatic Cell Genet., cited supra; Lerner, Yale J. Biol. Med., cited supra; Kenneth, Monoclonal Antibodies, cited supra). Moreover, the skilled worker will appreciate that there are diverse variations of such methods, which are likewise useful. Typically, the immortalized cell line (e.g. a myeloma cell line) is derived from the same mammalian species as the lymphocytes. For example, murine hybridomas may be established by fusing lymphocytes from a mouse immunized with an immunogenic preparation of the invention with an immortalized mouse cell line. Preferred immortalized cell lines are mouse myeloma cell lines which are sensitive to culture medium containing hypoxanthine, aminopterin and thymidine (HAT medium). Any of a number of myeloma cell lines may be used by default as fusion partner, for example the P3-NS1/1-Ag4-1, P3-X63-Ag8.653 or Sp2/O—Ag14 myeloma lines. These myeloma cell lines are available from the American Type Culture Collection (ATCC), Rockville, Md. Typically, HAT-sensitive mouse myeloma cells are fused to mouse splenocytes using polyethylene glycol (PEG). Hybridoma cells resulting from the fusion are then selected using HAT medium, thereby killing unfused and unproductively fused myeloma cells (unfused splenocytes die after several days because they are not transformed). Hybridoma cells producing monoclonal antibodies of the invention are identified by screening the hybridoma culture supernatants for such antibodies, for example by using a dot blot assay in order to select those antibodies which have the binding affinities as defined herein.

Likewise, said hybridoma can be used as a source of nucleic acid encoding light and/or heavy chains in order to recombinantly produce antibodies of the present invention, as is described below in further detail.

### In-Vitro Approaches

As an alternative to producing antibodies of the invention by immunization and selection, antibodies of the invention may be identified and isolated by screening recombinant combinatorial immunoglobulin libraries with the amyloid β peptide analogue or oligomer to thereby isolate immunoglobulin library members which have the required binding affinity. Kits for generating and screening display libraries are commercially available (e.g. the Pharmacia Recombinant Phage Antibody System, catalog No. 27-9400-01; and the Stratagene SurfZAP® Phage Display Kit, catalog No. 240612). In many embodiments, the display library is an scFv library or an Fab library. The phage display technique for screening recombinant antibody libraries has been adequately described. Examples of methods and compounds which can be used particularly advantageously for generating and screening antibody display libraries can be found, for example, in McCafferty et al. WO 92/01047, U.S. Pat. No. 5,969,108 and EP 589 877 (describes in particular scFv display), Ladner et al. U.S. Pat. No. 5,223,409, U.S. Pat. No. 5,403,484, U.S. Pat. No. 5,571,698, U.S. Pat. No. 5,837,500 and EP 436 597 (describes pill fusion, for example); Dower et al. WO 91/17271, U.S. Pat. No. 5,427,908, U.S. Pat. No. 5,580,717 and EP 527 839 (describes in particular Fab display); Winter et al. International Publication WO 92/20791 and EP 368,684 (describes in particular the cloning of sequences for variable immunoglobulin domains); Griffiths et al. U.S. Pat. No. 5,885,793 and EP 589 877 (describes in particular isolation of human antibodies to human antigens by using recombinant libraries); Garrard et al. WO 92/09690 (describes in particular phage expression techniques); Knappik et al. WO 97/08320 (describes the human recombinant antibody library HuCal); Salfeld et al. WO 97/29131, (describes production of a recombinant human antibody to a human antigen (human tumor necrosis factor alpha) and also in-vitro affinity maturation of the recombinant antibody) and Salfeld et al. U.S. Provisional Application No. 60/126,603 and the patent applications based hereupon (likewise describes production of recombinant human antibodies to human antigen (human interleukin-12), and also in-vitro affinity maturation of the recombinant antibody).

Further descriptions of screenings of recombinant antibody libraries can be found in scientific publications such as Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clarkson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; Barbas et al. (1991) PNAS 88:7978-7982; McCafferty et al. Nature (1990) 348:552-554; and Knappik et al. (2000) J. Mol. Biol. 296:57-86.

As an alternative to using bacteriophage display systems, recombinant antibody libraries may be expressed on the surface of yeast cells or of bacterial cells. WO 99/36569 describes methods of preparing and screening libraries expressed on the surface of yeast cells. WO 98/49286 describes in more detail methods of preparing and screening libraries expressed on the surface of bacterial cells.

In all in vitro approaches, a selection process for enriching recombinant antibodies with the desired properties form an integral part of the process, which is generally referred to as “panning” and often takes the form of affinity chromatography over columns to whose matrix the target structure has been attached. Promising candidate molecules are then subjected to individual determination of their absolute and/or relative affinities, preferably by means of a standardized dot blot assay, as described above.

As may be appreciated by skilled workers, such in vitro methods for selection and enrichment may also be applied towards obtaining non-immunoglobulin related antigen-binding moieties.

Once an antibody of interest of a combinatorial library has been identified and sufficiently characterized, the DNA sequences encoding the light and heavy chains of said antibody are isolated by means of standardized molecular-biological techniques, for example by means of PCR amplification of DNA from the display package (e.g. the phage) which has been isolated during library screening. Nucleotide sequences of genes for light and heavy antibody chains, which may be used for preparing PCR primers, are known to the skilled worker. A multiplicity of such sequences are described, for example, in Kabat, E. A., et al. (1991) Sequences of Proteins of Immunological Interest, Fifth Edition, U.S. Department of Health and Human Services, NIH Publication No. 91-3242 and in the database of sequences of the human germ line VBASE.

An antibody or antibody-binding moiety of the invention may be produced by recombinantly expressing the genes for light and heavy immunoglobulin chains in a host cell. In order to recombinantly express an antibody, a host cell is transfected with one or more recombinant expression vectors carrying DNA fragments encoding the light and heavy immunoglobulin chains of said antibody, thereby expressing the light and heavy chains in the host cell and secreting them preferably into the medium in which said host cells are cultured. The antibodies can be isolated from this medium. Standardized recombinant DNA methods are used in order to obtain genes for heavy and light antibody chains, to insert said genes into recombinant expression vectors and to introduce said vectors into host cells. Methods of this kind are described, for example, in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

Once DNA fragments encoding VH and VL segments of the antibody of interest have been obtained, said DNA fragments may be further manipulated using standardized recombinant DNA techniques, for example in order to convert the genes for variable regions to genes for full length antibody chains, to genes for Fab fragments or to an scFv gene. These manipulations comprise linking a VL- or VH-encoding DNA fragment operatively to another DNA fragment encoding another protein, for example a constant antibody region or a flexible linker. The term “operatively linked” is to be understood here as meaning that the two DNA fragments are linked in such a way that the amino acid sequences encoded by said two DNA fragments remain in frame.

The isolated DNA encoding the VH region may be converted to a gene for a full length heavy chain by operatively linking the VH-region encoding DNA with another DNA molecule encoding heavy chain constant regions (CH1, CH2 and CH3). The sequences of human heavy chain constant region genes are well known (see, for example, Kabat, E. A., et al. (1991) Sequences of Proteins of Immunological Interest, Fifth Edition, U.S. Department of Health and Human Services, NIH Publication No. 91-3242), and DNA fragments spanning said regions may be obtained by means of standardized PCR amplification. The heavy chain constant region may be a constant region from IgG1, IgG2, IgG3, IgG4, IgM, IgA, IgE or IgD, with preference being given to a constant region from IgG, in particular IgG1 or IgG4. To obtain a gene for a heavy chain Fab fragment, the VH-encoding DNA may be operatively linked to another DNA molecule encoding merely the heavy chain constant region CH1.

The isolated DNA encoding the VL region may be converted to a gene for a full length light chain (and a gene for an Fab light chain) by operatively linking the VL-encoding DNA to another DNA molecule encoding the light chain constant region CL. The sequences of genes of the constant region of human light chain are well known (see Kabat, E. A., et al. (1991) Sequences of Proteins of Immunological Interest, Fifth Edition, U.S. Department of Health and Human Services, NIH Publication No. 91-3242), and DNA fragments spanning said regions may be obtained by means of standardized PCR amplification. The light chain constant region may be a constant kappa or lambda region, a constant kappa region being preferred.

In order to generate an scFv gene, the VH- and VL-encoding DNA fragments may be operatively linked to another fragment encoding a flexible linker, for example the amino acid sequence (Gly₄-Ser)₃ so that the VH and VL sequences are expressed as a continuous single-chain protein, with the VL and VH regions being linked to one another via said flexible linker (see Bird et al. (1988) Science 242:423-426; Huston et al. (1988) Proc. Natl. Acad. Sci. USA 85:5879-5883; McCafferty et al., Nature (1990) 348:552-554).

Single domain VH and VL having the binding affinities as described above may be isolated from single domain libraries by the above-described methods. Two VH single-domain chains (with or without CH1) or two VL chains or a pair of one VH chain and one VL chain with the desired binding affinity may be useful as described herein for the antibodies of the invention.

In order to express the recombinant antibodies or antibody moieties of the invention, the DNAs encoding partial or full length light and heavy chains may be inserted into expression vectors so as to operatively link the genes to appropriate transcriptional and translational control sequences. In this context, the term “operatively linked” is to be understood as meaning that an antibody gene is ligated in a vector in such a way that transcriptional and translational control sequences within the vector fulfill their intended function of regulating transcription and translation of said antibody gene.

Expediently, the expression vector and the expression control sequences are chosen so as to be compatible with the expression host cell used. The gene for the antibody light chain and the gene for the antibody heavy chain may be inserted into separate vectors or both genes are inserted into the same expression vector, this being the usual case. The antibody genes are inserted into the expression vector by means of standardized methods (for example by ligation of complementary restriction cleavage sites on the antibody gene fragment and the vector, or by ligation of blunt ends, if no restriction cleavage sites are present). The expression vector may already carry sequences for antibody constant regions prior to insertion of the sequences for the light and heavy chains. For example, one approach is to convert the VH and VL sequences to full length antibody genes by inserting them into expression vectors already encoding the heavy and, respectively, light chain constant regions, thereby operatively linking the VH segment to the CH segment(s) within the vector and also operatively linking the VL segment to the CL segment within the vector.

Additionally or alternatively, the recombinant expression vector may encode a signal peptide which facilitates secretion of the antibody chain from the host cell. The gene for said antibody chain may be cloned into the vector, thereby linking the signal peptide in frame to the N terminus of the gene for the antibody chain. The signal peptide may be an immunoglobulin signal peptide or a heterologous signal peptide (i.e. a signal peptide from a non-immunoglobulin protein). In addition to the genes for the antibody chain, the expression vectors of the invention may have regulatory sequences controlling expression of the genes for the antibody chain in a host cell.

The term “regulatory sequence” is intended to include promoters, enhancers and further expression control elements (e.g. polyadenylation signals) which control transcription or translation of the genes for the antibody chain. Regulatory sequences of this kind are described, for example, in Goeddel; Gene Expression Technology: Methods in Enzymology 185, Academic Press, San Diego, Calif. (1990). The skilled worker will appreciate that the expression vector design which includes selection of regulatory sequences may depend on factors such as the choice of the host cell to be transformed, the desired strength of expression of the protein, etc. Preferred regulatory sequences for expression in mammalian host cells include viral elements resulting in strong and constitutive protein expression in mammalian cells, such as promoters and/or enhancers derived from cytomegalovirus (CMV) (such as the CMV promoter/enhancer), simian virus 40 (SV40) (such as the SV40 promoter/enhancer), adenovirus (e.g. the adenovirus major late promoter (AdMLP)) and polyoma. For a further description of viral regulatory elements and sequences thereof, see, for example, U.S. Pat. No. 5,168,062 to Stinski, U.S. Pat. No. 4,510,245 to Bell et al. and U.S. Pat. No. 4,968,615 to Schaffner et al.

Apart from the genes for the antibody chain and the regulatory sequences, the recombinant expression vectors of the invention may have additional sequences such as those which regulate replication of the vector in host cells (e.g. origins of replication) and selectable marker genes. The selectable marker genes facilitate the selection of host cells into which the vector has been introduced (see, for example, U.S. Pat. Nos. 4,399,216, 4,634,665 and 5,179,017, all to Axel et al.). For example, it is common for the selectable marker gene to render a host cell into which the vector has been inserted resistant to cytotoxic drugs such as G418, hygromycin or methotrexate. Preferred selectable marker genes include the gene for dihydrofolate reductase (DHFR) (for use in dhfr⁻ host cells with methotrexate selection/amplification) and the neo gene (for G418 selection).

For expression of the light and heavy chains, the expression vector(s) encoding said heavy and light chains is(are) transfected into a host cell by means of standardized techniques. The various forms of the term “transfection” are intended to comprise a multiplicity of techniques customarily used for introducing exogenous DNA into a prokaryotic or eukaryotic host cell, for example electroporation, calcium phosphate precipitation, DEAE-dextran transfection, and the like. Although it is theoretically possible to express the antibodies of the invention either in prokaryotic or eukaryotic host cells, preference is given to expressing the antibodies in eukaryotic cells and, in particular, in mammalian host cells, since the probability of a correctly folded and immunologically active antibody being assembled and secreted is higher in such eukaryotic cells and in particular mammalian cells than in prokaryotic cells. Prokaryotic expression of antibody genes has been reported as being ineffective for production of high yields of active antibody (Boss, M. A. and Wood, C. R. (1985) Immunology Today 6:12-13).

Preferred mammalian host cells for expressing recombinant antibodies of the invention include CHO cells (including dhfr⁻ CHO cells described in Urlaub and Chasin, (1980) Proc. Natl. Acad. Sci. USA 77:4216-4220, which are used together with a DHFR-selectable marker, as described, for example, in R. J. Kaufman and P. A. Sharp (1982) Mol. Biol. 159:601-621), NS0 myeloma cells, COS cells and SP2 cells. When introducing recombinant expression vectors encoding the antibody genes into mammalian host cells, the antibodies are produced by culturing the host cells until the antibody is expressed in said host cells or, preferably, the antibody is secreted into the culture medium in which the host cells grow. The antibodies may then be isolated from the culture medium by using standardized protein purification methods.

It is likewise possible to use host cells in order to produce moieties of intact antibodies, such as Fab fragments or scFv molecules. Variations of the above-described procedure are of course included in the invention. For example, it may be desirable to transfect a host cell with DNA encoding either the light chain or the heavy chain (but not both) of an antibody of the invention. If either light or heavy chains are present which are not required for binding of the antigen of interest, then the DNA encoding either such a light or such a heavy chain or both may be removed partially or completely by means of recombinant DNA technology. Molecules expressed by such truncated DNA molecules are likewise included in the antibodies of the invention. In addition, it is possible to produce bifunctional antibodies in which a heavy chain and a light chain are an antibody of the invention and the other heavy chain and the other light chain have specificity for an antigen different from the antigen of interest, by crosslinking an antibody of the invention to a second antibody by means of standardized chemical methods.

In a preferred system for recombinant expression of an antibody of the invention or an antigen-binding moiety thereof, a recombinant expression vector encoding both the antibody heavy chain and the antibody light chain is introduced into dhfr⁻ CHO cells by means of calcium phosphate-mediated transfection. Within the recombinant expression vector, the genes for the heavy and light antibody chains are in each case operatively linked to regulatory CMV enhancer/AdMLP-promoter elements in order to effect strong transcription of said genes. The recombinant expression vector also carries a DHFR gene which can be used for selecting dhfr⁻ CHO cells transfected with the vector by using methotrexate selection/amplification. The selected transformed host cells are cultured so that the heavy and light antibody chains are expressed, and intact antibody is isolated from the culture medium. Standardized molecular-biological techniques are used in order to prepare the recombinant expression vector, to transfect the host cells, to select the transformants, to culture said host cells, and to obtain the antibody from the culture medium. Thus the invention also provides a method of synthesizing a recombinant antibody of the invention by culturing a host cell of the invention in a suitable culture medium until a recombinant antibody of the invention has been synthesized. The method may furthermore comprise isolating said recombinant antibody from said culture medium.

As an alternative to screening recombinant antibody libraries by phage display, other methods known to the skilled worker may be used for screening large combinatorial libraries to identify the antibodies of the invention. Basically, any expression system in which a close physical linkage between a nucleic acid and the antibody encoded thereby is established and may be used to select a suitable nucleic acid sequence by virtue of the properties of the antibody it encodes may be employed.

In one type of an alternative expression system, the recombinant antibody library is expressed in the form of RNA-protein fusions, as described in WO 98/31700 to Szostak and Roberts, and in Roberts, R. W. and Szostak, J. W. (1997) Proc. Natl. Acad. Sci. USA 94:12297-12302. In this system, in-vitro translation of synthetic mRNAs carrying on their 3′ end puromycin, a peptidyl acceptor antibiotic, generates a covalent fusion of an mRNA and the peptide or protein encoded by it. Thus a specific mRNA of a complex mixture of mRNAs (e.g. a combinatorial library) may be concentrated on the basis of the properties of the encoded peptide or protein (e.g. of the antibody or a moiety thereof), such as binding of said antibody or said moiety thereof to the amyloid β peptide analogue or oligomer. Nucleic acid sequences which encode antibodies or moieties thereof and which are obtained by screening of such libraries may be expressed by recombinant means in the above-described manner (e.g. in mammalian host cells) and may, in addition, be subjected to further affinity maturation by either screening in further rounds mRNA-peptide fusions, introducing mutations into the originally selected sequence(s), or using other methods of in-vitro affinity maturation of recombinant antibodies in the above-described manner.

### Combinations of In-Vivo and In-Vitro Approaches

The antibodies of the invention may likewise be produced by using a combination of in-vivo and in-vitro approaches such as methods in which the amyloid β peptide analogue or oligomer is first allowed to act on an antibody repertoire in a host animal in vivo to stimulate production of amyloid β peptide analogue- or oligomer-binding antibodies and then further antibody selection and/or antibody maturation (i.e. optimization) are accomplished with the aid of one or more in-vitro techniques. According to one embodiment, a combined method of this kind may comprise firstly immunizing a nonhuman animal (e.g. a mouse, rat, rabbit, chicken, camelid, sheep or goat or a transgenic version thereof or a chimeric mouse) with said oligomer or derivative to stimulate an antibody response to the antigen and then preparing and screening a phage display antibody library by using immunoglobulin sequences of lymphocytes which have been stimulated in vivo by the action of said oligomer or derivative. The first step of this combined procedure may be carried out in the manner described above in connection with the in-vivo approaches, while the second step of this procedure may be carried out in the manner described above in connection with the in-vitro approaches. Preferred methods of hyperimmunizing nonhuman animals with subsequent in-vitro screening of phage display libraries prepared from said stimulated lymphocytes include those described by BioSite Inc., see, for example, WO 98/47343, WO 91/17271, U.S. Pat. No. 5,427,908 and U.S. Pat. No. 5,580,717.

According to another embodiment, a combined method comprises firstly immunizing a non-human animal (e.g. a mouse, rat, rabbit, chicken, camelid, sheep, goat or a knockout and/or transgenic version thereof, or a chimeric mouse) with an amyloid β peptide analogue or oligomer of the invention to stimulate an antibody response to said amyloid β peptide analogue or oligomer and selecting the lymphocytes which produce the antibodies having the desired specificity by screening hybridomas (prepared, for example, from the immunized animals). The genes for the antibodies or single domain antibodies are isolated from the selected clones (by means of standardized cloning methods such as reverse transcriptase polymerase chain reaction) and subjected to in-vitro affinity maturation in order to improve thereby the binding properties of the selected antibody or the selected antibodies. The first step of this procedure may be conducted in the manner described above in connection with the in-vivo approaches, while the second step of this procedure may be conducted in the manner described above in connection with the in-vitro approaches, in particular by using methods of in-vitro affinity maturation, such as those described in WO 97/29131 and WO 00/56772.

In a further combined method, the recombinant antibodies are generated from individual isolated lymphocytes by using a procedure which is known to the skilled worker as selected lymphocyte antibody methods (SLAM) and which is described in U.S. Pat. No. 5,627,052, WO 92/02551 and Babcock, J. S. et al. (1996) Proc. Natl. Acad. Sci. USA 93:7843-7848. In this method, a nonhuman animal (e.g. a mouse, rat, rabbit, chicken, camelid, sheep, goat, or a transgenic version thereof, or a chimeric mouse) is firstly immunized in vivo with the amyloid β peptide analogue or oligomer to stimulate an immune response to said amyloid β peptide analogue or oligomer, and then individual cells secreting antibodies of interest are selected by using an antigen-specific haemolytic plaque assay. To this end, the globulomer or derivative thereof or structurally related molecules of interest may be coupled to sheep erythrocytes, using a linker such as biotin, thereby making it possible to identify individual cells secreting antibodies with suitable specificity by using the haemolytic plaque assay. Following the identification of cells secreting antibodies of interest, cDNAs for the variable regions of the light and heavy chains are obtained from the cells by reverse transcriptase PCR, and said variable regions may then be expressed in association with suitable immunoglobulin constant regions (e.g. human constant regions) in mammalian host cells such as COS or CHO cells. The host cells transfected with the amplified immunoglobulin sequences derived from in vivo-selected lymphocytes may then be subjected to further in-vitro analysis and in-vitro selection by spreading out the transfected cells, for example, in order to isolate cells expressing antibodies with the binding affinity. The amplified immunoglobulin sequences may furthermore be manipulated in vitro.

Antibodies having the required affinities defined herein can be selected by performing a dot blot essentially as described above. Briefly, the antigen is attached to a solid matrix, preferably dotted onto a nitrocellulose membrane, in serial dilutions. The immobilized antigen is then contacted with the antibody of interest followed by detection of the latter by means of an enzyme-conjugated secondary antibody and a colorimetric reaction; at defined antibody and antigen concentrations, the amount of antibody bound allows affinity determination. Thus the relative affinity of two different antibodies to one target, or of one antibody to two different targets, is here defined as the relation of the respective amounts of target-bound antibody observed with the two antibody-target combinations under otherwise identical dot blot conditions.

Antibody moieties such as Fab and F(ab′)₂ fragments may be produced from whole antibodies by using conventional techniques such as digestion with papain or pepsin. In addition, antibodies, antibody moieties and immunoadhesion molecules may be obtained by using standardized recombinant DNA techniques.

In a further aspect, the invention also relates to the use of the amyloid β peptide analogue or oligomer of the invention for providing an aptamer that binds to the amyloid β peptide analogue or oligomer (hereinafter also referred to as anti-amyloid β peptide analogue or anti-oligomer aptamer). Accordingly, the invention relates also to a method for providing an aptamer having specificity for the amyloid β peptide analogue or oligomer as defined herein, which method comprises at least the steps of

a) providing a binding target comprising the amyloid β peptide analogue or oligomer;
b) exposing an aptamer repertoire or potential aptamer repertoire to said binding target; and
c) selecting from said repertoire an aptamer which specifically binds to said amyloid β peptide analogue or oligomer.

An “aptamer” herein refers to oligonucleic acid or peptide molecules that are capable of specific, non-covalent binding to its target. Aptamer preferably comprise peptide, DNA or RNA sequence, more preferably peptide, DNA or RNA sequence of about 3 to 100 monomers, which may at one end or both ends be attached to a larger molecule, preferably a larger molecule mediating biochemical functions, more preferably a larger molecule inducing inactivation and/or degradation, most preferably ubiquitin, or preferably a larger molecule facilitating destruction, more preferably an enzyme or a fluorescent protein.

Here it is to be understood that a “potential aptamer repertoire” refers to any library, collection, assembly or set of amino acid sequences or nucleic acid sequences or to any generator of such a library, collection, assembly or set of amino acid sequences that can be used for producing an aptamer repertoire in vivo or in vitro.

In another aspect, the invention also provides aptamers that bind to the amyloid β peptide analogue or oligomer as defined herein.

In a preferred embodiment of the invention, the aptamer is obtainable by a method comprising selecting the aptamer from a repertoire or potential repertoire as described herein.

According to a particularly preferred embodiment, the present invention provides amyloid β peptide analogue- or oligomer-specific aptamers. These include in particular aptamers having a comparatively smaller affinity for both the monomeric and fibrillomeric forms of Aβ peptide than for the amyloid β peptide analogue or oligomer of the invention.

The agents that are capable of binding to the amyloid β peptide analogue or oligomer of the invention also have many potential applications, some of which are described in the following. They are especially useful for therapeutic and diagnostic purposes.

Antibodies that specifically bind to the globulomer epitope have proven to be useful agents in therapeuitc and disgnostic applications. As the amyloid β peptide analogues or oligomers of the present invention react with said antibodies the amyloid β peptide analogues or oligomers are believed to display the same or a very similar epitop.

Thus, the invention also provides agents that are capable of binding to the amyloid β peptide analogue or oligomer of the invention for therapeutic uses.

In one aspect, the invention also provides therapeutic compositions comprising an agent that is capable of binding to the amyloid β peptide analogue or oligomer of the invention. According to a particular embodiment, said compositions are pharmaceutical compositions which further comprise a pharmaceutical acceptable carrier.

Said pharmaceutical compositions of the invention may furthermore contain at least one additional therapeutic agent, for example one or more additional therapeutic agents for the treatment of a disease for whose relief the agents of the invention are useful. If, for example, the agent of the invention binds to an amyloid β peptide analogue or oligomer of the invention, the pharmaceutical composition may furthermore contain one or more additional therepeutic agents useful for the treatment of disorders in which the activity of said amyloid β peptide analogue or oligomer is important.

Pharmaceutically suitable carriers include any solvents, dispersing media, coatings, antibacterial and antifungal agents, isotonic and absorption-delaying agents, and the like, as long as they are physiologically compatible. Pharmaceutically acceptable carriers include, for example, water, saline, phosphate-buffered saline, dextrose, glycerol, ethanol and the like, and combinations thereof. In many cases, preference is given to using isotonic agents, for example sugars, polyalcohols such as mannitol or sorbitol, or sodium chloride in addition. Pharmaceutically suitable carriers may furthermore contain relatively small amounts of auxiliary substances such as wetting agents or emulsifiers, preservatives or buffers, which increase the half life or efficacy of the antibodies.

The pharmaceutical compositions may be suitable for parenteral administration, for example. Here, the agents are prepared preferably as injectable solutions with an agent, e.g. antibody, content of 0.1-250 mg/ml. The injectable solutions may be prepared in liquid or lyophilized form, the dosage form being a flint glass or vial, an ampoule or a filled syringe. The buffer may contain L-histidine (1-50 mM, preferably 5-10 mM) and have a pH of 5.0-7.0, preferably of 6.0. Further suitable buffers include, without being limited thereto, sodium succinate, sodium citrate, sodium phosphate or potassium phosphate buffers. Sodium chloride may be used in order to adjust the tonicity of the solution to a concentration of 0-300 mM (preferably 150 mM for a liquid dosage form). Cryoprotectants, for example sucrose (e.g. 0-10%, preferably 0.5-1.0%) may also be included for a lyophilized dosage form. Other suitable cryoprotectants are trehalose and lactose. Fillers, for example mannitol (e.g. 1-10%, preferably 2-4%) may also be included for a lyophilized dosage form. Stabilizers, for example L-methionine (e.g. 51-50 mM, preferably 5-10 mM) may be used both in liquid and lyophilized dosage forms. Further suitable fillers are glycine and arginine. Surfactants, for example polysorbate 80 (e.g. 0-0.05%, preferably 0.005-0.01%), may also be used. Further surfactants are polysorbate 20 and BRIJ surfactants.

The compositions of the invention may have a multiplicity of forms. These include liquid, semisolid and solid dosage forms, such as liquid solutions (e.g. injectable and infusible solutions), dispersions or suspensions, tablets, pills, powders, liposomes and suppositories. The preferred form depends on the intended type of administration and on the therapeutic application. Typically, preference is given to compositions in the form of injectable or infusible solutions, for example compositions which are similar to antibodies for passive immunization of humans. The preferred route of administration is parenteral (e.g. intravenous, subcutaneous, intraperitoneal or intramuscular). According to a preferred embodiment, the agent is administered by intravenous infusion or injection. According to another preferred embodiment, the agent is administered by intramuscular or subcutaneous injection.

Therapeutic compositions must typically be sterile and stable under preparation and storage conditions. The compositions may be formulated as solutions, microemulsions, dispersions, liposomes or other ordered structures suitable for high concentrations of active substance. Sterile injectable solutions may be prepared by introducing the active compound (i.e. the agent such as an antibody) in the required amount into a suitable solvent, where appropriate with one or a combination of the abovementioned ingredients, as required, and then sterile-filtering said solution. Dispersions are usually prepared by introducing the active compound into a sterile vehicle containing a basic dispersion medium and, where appropriate, other required ingredients. In the case of a sterile lyophilized powder for preparing sterile injectable solutions, vacuum drying and spray drying are preferred methods of preparation, which produces a powder of the active ingredient and, where appropriate, of further desired ingredients from a previously sterile-filtered solution. The correct flowability of a solution may be maintained by using, for example, a coating such as lecithin, by maintaining, in the case of dispersions the required particle size or by using surfactants. A prolonged absorption of injectable compositions may be achieved by additionally introducing into the composition an agent which delays absorption, for example monostearate salts and gelatine.

The agents of the invention may be administered by a multiplicity of methods known to the skilled worker, although the preferred type of administration for many therapeutic applications is subcutaneous injection, intravenous injection or infusion. The skilled worker will appreciate that the route and/or type of administration depend on the result desired. According to particular embodiments, the active compound may be prepared with a carrier which protects the compound against rapid release, such as, for example, a formulation with sustained or controlled release, which includes implants, transdermal plasters and microencapsulated release systems. Biologically degradable biocompatible polymers such as ethylene vinyl acetate, polyanhydrides, polyglycolic acid, collagen, polyorthoesters and polylactic acid may be used. The methods of preparing such formulations are well known to the skilled worker; see, for example, Sustained and Controlled Release Drug Delivery Systems, J. R. Robinson, ed., Marcel Dekker, Inc., New York, 1978.

According to particular embodiments, an agent of the invention may be administered orally, for example in an inert diluent or a metabolizable edible carrier. The agent (and further ingredients, if desired) may also be enclosed in a hard or soft gelatine capsule, compressed to tablets or added directly to food. For oral therapeutic administration, the agents may be mixed with excipients and used in the form of oral tablets, buccal tablets, capsules, elixirs, suspensions, syrups and the like. If it is intended to administer an agent of the invention via a route other than the parenteral one, it may be necessary to choose a coating from a material which prevents its inactivation.

In a further aspect, the invention provides the use of an agent that is capable of binding to the amyloid β peptide analogue or oligomer of the invention for preparing a pharmaceutical composition for treating or preventing an amyloidosis.

In a preferred embodiment of the invention, the pharmaceutical composition is for passive immunization.

Accordingly, the invention also provides a method of treating or preventing an amyloidosis in a subject in need thereof, which comprises administering the agent that is capable of binding to the amyloid β peptide analogue or oligomer of the invention to the subject.

In a preferred embodiment of the invention, administering the agent that is capable of binding to the Aβ(amyloid β peptide analogue or oligomer of the invention is for passively immunizing the subject against an amyloidosis.

The screening of biological samples revealed that such samples may contain substances that react with agents that are capable of binding to the amyloid β peptide analogue or oligomer such as anti-amyloid β peptide analogue or anti-oligomer antibodies of the invention. Such substances which have a certain binding affinity to said agents but which cannot be said to correspond to the amyloid β peptide analogues or oligomers of the invention, are hereinafter referred to as antigens comprising the amyloid β peptide analogue or oligomer epitope.

Thus, the agents that are capable of binding to the amyloid β peptide analogue or oligomer of the invention are also capable of detecting, both in vitro and in vivo, antigens comprising amyloid β peptide analogue or oligomer epitopes to which they bind. Said agents may therefore be used for detecting said antigens, for instance a sample that is derived from a subject suspect of having an amyloidosis, or in a subject suspect of having an amyloidosis, for instance a human individual or other mammal.

The invention thus also provides agents that are capable of binding to the amyloid β peptide analogue or oligomer of the invention for diagnostic uses.

In one aspect, the invention provides diagnostic compositions comprising an agent that is capable of binding to the amyloid β peptide analogue or oligomer of the invention. According to a particular embodiment, said compositions are pharmaceutical compositions which further comprise a pharmaceutical acceptable carrier.

In a further aspect, the invention provides the use of an agent that is capable of binding to the amyloid β peptide analogue or oligomer of the invention for preparing a composition for diagnosing an amyloidosis.

Accordingly, the invention also provides a method of diagnosing an amyloidosis which comprises providing a sample from the subject suspected of having the amyloidosis, contacting the sample with an agent that is capable of binding to the amyloid β peptide analogue or oligomer of the invention for a time and under conditions sufficient for the formation of a complex comprising the agent that is capable of binding to the amyloid β peptide analogue or oligomer of the invention and an antigen comprising the amyloid β peptide analogue or oligomer epitope, the presence of the complex indicating the subject has the amyloidosis. According to a particular embodiment, at least the step of contacting the sample is carried out ex vivo and in particular in vitro.

Thus, the agents that are capable of binding to the amyloid β peptide analogue or oligomer of the invention may be used in a variety of diagnostic methods and assays.

According to one embodiment, the method of diagnosing an amyloidosis in a patient suspected of having this disease comprises the steps of: 1) isolating a biological sample from the patient; 2) contacting the biological sample with at least one of the agents that are capable of binding to the amyloid β peptide analogue or oligomer of the invention for a time and under conditions sufficient for formation of antigen/agent complexes; and 3) detecting presence of the antigen/agent complexes in said sample, presence of the complexes indicating a diagnosis of an amyloidosis, e.g. Alzheimer's disease, in the patient. The antigen is one comprising the amyloid β peptide analogue or oligomer epitope. According to a particular embodiment, at least one of said steps 2) and 3) is carried out ex vivo and in particular in vitro. According to a further particular embodiment, the method does not comprise step 1).

According to a further embodiment, the method of diagnosing an amyloidosis in a patient suspected of having this disease comprises the steps of: 1) isolating a biological sample from the patient; 2) contacting the biological sample with an antigen for a time and under conditions sufficient for the formation of antibody/antigen complexes; 3) adding a conjugate to the resulting antibody/antigen complexes for a time and under conditions sufficient to allow the conjugate to bind to the bound antibody, wherein the conjugate comprises one of the agents that are capable of binding to amyloid β peptide analogue or oligomer of the invention, attached to a signal generating compound capable of generating a detectable signal; and 4) detecting the presence of an antibody which may be present in the biological sample, by detecting a signal generated by the signal generating compound, the signal indicating a diagnosis of an amyloidosis, e.g. Alzheimer's disease in the patient. The antigen is one comprising the amyloid β peptide analogue or oligomer epitope. According to a particular embodiment, at least one of said steps 2), 3) and 4) is carried out ex vivo and in particular in vitro. According to a further particular embodiment, the method does not comprise step 1).

According to a further embodiment, the method of diagnosing an amyloidosis in a patient suspected of having this disease comprises the steps of: 1) isolating a biological sample from said patient; 2) contacting the biological sample with anti-antibody, wherein the anti-antibody is specific for one of the agents that are capable of binding to the amyloid β peptide analogue or oligomer of the invention, for a time and under conditions sufficient to allow for formation of anti-antibody/agent complexes, the complexes containing agent present in the biological sample; 3) adding a conjugate to resulting anti-antibody/agent complexes for a time and under conditions sufficient to allow the conjugate to bind to bound agent, wherein the conjugate comprises an antigen comprising the amyloid β peptide analogue or oligomer epitope, which binds to a signal generating compound capable of generating a detectable signal; and 4) detecting a signal generated by the signal generating compound, the signal indicating a diagnosis of an amyloidosis, e.g. Alzheimer's disease in the patient. According to a particular embodiment, at least one of said steps 2), 3) and 4) is carried out ex vivo and in particular in vitro. According to a further particular embodiment, the method does not comprise step 1).

In one diagnostic embodiment of the present invention, the agent that is capable of binding to the amyloid β peptide analogue or oligomer of the invention, or a portion thereof, is coated on a solid phase (or is present in a liquid phase). The test or biological sample (e.g., whole blood, cerebrospinal fluid, serum, etc.) is then contacted with the solid phase. If antigen (e.g., globulomer) is present in the sample, such antigens bind to the agents that are capable of binding to the amyloid β peptide analogue or oligomer of the invention on the solid phase and are then detected by either a direct or indirect method. The direct method comprises simply detecting presence of the complex itself and thus presence of the antigens. In the indirect method, a conjugate is added to the bound agent. The conjugate comprises a second antibody, which binds to the bound antigen, attached to a signal-generating compound or label. Should the second antibody bind to the bound antigen, the signal-generating compound generates a measurable signal. Such signal then indicates presence of the antigen in the test sample.

Examples of solid phases used in diagnostic immunoassays are porous and non-porous materials, latex particles, magnetic particles, microparticles (see U.S. Pat. No. 5,705,330), beads, membranes, microtiter wells and plastic tubes. The choice of solid phase material and method of labeling the antigen or antibody present in the conjugate, if desired, are determined based upon desired assay format performance characteristics.

As noted above, the conjugate (or indicator reagent) will comprise an antibody (or perhaps anti-antibody, depending upon the assay), attached to a signal-generating compound or label. This signal-generating compound or “label” is itself detectable or may be reacted with one or more additional compounds to generate a detectable product. Examples of signal-generating compounds include chromogens, radioisotopes (e.g., 125I, 131I, 32P, 3H, 35S and 14C), chemiluminescent compounds (e.g., acridinium), particles (visible or fluorescent), nucleic acids, complexing agents, or catalysts such as enzymes (e.g., alkaline phosphatase, acid phosphatase, horseradish peroxidase, beta-galactosidase and ribonuclease). In the case of enzyme use (e.g., alkaline phosphatase or horseradish peroxidase), addition of a chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

Examples of biological fluids which may be tested by the above immunoassays include plasma, whole blood, dried whole blood, serum, cerebrospinal fluid or aqueous or organo-aqueous extracts of tissues and cells.

Kits are also included within the scope of the present invention. More specifically, the present invention includes kits for determining the presence of antigens comprising the amyloid β peptide analogue or oligomer epitope in a subject. In particular, a kit for determining the presence of said antigens in a sample comprises a) an agent that is capable of binding to the amyloid β peptide analogue or oligomer of the invention; and optionally b) a conjugate comprising an antibody that binds to the agent, attached to a signal generating compound capable of generating a detectable signal. The kit may also contain a control or calibrator which comprises a reagent which binds to the antigen.

The present invention also includes another type of kit for detecting antibodies such as auto-antibodies in a sample. The kit may comprise a) an antibody specific for the agent that is capable of binding to the amyloid β peptide analogue or oligomer of the invention (e.g. an anti-antibody), and b) an antigen comprising the amyloid β peptide analogue or oligomer epitope as defined herein. A control or calibrator comprising a reagent which binds to the antigen may also be included. More specifically, the kit may comprise a) an anti-antibody specific for the auto-antibody and b) a conjugate comprising antigen comprising the amyloid β peptide analogue or oligomer epitope as defined herein, the conjugate being attached to a signal generating compound capable of generating a detectable signal. Again, the kit may also comprise a control or calibrator comprising a reagent which binds to the antigen.

The kit may also comprise one container such as vial, bottles or strip, with each container with a pre-set solid phase, and other containers containing the respective conjugates. These kits may also contain vials or containers of other reagents needed for performing the assay, such as washing, processing and indicator reagents.

Due to their binding affinity to the agents that are capable of binding to the amyloid β peptide analogue or oligomer of the invention, said antigens comprising amyloid β peptide analogue or oligomer epitopes can be detected in preparations suspected of containing such epitopes, their amount can be determined in said preparations and they can be enriched. Accordingly, the present invention also provides a method for detecting, for determining the amount of and/or for enriching amyloid β peptide analogue or oligomer epitopes in preparations suspected or know to comprise such epitopes. Once detected and enriched, said substances may have potential applications similar to those described herein with respect to the amyloid β peptide analogue or oligomer of the invention.

Moreover, the present invention includes a method of designing agents such as antibodies, non-antibody biological agents or small molecules useful in the treatment or prevention of an amyloidosis in a patient. This method comprises the steps of: a) analyzing the three-dimensional structure of the amyloid β peptide analogue or oligomer described herein; b) identifying one or more epitopes on the surface of the amyloid β peptide analogue or oligomer of step a); and c) designing an agent such as an antibody, non-antibody biological agent or a small molecule which will bind to the identified epitope or epitopes of step b), the antibody, non-antibody biological agent or a small molecule to be used in the treatment or prevention of amyloidosis.

### ADVANTAGES OF THE INVENTION

The amino acid composition of the amyloid β peptide analogues and oligomers of the present invention are well-defined and reproducible.

The amyloid β peptide analogues and oligomers of the present invention display a stable conformation.

The amyloid β peptide analogues and oligomers of the present invention display better hydrodynamic properties.

Active immunization with the amyloid β peptide analogues or oligomers of the present invention is expected to elicit a highly selective immune response for Aβ globulomers. Because the amyloid β peptide analogues and oligomers of the present invention can easily be designed to lack N-terminal sequences, the risk of eliciting an unspecific N-terminal Aβ peptide directed immune response can be eliminated. The amyloid β peptide analogues and oligomers of the present invention are therefore capable of eliciting an immune response that discriminates other forms of Aβ peptides, particularly monomers and fibrils.

Further, it is expected that active immunization with the amyloid β peptide analogues or oligomers of the present invention will be effective in reversing cognitive deficits in AD transgenic mouse models as the elicited antibody response is comparable to active immunization with Aβ(20-42) truncated globulomer. The latter has been proven to reverse deficits in novel object recognition task.

All patents, patent applications and publications referred to herein are hereby incorporated in their entirety by reference.

Deposit Information: The hybridoma which produces monoclonal antibody 5F7 was deposited with the American Type Culture Collection, 10801 University Boulevard, Manassas, Va. 20110 on Dec. 1, 2005 under the terms of the Budapest Treaty and received designation PTA-7241. Further, the hybridoma which produces monoclonal antibody 10F11 was deposited with the American Type Culture Collection, 10801 University Boulevard, Manassas, Va. 10801 on Dec. 1, 2005 under the terms of the Budapest Treaty and received designation PTA-7239. Additionally, the hybridoma which produces monoclonal antibody 4B7 was deposited with the American Type Culture Collection, 10801 University Boulevard, Manassas, Va. 10801 on Dec. 1, 2005 under the terms of the Budapest Treaty and received designation PTA-7242, and the hybridoma which produces monoclonal antibody 7C6 was deposited with the American Type Culture Collection, 10801 University Boulevard, Manassas, Va. 10801 on Dec. 1, 2005 under the terms of the Budapest Treaty and received designation PTA-7240. Additionally, the hybridoma which produces monoclonal antibody 6A2 was deposited with the American Type Culture Collection, 10801 University Boulevard, Manassas, Va. 10801 on Feb. 28, 2006 under the terms of the Budapest Treaty and received designation PTA-7409, and the hybridoma which produces monoclonal antibody 2F2 was deposited with the American Type Culture Collection, 10801 University Boulevard, Manassas, Va. 10801 on Feb. 28, 2006 under the terms of the Budapest Treaty and received designation PTA-7408. The hybridoma which produces monoclonal antibody 4D10 was deposited with the American Type Culture Collection, 10801 University Boulevard, Manassas, Va. 10801 on Feb. 28, 2006 under the terms of the Budapest Treaty and received designation PTA-7405. The hybridoma which produces monoclonal antibody 7E5 was deposited with the American Type Culture Collection, 10801 University Boulevard, Manassas, Va. 10801 on Aug. 16, 2006 under the terms of the Budapest Treaty and received designation PTA-7809. The hybridoma which produces monoclonal antibody 10C1 was deposited with the American Type Culture Collection, 10801 University Boulevard, Manassas, Va. 10801 on Aug. 16, 2006 under the terms of the Budapest Treaty and received designation PTA-7810. The hybridoma which produces monoclonal antibody 3B10 was deposited with the American Type Culture Collection, 10801 University Boulevard, Manassas, Va. 10801 on Sep. 1, 2006 under the terms of the Budapest Treaty and received designation PTA-7851. All deposits have been made on behalf of Abbott Laboratories, 100 Abbott Park Road, Abbott Park, Ill. 60064 (US).

All these monoclonal antibodies are murine monoclonal antibodies.

The following examples are intended to illustrate the invention, without limiting its scope.

### EXAMPLES

### Example 1

### Peptide Synthesis

All reagents were used as obtained from the vendor unless otherwise specified. Peptide synthesis reagents including diisopropylethylamine (DIEA), N-methylpyrrolidone (NMP), dichloromethane (DCM), (2-(7-Aza-1H-benzotriazole-1-yl)-1,1,3,3-tetramethyluronium hexafluorophosphate) (HATU), 2-(1H-benzotriazole-1-yl)-1,1,3,3-tetramethyl-uronium-hexafluorophosphate (HBTU), 1-hydrobenzotriazole (HOBt), and piperidine were obtained from Applied Biosystems, Inc. (ABI), Foster City, Calif.; or American Bioanalytical, Natick, Mass. Standard 9-Fluorenylmethyloxycarbonyl (Fmoc) amino acid derivatives (Fmoc-Ala-OH, Fmoc-Cys(Trt)-OH, Fmoc-Cys(ACM)-OH, Fmoc-Asp(tBu)-OH, Fmoc-Glu(tBu)-OH, Fmoc-Phe-OH, Fmoc-Gly-OH, Fmoc-His(Trt)-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Val-OH, Fmoc-Trp(Boc)-OH, Fmoc-Tyr(tBu)-OH) were obtained from Anaspec, San Jose, Calif.; or ABI. Fmoc-3-amino-1-carboxymethyl-pyridin-2-one and Fmoc-cis-3-phenyl-pyrrolidine-2-carboxylic acid were obtained from NeoSystem, Strasbourg, France. Peptide synthesis resins (Fmoc-Ala-PEG polystyrene resin, Fmoc-Ala-Wang resin, Rink amide MBHA resin) were obtained from ABI, CS-Bio, Menlo Park, Calif. or Novabiochem, Hohenbrunn, Germany. Trifluoroacetic acid (TFA) was obtained from Oakwood Products, West Columbia, S.C. Thioanisole, phenol, triisopropylsilane (TIS), 3,6-dioxa-1,8-octanedithiol (DODT), potassium ferricyanide, and isopropanol, were obtained from Aldrich Chemical Co., Milwaukee, Wis. Matrix-assisted laser desorption ionization mass-spectra (MALDI-MS) were recorded on an Applied Biosystems Voyager DE-PRO MS). Electrospray mass-spectra (ESI-MS) were recorded on Finnigan SSQ7000 (Finnigan Corp., San Jose, Calif.) in both positive and negative ion mode.

### General Procedure for Solid-Phase Peptide Synthesis:

Peptides were synthesized with either 100 μmol preloaded resin/vessel on an ABI Pioneer peptide synthesizer using standard 0.1 mM coupling cycles, or 250 μmol resin/vessel on an ABI 433 peptide synthesizer. On the ABI Pioneer synthesizer coupling standard Fmoc-amino acids, preloaded tubes containing 0.4 mmol reagent were used with single-coupling with 4:4:8 Fmoc-amino acid:HATU:DIEA. On the ABI 433 synthesizer, preloaded cartridges containing 1 mM Fmoc-amino acid was used with single-coupling preactivated as a 4:4:4:8 Fmoc-amino acid:HBTU:HOBt:DIEA. On each synthesizer following coupling the Fmoc protecting group was removed by treatment with piperidine. When the synthesis was complete, the resin was washed with 3×DCM and 3× isopropanol, and dried in vacuo to give the protected peptide resin.

### General Procedure for Cleavage and Deprotection of Resin-Bound Peptide:

The peptides were cleaved from the resin by shaking the resin for 3 h at ambient temperature in a cleavage cocktail consisting of 80% TFA, 5% water, 5% thioanisole, 5% phenol, 2.5% TIS, and 2.5% DODT (1 mL/0.1 g resin). The resin was removed by filtration, rinsed with 2×TFA, the TFA evaporated from the filtrates, the residue precipitated with ether (10 mL/0.1 g resin), recovered by centrifugation, washed with 2× ether (10 mL/0.1 g resin) and dried to give the crude peptide.

### General Procedure for Purification of Peptides:

The crude peptides were purified on a Gilson preparative HPLC system running Unipoint® analysis software (Gilson, Inc., Middleton, Wis.) on an Agilent 21.2×250 mm column packed with Zorbax®-C3, 7 μm particles, 300 Å pore size. The temperature of the column was maintained at >60° C. throughout the purification. Three milliliters of crude peptide solution (5 mg/mL in 75:25 formic acid:water) was purified per injection. The peaks containing the product(s) from each run were pooled and lyophilized. All preparative runs were run at 20 mL/min with eluents as buffer A: 0.1% TFA-water and buffer B: acetonitrile using a 1%/minute gradient of B until the products eluted.

### General Procedure for Analytical HPLC:

Analytical HPLC was performed on a Hitachi D-7000 analytical HPLC system with a dual wavelength detector running D-7000 software on an Agilent 0.46×250 mm column packed with Zorbax®-C3, 5 μm particles, 300 Å pore size eluted with one of the gradient methods listed below after preequilibrating at the starting conditions for 7 min. All analytical runs were run at 1 mL/min with eluents as buffer A: 0.1% TFA-water and buffer B: acetonitrile using a 2%/minute gradient of B for 45 min at 75° C.

In the following, Aβ peptides and oligomers are referred to as (xXaa1, yXaa2) Aβ(X-Y) peptides and oligomers, wherein Aβ(X-Y) refers to the amino acid sequence from amino acid position X to amino acid position Y of the human amyloid β protein including both X and Y as set forth in SEQ ID NO:1 (corresponding to amino acid positions 1 to 43), and Xaa1 and Xaa2 designate amino acids which replace the amino acid at position x and y, respectively, in SEQ ID NO:1. The term “(xXaa1, yXaa2) Aβ(X-Y)” is synonymous with the term “Aβ(X-Y) (xXaa1/yXaa2)”.

### a) (17C, 34C) N-Met Aβ(1-42) (1a)

The amyloid β peptide analogue having the amino acid sequence MDAEFRHDSGYEVHHQKCVFFAEDVGSNKGAIIGCMVGGVVIA (SEQ ID NO:961) was prepared as follows.

Preloaded resin (0.59 g, 100 μmol) was extended using the general peptide synthesis procedure to give the protected resin-bound peptide (0.989 g, 57%). The resin was cleaved and deprotected using the general procedure to give the crude peptide 1a as a white solid (0.319 g, 62.6%). Crude peptide 1a was dissolved in neat formic acid (6.7 mg/mL) and diluted with water (to 5 mg/mL) immediately before HPLC purification with collection based on absorbance at 260 nm. The main peak was isolated and lyophilized giving 1a as a white solid (0.0274 g, 8.5%); deconvoluted ESI-MS m/z=4625.4 [(M+H)⁺].

The following peptides (1b) to (1j) were synthesized by standard Fmoc Solid Phase synthesis from the C-terminus to the N-terminus. Following synthesis, the peptides are purified by Reverse Phase HPLC. In order to maintain the free sulfhydryl groups on the cysteines (avoid oxidation), an acidic pH was maintained during production (normal procedure). Briefly, the FMOC protecting group was removed from the amino acid that is on the resin. The next amino acid in the sequence was added and coupled to the first amino acid using HBTU. The FMOC protecting group was removed from the 2nd amino acid. The next amino acid in the sequence was added and coupled to the previous amino acid using HBTU. Steps 4 and 5 were repeated until the sequence was complete. The peptide was cleaved from the resin using TFA. The peptide was purified via Reverse Phase HPLC using a TFA/Acetonitrile buffer system (acidic buffer system). The purification fractions which meet Mass and purity spec, were pooled and lyophilized. Mass Spec Analysis and RP-HPLC confirmed identity of peptide.

### b) (14C, 37C) N-Met Aβ(1-42) (1b)

The amyloid β peptide analogue having the amino acid sequence MDAEFRHDSGYEVHCQKLVFFAEDVGSNKGAIIGLMVCGWIA (SEQ ID NO:962) was prepared using standard peptide chemistry.

### c) (15C, 36C) N-Met Aβ(1-42) (1c)

The amyloid β peptide analogue having the amino acid sequence MDAEFRHDSGYEVHHCKLVFFAEDVGSNKGAIIGLMCGGVVIA (SEQ ID NO:963) was prepared using standard peptide chemistry.

### d) (16C, 35C) N-Met Aβ(1-42) (1d)

The amyloid β peptide analogue having the amino acid sequence MDAEFRHDSGYEVHHQCLVFFAEDVGSNKGAIIGLCVGGVVIA (SEQ ID NO:964) was prepared using standard peptide chemistry.

### e) (17C, 34C) N-Met Aβ(1-42) (1e)

The amyloid β peptide analogue having the amino acid sequence MDAEFRHDSGYEVHHQKCVFFAEDVGSNKGAIIGCMVGGVVIA (SEQ ID NO:965) was prepared using standard peptide chemistry.

### f) (18C, 33C) N-Met Aβ(1-42) (1f)

The amyloid β peptide analogue having the amino acid sequence MDAEFRHDSGYEVHHQKLCFFAEDVGSNKGAIICLMVGGVVIA (SEQ ID NO:966) was prepared using standard peptide chemistry.

### g) (19C, 32C) N-Met Aβ(1-42) (1g)

The amyloid β peptide analogue having the amino acid sequence MDAEFRHDSGYEVHHQKLVCFAEDVGSNKGAICGLMVGGVVIA (SEQ ID NO:967) was prepared using standard peptide chemistry.

### h) (20C, 31C) N-Met Aβ(1-42) (1h)

The amyloid β peptide analogue having the amino acid sequence MDAEFRHDSGYEVHHQKLVFCAEDVGSNKGACIGLMVGGWIA (SEQ ID NO:968) was prepared using standard peptide chemistry.

### i) (21C, 30C) N-Met Aβ(1-42) (1i)

The amyloid β peptide analogue having the amino acid sequence MDAEFRHDSGYEVHHQKLVFFCEDVGSNKGCIIGLMVGGVVIA (SEQ ID NO:969) was prepared using standard peptide chemistry.

### j) (22C, 29C) N-Met Aβ(1-42) (1j)

The amyloid β peptide analogue having the amino acid sequence MDAEFRHDSGYEVHHQKLVFFACDVGSNKCAIIGLMVGGVVIA (SEQ ID NO:970) was prepared using standard peptide chemistry.

### k) (17C, 34C) Aβ(12-42) (1k)

The amyloid β peptide analogue having the amino acid sequence VHHQKCVFFAEDVGSNKGAIIGCMVGGWIA (SEQ ID NO:971) was prepared by the general procedure outlined above using a Fmoc-Ala-Wang resin. Calculated MW [g/mol]: 3186.73; MALDI-MS: obs 3186.7 [(M+H)⁺].

l) (17C(ACM), 34C(ACM)) Aβ(16-35)-amide (1l)

The amyloid β peptide analogue having the amino acid sequence KC(ACM)VFFAEDVGSNKGAIIGC(ACM)M-amide (SEQ ID NO:972) was prepared by the general procedure outlined above using a Rink-Amide-MBHA resin. Calculated MW [g/mol]: 2230, 67; MALDI-MS: obs 2231.4, 2228.9 [(M+H)⁺].

m) Aβ(16-35)-amide (1m)

The amyloid β peptide analogue having the amino acid sequence KLVFFAEDVGSNKGAIIGLM-amide (SEQ ID NO:973) was prepared by the general procedure outlined above using a Rink-Amide-MBHA resin. Calculated MW [g/mol]: 2108, 5; MALDI-MS: obs 2108.1 [(M+H)⁺], 2130 [(M+Na)⁺].

n) (17K, 34K) Aβ(16-35)-amide (1n)

The amyloid β peptide analogue having the amino acid sequence KKVFFAEDVGSNKGAIIGKM-amide (SEQ ID NO:974) was prepared by the general procedure outlined above using a Rink-Amide-MBHA resin. Calculated MW [g/mol]: 2135, 53; MALDI-MS: obs 2137.96 2137.91 [(M+H)⁺].

o) (17C, 34C) Aβ(16-35)-amide, cyclic (1o)

The amyloid β peptide analogue having the amino acid sequence KC*VFFAEDVGSNKGAIIGC*M-amide (SEQ ID NO:975) was prepared by the general procedure outlined above. Disulfide cyclization was accomplished by dissolving the linear precursor peptide (17C, 34C) Aβ(16-35)-amide (1p) at 18 mg/mL in dimethylsulfoxide and added in one aliquot to vacuum degassed 3:2 100 mM ammonium bicarbonate:acetonitrile (200 mL, v:v). Potassium ferricyanide solution (0.05% w/v in water, 44.3 mL) was added in one portion and the reaction stirred for 2 h at ambient. The yellow solution was lyophilized to dryness prior to purification and analysis using the general procedures described above to give the cyclized (17C, 34C) Aβ(16-35)-amide. Calculated MW [g/mol]: 2086.46; MALDI-MS: obs 2086.6 [(M+H)⁺], 2108.6 [(M+Na)⁺].

p) (17C, 34C) Aβ(16-35)-amide (1p)

The amyloid β peptide analogue having the amino acid sequence KCVFFAEDVGSNKGAIIGCM-amide (SEQ ID NO:976) was prepared by the general procedure outlined above using a Rink-Amide-MBHA resin. Calculated MW [g/mol]: 2088.47; MALDI-MS: obs 2087.99 [(M+H)⁺], 2109.98 [(M+Na)⁺].

### q) (17K, 34K) N-Met Aβ(1-42) (1q)

The amyloid β peptide analogue having the amino acid sequence MDAEFRHDSGYEVHHQKKVFFAEDVGSNKGAIIGKMVGGVVIA (SEQ ID NO:977) was prepared by the general procedure outlined above using a H-Ala-HMPB NovaPEG resin. Calculated MW [g/mol]: 4675, 26; MALDI-MS: obs 4680.16 [(M+H)⁺].

### r) (17KC, 34C) Aβ(13-42) (1r)

The amyloid β peptide analogue having the amino acid sequence HHQKKCVFFAEDVGSNKGAIIGCMVGGWIA (SEQ ID NO:978) was prepared by the general procedure outlined above using a Fmoc-Ala-Wang resin. Calculated MW [g/mol]: 3215.77; MALDI-MS: obs 3214.72 [(M+H)⁺].

### s) (17C, 34C) Aβ(16-42) (1s)

The amyloid β peptide analogue having the amino acid sequence KCVFFAEDVGSNKGAIIGCMVGGVVIA (SEQ ID NO:979) was prepared by the general procedure outlined above using a Fmoc-Ala-Wang resin. Calculated MW [g/mol]: 2685.19; MALDI-MS: obs 2683.82 [(M+H)⁺], 2705.84 [(M+Na)⁺].

t) (17C(ACM), 34C(ACM), 35M(S-oxide)) Aβ(16-35)-amide (1t)

The amyloid β peptide analogue having the amino acid sequence KC(ACM)VFFAEDVGSNKGAIIGC(ACM)M(S-oxide)-amide (SEQ ID NO:980) was prepared by the general procedure outlined above using a Fmoc-Ala-Wang resin. Calculated MW [g/mol]: 2246.67; MALDI-MS: obs 2245.48 [(M+H)⁺].

u) (17C(ACM), 34C(ACM)) Aβ(16-35)-amide (1u)

The amyloid β peptide analogue having the amino acid sequence KC(ACM)VFFAEDVGSNKGAIIGC(ACM)M-amide (SEQ ID NO:981) was prepared by the general procedure outlined above using a Rink Amide MBHA resin. Calculated MW [g/mol]: 2230.67; MALDI-MS: obs 2229.45 [(M+H)⁺].

v) (17K, 34E) Aβ(16-35)-amide (1v)

The amyloid β peptide analogue having the amino acid sequence KKVFFAEDVGSNKGAIIGEM-amide (SEQ ID NO:982) was prepared by the general procedure outlined above using a PEGA-Novabiochem resin. Calculated MW [g/mol]: 2139.47; MALDI-MS: obs 2161.1 [(M+Na)⁺].

### w) (17K, 34C) N-Met Aβ(1-42) (1w)

The amyloid β peptide analogue having the amino acid sequence MDAEFRHDSGYEVHHQKKVFFAEDVGSNKGAIIGCMVGGWIA (SEQ ID NO:983) was prepared by the general procedure outlined above using a Fmoc-Ala-Wang resin. Calculated MW [g/mol]: 4650.23; MALDI-MS: obs 4651.07 [(M+H)⁺].

### x) (17K, 34E) N-Met Aβ(1-42) (1x)

The amyloid β peptide analogue having the amino acid sequence MDAEFRHDSGYEVHHQKKVFFAEDVGSNKGAIIGEMVGGWIA (SEQ ID NO:984) was prepared by the general procedure outlined above using a Fmoc-Ala-Wang resin. Calculated MW [g/mol]: 4676.21; MALDI-MS: obs 4676.2 [(M+H)⁺].

### y) (17C, 34C) N-Met Aβ(1-42) (1y)

The amyloid β peptide analogue having the amino acid sequence MDAEFRHDSGYEVHHQKCVFFAEDVGSNKGAIIGCMVGGVVIA (SEQ ID NO:985) was prepared by the general procedure outlined above using a Fmoc-Ala-Wang resin. Calculated MW [g/mol]: 4625.21; MALDI-MS: obs 4625.52 [(M+H)⁺].

z) (22C*, 29C*) Aβ(16-35)-amide, cyclised (1z)

The amyloid β peptide analogue having the amino acid sequence KKVFFAC*DVGSNKC*AIIGKM-amide (SEQ ID NO:986) can be prepared by the general procedure outlined above and using the cyclization method described for peptide (1o).

aa) (22C, 29C) Aβ(16-35)-amide (1aa)

The amyloid β peptide analogue having the amino acid sequence KKVFFACDVGSNKCAIIGKM-amide (SEQ ID NO:987) can be prepared by the general procedure outlined above.

### ab) (F20->FA19501, L17C, L34C) N-Met Aβ(1-42) (1ab)

The amyloid β peptide analogue having the amino acid sequence MDAEFRHDSGYEVHHQKCVF-FA19501-AEDVGSNKGAIIGCMVGGVVIA (SEQ ID NO:988) can be prepared by the general procedure outlined above using a Fmoc-Ala-Wang resin. FA19501 is the catalog number for Fmoc-cis-3-phenyl-pyrrolidine-2-carboxylic acid that can be incorporated as for the normal amino acids in the peptide.

### ac) (22C, 29C) Aβ(20-35) (1ac)

The amyloid β peptide analogue having the amino acid sequence FACDVGSNKCAIIGLM (SEQ ID NO:989) can be prepared by the general procedure outlined above using a Fmoc-Ala-Wang resin.

### ad) (22C, 29C) Aβ(20-42) (1ad)

The amyloid β peptide analogue having the amino acid sequence FACDVGSNKCAIIGLMVGGVVIA (SEQ ID NO:990) can be prepared by the general procedure outlined above using a Fmoc-Ala-Wang resin.

### ae) (17C, 22C, 29C, 34C) Aβ(20-42) N-Met Aβ(1-42) (1ae)

The amyloid β peptide analogue having the amino acid sequence MDAEFRHDSGYEVHHQKCVFFACDVGSNKCAIIGCMVGGWIA (SEQ ID NO:991) can be prepared by the general procedure outlined above using a Fmoc-Ala-Wang resin.

### af) (K28G29->FA12401, L17C, L34C) N-Met Aβ(1-42) (1af)

The amyloid β peptide analogue having the amino acid sequence MDAEFRHDSGYEVHHQKCVFFAEDVGSN-FA12401-AIIGCMVGGVVIA (SEQ ID NO:992) can be prepared by the general procedure outlined above using a Fmoc-Ala-Wang resin. FA12401 is the catalog number for Fmoc-3-amino-1-carboxymethyl-pyridin-2-one can be incorporated as for the normal amino acids in the peptide.

### Example 2

### Preparation of Oligomer

a) Dissulfide stabilized (17C, 34C) N-Met Aβ(1-42) oligomer (2a)

83.1 mg synthetic (17C, 34C) N-Met Aβ(1-42) (1a) of example 1a, TFA salt was treated with HFIP, (1 ml for every 6 mg peptide) and the solvent removed by lyophilization. This was dissolved into 4.0 ml of DMSO. This DMSO solution of the peptide was then added slowly to 45 mL of 20 mM PBS (20 mM NaH₂PO₄, 140 mM NaCl, pH 7.4), containing 0.2% SDS (sodium dodecylsulfate), with stirring. This solution was then made 5 mM in DTT (dithiothrietol) and incubated 6 hours 37° C.

The sample was then diluted with 3 parts water, and dialyzed overnight at room temperature against ¼th strength PBS with 0.05% SDS, using 3500 MWCO dialysis membrane. The dialysis was continued the next morning against 1 L fresh buffer at 4° C. for 2 hours.

The sample was then concentrated using a YM10 membrane in an Amicon stirred cell. A 0.5 ml aliquot was dialyzed overnight at 4° C. against/4th strength PBS with no SDS using a Pierce 10K slidelyzer.

### Example 3

### Preparation of Oligomers

a) (14C, 37C) N-Met Aβ(1-42) oligomer (3a)

(14C, 37C) N-Met Aβ(1-42) peptide (1b) of example 1b was suspended in 100% 1,1,1,3,3,3-hexafluoro-2-propanol (HFIP) at 4 mg/mL and incubated for complete solubilization under shaking at 37° C. for 2 h. The HFIP acts as a hydrogen-bond breaker and is used to eliminate pre-existing structural inhomogeneities in the Aβ peptide. HFIP was removed by evaporation in a SpeedVac and the Aβ peptide dissolved or suspended at a concentration of 5 mM in dimethylsulfoxide and sonicated for 20 s. 20 μl of the HFIP-pre-treated Aβ peptide in DMSO were diluted to 400 μM peptide concentration with 230 μl phosphate-buffered saline (PBS)+0.2% SDS+2 mM DTT (9.8 ml Helium aerated 20 mM NaH₂PO₄, 140 mM NaCl, pH 7.4+0.2 ml 10% SDS solution dissolved in H₂O+3 mg DTT, Serva, Cat. no.: 20710). An incubation for 6 h at 37° C. resulted in the 16/20-kDa (xC, yC) N-Met Aβ(1-42) intermediate and Aβ(1-42) intermediate. The 38/48-kDa (xC, yC) N-Met Aβ(1-42) oligomer was generated by a further dilution with three volumes of Helium aerated H₂O and incubation for 18 h at 37° C. After centrifugation at 10,000 g for 10 min the supernatants were removed and stored at −30° C. until further use.

b) (15C, 36C) N-Met Aβ(1-42) oligomer (3b)

(15C, 36C) N-Met Aβ(1-42) peptide (1c) of example 1c was subjected to oligomerization using the procedure described example 3a.

c) (16C, 35C) N-Met Aβ(1-42) oligomer (3c)

(16C, 35C) N-Met Aβ(1-42) peptide (1d) of example 1d was subjected to oligomerization using the procedure described example 3a.

d) (17C, 34C) N-Met Aβ(1-42) oligomer (3d)

(17C, 34C) N-Met Aβ(1-42) peptide (1e) of example 1e was subjected to oligomerization using the procedure described example 3a.

e) (18C, 33C) N-Met Aβ(1-42) oligomer (3e)

(18C, 33C) N-Met Aβ(1-42) peptide (1f) of example if was subjected to oligomerization using the procedure described example 3a.

f) (19C, 32C) N-Met Aβ(1-42) oligomer (3f)

(19C, 32C) N-Met Aβ(1-42) peptide (1g) of example 1g was subjected to oligomerization using the procedure described example 3a.

g) (20C, 31C) N-Met Aβ(1-42) oligomer (3g)

(20C, 31C) N-Met Aβ(1-42) peptide (1 h) of example 1h was subjected to oligomerization using the procedure described example 3a.

h) (21C, 30C) N-Met Aβ(1-42) oligomer (3h)

(21C, 30C) N-Met Aβ(1-42) peptide (11) of example 11 was subjected to oligomerization using the procedure described example 3a.

i) (22C, 29C) N-Met Aβ(1-42) oligomer (3i)

(22C, 29C) N-Met Aβ(1-42) peptide (1j) of example 1j was subjected to oligomerization using the procedure described example 3a.

j) Aβ(1-42) globulomer (3j)

Aβ(1-42) wilde type peptide (Bachem H-1368) was subjected to oligomerization using the procedure described example 3a.

k) (17C, 34C) Aβ(12-42) oligomer (3k)

(17C, 34C) Aβ(12-42) peptide (1k) of example 1k was subjected to oligomerization using the procedure described example 2a, with the following modification. Following HFIP treatment, the peptide was dissolved in 1% ammonia in 35% acetonitrile/65% water and incubated for about 30 min at RT to form the ammonium salt. This salt was then shell frozen and lyophilized to dryness overnight. Oligomerization was then completed to the 0.05% SDS step as described in example 2a.

(17C, 34C) Aβ(12-42) peptide (1k) of example 1k was treated with HFIP (1 ml for every 6 mg peptide) and the solvent removed by lyophilization. Following HFIP treatment, the peptide was dissolved in 1% ammonia in 35% acetonitrile/65% water and incubated for about 30 min at RT to form the ammonium salt. This salt was then shell frozen and lyophilized to dryness overnight. This was then dissolved into 4.0 ml of DMSO. This DMSO solution of the peptide was then added slowly to 45 mL of 20 mM PBS (20 mM NaH₂PO₄, 140 mM NaCl, pH 7.4), containing 0.2% SDS (sodium dodecylsulfate), with stirring. This solution was then made 5 mM in DTT (dithiothrietol) and incubated 6 hours 37° C.

l) (17C(ACM), 34C(ACM)) Aβ(16-35) oligomer (3l)

(17C(ACM), 34C(ACM)) Aβ(16-35) peptide (1l) was subjected to oligomerization using the procedure described example 3k, with the exception that no DTT was used.

m) Aβ(16-35)-amide oligomer (3m)

Aβ(16-35)-amide peptide (1 m) was subjected to oligomerization using the procedure described example 3k, with the exception that no DTT was used.

n) (17K, 34K) Aβ(16-35)-amide oligomer (3n)

Subjecting (17K, 34K) Aβ(16-35)-amide peptide (1n) to oligomerization using the procedure described example 3k (without DTT being used) will yield the oligomer.

o) [(17C, 34C) Aβ(16-35)-amide, cyclic] oligomer (3o)

[(17C, 34C) Aβ(16-35)-amide, cyclic] peptide (1o) was subjected to oligomerization using the procedure described example 3k, with the exception that no DTT was used.

p) (17C, 34C) Aβ(16-35)-amide oligomer (3p)

(17C, 34C) Aβ(16-35)-amide peptide (1p) was subjected to oligomerization using the procedure described example 3k.

q) (17K, 34K) N-Met Aβ(1-42) oligomer (3q)

Subjecting (17K, 34K) N-Met Aβ(1-42) peptide (1q) to oligomerization using the procedure described example 3k (without DTT being used) will yield the oligomer.

r) (17KC, 34C) Aβ(13-42) oligomer (3r)

(17KC, 34C) Aβ(13-42) peptide (1r) was subjected to oligomerization using the procedure described example 3k.

s) (17C, 34C) Aβ(16-42) oligomer (3s)

(17C, 34C) Aβ(16-42) peptide (1s) was subjected to oligomerization using the procedure described example 3k.

t) (17C(ACM), 34C(ACM), 35M(S-oxide)) Aβ(16-35)-amide oligomer (3t)

Subjecting (17C(ACM), 34C(ACM), 35M(S-oxide)) peptide (1t) to oligomerization using the procedure described example 3k (without DTT being used) will yield the oligomer.

u) (17C(ACM), 34C(ACM)) Aβ(16-35)-amide oligomer (3u)

(17C(ACM), 34C(ACM)) peptide (1u) was subjected to oligomerization using the procedure described example 3k, with the exception that no DTT was used.

v) (17K, 34E) Aβ(16-35) oligomer (3v)

(17K, 34E) Aβ(16-35) peptide (1v) was subjected to oligomerization using the procedure described example 3k, with the exception that no DTT was used.

w) (17K, 34C) N-Met Aβ(1-42) oligomer (3w)

(17K, 34C) N-Met Aβ(1-42) peptide (1w) was subjected to oligomerization using the procedure described example 3k, with the exception that no DTT was used.

x) (17K, 34E) N-Met Aβ(1-42) oligomer (3x)

(17K, 34E) N-Met Aβ(1-42) peptide (1x) was subjected to oligomerization using the procedure described example 3k, with the exception that no DTT was used.

y) (17C, 34C) N-Met Aβ(1-42) oligomer (3y)

(17C, 34C) N-Met Aβ(1-42) peptide (1y) was subjected to oligomerization using the procedure described example 3k.

z) [(22C*, 29C*) Aβ(16-35)-amide, cyclised] oligomer (3z)

Subjecting [(E22C*, G29C*) Aβ(16-35)-amide, cyclised] peptide (1z) to oligomerization using the procedure described example 3k will yield the oligomer.

aa) (22C, 29C) Aβ(16-35)-amide oligomer (3aa)

Subjecting (22C, 29C) Aβ(16-35)-amide peptide (1 aa) to oligomerization using the procedure described example 3k will yield the oligomer.

ab) (F20->FA19501, L17C, L34C) N-Met Aβ(1-42) oligomer (3ab)

Subjecting (F20->FA19501, L17C, L34C) N-Met Aβ(1-42) peptide (1ab) to oligomerization using the procedure described example 3k will yield the oligomer.

ac) (22C, 29C) Aβ(20-35) oligomer (3ac)

Subjecting (22C, 29C) Aβ(20-35) peptide (1 ac) to oligomerization using the procedure described example 3k will yield the oligomer.

ad) (22C, 29C) Aβ(20-42) oligomer (3ad)

Subjecting (22C, 29C) Aβ(20-42) peptide (lad) to oligomerization using the procedure described example 3k will yield the oligomer.

ae) (17C, 22C, 29C, 34C) Aβ(20-42) N-Met Aβ(1-42) oligomer (3ae)

Subjecting (17C, 22C, 29C, 34C) Aβ(20-42) N-Met Aβ(1-42) peptide (1ae) to oligomerization using the procedure described example 3k will yield the oligomer.

af) (K28G29->FA12401, L17C, L34C) N-Met Aβ(1-42) oligomer (3af)

Subjecting (K28G29->FA12401, L17C, L34C) N-Met Aβ(1-42) peptide (1af) to oligomerization using the procedure described example 3k will yield the oligomer.

### Example 4

### Linkage Formation

a) Disulfide-stabilized (14C, 37C) N-Met Aβ(1-42) oligomer (4a)

1 ml of the (14C, 37C) N-Met Aβ(1-42) oligomer (3a) of example 3a was thawed and dialysed in a dialysis tube two times for 2 h at room temperature against 2 L 5 mM NaH₂PO₄, 35 mM NaCl, pH 7.4. Subsequently the dialysates were removed and the protein concentrations were determined by the Bio-Rad Protein Assay, BioRad, Cat. no.: 500-0006.

b) Disulfide-stabilized (15C, 36C) N-Met Aβ(1-42) oligomer (4b)

(15C, 36C) N-Met Aβ(1-42) oligomer (3b) of example 3b was subjected to oxidation using the procedure described example 4a.

c) Disulfide-stabilized (16C, 35C) N-Met Aβ(1-42) oligomer (4c)

(16C, 35C) N-Met Aβ(1-42) oligomer (3c) of example 3c was subjected to oxidation using the procedure described example 4a.

d) Disulfide-stabilized (17C, 34C) N-Met Aβ(1-42) oligomer (4d)

(17C, 34C) N-Met Aβ(1-42) oligomer (3d) of example 3d was subjected to oxidation using the procedure described example 4a.

e) Disulfide-stabilized (18C, 33C) N-Met Aβ(1-42) oligomer (4e)

(18C, 33C) N-Met Aβ(1-42) oligomer (3e) of example 3e was subjected to oxidation using the procedure described example 4a.

f) Disulfide-stabilized (19C, 32C) N-Met Aβ(1-42) oligomer (4f)

(19C, 32C) N-Met Aβ(1-42) oligomer (3f) of example 3f was subjected to oxidation using the procedure described example 4a.

g) Disulfide-stabilized (20C, 31C) N-Met Aβ(1-42) oligomer (4g)

(20C, 31C) N-Met Aβ(1-42) oligomer (3g) of example 3g was subjected to oxidation using the procedure described example 4a.

h) Disulfide-stabilized (21C, 30C) N-Met Aβ(1-42) oligomer (4h)

(21C, 30C) N-Met Aβ(1-42) oligomer (3h) of example 3h was subjected to oxidation using the procedure described example 4a.

i) Disulfide-stabilized (22C, 29C) N-Met Aβ(1-42) oligomer (4i)

(22C, 29C) N-Met Aβ(1-42) oligomer (31) of example 31 was subjected to oxidation using the procedure described example 4a.

j) Aβ(1-42) globulomer (4j)

Aβ(1-42) wilde type globulomer (Bachem H-1368) (3j) of example 3j was subjected to oxidation using the procedure described example 4a.

k) Disulfide-stabilized (17C, 34C) Aβ(12-42) oligomer (4k)

(17C, 34C) Aβ(12-42) oligomer (3k) was diluted with 3 parts water, and dialyzed overnight at room temperature against ¼th strength PBS with 0.05% SDS, using 3500 MWCO dialysis membrane. The dialysiswas continued the next morning against 1 L fresh buffer at 4° C. for 2 hours.

The sample was then concentrated using a YM10 membrane in an Amicon stirred cell. The sample can also be concentrated using Millipore UltraMax centrifugal concentrators with a 10 kDa cut-off membrane.

l) (17C(ACM), 34C(ACM)) Aβ(16-35) oligomer (4l)

(17C(ACM), 34C(ACM)) Aβ(16-35) oligomer (3l) does not form cysteine bridges.

m) Aβ(16-35)-amide oligomer (4m)

Aβ(16-35)-amide oligomer (3m) does not form cross-links.

n) Cross-linked (17K, 34K) Aβ(16-35)-amide oligomer (4n)

Subjecting (17K, 34K) Aβ(16-35)-amide oligomer (3n) to conditions suitable for cross-linking two amine groups will yield the product.

o) [(17C, 34C) Aβ(16-35)-amide, cyclic] oligomer (4o)

[(17C, 34C) ARO 6-35)-amide, cyclic] oligomer (3o) already comprises a linkage.

p) Disulfide-stabilized (17C, 34C) ARO 6-35)-amide oligomer (4p)

(17C, 34C) Aβ(16-35)-amide oligomer (3p) was subjected to dialysis in order to remove the DTT and effect disulfide formation, as described in example 4k.

q) Cross-linked (17K, 34K) N-Met Aβ(1-42) oligomer (4q)

Subjecting (17K, 34K) N-Met Aβ(1-42) oligomer (3q) to conditions suitable for cross-linking two amine groups will yield the product.

r) Disulfide-stabilized (17KC, 34C) Aβ(13-42) oligomer (4r)

(17KC, 34C) Aβ(13-42) oligomer (3r) was subjected to dialysis in order to remove the DTT and effect disulfide formation, as described in example 4k.

s) Disulfide-stabilized (17C, 34C) Aβ(16-42) oligomer (4s)

(17C, 34C) Aβ(16-42) oligomer (3s) was subjected to dialysis in order to remove the DTT and effect disulfide formation, as described in example 4k.

t) (17C(ACM), 34C(ACM), 35M(S-oxide)) Aβ(16-35)-amide oligomer (4t)

(17C(ACM), 34C(ACM), 35M(S-oxide)) Aβ(16-35)-amide oligomer (3t) does not form cysteine bridges.

u) (17C(ACM), 34C(ACM)) Aβ(16-35)-amide oligomer (4u)

(17C(ACM), 34C(ACM)) Aβ(16-35)-amide oligomer (3u) does not form cysteine bridges.

v) EDC/NHS-linked (17K, 34E) Aβ(16-35) oligomer (4v)

Subjecting (17K, 34E) Aβ(16-35) oligomer (3v) to the procedure of example 4x will yield the product.

w1) SMCC-linked (17K, 34C) N-Met Aβ(1-42) oligomer (4w1)

(17K, 34C) N-Met Aβ(1-42) oligomer (3w) of example 3w in 0.05% SDS (2.37 mM peptide) were cross-linked with 11 mM sulfo-SMCC (Pierce cat #22622, supplied from a 100 mM stock in anhydrous DMSO) for 1 hr at room temperature. The reaction was quenched by the addition of 10 mM ethanolamine from a 100 mM, pH 8 aqueous stock solution. The excess reagent and reaction products were removed by dialysis vs. 5 mM NaPO₄, 35 mM NaCl, 0.05% SDS, pH 7.4 using a Slide-A-Lyzer with a 2000 Da mw cut-off membrane. The final concentration was determined using the BCA protein Assay (Pierce).

w2) MBS-linked (17K, 34C) N-Met Aβ(1-42) oligomer (4w2)

(17K, 34C) N-Met Aβ(1-42) oligomer (3w) of example 3w in 0.05% SDS (2.47 mM peptide) were cross-linked with 12 mM sulfo-MBS (Pierce cat #22312, supplied from a 100 mM stock in anhydrous DMSO) for 1 hr at room temperature. The reaction was quenched by the addition of 10 mM ethanolamine from a 100 mM, pH 8 aqueous stock solution. The excess reagent and reaction products were removed by dialysis vs. 5 mM NaPO₄, 35 mM NaCl, 0.05% SDS, pH 7.4 using a Slide-A-Lyzer with a 2000 Da MW cut-off membrane. The final concentration was determined using the BCA protein Assay (Pierce).

w3) SIAB-linked (17K, 34C) N-Met Aβ(1-42) oligomer (4w3)

(17K, 34C) N-Met Aβ(1-42) oligomer (3w) of example 3w in 0.05% SDS (2.47 mM peptide) were cross-linked with 12 mM sulfo-SIAB (Pierce cat #22327, supplied from a 100 mM stock in anhydrous DMSO) for 1 hr at room temperature. The reaction was quenched by the addition of 10 mM ethanolamine from a 100 mM, pH 8 aqueous stock solution. The excess reagent and reaction products were removed by dialysis vs. 5 mM NaPO₄, 35 mM NaCl, 0.05% SDS, pH 7.4 using a Slide-A-Lyzer with a 2000 Da MW cut-off membrane. The final concentration was determined using the BCA protein Assay (Pierce).

x) EDC/NHS-linked (17K, 34E) N-Met Aβ(1-42) oligomer (4x)

(17K, 34E) N-Met Aβ(1-42) oligomer (3x) of example 3x in 0.05% SDS (2.14 mM peptide) were cross-linked using 20 mM EDC (Pierce cat #22980, supplied from a 100 mM aqueous stock) and 50 mM sulfo-NHS (Pierce cat #24520, supplied from a 100 mM aqueous stock) for 1 hr at room temperature. The excess reagent and reaction products were removed by dialysis vs. 5 mM NaPO₄, 35 mM NaCl, 0.05% SDS, pH 7.4 using a Slide-A-Lyzer with a 2000 Da MW cut-off membrane. The final concentration was determined using the BCA protein Assay (Pierce).

y) Disulfide-stabilized (17C, 34C) N-Met Aβ(1-42) oligomer (4y)

(17C, 34C) N-Met Aβ(1-42) oligomer (3y) was subjected to dialysis in order to remove the DTT and effect disulfide formation, as described in example 4k.

z) [(E22C*, G29C*) Aβ(16-35)-amide, cyclised] oligomer (4z)

[(22C*, 29C*) Aβ(16-35)-amide, cyclised] oligomer (3z) already comprises a linkage.

aa) Disulfide-stabilized (22C, 29C) Aβ(16-35)-amide oligomer (4aa)

Subjecting (22C, 29C) Aβ(16-35)-amide oligomer (3aa) to dialysis in order to remove the DTT and effect disulfide formation, as described in example 4k, will yield the product.

ab) Disulfide-stabilized (F20->FA19501, 17C, 34C) N-Met Aβ(1-42) oligomer (4ab)

Subjecting (F20->FA19501, L17C, L34C) N-Met Aβ(1-42) oligomer (3ab) to dialysis in order to remove the DTT and effect disulfide formation, as described in example 4k, will yield the product.

ac) Disulfide-stabilized (22C, 29C) Aβ(20-35) oligomer (4ac)

Subjecting (22C, 29C) Aβ(20-35) oligomer (3ac) to dialysis in order to remove the DTT and effect disulfide formation, as described in example 4k, will yield the product.

ad) Disulfide-stabilized (22C, 29C) Aβ(20-42) oligomer (3ad)

Subjecting (22C, 29C) Aβ(20-42) oligomer (3ad) to dialysis in order to remove the DTT and effect disulfide formation, as described in example 4k, will yield the product.

ae) Disulfide-stabilized (17C, 22C, 29C, 34C) Aβ(20-42) N-Met Aβ(1-42) oligomer (4ae)

Subjecting 17C, 22C, 29C, 34C) Aβ(20-42) N-Met Aβ(1-42) oligomer (3ae) to dialysis in order to remove the DTT and effect disulfide formation, as described in example 4k, will yield the product.

af) Disulfide-stabilized (K28G29->FA12401, 17C, 34C) N-Met Aβ(1-42) oligomer (4af)

Subjecting (K28G29->FA12401, 17C, 34C) N-Met Aβ(1-42) oligomer (3af) to dialysis in order to remove the DTT and effect disulfide formation, as described in example 4k, will yield the product.

### Example 5

### Thermolysin Truncation

a) Thermolysin truncation of disulfide-stabilized (14C, 37C) N-Met Aβ(1-42) oligomer (5a)

0.5 ml of disulfide-stabilized (14C, 37C) N-Met Aβ(1-42) oligomer (4a) of example 4a was added with 1/91 Thermolysin w/w (Roche, Cat. no.: 161586). Thermolysin was dissolved with a concentration of 1 mg/ml in H₂O (freshly prepared). The samples were incubated under shaking for 20 h at 30° C. Then 2.5 μl of a 100 mM EDTA solution, pH 7.4, in water were added and the samples were shaken for 5 min at room temperature. The samples were subsequently adjusted to an SDS content of 0.1% with 5 μl of a 10% strength SDS solution. Samples were shaken for 10 min at room temperature. Subsequently the samples were concentrated to approx. 20 μl via a 0.4 ml 30 kDa Ultrafree-MC tube (Amicon, Cat. no.: UFC3LTK00). The concentrates were admixed with 0.2 ml of buffer (5 mM NaH₂PO₄, 35 mM NaCl, pH 7.4) and again concentrated to 20 μl using the 30 kDa Ultrafree-MC tubes (Amicon, Cat. no.: UFC3LTK00). Then the concentrates were adjusted with 0.38 ml of buffer (5 mM NaH₂PO₄, 35 mM NaCl, pH 7.4) to a final volume of 0.4 ml. Subsequently, the samples were adjusted to an SDS content of 0.05% with 2 μl of a 10% strength SDS solution and stored at −30° C. until further use.

b) Thermolysin truncation of disulfide-stabilized (15C, 36C) N-Met Aβ(1-42) oligomer (5b)

Disulfide-stabilized (15C, 36C) N-Met Aβ(1-42) oligomer (4b) of example 4b was subjected to thermolysin truncation using the procedure described example 5a.

c) Thermolysin truncation of disulfide-stabilized (16C, 35C) N-Met Aβ(1-42) oligomer (5c)

Disulfide-stabilized (16C, 35C) N-Met Aβ(1-42) oligomer (4c) of example 4c was subjected to thermolysin truncation using the procedure described example 5a.

d) Thermolysin truncation of disulfide-stabilized (17C, 34C) N-Met Aβ(1-42) oligomer (5d)

Disulfide-stabilized (17C, 34C) N-Met Aβ(1-42) oligomer (4d) of example 4d was subjected to thermolysin truncation using the procedure described example 5a.

e) Thermolysin truncation of disulfide-stabilized (18C, 33C) N-Met Aβ(1-42) oligomer (5e)

Disulfide-stabilized (18C, 33C) N-Met Aβ(1-42) oligomer (4e) of example 4e was subjected to thermolysin truncation using the procedure described example 5a.

f) Thermolysin truncation of disulfide-stabilized (19C, 32C) N-Met Aβ(1-42) oligomer (5f)

Disulfide-stabilized (19C, 32C) N-Met Aβ(1-42) oligomer (4f) of example 4f was subjected to thermolysin truncation using the procedure described example 5a.

g) Thermolysin truncation of disulfide-stabilized (20C, 31C) N-Met Aβ(1-42) oligomer (5g)

Disulfide-stabilized (20C, 31C) N-Met Aβ(1-42) oligomer (4g) of example 4g was subjected to thermolysin truncation using the procedure described example 5a.

h) Thermolysin truncation of disulfide-stabilized (21C, 30C) N-Met Aβ(1-42) oligomer (5h)

Disulfide-stabilized (21C, 30C) N-Met Aβ(1-42) oligomer (4h) of example 4h was subjected to thermolysin truncation using the procedure described example 5a.

i) Thermolysin truncation of disulfide-stabilized (22C, 29C) N-Met Aβ(1-42) oligomer (5i)

Disulfide-stabilized (22C, 29C) N-Met Aβ(1-42) oligomer (4i) of example 41 was subjected to thermolysin truncation using the procedure described example 5a.

j) Thermolysin truncation of Aβ(1-42) globulomer (5j)

Aβ(1-42) wilde type globulomer (Bachem H-1368) (4j) of example 4j was subjected to thermolysin truncation using the procedure described example 5a.

k) Thermolysin truncation of disulfide stabilized (17C, 34C) Aβ(12-42) oligomer (5k)

Disulfide stabilized (17C, 34C) Aβ(12-42) oligomer (4k) of example 4k was subjected to thermolysin truncation using the procedure described example 5a.

l) Thermolysin truncation of (17C(ACM), 34C(ACM)) Aβ(16-35) oligomer (5l)

(17C(ACM), 34C(ACM)) Aβ(16-35) oligomer (31) was subjected to thermolysin truncation using the procedure described example 5a.

m) Thermolysin truncation of Aβ(16-35)-amide oligomer (5m)

Aβ(16-35)-amide oligomer (3m) of example 3m was subjected to thermolysin truncation using the procedure described example 5a.

n) Thermolysin truncation of (17K, 34K) Aβ(16-35)-amide oligomer (5n)

Subjecting (17K, 34K) Aβ(16-35)-amide oligomer (3n) to thermolysin truncation using the procedure described example 5a will yield the product.

o) Thermolysin truncation of [(17C, 34C) Aβ(16-35)-amide, cyclic] oligomer (5o)

[(17C, 34C) Aβ(16-35)-amide, cyclic] oligomer (3o) of example 3o was subjected to thermolysin truncation using the procedure described example 5a.

p) Thermolysin truncation of disulfide stabilized (17C, 34C) Aβ(16-35)-amide oligomer (5p)

Disulfide stabilized (17C, 34C) Aβ(16-35)-amide oligomer (4p) of example 4p was subjected to thermolysin truncation using the procedure described example 5a.

q) Thermolysin truncation of (17K, 34K) N-Met Aβ(1-42) oligomer (5q)

Subjecting (17K, 34K) N-Met Aβ(1-42) oligomer (3q) to thermolysin truncation using the procedure described example 5a will yield the product.

r) Thermolysin truncation of disulfide stabilized (17KC, 34C) Aβ(13-42) oligomer (5r)

Subjecting disulfide stabilized (17KC, 34C) Aβ(13-42) oligomer (4r) of example 4r to thermolysin truncation using the procedure described example 5a will yield the product.

s) Thermolysin truncation of disulfide stabilized (17C, 34C) Aβ(16-42) oligomer (5s)

Disulfide stabilized (17C, 34C) Aβ(16-42) oligomer (4s) of example 4s was subjected to thermolysin truncation using the procedure described example 5a.

t) Thermolysin truncation of (17C(ACM), 34C(ACM), 35M(S-oxide)) Aβ(16-35)-amide oligomer (53t)

Subjecting (17C(ACM), 34C(ACM), 35M(S-oxide)) Aβ(16-35)-amide oligomer (3t) to thermolysin truncation using the procedure described example 5a will yield the product.

u) Thermolysin truncation of (17C(ACM), 34C(ACM)) Aβ(16-35)-amide oligomer (5u)

Subjecting (17C(ACM), 34C(ACM)) Aβ(16-35)-amide oligomer (3u) to thermolysin truncation using the procedure described example 5a will yield the product.

v) Thermolysin truncation of (17K, 34E) Aβ(16-35) oligomer (5v)

Subjecting (17K, 34E) Aβ(16-35) oligomer (3v) to thermolysin truncation using the procedure described example 5a will yield the product.

w1) Thermolysin truncation of SMCC-linked (17K, 34C) N-Met Aβ(1-42) oligomer (5w)

Subjecting SMCC-linked (17K, 34C) N-Met Aβ(1-42) oligomer (4w1) to thermolysin truncation using the procedure described example 5a will yield the product.

w2) Thermolysin truncation of MBS-linked (17K, 34C) N-Met Aβ(1-42) oligomer (5w)

Subjecting MBS-linked (17K, 34C) N-Met Aβ(1-42) oligomer (4w2) to thermolysin truncation using the procedure described example 5a will yield the product.

w3) Thermolysin truncation of SIAB-linked (17K, 34C) N-Met Aβ(1-42) oligomer (5w)

Subjecting SIAB-linked (17K, 34C) N-Met Aβ(1-42) oligomer (4w3) to thermolysin truncation using the procedure described example 5a will yield the product.

x) Thermolysin truncation of EDC/NHS-linked (17K, 34E) N-Met Aβ(1-42) oligomer (5x)

Subjecting EDC/NHS-linked (17K, 34E) N-Met Aβ(1-42) oligomer (4x) to thermolysin truncation using the procedure described example 5a will yield the product.

y) Thermolysin truncation of disulfide stabilized (17C, 34C) N-Met Aβ(1-42) oligomer (5y)

Disulfide stabilized (17C, 34C) N-Met Aβ(1-42) oligomer (4y) of example 4y was subjected to thermolysin truncation using the procedure described example 5a.

z) Thermolysin truncation of [(22C*, 29C*) Aβ(16-35)-amide, cyclised] oligomer (5z)

Subjecting [(22C*, 29C*) Aβ(16-35)-amide, cyclised] oligomer (3z) to thermolysin truncation using the procedure described example 5a will yield the product.

aa) Thermolysin truncation of disulfide stabilized (22C, 29C) Aβ(16-35)-amide oligomer (5aa)

Subjecting disulfide stabilized (22C, 29C) Aβ(16-35)-amide oligomer 22C, 29C) Aβ(16-35)-amide oligomer (4aa) to thermolysin truncation using the procedure described example 5a will yield the product.

ab) Thermolysin truncation of disulfide stabilized (F20->FA19501, 17C, 34C) N-Met Aβ(1-42) oligomer (5ab)

Subjecting disulfide stabilized (F20->FA19501, 17C, 34C) N-Met Aβ(1-42) oligomer (4ab) to thermolysin truncation using the procedure described example 5a will yield the product.

ac) Thermolysin truncation of disulfide stabilized (22C, 29C) Aβ(20-35) oligomer (5ac)

Subjecting disulfide stabilized (22C, 29C) Aβ(20-35) oligomer (4ac) to thermolysin truncation using the procedure described example 5a will yield the product.

ad) Thermolysin truncation of disulfide stabilized (22C, 29C) Aβ(20-42) oligomer (5ad)

Subjecting disulfide stabilized (22C, 29C) Aβ(20-42) oligomer (4ad) to thermolysin truncation using the procedure described example 5a will yield the product.

ae) Thermolysin truncation of disulfide stabilized (17C, 22C, 29C, 34C) Aβ(20-42) N-Met Aβ(1-42) oligomer (5ae)

Subjecting disulfide stabilized (17C, 22C, 29C, 34C) Aβ(20-42) N-Met Aβ(1-42) oligomer (4ae) to thermolysin truncation using the procedure described example 5a will yield the product.

af) Thermolysin truncation of disulfide stabilized (K28G29->FA12401, 17C, 34C) N-Met Aβ(1-42) oligomer (5af)

Subjecting disulfide stabilized (K28G29->FA12401, 17C, 34C) N-Met Aβ(1-42) oligomer (4af) to thermolysin truncation using the procedure described example 5a will yield the product.

### Biophysical and Biochemical Characterization

### Example 7

### SDS PAGE Analysis

a) SDS-PAGE (10-20% tricine gel) of (17C, 34C) N-Met Aβ(1-42) oligomer

SDS-PAGE was run using a 10-20% tricine gel, 1.0 mm, 15 well (Invitrogen, Cat # EC66255), employing 2-x tricine sample buffer (Invitrogen, Cat # LC1676) and tricine running buffer (Invitrogen, Cat # LC1675). Samples were mixed with equal parts sample buffer and loaded without heating. The gel was developed at ambient temperature for 100 minutes using a constant voltage of 125 V. After development, the gel was stained using a methanolacetic acid based Coomassie R-250 stain (0.1% Coomassie Brilliant Blue R250, 30% methanol, 10% acetic acid, in water), and destained using 30% methanol/10% acetic acid/water. Standards used: SeeBlue Plus2 Pre-Stained Standards (Invitrogen, Cat # LC5925), consisting of myosin (210 kDa), phosphorylase (98 kDa), BSA (78 kDa), Glutamic Dehydrogenase (55 kDa), Alcohol Dehydrogenase (45 kDa), Carbonic Anhydrase (34 kDa), Myoglobin Red (17 kDa), Lysozyme (16 kDa), Aprotinin (7 kDa) and Insulin (4 kDa).

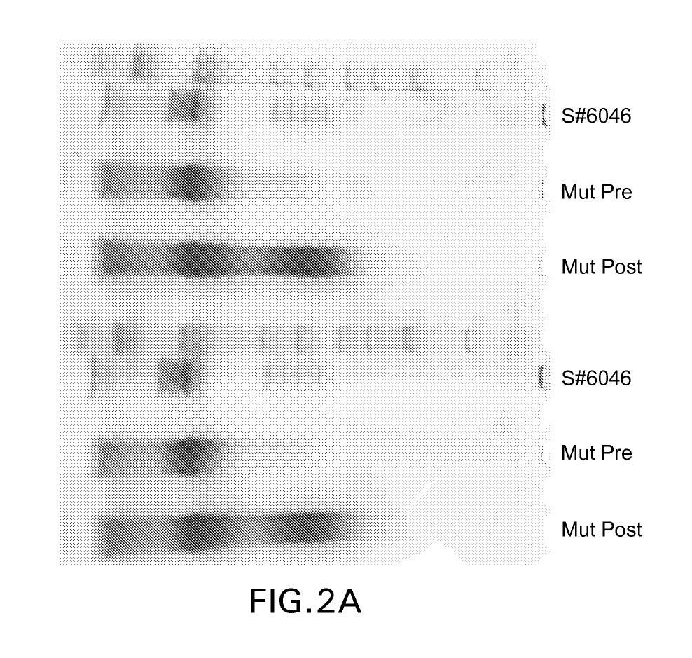

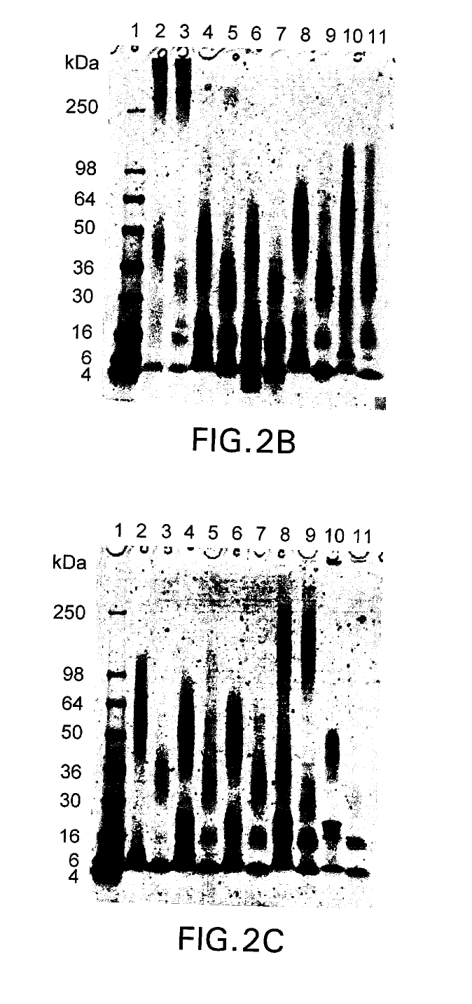

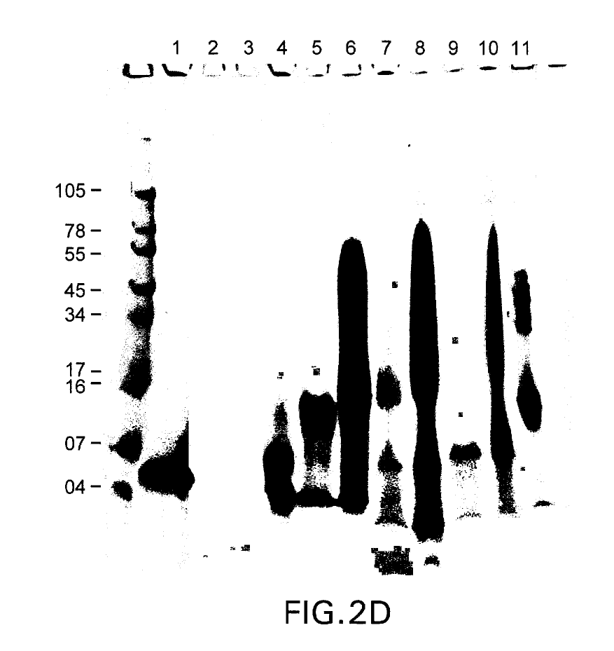

(17C, 34C) N-Met Aβ(1-42) oligomer (2a) from example 2a showed the typical Aβ globulomer banding pattern (FIG. 2A).

b) SDS-PAGE (4-20% Tris/Glycine gel) of (xC, yC) N-Met-Aβ(1-42) oligomers SDS-PAGE was run using the following parameters:
SDS-sample buffer:
   0.3 g SDS
   4 mL 1 M Tris/HCl pH 6.8
   8 mL glycerol
   70 μL 1% bromphenolblue in ethanol
   add H₂O to 50 mL
Running buffer:
   7.5 g Tris
   36 g Glycine
   2.5 g SDS
   add H₂O to 2.5 L
SDS-PAGE gel system:
   4-20% Tris/Glycine Gel: (Invitrogen Inc., Cat. no.: EC60255BOX)

10 μL (xC, yC) N-Met-Aβ(1-42) oligomer or Aβ(1-42) globulomer preparation before and after the thermolysin digestion were added to 10 μL SDS-sample buffer. The resulting 20 μL sample are loaded onto a 4-20% Tris/Glycin Gel (Invitrogen Inc., Cat. no.: EC60255BOX). The SDS-PAGE is conducted at a constant current of 25 mA.
Coomassie Staining:
Staining solution:
   2500 ml Methanol
   500 ml Acetic acid
   5 g Coomassie Brillant Blue R250, Fa. Bio-Rad, Cat. no. 161-0400
   2000 ml H₂O
Destaining solution:
   875 ml Methanol
   250 ml Acetic acid
   3925 ml H₂O

Subsequent to electrophoresis the gel was incubated in staining solution for 30 min at room temperature under shaking on a rocking platform. After staining the background of the gel was destained over night at room temperature in the destaining solution.

(14C, 37C) N-Met Aβ(1-42) oligomer from example 4a, thermolysin truncated (14C, 37C)N-Met Aβ(1-42) oligomer from example 5a, (15C, 36C) N-Met Aβ(1-42) oligomer from example 4b, thermolysin truncated (15C, 36C) N-Met Aβ(1-42) oligomer from example 5b, (16C, 35C) N-Met Aβ(1-42) oligomer from example 4c, thermolysin truncated (16C, 35C) N-Met Aβ(1-42) oligomer from example 5c, (17C, 34C) N-Met Aβ(1-42) oligomer from example 4d, thermolysin truncated (17C, 34C) N-Met Aβ(1-42) oligomer from example 5d, (18C, 33C)N-Met Aβ(1-42) oligomer from example 4e, and thermolysin truncated (18C, 33C) N-Met Aβ(1-42) oligomer from example 5e (FIG. 2B) as well as (19C, 32C) N-Met Aβ(1-42) oligomer from example 4f, thermolysin truncated (19C, 32C) N-Met Aβ(1-42) oligomer from example 5f, (20C, 31C) N-Met Aβ(1-42) oligomer from example 4g, thermolysin truncated (20C, 31C) N-Met Aβ(1-42) oligomer from example 5g, (21C, 30C) N-Met Aβ(1-42) oligomer from example 4h, thermolysin truncated (21C, 30C) N-Met Aβ(1-42) oligomer from example 5h, (22C, 29C) N-Met Aβ(1-42) oligomer from example 31, and thermolysin truncated (22C, 29C) N-Met Aβ(1-42) oligomer from example 5h (FIG. 2C) showed the typical Aβ globulomer banding pattern of Aβ(1-42) globulomer or Aβ(1-42) thermolysin truncated globulomer (although there are some apparent shifts of band height at which the (YC, YC) N-Met Aβ(1-42) oligomers and thermolysin truncated (YC, YC) N-Met Aβ(1-42) oligomers run).

Further, (17K, 34E) N-Met Aβ(1-42) oligomer (0.2% SDS) from example 3x, (17K, 34E) N-Met Aβ(1-42) oligomer (0.05% SDS) from example 3x, (17C(ACM), 34C(ACM)) Aβ(16-35) oligomer (0.2% SDS) from example 3u, (17C(ACM), 34C(ACM)) Aβ(16-35) oligomer (0.05% SDS) from example 3u, (17K, 34C) N-Met Aβ(1-42) oligomer (0.2% SDS) from example 3w, (17K, 34C) N-Met Aβ(1-42) oligomer (0.05% SDS) from example 3w, (17C, 34C) Aβ(16-42) oligomer (0.2% SDS) from example 3s, (17C, 34C) Aβ(16-42) oligomer (0.05% SDS) from example 3s, (17KC, 34C) Aβ(13-42) oligomer (0.2% SDS) from example 3r, and (17KC, 34C) Aβ(13-42) oligomer (0.05% SDS) from example 3r (FIG. 2D) showed the typical Aβ globulomer banding pattern of N-Met Aβ(1-42) globulomer.

### Example 8

### Direct ELISA

The immunoreactivity of (17C, 34C) N-Met Aβ(1-42) oligomer (2a) from example 2a was further characterized by using an antibody that is >1000 fold selective for the Aβ(1-42) globulomer form over fibril and free peptide (monoclonal antibody 5F7).

Materials: Microtiterplates were Nunc Immuno Plate, Maxi-Sorb Surface, flat bottom, (Catalogue #439454). The conjugate (secondary antibody) was Donkey anti-mouse HRPO conjugate, Jackson Immuno Research, (Catalogue #715-035-150). The HRPO Substrate was 3,3′,5′5′-Tetramethylbenzidine Liquid substrate (TMB), Sigma, (Catalogue #T4444). Non-fat Dry Milk (NFDM), was from BioRad, (Catalogue #170-6404). All other chemicals were from conventional sources.

Buffers and solutions: PBST buffer: Sigma PBS made with 0.05% Tween 20. PBST with 0.5% BSA was made by dissolving 0.5 mg BSA in 100 mL PBST. Blocking solution was 3% NFDM in PBST. Conjugate diluent was 1% NFDM in PBST. The coating buffer was 100 mM NaHCO₃ pH8.2. The HRPO Stop Solution was 2 M H₂SO₄.

Coating the plates: The Aβ globulomer to be tested was diluted to 1.0 μg/mL in coating buffer. 100 μL were added to each well to be coated. Plate was sealed with sealing film and left at 4° C. overnight.

Plate Blocking: The coating solution was removed from the wells. Each well was optionally washed 2-3× with 150 μL of PBST. 300 μL of blocking solution were added (3% NFDM in PBST). Plate was covered with plate sealing film and incubated for ˜2 hrs at room temperature, with agitation.

Primary Antibody: The blocking solution was removed from the wells. Each well was optionally washed 2-3× with 150 μL of PBST. 100 μL of primary antibody solutions were added. For the full length N-Met Aβ(1-42) globulomer, solutions of 0.04 to 100 μg/mL of antibody 5F7 were used. The antibody was diluted using PBST with 0.5% BSA. The plate was covered with plate sealing film and incubated for ˜2 to 3 hrs at room temperature with agitation.

Secondary Antibody (HRPO Conjugate): The primary antibody solution was removed from the wells. Each well was washed 2-3× with 150 μL of PBST. 200 μL of secondary antibody (HRPO conjugate) solution diluted 1:5000 in PBST with 1% NFDM were added. The plate was covered with plate sealing film and incubated for ˜1 hr at room temperature with agitation.

Substrate Development: The conjugate solution was removed from the wells. Each well was washed 2-3× with 200 μL of PBST. 100 μL of HRPO substrate solution were added to each well. Color was allowed to develop under observation. It turned blue. The reaction was usually done in 5-10 minutes at room temperature. 50 μL of stop solution were added to each well. The blue color turned yellow. Absorbance at 450 nm was read using a microtiterplate reader within 30 min of addition of the stop solution.

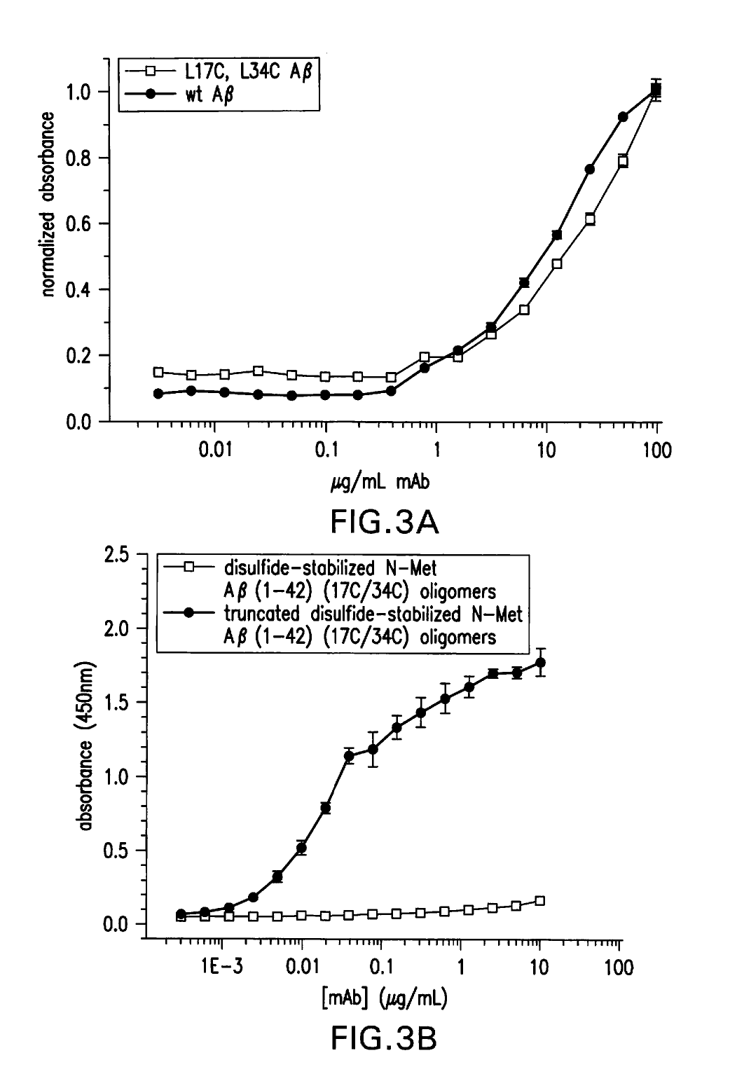

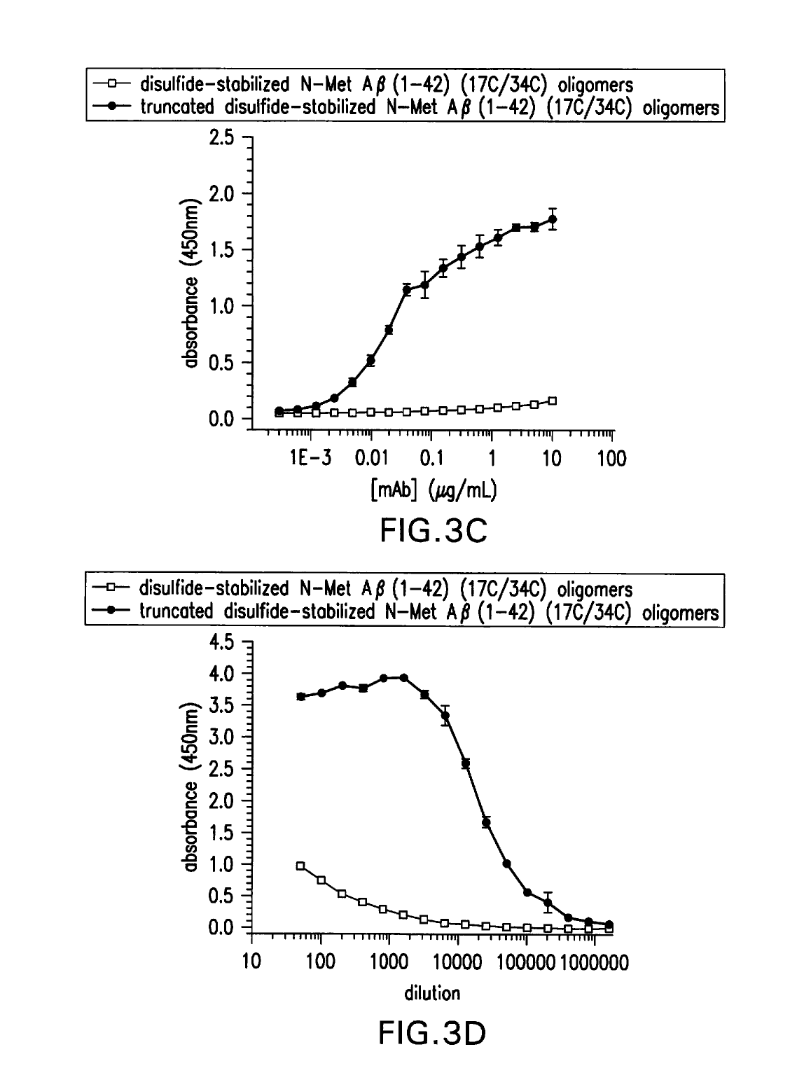

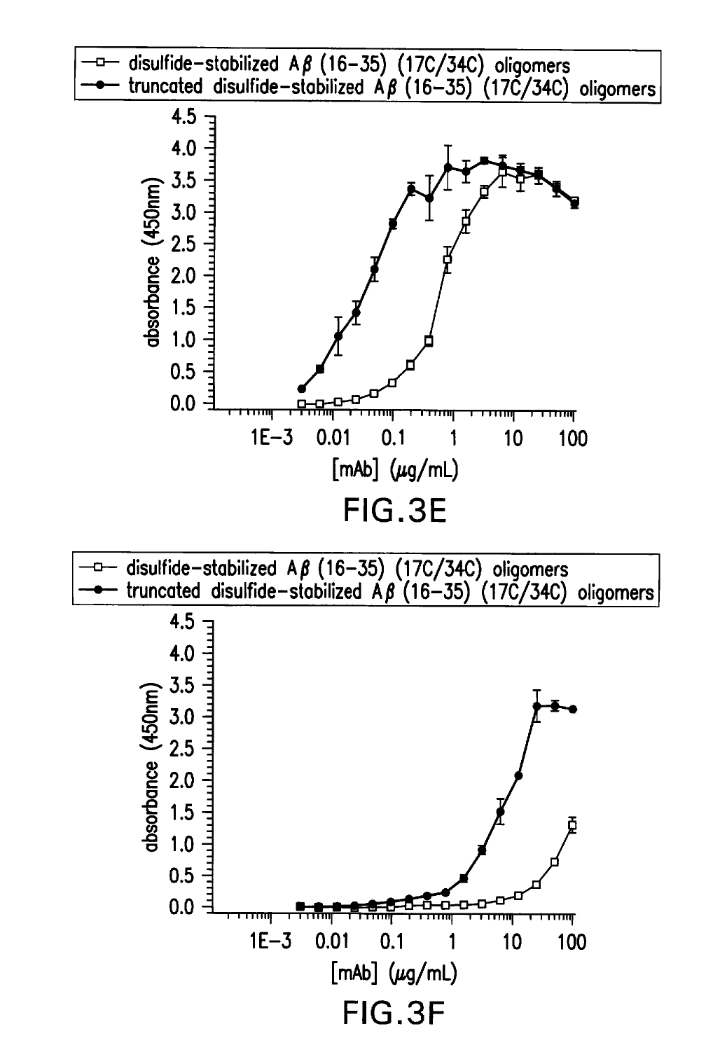

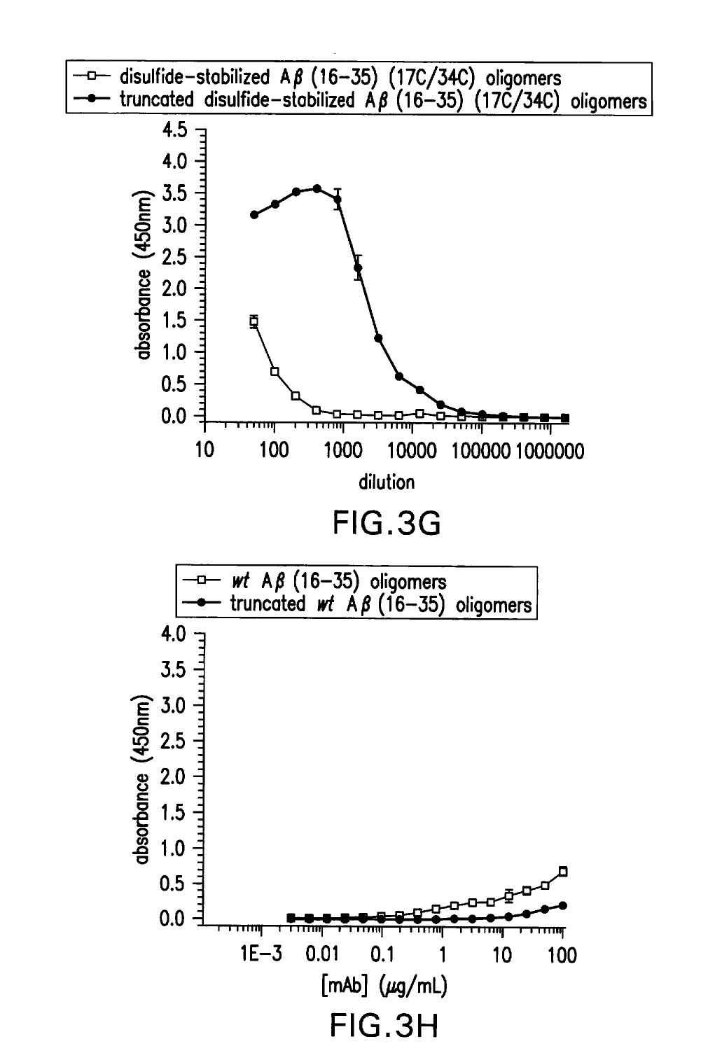

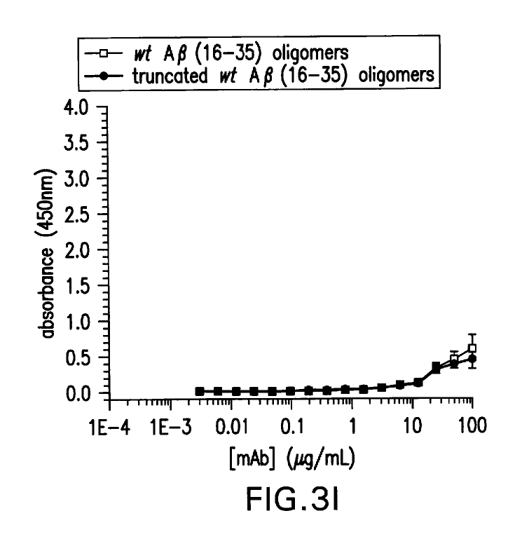

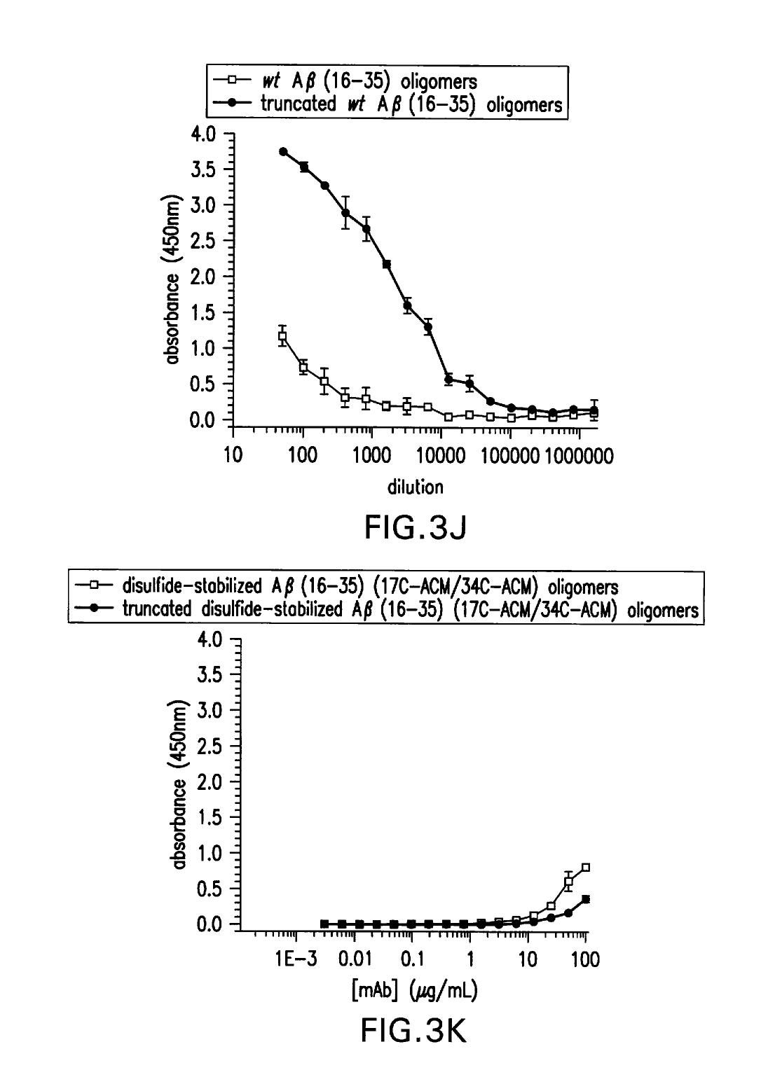

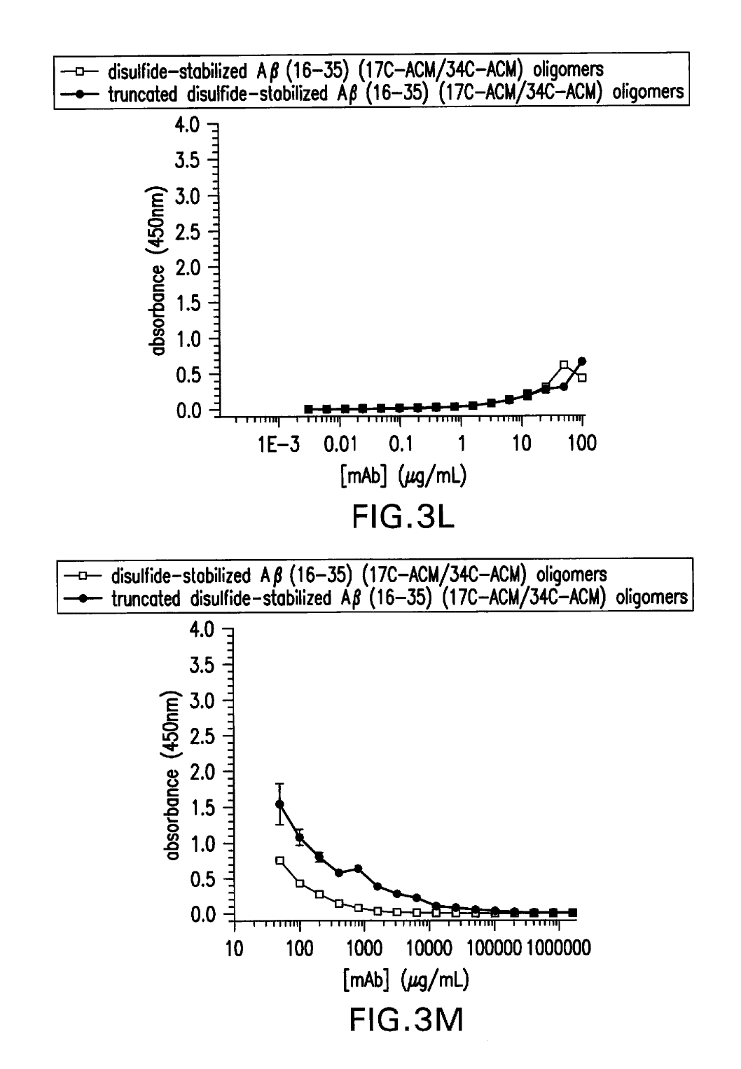

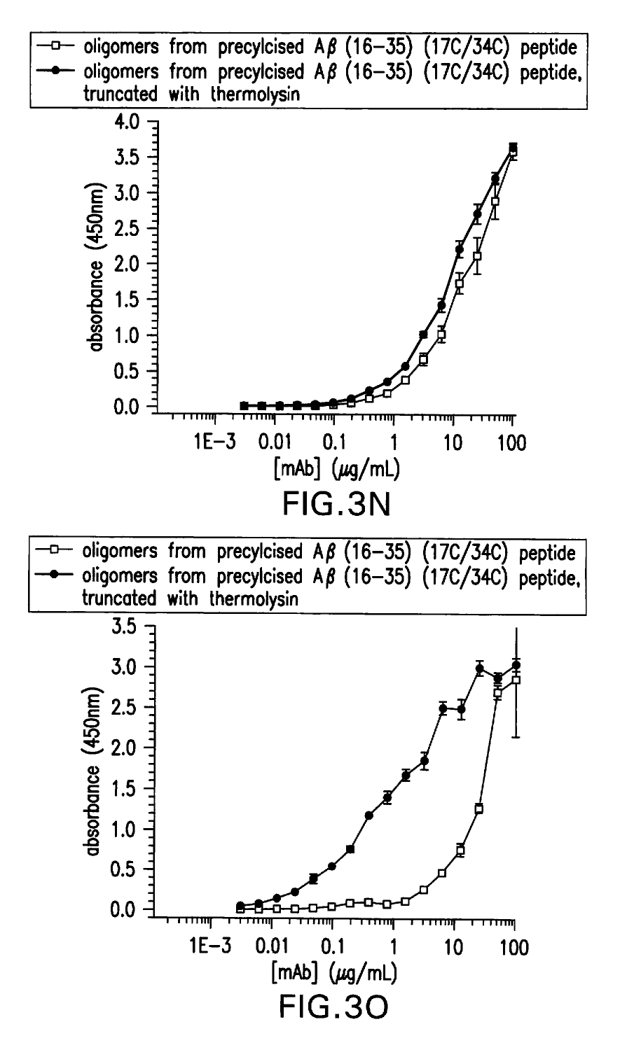

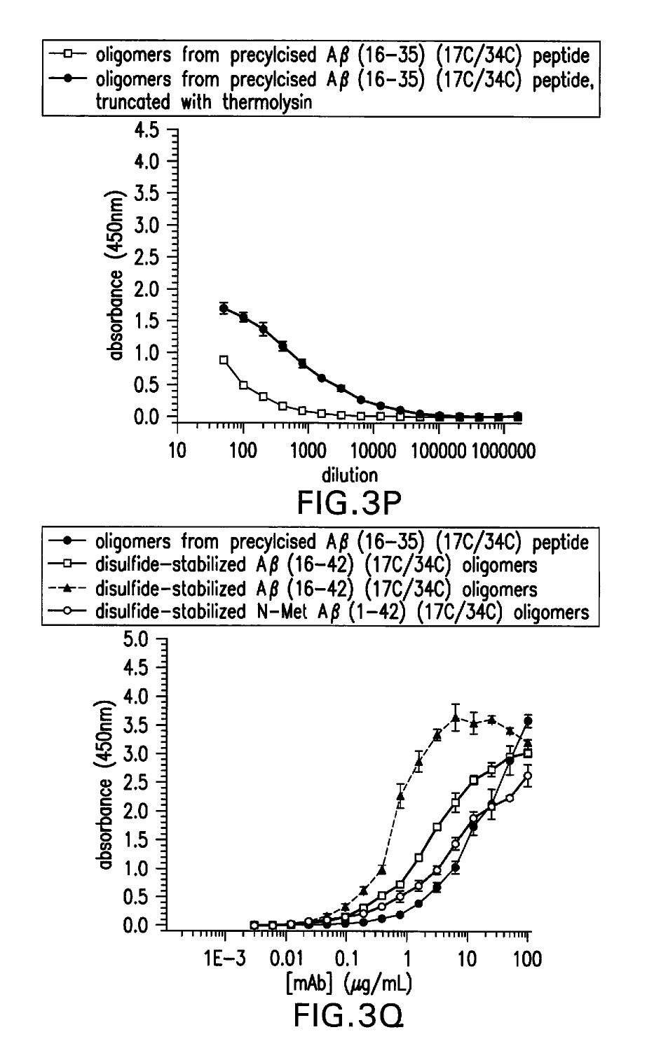

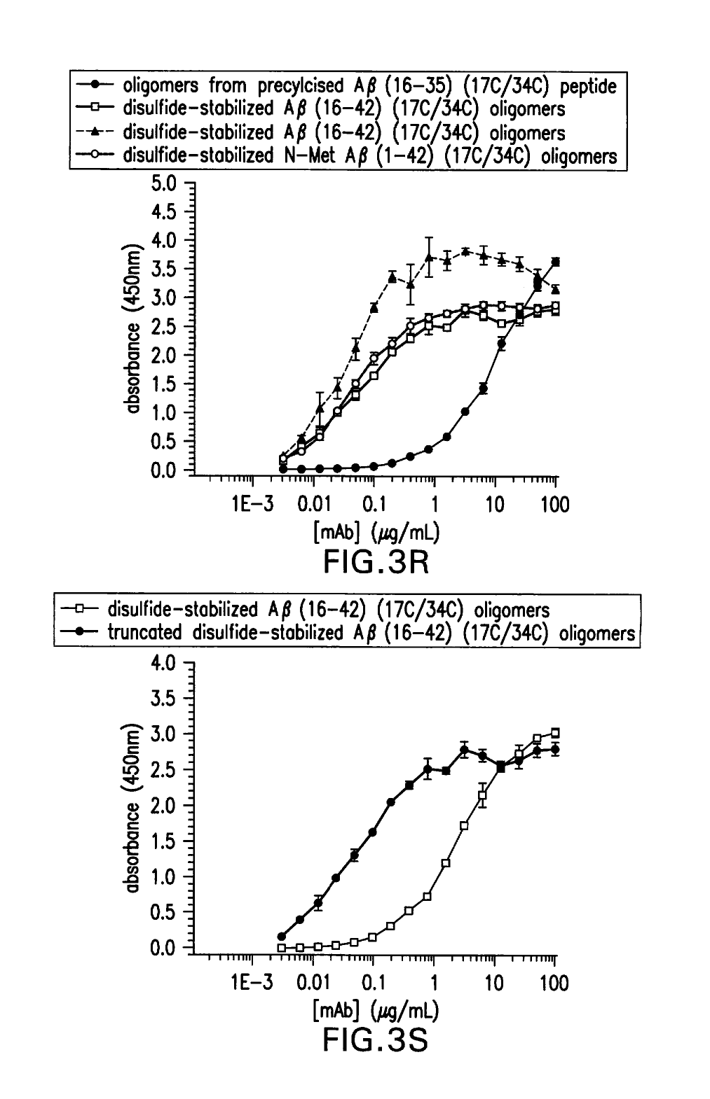

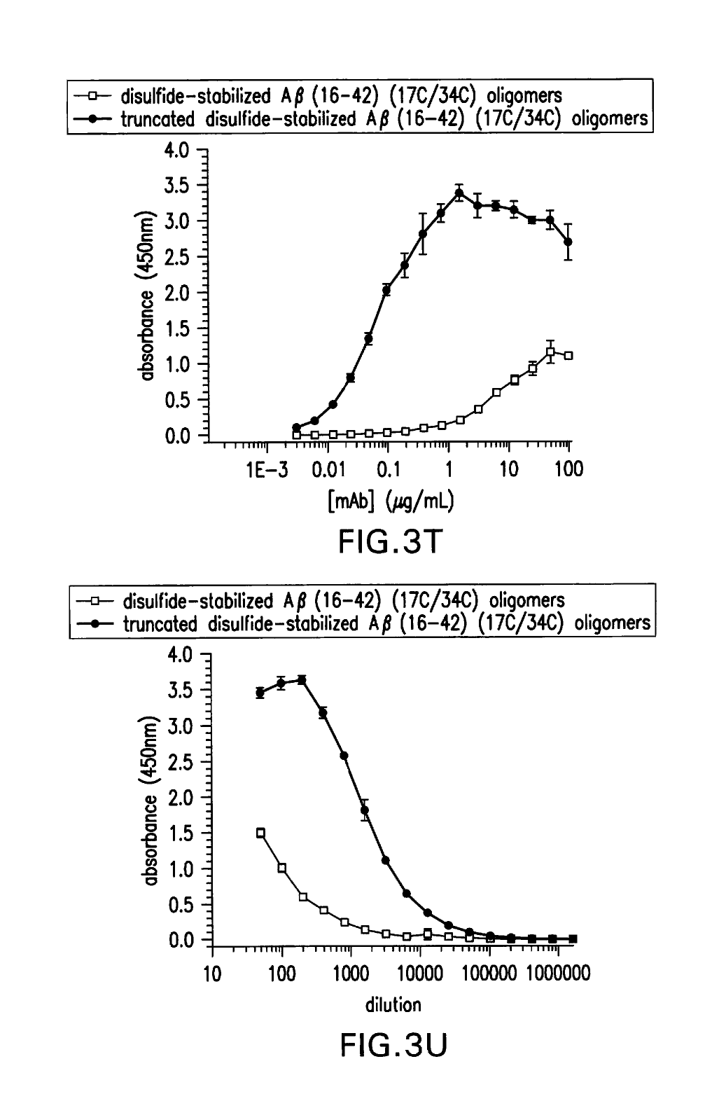

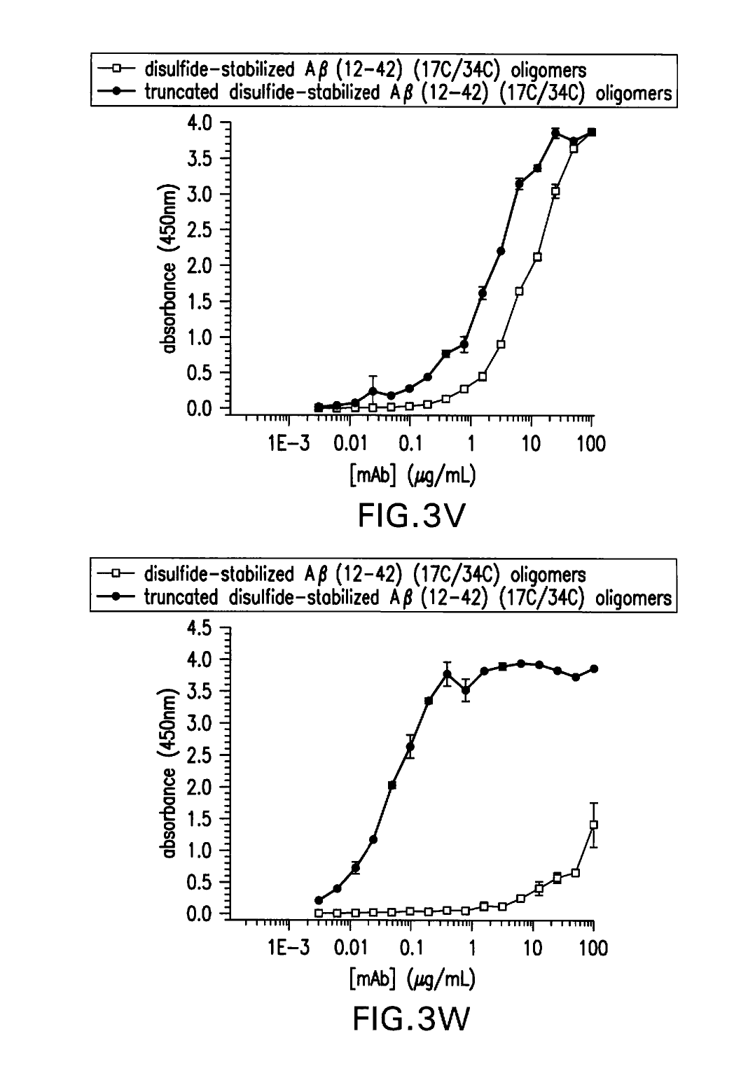

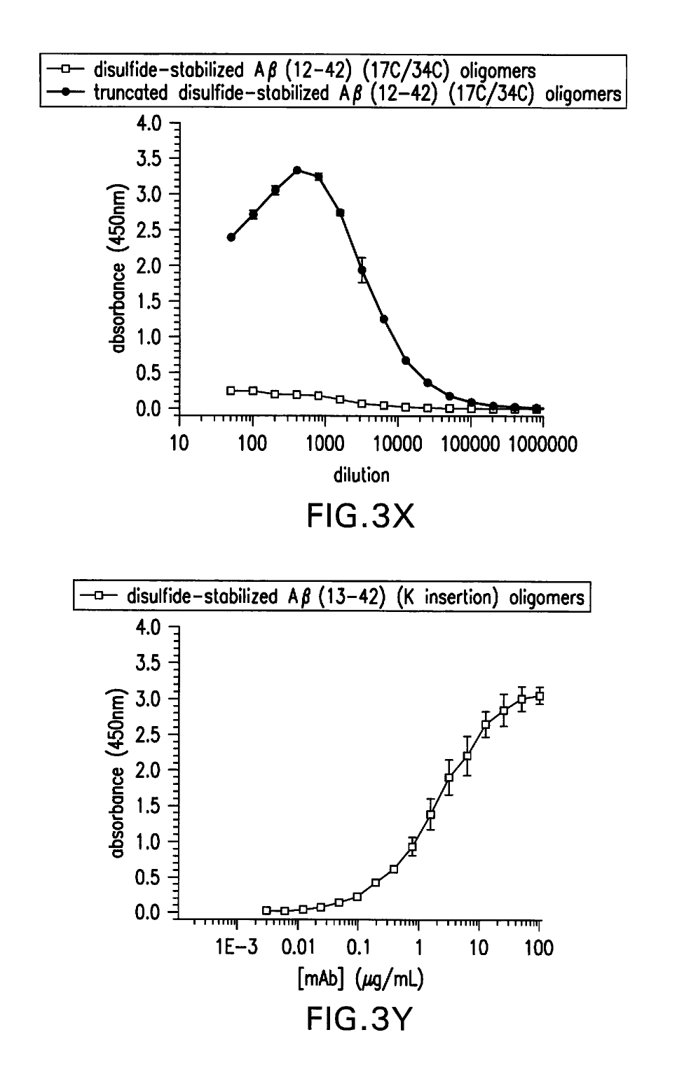

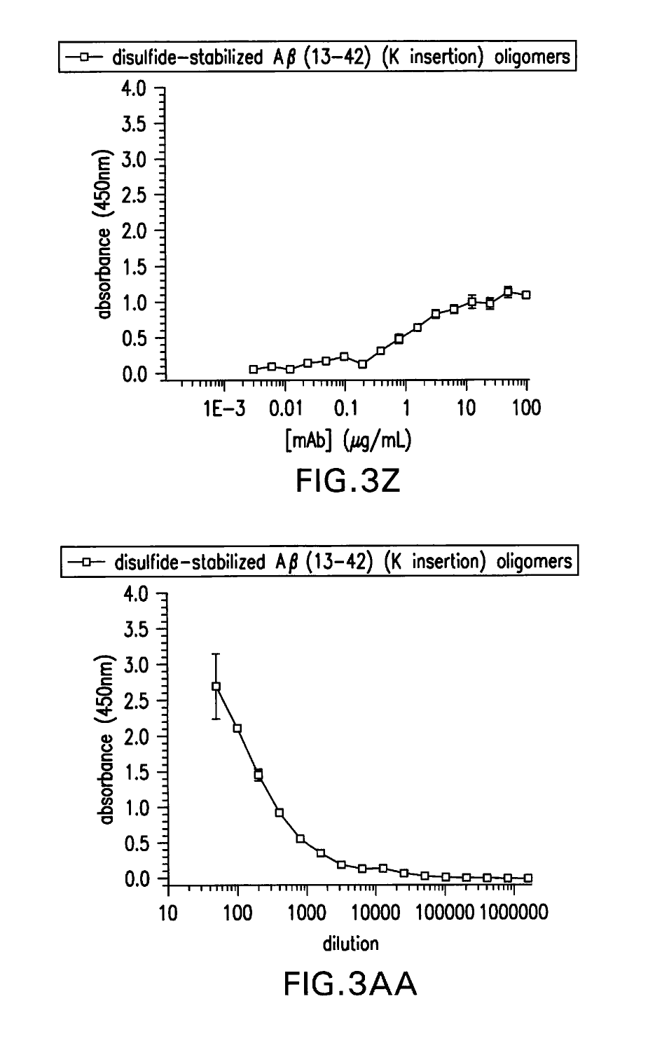

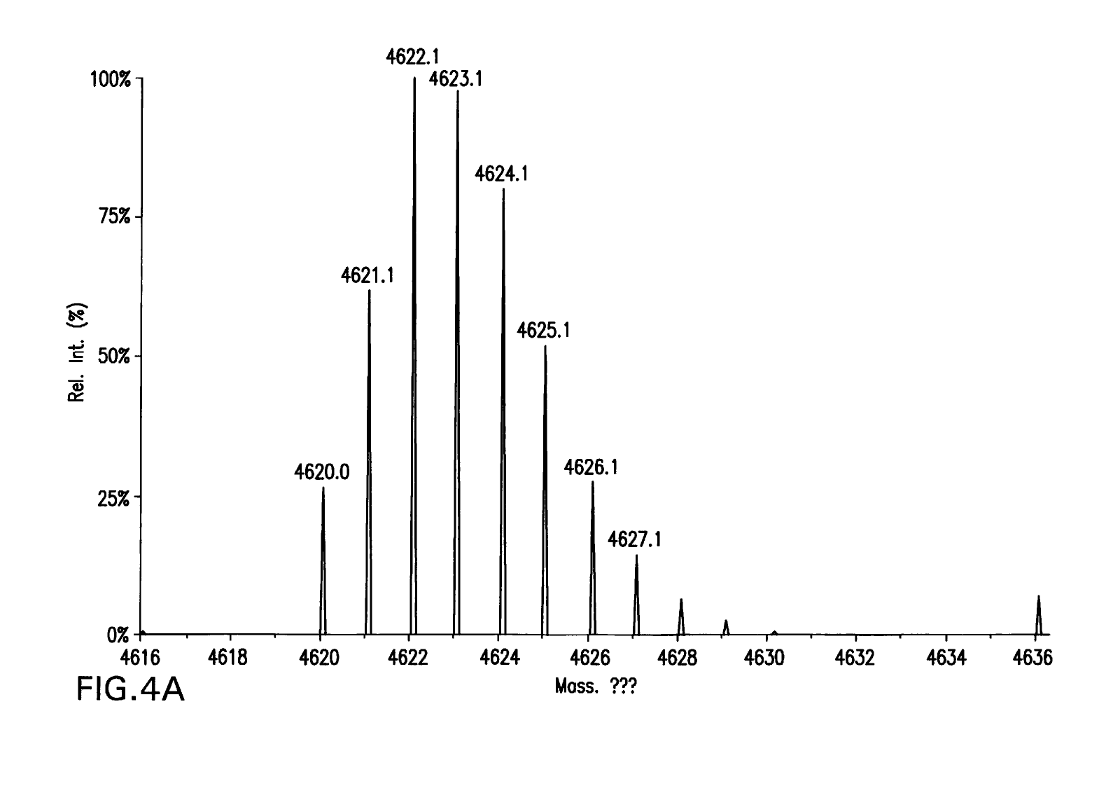

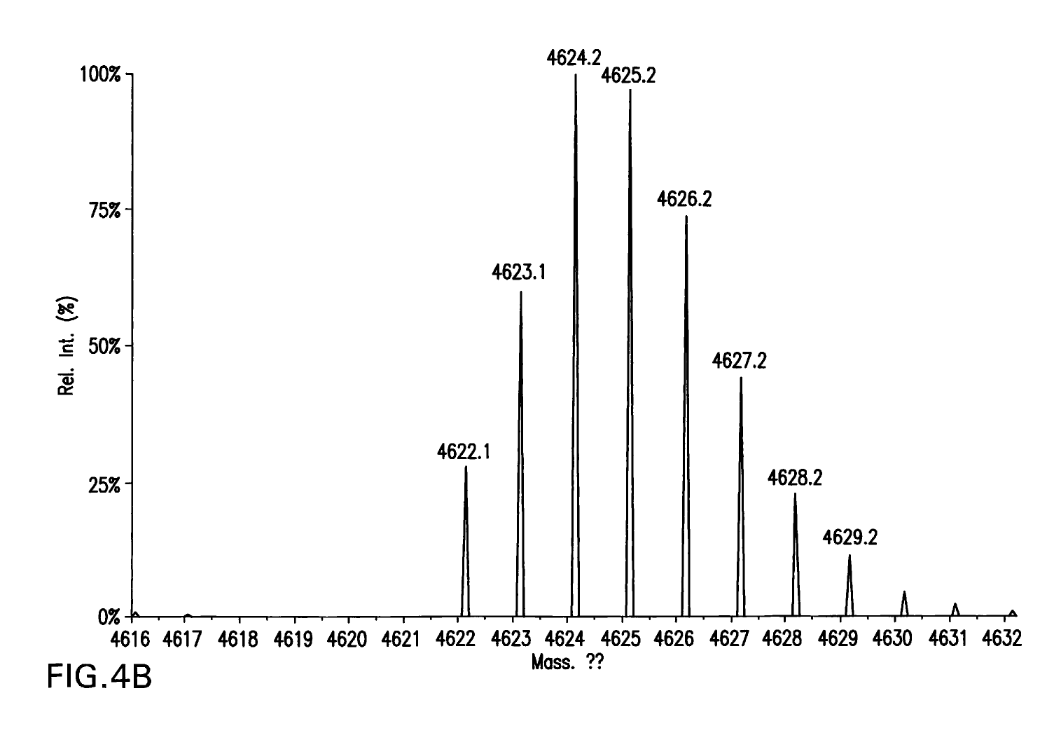

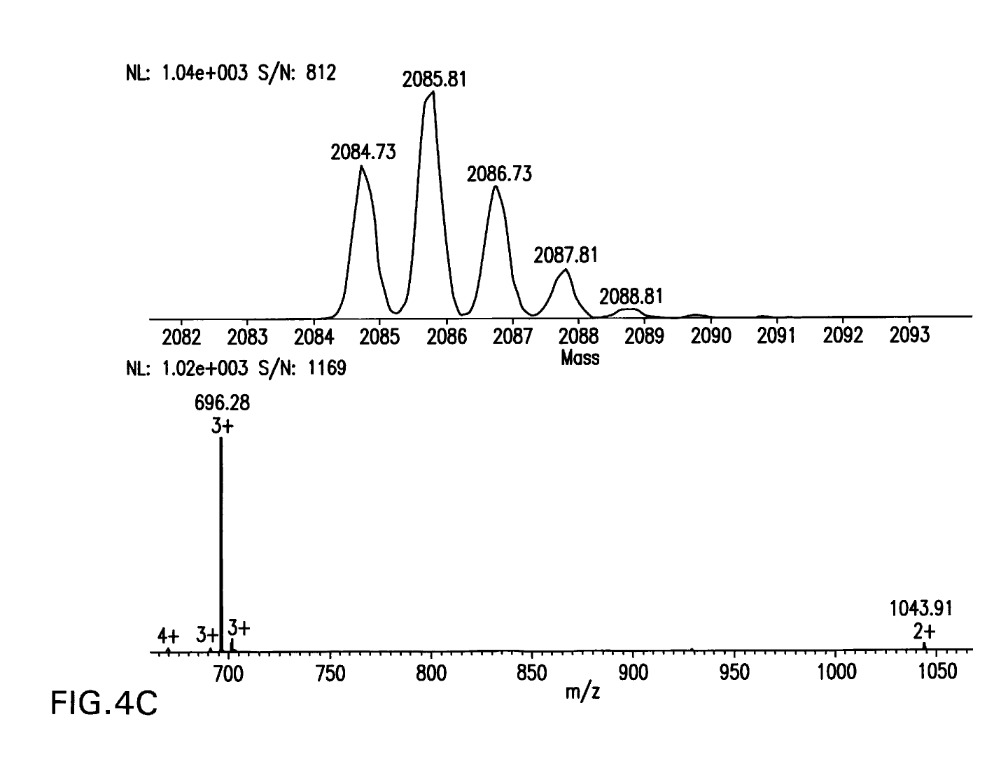

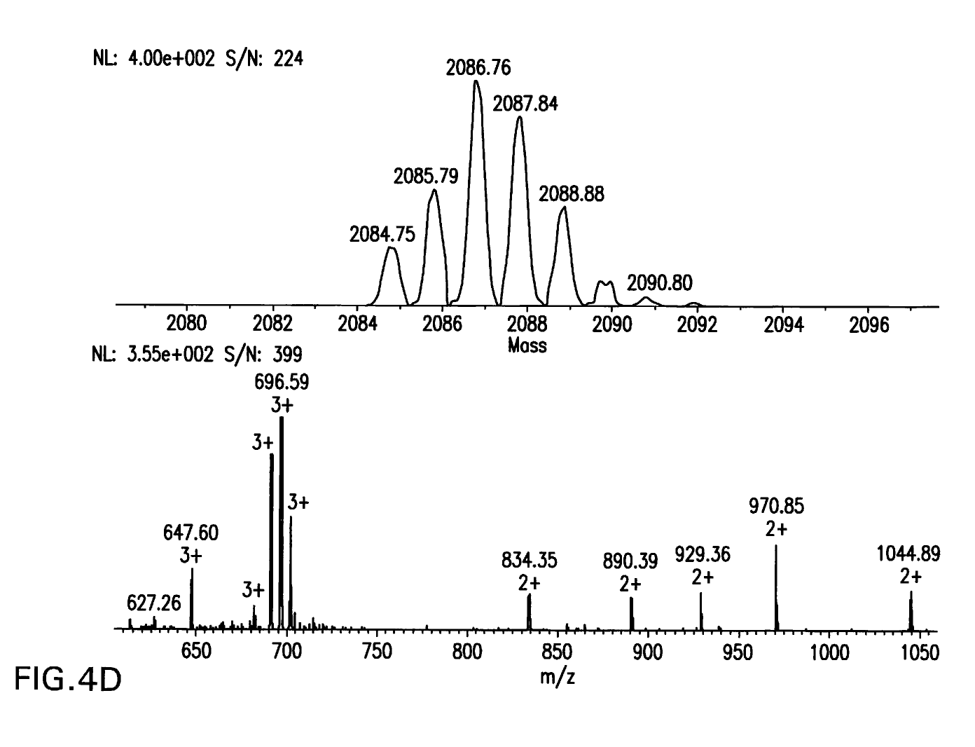

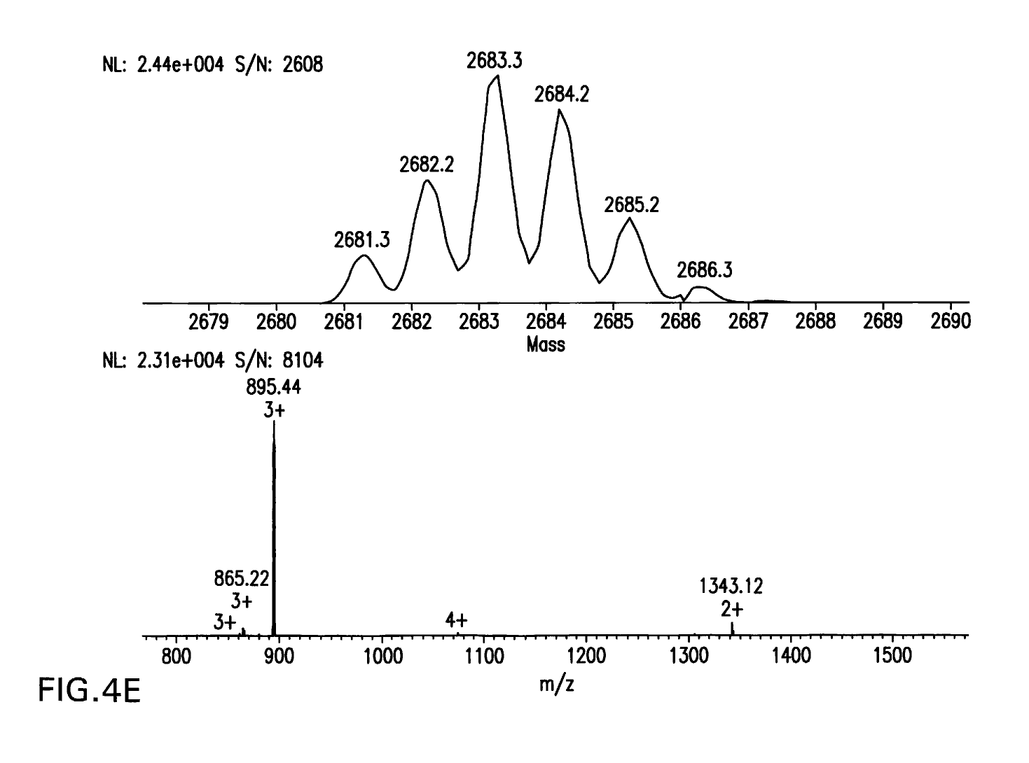

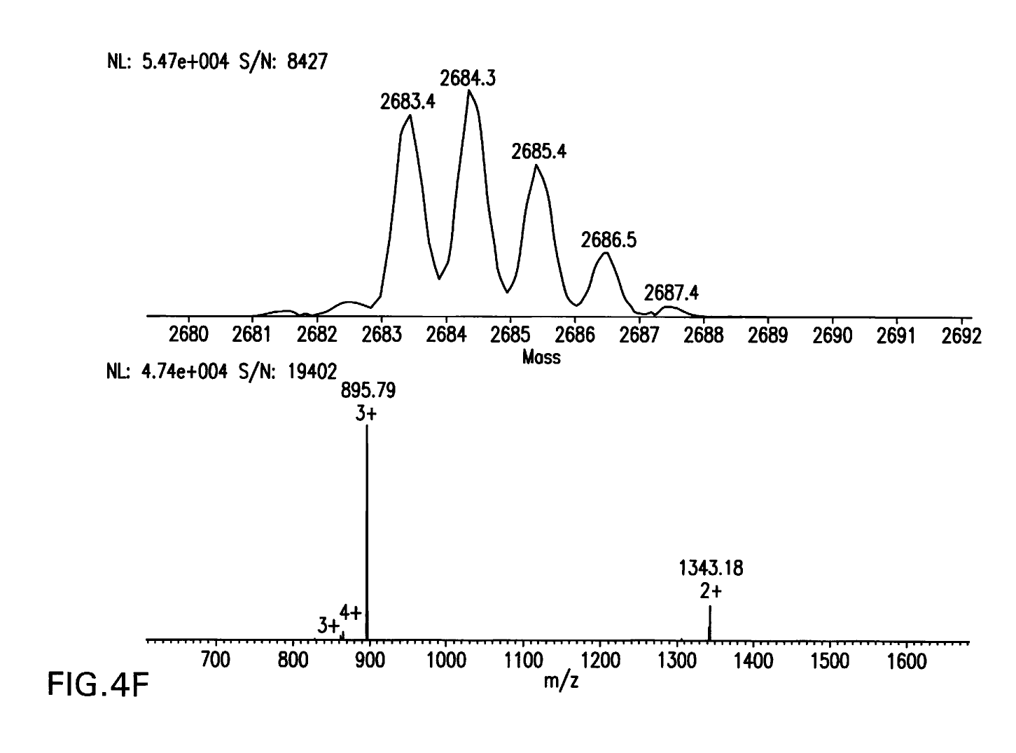

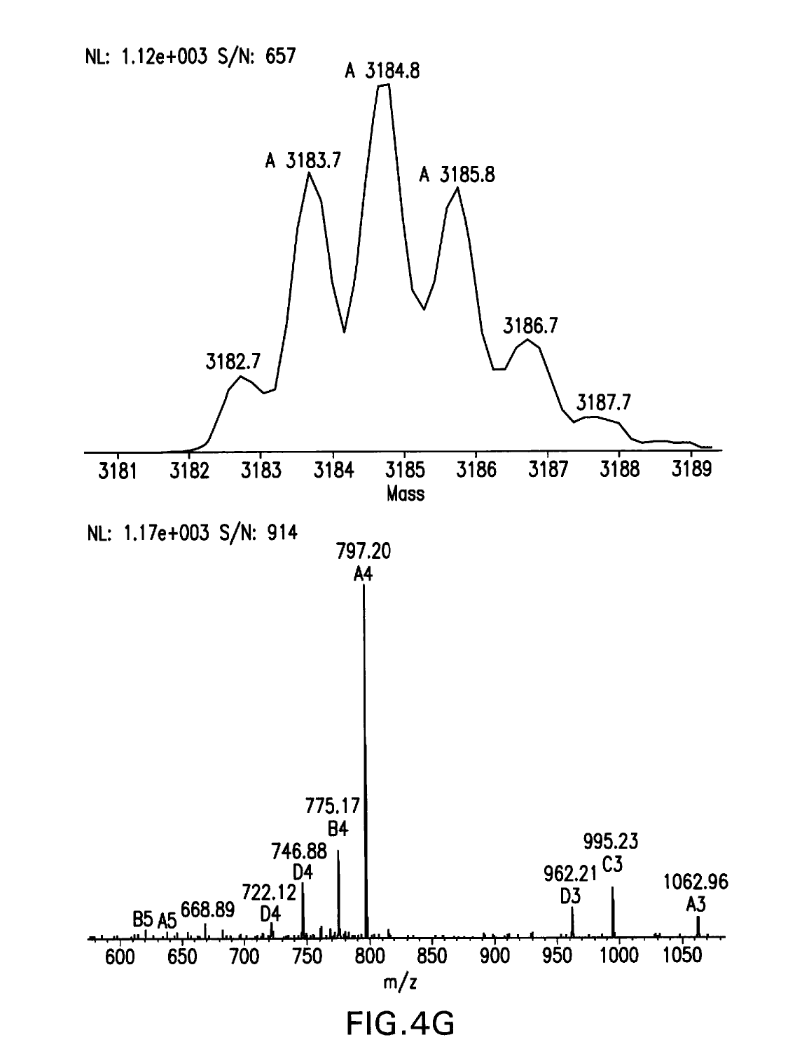

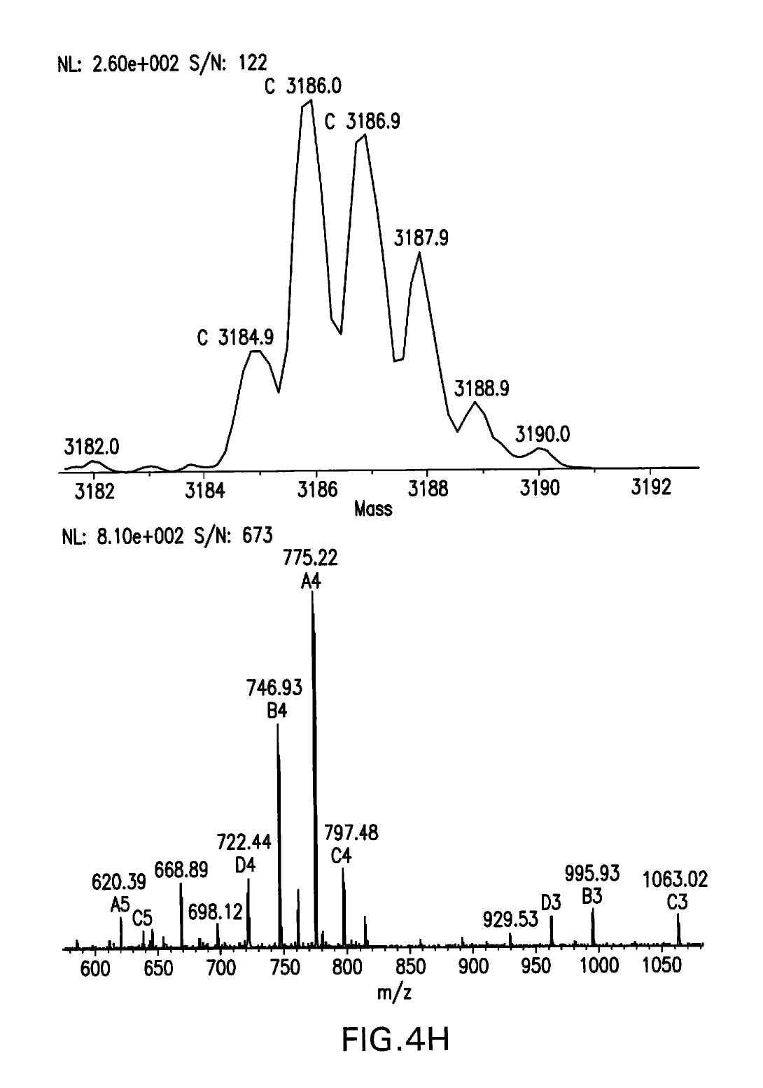

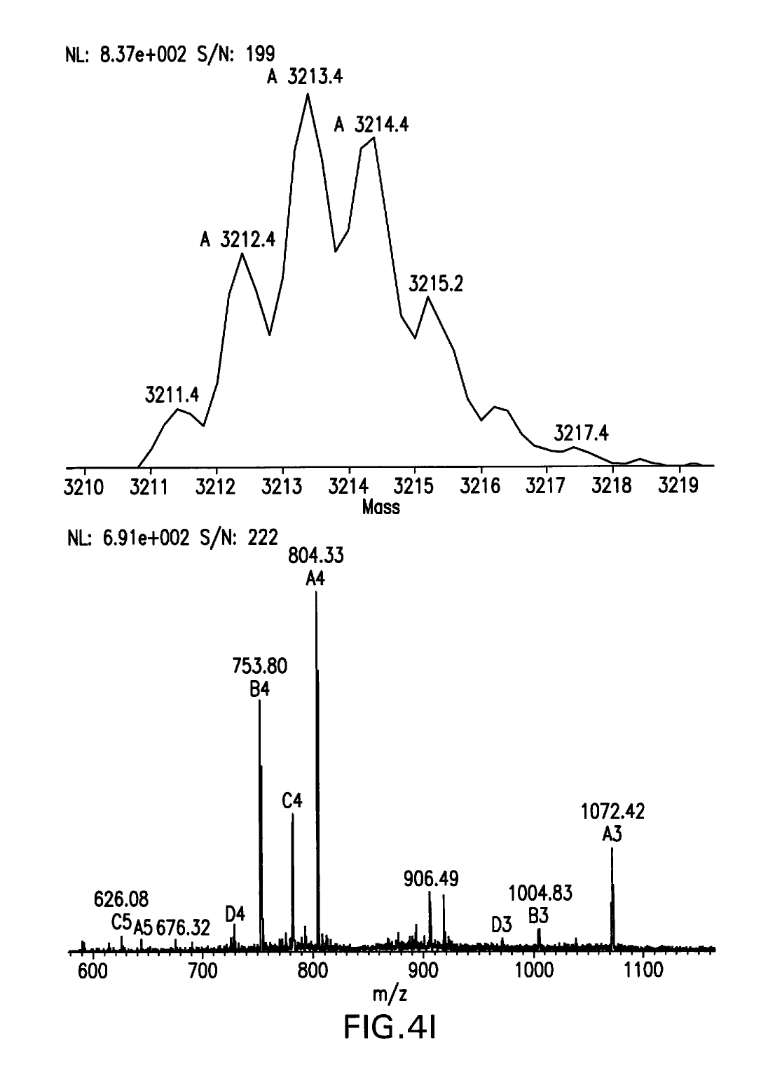

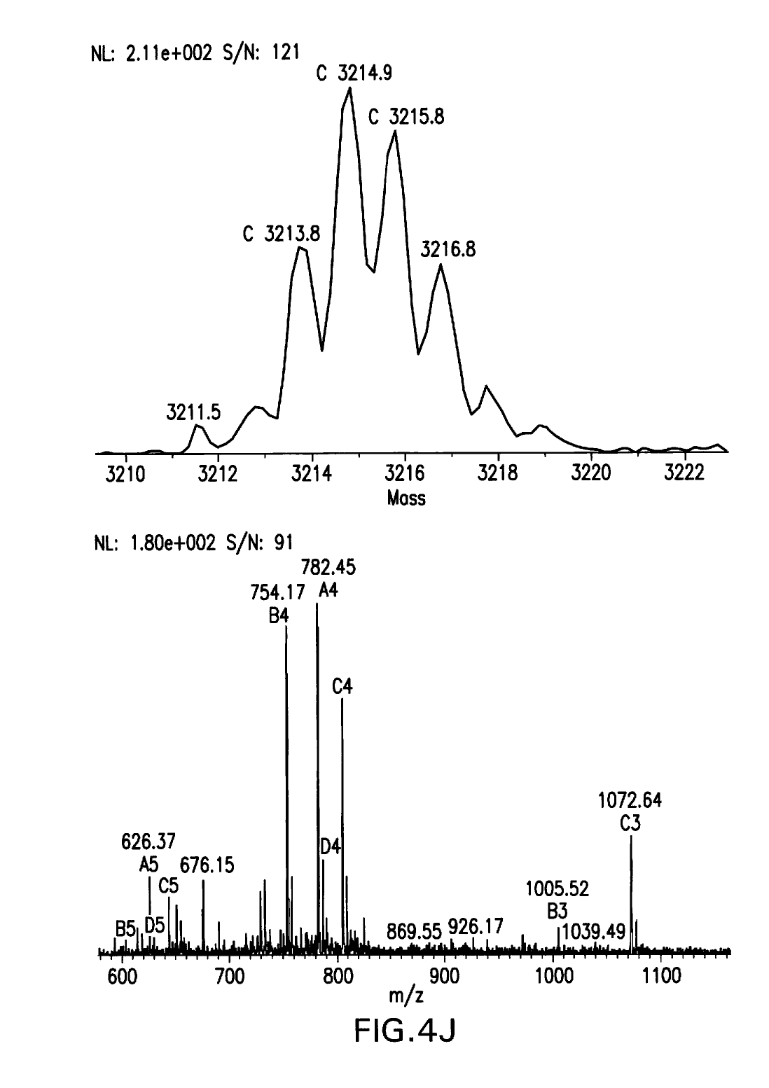

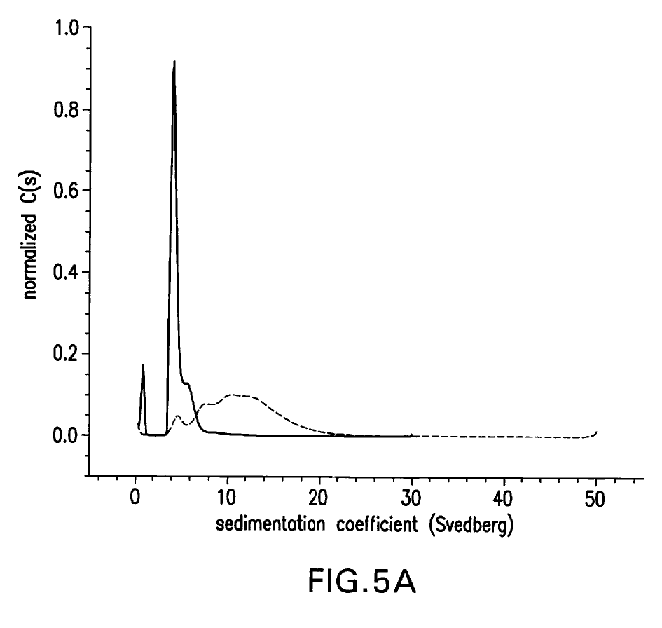

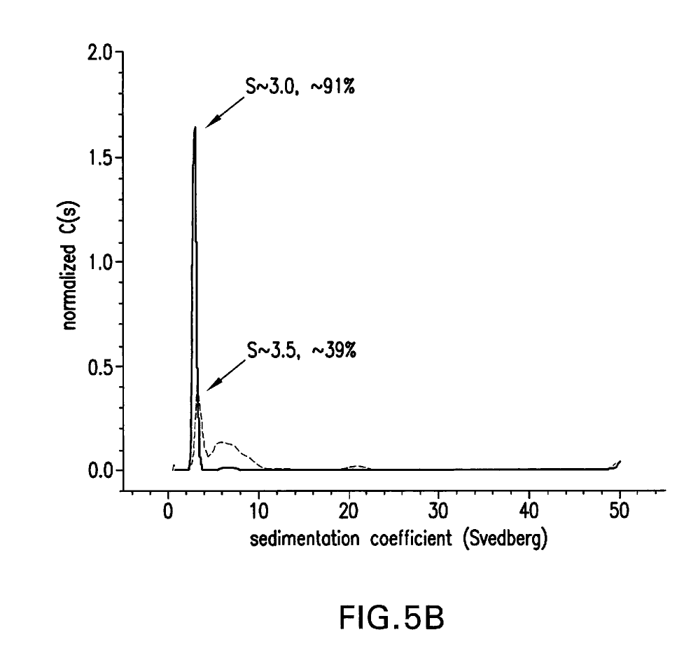

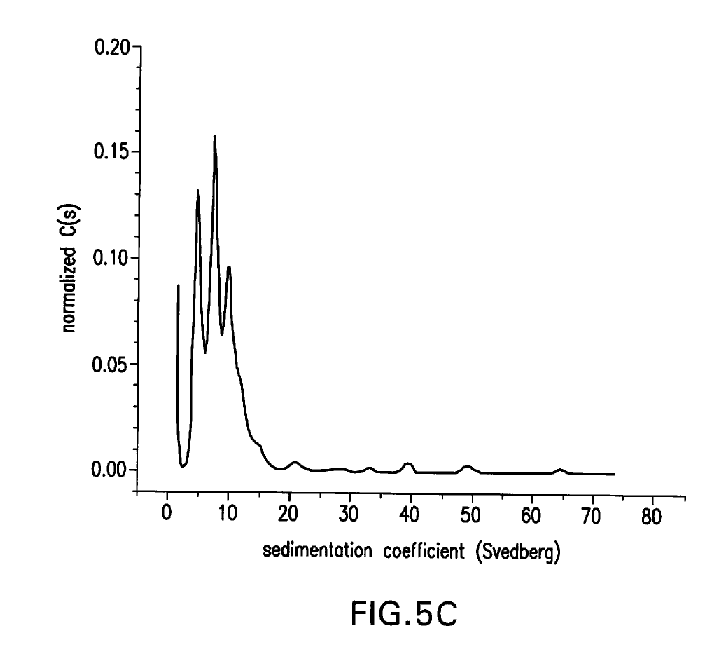

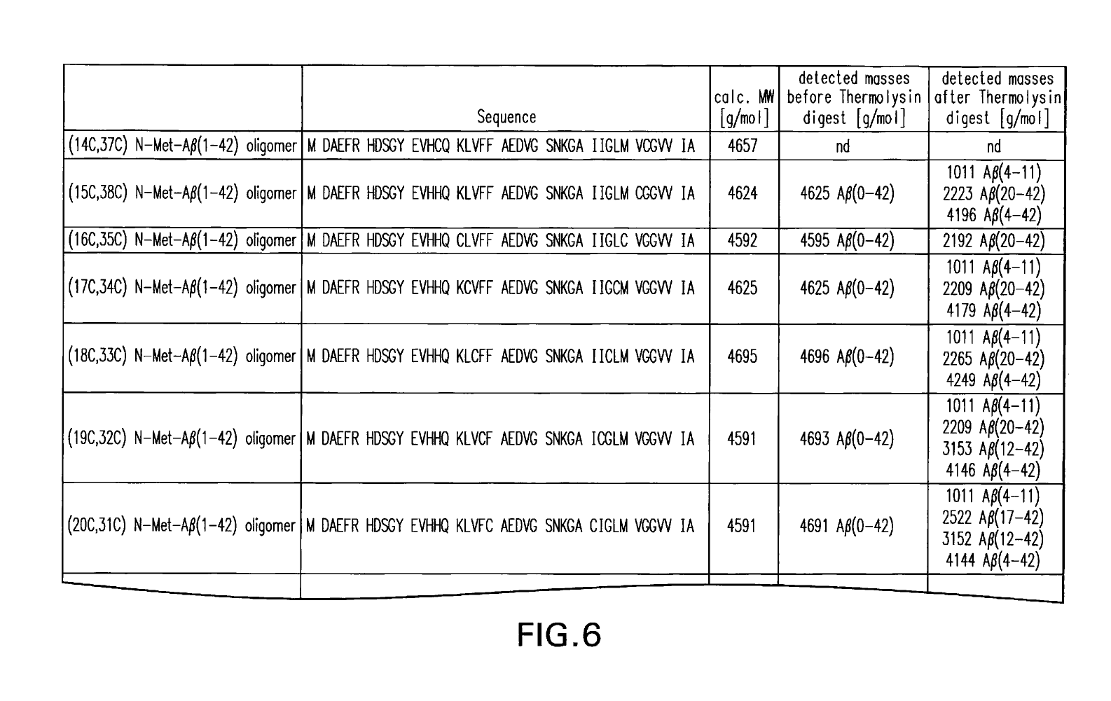

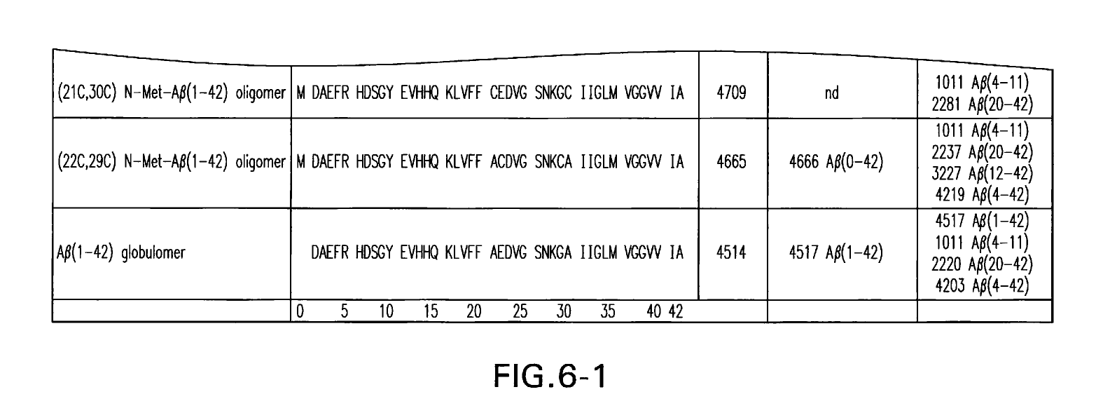

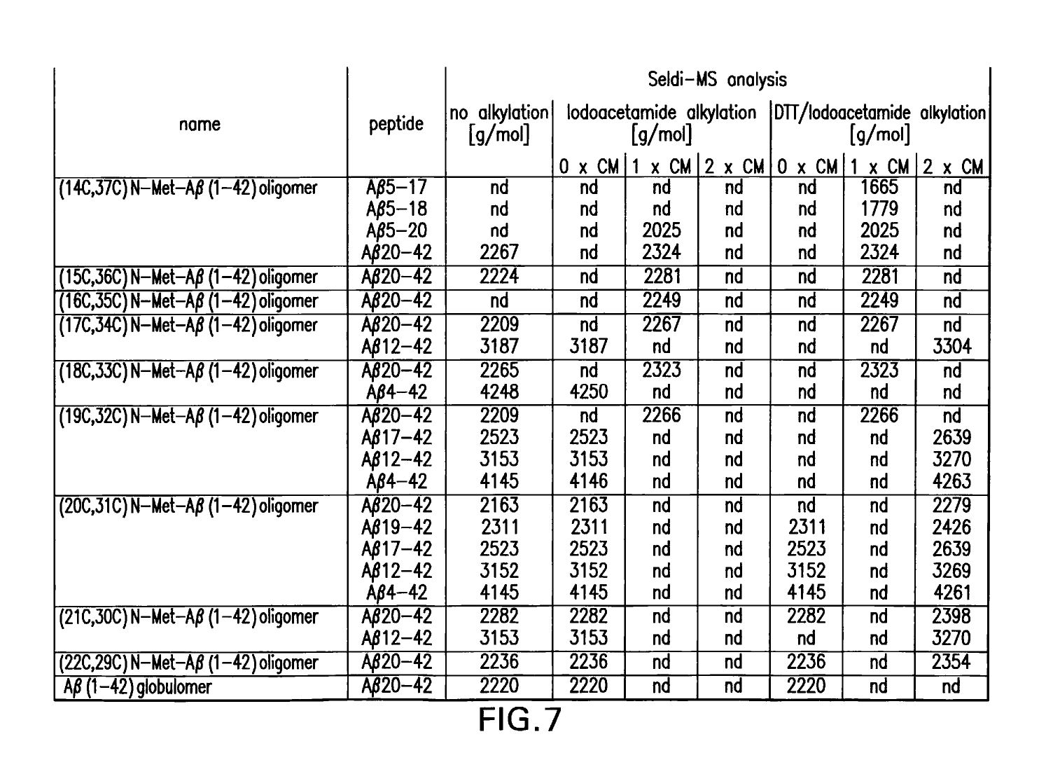

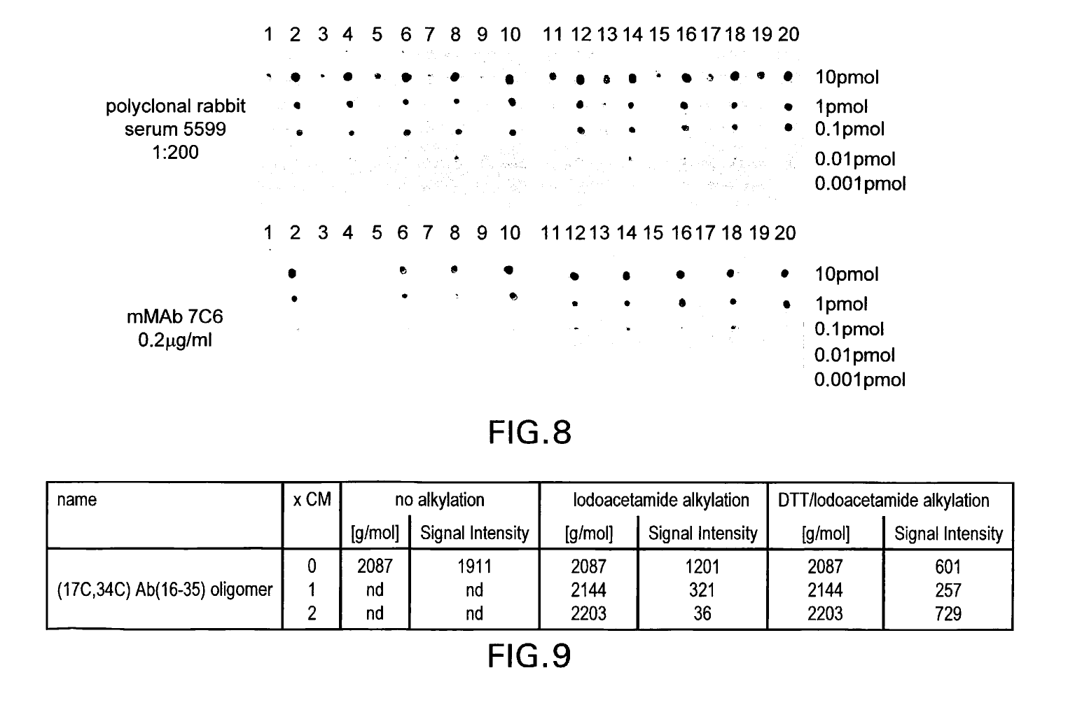

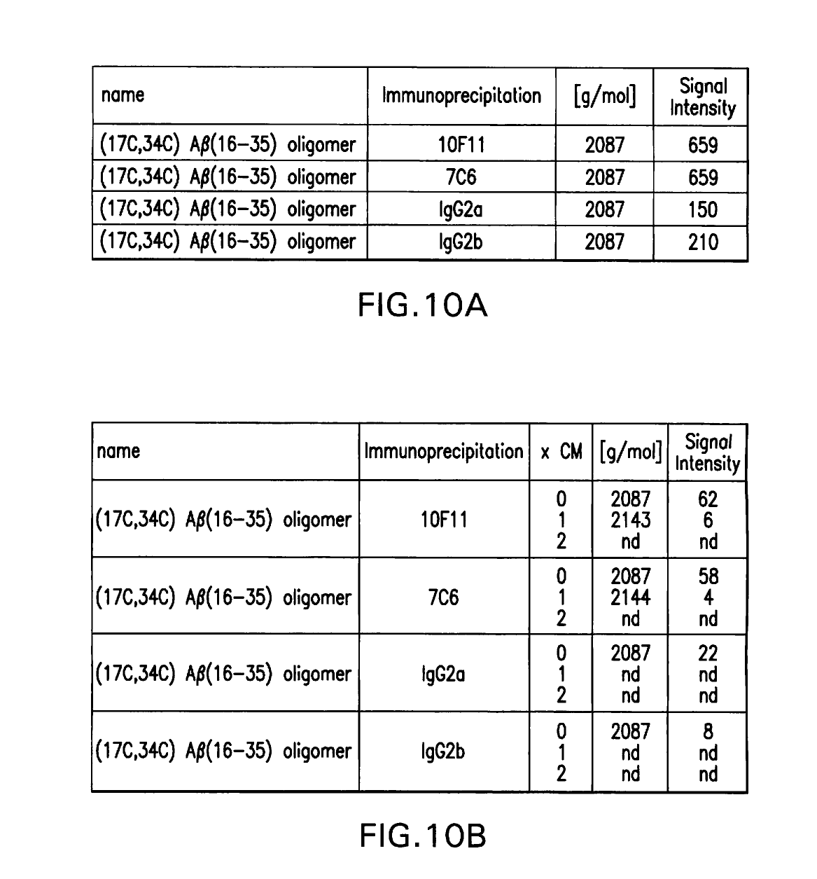

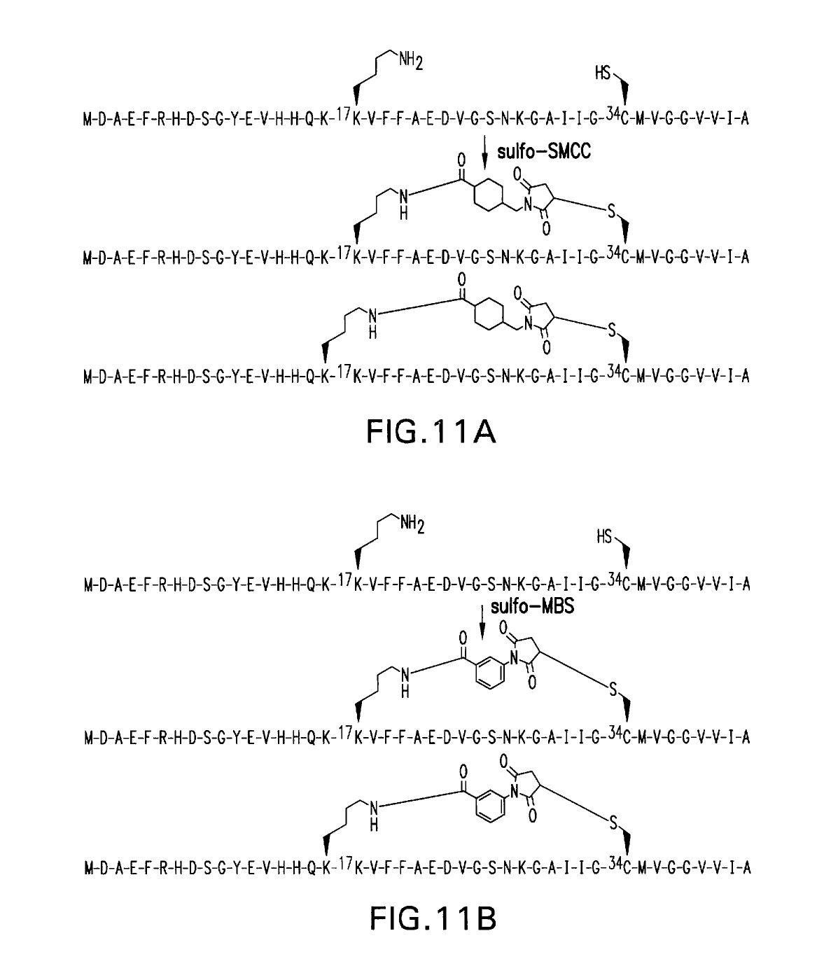

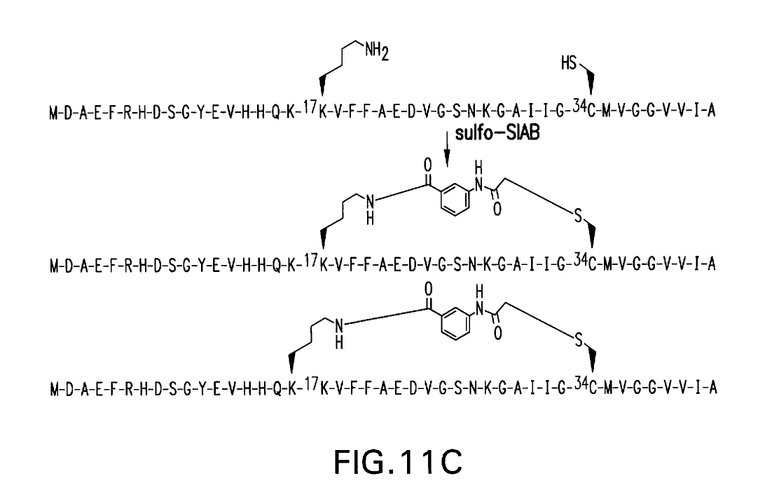

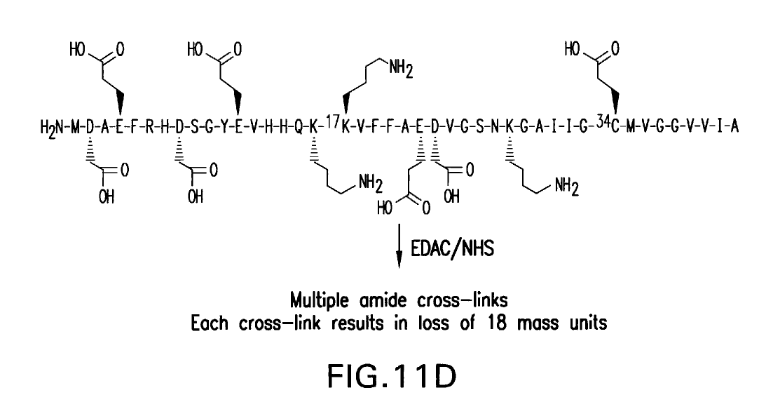

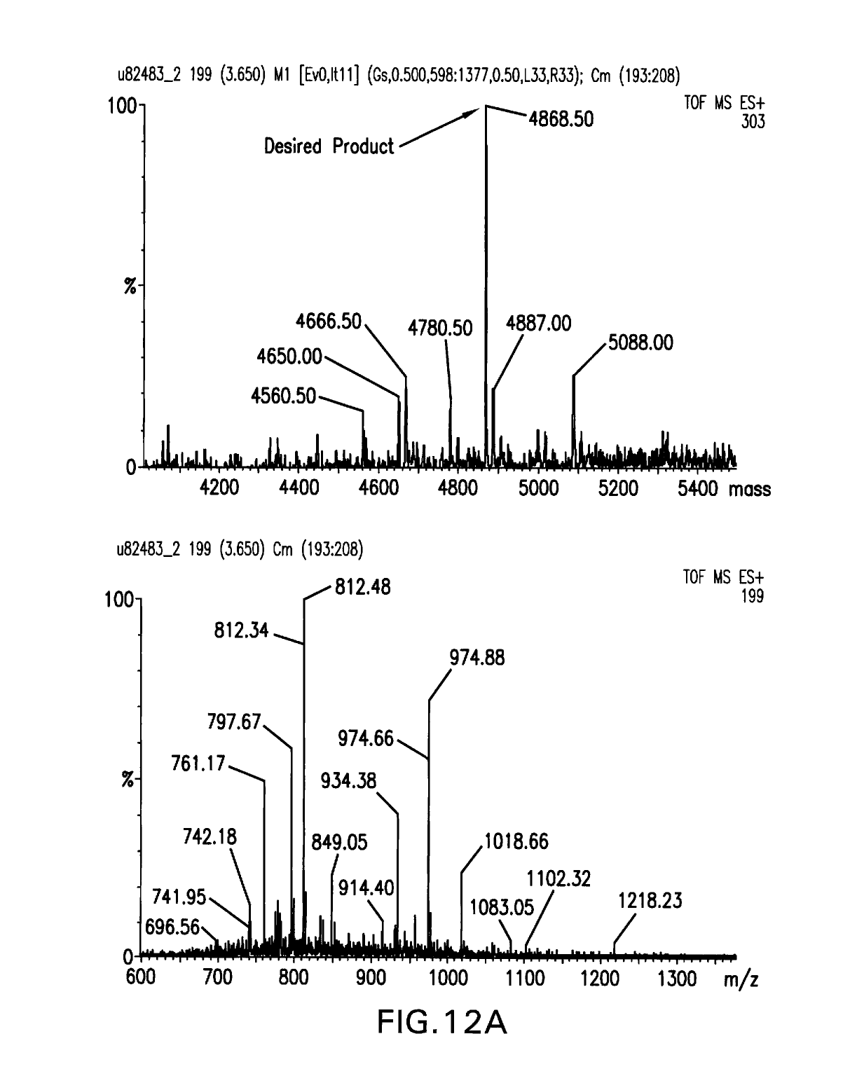

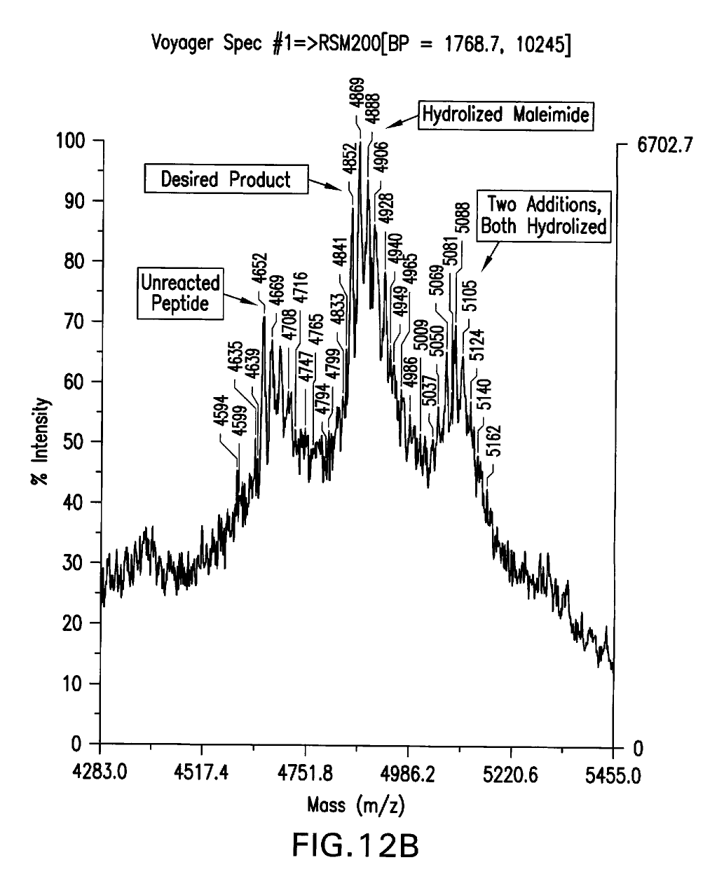

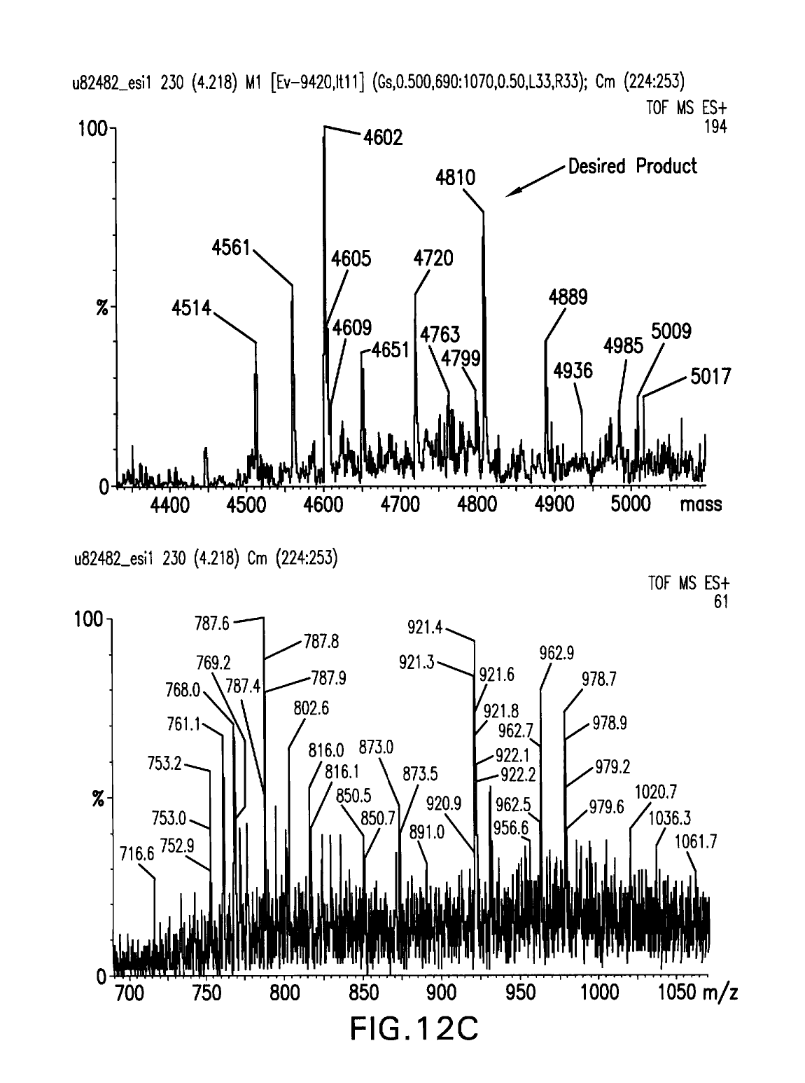

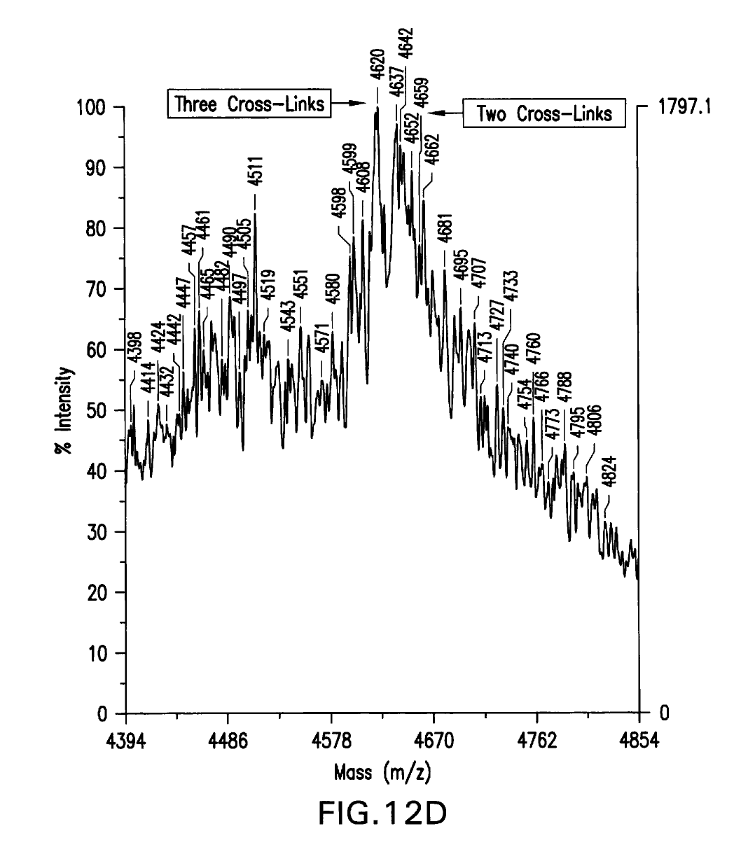

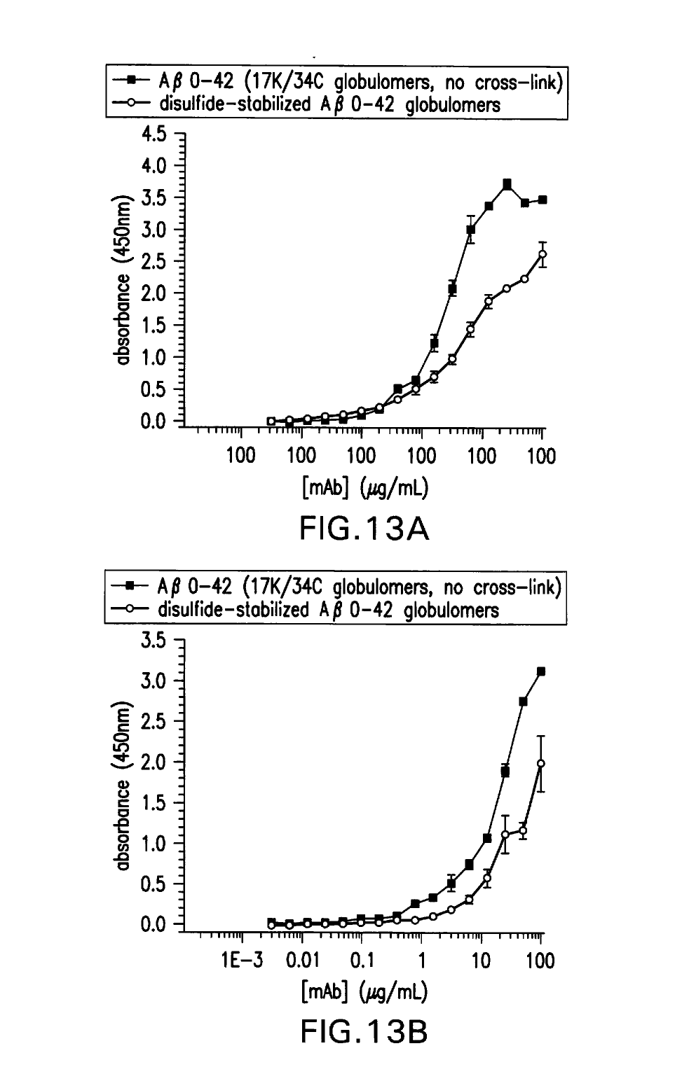

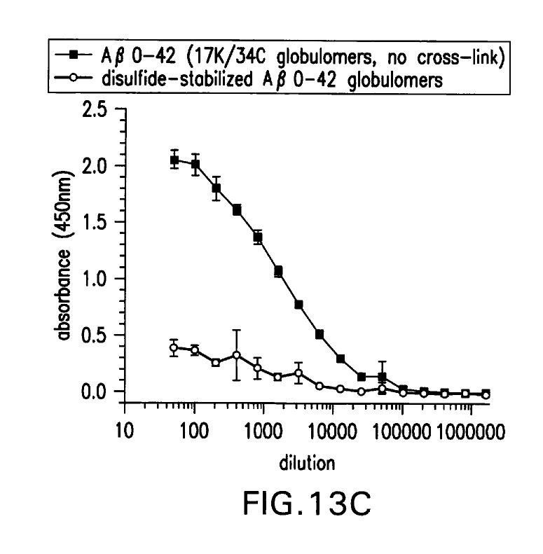

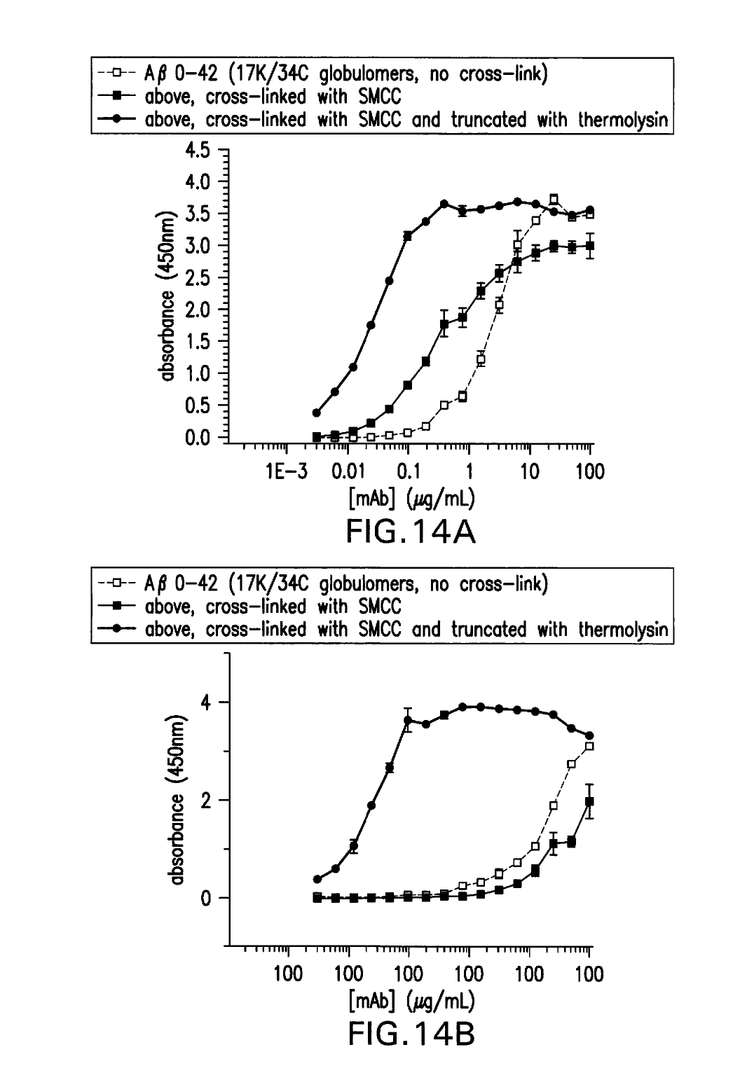

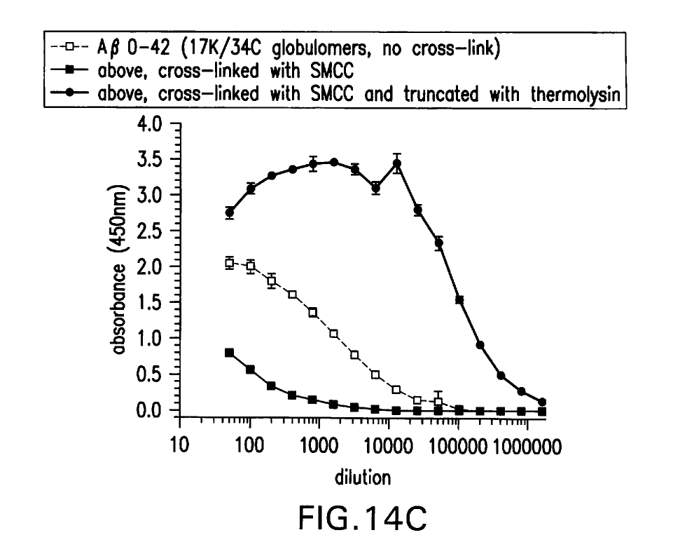

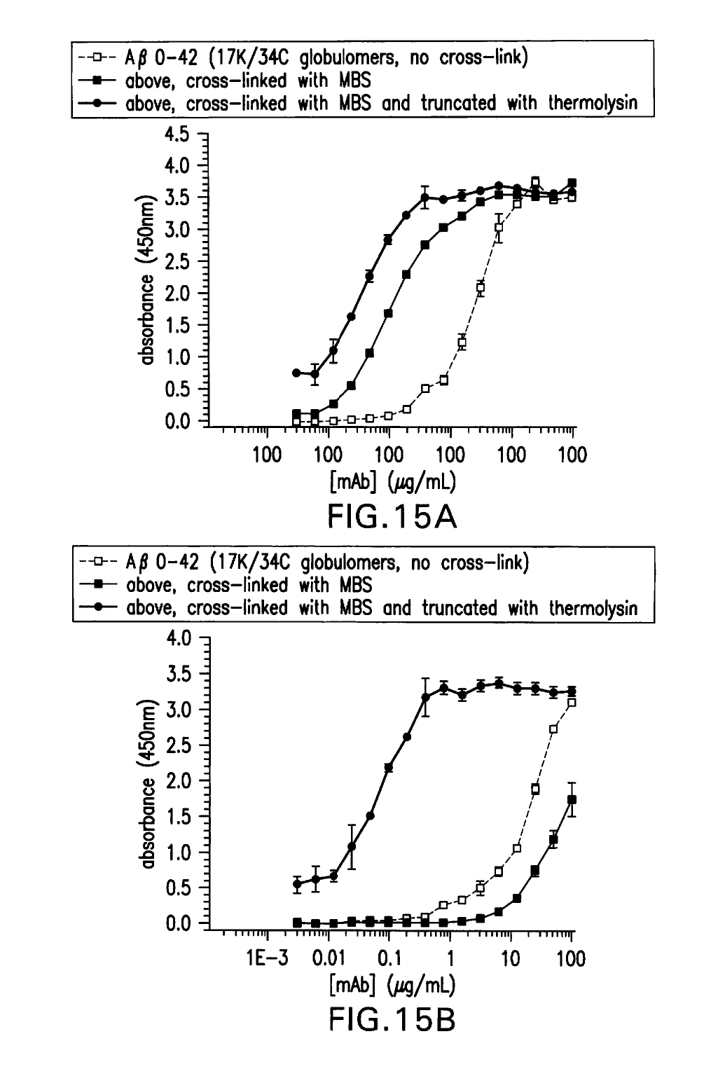

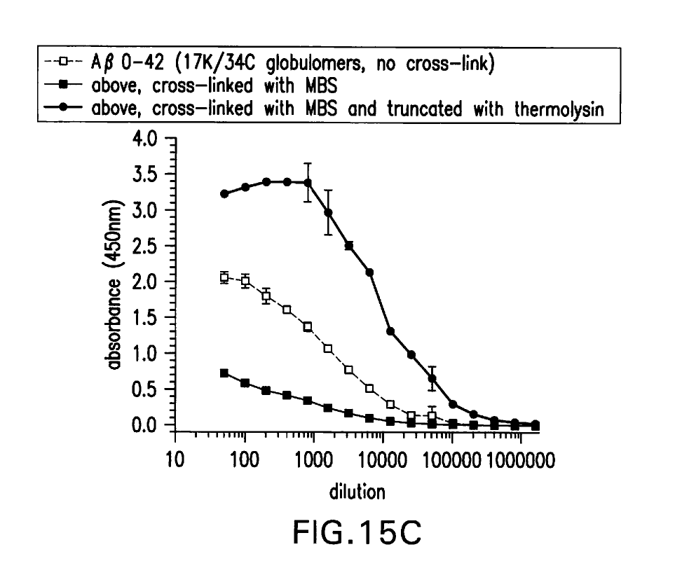

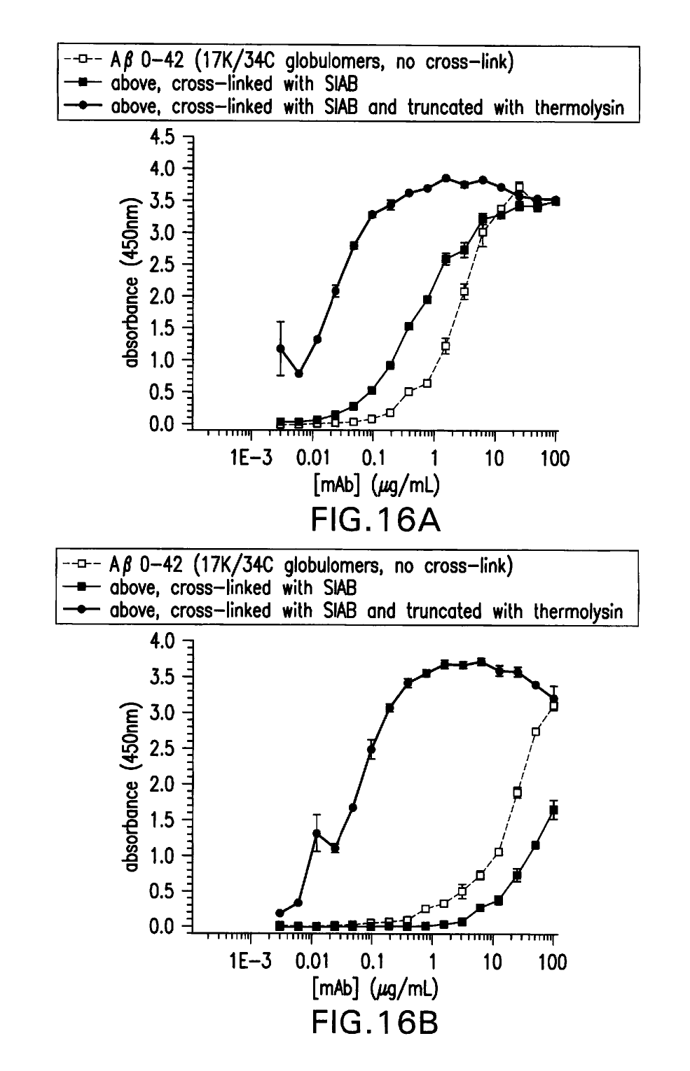

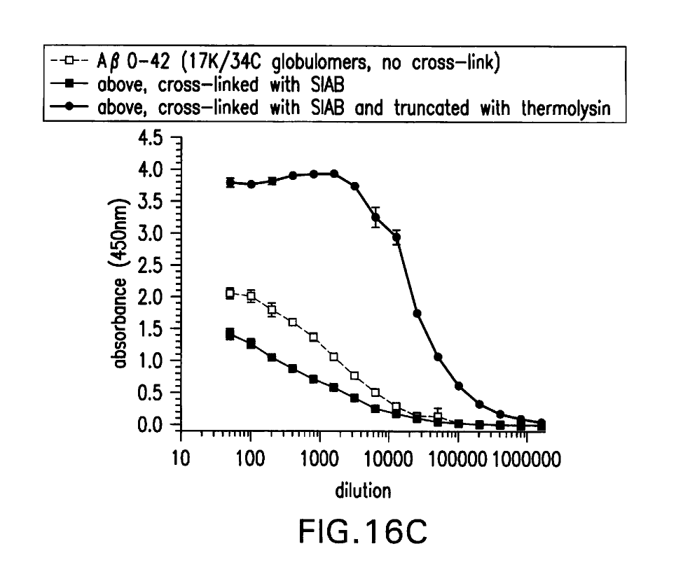

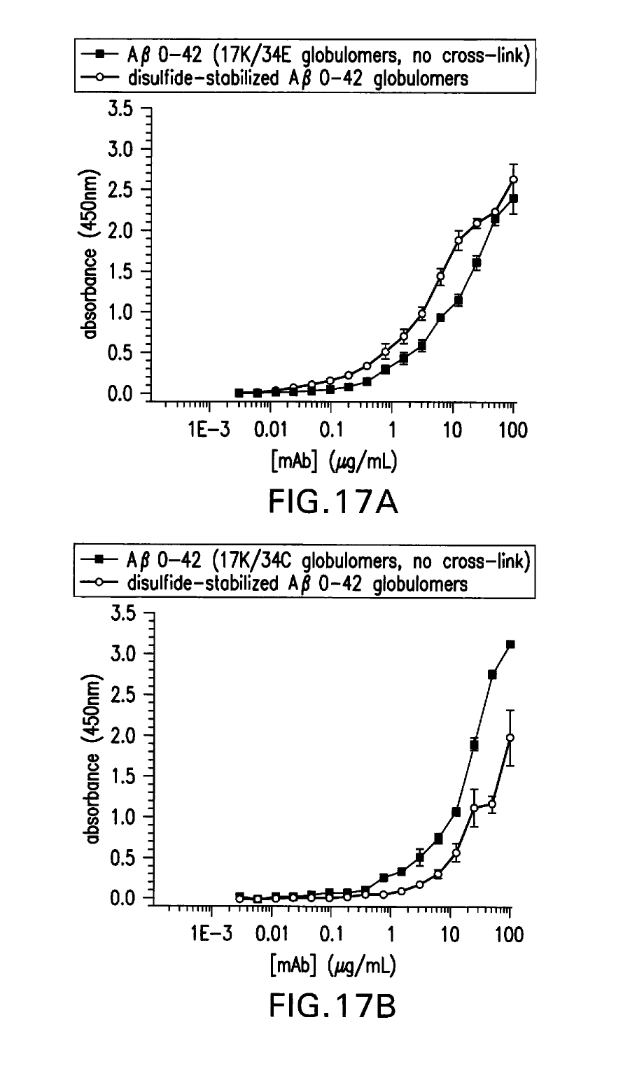

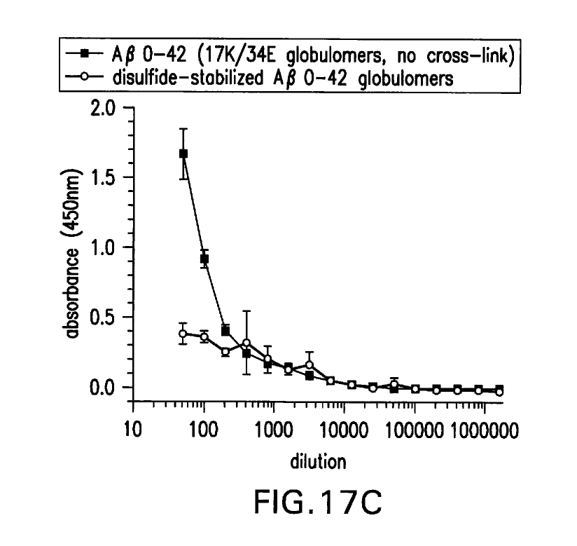

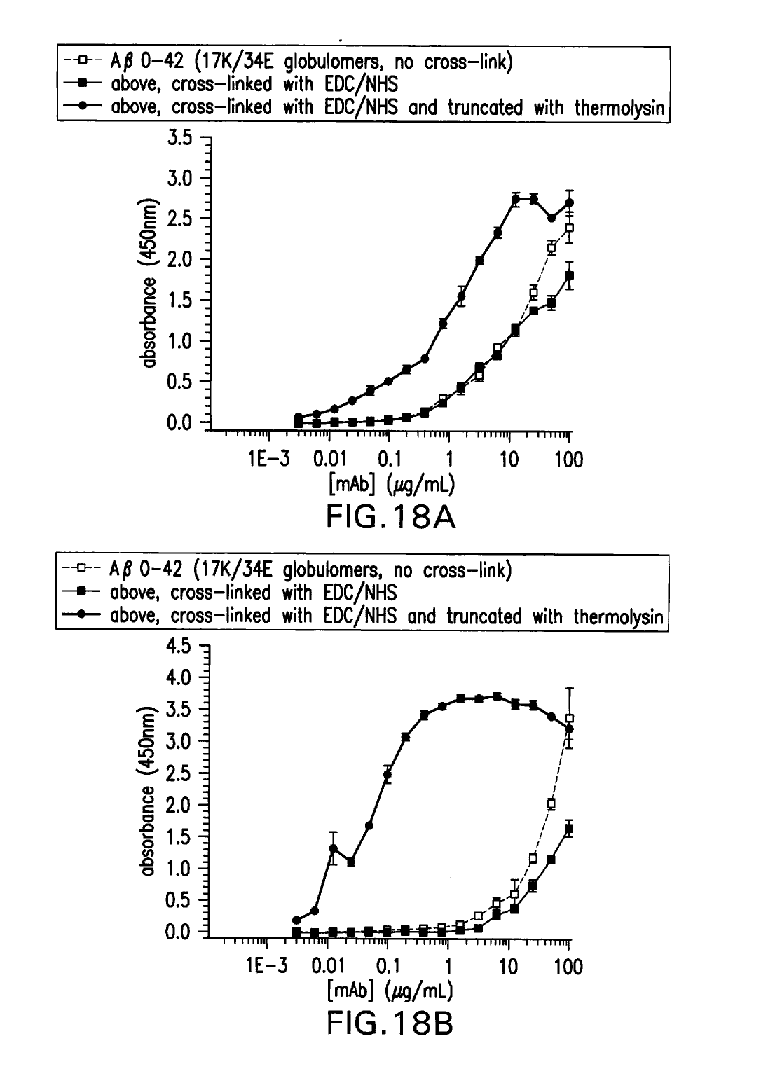

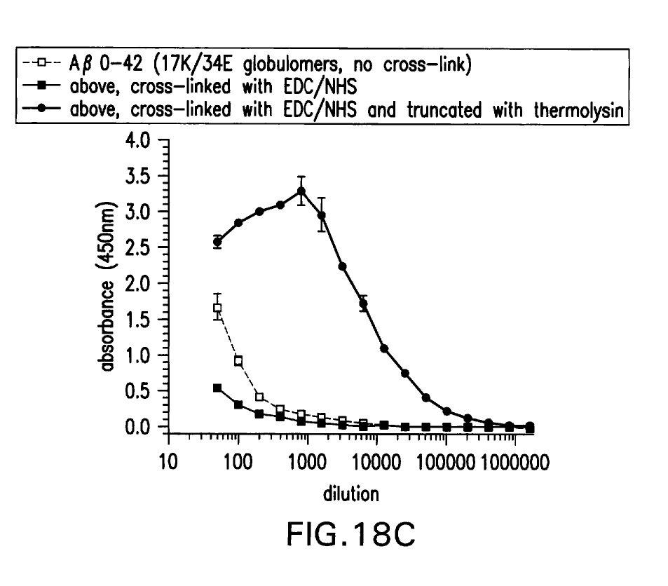

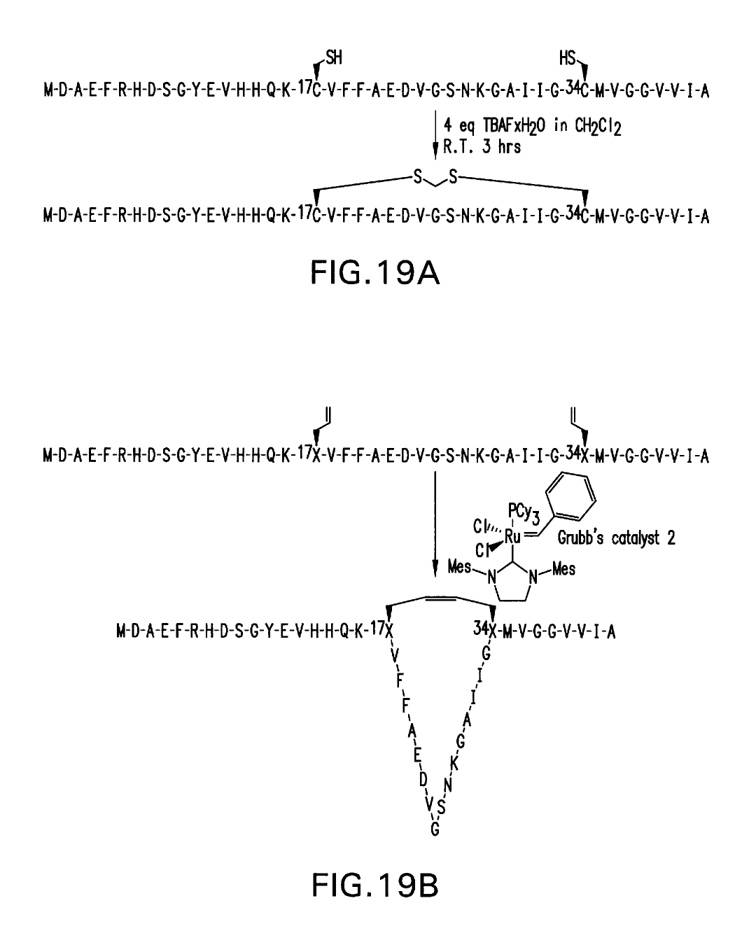

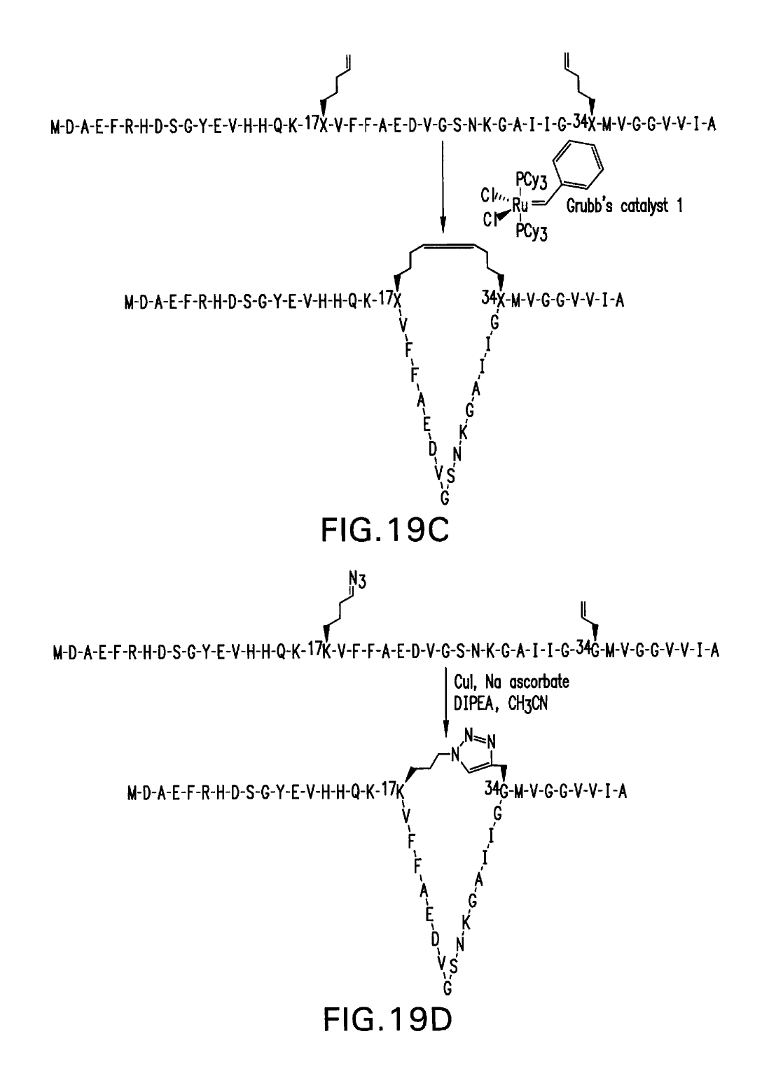

The binding of the globulomer-specific monoclonal antibody 5F7 to N-Met Aβ(1-42) globulomer and (L17C, L34C) N-Met Aβ(1-42) mutant oligomer to the 5F7 are almost identical (FIG. 3A) indicating that the (L17C, L34C) N-Met Aβ(1-42) mutant oligomer still displays the globulomer-specific epitope.

A comparison of direct ELISA results comparing the apparent binding affinity of the globulomer-specific mAb 5F7 to disulfide stabilized (17C, 34C) N-Met Aβ(1-42) oligomer from example 4y vs. the same oligomer truncated at residue 20 by enzymatic cleavage with thermolysin from example 5y is shown in FIG. 3B.

A comparison of direct ELISA results comparing the apparent binding affinity of the globulomer-specific mAb 7C6 to disulfide stabilized (17C, 34C) N-Met Aβ(1-42) oligomer from example 4y vs. the same oligomer truncated at residue 20 by enzymatic cleavage with thermolysin from example 5y is shown in FIG. 3C.

A comparison of direct ELISA results comparing the apparent binding affinity of the globulomer-specific rabbit polyclonal antiserum 5599 to disulfide stabilized (17C, 34C) N-Met Aβ(1-42) oligomer from example 4y vs. the same oligomer truncated at residue 20 by enzymatic cleavage with thermolysin from example 5y is shown in FIG. 3D.

A comparison of direct ELISA results comparing the apparent binding affinity of the globulomer-specific mAb 5F7 to disulfide stabilized (17C, 34C) Aβ(16-35) oligomer from example 4p vs. the same oligomer truncated at residue 20 by enzymatic cleavage with thermolysin from example 5p is shown in FIG. 3E.

A comparison of direct ELISA results comparing the apparent binding affinity of the globulomer-specific mAb 7C6 to disulfide stabilized (17C, 34C) Aβ(16-35) oligomer from example 4p vs. the same oligomer truncated at residue 20 by enzymatic cleavage with thermolysin from example 5p is shown in FIG. 3F.

A comparison of direct ELISA results comparing the apparent binding affinity of the globulomer-specific rabbit polyclonal antiserum 5599 to disulfide stabilized (17C, 34C) Aβ(16-35) oligomer from example 4p vs. the same oligomer truncated at residue 20 by enzymatic cleavage with thermolysin from example 5p is shown in FIG. 3G.

A comparison of direct ELISA results comparing the apparent binding affinity of the globulomer-specific mAb 5F7 to Aβ(16-35) oligomer from example 4m vs. the same oligomer truncated at residue 20 by enzymatic cleavage with thermolysin from example 5m is shown in FIG. 3H.

A comparison of direct ELISA results comparing the apparent binding affinity of the globulomer-specific mAb 7C6 to Aβ(16-35) oligomer from example 4m vs. the same oligomer truncated at residue 20 by enzymatic cleavage with thermolysin from example 5m is shown in FIG. 3I.

A comparison of direct ELISA results comparing the apparent binding affinity of the globulomer-specific rabbit polyclonal antiserum 5599 to Aβ(16-35) oligomer from example 4m vs. the same oligomer truncated at residue 20 by enzymatic cleavage with thermolysin from example 5m is shown in FIG. 3J.

A comparison of direct ELISA results comparing the apparent binding affinity of the globulomer-specific mAb 5F7 to (17C(ACM), 34C(ACM)) Aβ(16-35) oligomer from example 4l vs. the same oligomer truncated at residue 20 by enzymatic cleavage with thermolysin from example 51 is shown in FIG. 3K.

A comparison of direct ELISA results comparing the apparent binding affinity of the globulomer-specific mAb 7C6 to (17C(ACM), 34C(ACM)) Aβ(16-35) oligomer from example 4l vs. the same oligomer truncated at residue 20 by enzymatic cleavage with thermolysin from example 51 is shown in FIG. 3L.

A comparison of direct ELISA results comparing the apparent binding affinity of the globulomer-specific rabbit polyclonal antiserum 5599 to (17C(ACM), 34C(ACM)) Aβ(16-35) oligomer from example 41 vs. the same oligomer truncated at residue 20 by enzymatic cleavage with thermolysin from example 51 is shown in FIG. 3M.

A comparison of direct ELISA results comparing the apparent binding affinity of the globulomer-specific mAb 5F7 to disulfide stabilized (17C, 34C) Aβ(16-35) oligomer (cyclised before oligomer formation) from example 4o vs. the same oligomer truncated at residue 20 by enzymatic cleavage with thermolysin from example 5o is shown in FIG. 3N.

A comparison of direct ELISA results comparing the apparent binding affinity of the globulomer-specific mAb 7C6 to disulfide stabilized (17C, 34C) Aβ(16-35) oligomer (cyclised before oligomer formation) from example 4o vs. the same oligomer truncated at residue 20 by enzymatic cleavage with thermolysin from example 5o is shown in FIG. 3O;

A comparison of direct ELISA results comparing the apparent binding affinity of the globulomer-specific rabbit polyclonal antiserum 5599 to disulfide stabilized (17C, 34C) Aβ(16-35) oligomer (cyclised before oligomer formation) from example 4o vs. the same oligomer truncated at residue 20 by enzymatic cleavage with thermolysin from example 5o is shown in FIG. 3P;

A comparison of direct ELISA results comparing the apparent binding affinity of the globulomer-specific mAb 5F7 to disulfide stabilized (17C, 34C) Aβ(16-35) oligomer (cyclised before oligomer formation) from example 4o vs. disulfide stabilized (17C, 34C) Aβ(16-42), (17C, 34C) Aβ(16-35) and (17C, 34C) N-Met Aβ(1-42) oligomer (all cyclised after oligomer formation) from examples 4s, 4p and 4y is shown in FIG. 3Q.

A comparison of direct ELISA results comparing the apparent binding affinity of the globulomer-specific mAb 5F7 to thermolysin truncated disulfide stabilized (17C, 34C) Aβ(16-35) oligomer (cyclised before oligomer formation) from example 5o vs. thermolysin truncated disulfide stabilized (17C, 34C) Aβ(16-42), (17C, 34C) Aβ(16-35) and (17C, 34C) N-Met Aβ(1-42) oligomer (all cyclised after oligomer formation) from examples 5s, 5p and 5y is shown in FIG. 3R.

A comparison of direct ELISA results comparing the apparent binding affinity of the globulomer-specific mAb 5F7 to disulfide stabilized (17C, 34C) Aβ(16-42) oligomer from example 4s vs. the same oligomer truncated at residue 20 by enzymatic cleavage with thermolysin from example 5s is shown in FIG. 3S.

A comparison of direct ELISA results comparing the apparent binding affinity of the globulomer-specific mAb 7C6 to disulfide stabilized (17C, 34C) Aβ(16-42) oligomer from example 4s vs. the same oligomer truncated at residue 20 by enzymatic cleavage with thermolysin from example 5s is shown in FIG. 3T.

A comparison of direct ELISA results comparing the apparent binding affinity of the globulomer-specific rabbit polyclonal antiserum 5599 to disulfide stabilized (17C, 34C) Aβ(16-42) oligomer from example 4s vs. the same oligomer truncated at residue 20 by enzymatic cleavage with thermolysin from example 5s is shown in FIG. 3U.

A comparison of direct ELISA results comparing the apparent binding affinity of the globulomer-specific mAb 5F7 to disulfide stabilized (17C, 34C) Aβ(12-42) oligomer from example 4k vs. the same oligomer truncated at residue 20 by enzymatic cleavage with thermolysin from example 5k is shown in FIG. 3V.

A comparison of direct ELISA results comparing the apparent binding affinity of the globulomer-specific mAb 7C6 to disulfide stabilized (17C, 34C) Aβ(12-42) oligomer from example 4k vs. the same oligomer truncated at residue 20 by enzymatic cleavage with thermolysin from example 5k is shown in FIG. 3W.

A comparison of direct ELISA results comparing the apparent binding affinity of the globulomer-specific rabbit polyclonal antiserum 5599 to disulfide stabilized (17C, 34C) Aβ(12-42) oligomer from example 4k vs. the same oligomer truncated at residue 20 by enzymatic cleavage with thermolysin from example 5k is shown in FIG. 3X.

Direct ELISA results of the apparent binding affinity of the globulomer-specific mAb 5F7 to disulfide stabilized (17KC, 34C) Aβ(13-42) oligomer from example 4r are shown in FIG. 3Y.

Direct ELISA results of the apparent binding affinity of the globulomer-specific mAb 7C6 to disulfide stabilized (17KC, 34C) Aβ(13-42) oligomer from example 4r are shown in FIG. 3Z.

Direct ELISA results of the apparent binding affinity of the globulomer-specific rabbit polyclonal antiserum 5599 to disulfide stabilized (17KC, 34C) Aβ(13-42) oligomer from example 4r are shown in FIG. 3AA.

FIG. 13 shows a comparison of direct Elisa response of (17K, 34C) N-Met Aβ(1-42) oligomer without cross-link from example 3w with disulfide-stabilized (17C, 34C) N-Met Aβ(1-42) oligomer from example 4y to (A) the globulomer-specific monoclonal antibody 5F7; (B) the globulomer-specific monoclonal antibody 7C6; and (C) the globulomer-specific rabbit polyclonal antiserum 5599.

FIG. 14 shows (A) a comparison of direct Elisa response of (17K, 34C) N-Met Aβ(1-42) oligomer without cross-link from example 3w with (17K, 34C) N-Met Aβ(1-42) oligomer cross-linked with SMCC before (from example 4w1) and after (from example 5w1)thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 15 shows (A) a comparison of direct Elisa response of (17K, 34C) N-Met Aβ(1-42) oligomer without cross-link from example 3w with (17K, 34C) N-Met Aβ(1-42) oligomer cross-linked with MBS before (from example 4w2) and after (from example 5w2) thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 16 shows (A) a comparison of direct Elisa response of (17K, 34C) N-Met Aβ(1-42) oligomer without cross-link from example 3w with (17K, 34C) N-Met Aβ(1-42) oligomer cross-linked with SIAB before (from example 4w3) and after (from example 5w3) thermolysin truncation to (A) the globulomer-specific monoclonal antibody 5F7; (B) the globulomer-specific monoclonal antibody 7C6; and (C) the globulomer-specific rabbit polyclonal antiserum 5599.

FIG. 17 shows (A) a comparison of direct Elisa response of (17K, 34E) N-Met Aβ(1-42) oligomer from example 3x without cross-link with disulfide-stabilized (17C, 34C) N-Met Aβ(1-42) oligomer from example 3y to (A) the globulomer-specific monoclonal antibody 5F7; (B) the globulomer-specific monoclonal antibody 7C6; and (C) the globulomer-specific rabbit polyclonal antiserum 5599.

FIG. 18 shows (A) a comparison of direct Elisa response of (17K, 34C) N-Met Aβ(1-42) oligomer without cross-link with (17K, 34C) N-Met Aβ(1-42) oligomer cross-linked with EDC/NHS before and after thermolysin truncation to (A) the globulomer-specific monoclonal antibody 5F7; (B) the globulomer-specific monoclonal antibody 7C6; and (C) the globulomer-specific rabbit polyclonal antiserum 5599.

Some of the results obtained are:

The (17C, 34C) Aβ(16-42) peptide (example 3s) was shown to form oligomers (example 4s) that both contained the expected disulfide bond (FIGS. 4E & F) and displayed the globulomer epitope (FIGS. 3S-3U). Upon truncation with thermolysin (example 5s), the expected increase in affinity to the globulomer-specific antibodies was observed (FIG. 3S-3U).

The (17C, 34C) Aβ(12-42) peptide (example 3k) was shown to form oligomers (example 4k) that both contained the expected disulfide bond (FIGS. 4G & 4H) and displayed the globulomer epitope (FIGS. 3V-3X). Upon truncation with thermolysin (example 5k), the expected increase in affinity to the globulomer-specific antibodies was observed (FIGS. 3V-3X).

The (17C, 34C) Aβ(16-35) peptide (example 3o) was shown to form oligomers (example 4o) that both contained the expected disulfide bond (FIGS. 4C & D) and displayed the globulomer epitope (FIGS. 3E-3G). Upon truncation with thermolysin (example 5o), the expected increase in affinity to the globulomer-specific antibodies was observed (FIGS. 3E-G). However, Aβ(16-35) peptides that are precluded from forming this disulfide linkage, either through lack of cys residues (wt Aβ(16-35), example 3m), or ACM protection of the —SH of the cys residue ((17C ACM/34C ACM) Aβ(16-35), example 31), were not able to form oligomers that displayed the globulomer epitope (FIGS. 3H-3M).

By introducing a lysine at position 17 and a cysteine at position 34, a variety of heterobifunctional cross-linking reagents can be employed for a bridge between the side-chains of these two residues, including sulfo-SMCC, sulfo-MBS, and sulfo-SIAB. For instance, the (17K, 34C) N-Met Aβ(1-42) peptide (example 3w) can form oligomers that display the globulomer epitope (FIGS. 13A-13C). Moreover, these oligomers can be subjected to cross-linking using sulfo-SMCC (example 4w1), sulfo-MBS (example 4w2), and sulfo-SIAB (example 4w3), and the desired intro-molecular covalent cross-link formed (FIGS. 12A-12C). These cross-linked AR oligomers still display the appropriate reactivity to the globulomer-specific antibodies (FIGS. 14A-14C, 15A-15C, and 16A-16C, respectively). Furthermore, these AR oligomers can be subjected to thermolysin truncation (examples 4w1, 4w2, & 4w3), with the expected increase in affinity to the globulomer-specific antibodies resulting (FIGS. 14A-14C, 15A-15C, and 16A-16C).

By introducing a lysine at position 17 and a glutamic acid at position 34, a variety of cross-linking strategies can be employed to for a bridge between the side-chains of these two residues, including activation of the glutamate side chain by reaction with EDC, followed by sulfo-NHS. This activated side chain can subsequently react with primary amine of the lysine residue introduced at position 17. For instance, the (17K, 34E) N-Met Aβ(1-42) peptide (example 3x) can form oligomers that display the globulomer epitope (FIGS. 17A-17C). Moreover, these oligomers can be subjected to cross-linking using EDC and sulfo-NHS, with intra-molecular covalent cross-links resulting (FIG. 12D). These cross-linked AR oligomers still display the appropriate reactivity to the globulomer-specific antibodies (FIGS. 18A-18C). Furthermore, these AR oligomers can be subjected to thermolysin truncation (examples 4x), with the expected increase in affinity to the globulomer-specific antibodies resulting (FIGS. 18A-18C).

### Example 9

### Mass Spectroscopy

Mass spectrometry of the (17C, 34C) N-Met AN-Met Aβ(1-42) oligomer (2a) from example 2a confirmed that formation of oligomers with the (L17C, L34C) N-Met Aβ(1-42) mutant peptide (1a) of example 1a directed the efficient formation of intra-peptide disulfide bonds between the two Cys residues (FIG. 4A). The mass centered around 4622 Da, the expected mass for the (L17C, L34C) N-Met Aβ(1-42) mutant peptide, with a single disulfide bond formed. No sign of covalent dimer peptide was observed in the mass spectrum. After reduction with dithiothreitol (DTT), the mass centered around 4624 Da, as expected for the (L17C, L34C) N-Met Aβ(1-42) mutant peptide with no single disulfide bond, and each mass observed in the isotopic deconvolution gains two Da, as expected from the reduction of a disulfide bond (FIG. 4B).

Mass spectrometry of the (17C, 34C) Aβ(16-35) oligomer (3p) from example 3p confirmed that formation of oligomers with the (17C, 34C) Aβ(16-35) peptide (1p) of example 1p directed the efficient formation of intra-peptide disulfide bonds between the two Cys residues (FIG. 4C). The mass centered around 2085 Da, the expected mass for the (17C, 34C) Aβ(16-35) peptide, with a single disulfide bond formed. No sign of covalent dimer peptide was observed in the mass spectrum. After reduction with dithiothreitol (DTT), the mass centered around 2086 Da, as expected for the (17C, 34C) Aβ(16-35) peptide with no single disulfide bond, and each mass observed in the isotopic deconvolution gains one to two Da, as expected from the reduction of a disulfide bond (FIG. 4D).

Mass spectrometry of the (17C, 34C) Aβ(16-42) oligomer (3s) from example 3s confirmed that formation of oligomers with the (17C, 34C) Aβ(16-42) peptide (1s) of example 1s directed the efficient formation of intra-peptide disulfide bonds between the two Cys residues (FIG. 4E). The mass centered around 2683 Da, the expected mass for the (17C, 34C) Aβ(16-35) peptide, with a single disulfide bond formed. No sign of covalent dimer peptide was observed in the mass spectrum. After reduction with dithiothreitol (DTT), the mass centered around 2684 Da, as expected for the (17C, 34C) Aβ(16-42) peptide with no single disulfide bond, and each mass observed in the isotopic deconvolution gains one to two Da, as expected from the reduction of a disulfide bond (FIG. 4F).

Mass spectrometry of the (17C, 34C) Aβ(12-42) oligomer (3k) from example 3k confirmed that formation of oligomers with the (17C, 34C) Aβ(12-42) peptide (1k) of example 1k directed the efficient formation of intra-peptide disulfide bonds between the two Cys residues (FIG. 4G). No sign of covalent dimer peptide was observed in the mass spectrum. After reduction with dithiothreitol (DTT), the mass centered around 3186 Da, as expected for the (17C, 34C) Aβ(12-42) peptide with no single disulfide bond, and each mass observed in the isotopic deconvolution gains one to two Da, as expected from the reduction of a disulfide bond (FIG. 4H).

Mass spectrometry (ESI) of the (17KC, 34C) Aβ(13-42) oligomer (3r) from example 3r confirmed that formation of oligomers with the (17KC, 34C) Aβ(13-42) peptide (1r) of example 1r directed the efficient formation of intra-peptide disulfide bonds between the two Cys residues (FIG. 4I). While significant amounts of covalent dimer peptide were observed in the mass spectrum (data not shown), the desired intra-molecular disulfide bond is evident. After reduction with dithiothreitol (DTT), the mass centered around 3215 Da, as expected for the (17KC, 34C) Aβ(13-42) peptide with no single disulfide bond, and each mass observed in the iso-topic deconvolution gains one to two Da, as expected from the reduction of a disulfide bond (FIG. 4J).

The peptide masses of the (xC, yC) N-Met Aβ(1-42) oligomers before (from examples 4a-4j) and after (from examples 5a-5j) thermolysin digestion as detected by SELDI-MS are indicated in FIG. 6.

The mass spectrum (ESI) of oligomers made with (17K, 34C) N-Met Aβ(1-42) peptide after cross-linking reaction with the heterobifunctional cross-linking reagent sulfo-SMCC from example 4w1 is shown in FIG. 12A. The predominant species appears to be the desired product.

The mass spectrum (MALDI) of globulomers made with (17K, 34C) N-Met Aβ(1-42) peptide after cross-linking reaction with the heterobifunctional cross-linking reagent sulfo-MBS from example 4w2 is shown in FIG. 12B. There is evidence for the desired cross-link to have formed (4852 Da), however masses that correspond to addition of the MBS followed by hydrolysis of the maleimide (4869 Da), addition of two MBS adducts followed by hydrolysis (5088 Da), and unreacted peptide (4652 Da) are also observed.

The mass spectrum (ESI) of globulomers made with (17K, 34C) N-Met Aβ(1-42) peptide after cross-linking reaction with the heterobifunctional cross-linking reagent sulfo-SIAB from example 4w3 is shown in FIG. 12C. The arrow indicates the expected mass after the desired cross-link forms (mw=4810 Da).

The mass spectrum (MALDI) of globulomers made with (17K, 34E) N-Met Aβ(1-42) peptide after cross-linking reaction with the heterobifunctional cross-linking reagent EDC and NHS from example 4x is shown in FIG. 12D. The arrows indicate masses that suggest two cross-links (mw=4637 Da), and three cross-links (mw=4620 Da) have formed.

### Example 10

### Hydrodynamic Analysis

Samples were loaded into standard two-sector cells using sapphire windows. All samples were examined using either a 4-hole or an 8-hole rotor.

Conditions were as follows: temperature: 20° C., rotor speed: 42,000 rpm, interference data was collected, absorbance data was collected at 280 nm in continuous mode with a radial step size of 0.003 cm. One data point was collected per step (no signal averaging). Typically, 200 scans or less were collected over the course of no more than 9 hrs.

A continuous S distribution analysis (C(s) analysis) was done as implemented in Sedfit v8.9 (P. Schuck (2000), “Size distribution analysis of macromolecules by sedimentation velocity ultracentrifugation and Lamm equation modeling”, Biophysical Journal 78:1606-1619) to understand the overall heterogeneity of the samples. Both radial and time independent noise were fit and removed from the data using a maximal entropy algorithm.

The sedimentation velocity experiments showed that both N-Met Aβ(1-42) globulomer and the (L17C, L34C) N-Met Aβ(1-42) mutant oligomer (2a) from example 2a formed homogeneous oligomers (FIG. 5A). However, (L17C, L34C) N-Met Aβ(1-42) mutant oligomer displayed better hydrodynamic properties.

The sedimentation velocity experiments also showed that both N-Met Aβ(1-42) truncated globulomer and the thermolysin truncated (L17C, L34C) N-Met Aβ(1-42) oligomer (5y) from example 5y formed homogeneous oligomers (FIGS. 5B and C). However, thermolysin truncated (L17C, L34C) N-Met Aβ(1-42) oligomer displayed better hydrodynamic properties.

Introduction of a disulfide bond in the N-Met (17C/34C) Aβ(1-42) oligomer (example 4y) does stabilize further aggregation relative to wt N-Met Aβ(1-42) oligomer, in 5 mM Na—PO₄, 35 mM NaCl, pH 7.4 (FIG. 5A). Moreover, the disulfide stabilized (17C/34C) N-Met Aβ (1-42) oligomer remains significantly more hydrodynamically homogeneous after truncation at residue 20 by thermolysin (example 5y), relative to the wt N-Met Aβ(1-42) oligomer. This is evident both in the presence (FIG. 5B) and absence (FIG. 5C) of 0.05% SDS. In fact, after thermolysin treatment of the wt N-Met Aβ(1-42) oligomer results in a preparation that is too heterogeneous to be examined in the absence of 0.05% SDS (data not shown).

### Example 11

### Iodoacetamide Alkylation of Thermolysin Truncated (xC, yC) N-Met Aβ(1-42) Oligomers and SELDI-MS Analysis

### A: Thermolysin Truncated (xC, yC) N-Met Aβ(1-42) Oligomers and N-Met Aβ(1-42) Globulomers Used for Alkylation:

The following thermolysin truncated oligomers and globulomers were subjected to iodoacetamide alkylation
1) Thermolysin truncated (14C, 37C) N-Met Aβ(1-42) oligomer of example 5a
2) Thermolysin truncated (15C, 36C) N-Met Aβ(1-42) oligomer of example 5b
3) Thermolysin truncated (16C, 35C) N-Met Aβ(1-42) oligomer of example 5c
4) Thermolysin truncated (17C, 34C) N-Met Aβ(1-42) oligomer of example 5d
5) Thermolysin truncated (18C, 33C) N-Met Aβ(1-42) oligomer of example 5e
6) Thermolysin truncated (19C, 32C) N-Met Aβ(1-42) oligomer of example 5f
7) Thermolysin truncated (20C, 31C) N-Met Aβ(1-42) oligomer of example 5g
8) Thermolysin truncated (21C, 30C) N-Met Aβ(1-42) oligomer of example 5h
9) Thermolysin truncated (22C, 29C) N-Met Aβ(1-42) oligomer of example 5i
10) Aβ(1-42) thermolysin truncated globulomer of example 5j

### B: Iodoacetamide Alkylation

### B1: Control Samples

2.5 μl of the thermolysin truncated oligomer or globulomer were diluted with 5 μl 50 mM Tris/HCl, 1 mM EDTA, pH 8.6 (5 min Helium aerated)
20 min incubation at 37° C.
2 μl H₂O were added
1 h incubation at room temperature
115 μl 50% CH₃CN, 0.5% TFA were added

### B2: Iodoacetamide Alkylation Samples

2.5 μl of the thermolysin truncated oligomer or globulomer were diluted with 5 μl 50 mM Tris/HCl, 1 mM EDTA, pH 8.6 (Helium aerated)
20 min incubation at 37° C.
2 μl 100 mM iodoactamide (Sigma; Cat. no. 11149) solution in H₂O were added
1 h incubation at room temperature
115 μl 50% CH₃CN, 0.5% TFA were added

### B3: DTT Reduction Followed by Iodoacetamide Alkylation Samples

2.5 μl of the thermolysin truncated oligomer or globulomer were diluted with 5 μl 50 mM Tris/HCl, 1 mM EDTA, 2 mM DTT, pH 8.6 (Helium aerated)
20 min incubation at 37° C.
2 μl 100 mM Iodoactamide (Sigma; Cat. no. 11149) solution in H₂O were added
1 h incubation at room temperature
115 μl 50% CH₃CN, 0.5% TFA were added

### C: Surface-Enhanced Laser Desorption Ionization-Mass Spectrometry (SELDI-MS) Quantification of Immunoprecipitated Aβ(20-42) Peptide

2 μL sample was spotted onto a H4 Protein Chip Array (BioRad; Cat. no. C573-0028).
The spots were allowed to dry on a warm incubator plate.
CHCA-solution:
   5 mg CHCA were dissolved in 150 μL acetonitrile+150 μL 1% TFA=stock solution; stored at −20° C.
   Of the stock solution 10 μL were diluted with 20 μL acetonitrile and 20 μL 1% TFA=working CHCA-solution.
   2 μL of the working CHCA-solution was applied onto the spots
The spots were allowed to dry on a warm incubator plate and analyzed by SELDI-MS (Surface-Enhanced Laser Desorption Ionization-Mass Spectrometry; BioRad, Protein chip SELDI system enterprise edition)).
   Conditions: mass range: 800 to 10000 Da; focus mass: 2220 Da; matrix attenuation: 500 Da; sampling rate: 400 MHz; warming shots: 2 with energy: 1100 nj; data shots: 10 with energy 1000 nJoule; Partition 1 of 3.
   Analysis: the peak intensity of the respective double alkylated (2×CM), alkylated (1×CM) or non-alkylated (0×CM) peptide mass peaks were detected.

The results are shown in FIG. 7.

In the absence of DTT as reducing agent iodoacetamide can not alkylate the —SH group of cysteine if this has formed a covalent S—S bridge, the cystine, with another cysteine. Hence, absence of iodoacetamide alkylation reaction (as indicated by 0×CM in FIG. 7) of (xC, yC) N-Met-Aβ(1-42) oligomer after thermolysin treatment analyzed by SELDI-MS shows S—S formation has occurred in (xC, yC) N-Met-Aβ(1-42) oligomer. That iodoacetamide alkylation reaction can in principle occur was verified under reducing conditions using DTT to destroy S—S and generate free —SH groups which then were found to be amenable for iodoacetamide alkylation (FIG. 7).

### Example 12

### Dot Blot Analysis of (xC, yC) N-Met Aβ(1-42) Oligomers and Aβ(1-42) Globulomers Before and After Thermolysin Truncation

### Method:

### Aβ Standards for Dot Blot:

1. (14C, 37C) N-Met Aβ(1-42) oligomer of example 4a
2. Thermolysin truncated (14C, 37C) N-Met Aβ(1-42) oligomer of example 5a
3. (15C, 36C) N-Met Aβ(1-42) oligomer of example 4b
4. Thermolysin truncated (15C, 36C) N-Met Aβ(1-42) oligomer of example 5b
5. (16C, 35C) N-Met Aβ(1-42) oligomer of example 4c
6. Thermolysin truncated (16C, 35C) N-Met Aβ(1-42) oligomer of example 5c
7. (17C, 34C) N-Met Aβ(1-42) oligomer of example 4d
8) Thermolysin truncated (17C, 34C) N-Met Aβ(1-42) oligomer of example 5d
9. (18C, 33C) N-Met Aβ(1-42) oligomer of example 4e
10. Thermolysin truncated (18C, 33C) N-Met Aβ(1-42) oligomer of example 5e
11. (19C, 32C) N-Met Aβ(1-42) oligomer of example 4f
12. Thermolysin truncated (19C, 32C) N-Met Aβ(1-42) oligomer of example 5f
13. (20C, 31C) N-Met Aβ(1-42) oligomer of example 4g
14. Thermolysin truncated (20C, 31C) N-Met Aβ(1-42) oligomer of example 5g
15. (21C, 30C) N-Met Aβ(1-42) oligomer of example 4h
16. Thermolysin truncated (21C, 30C) N-Met Aβ(1-42) oligomer of example 5h
17. (22C, 29C) N-Met Aβ(1-42) oligomer of example 4i
18. Thermolysin truncated (22C, 29C) N-Met Aβ(1-42) oligomer of example 5i
19. Aβ(1-42) globulomer of example 4j
20. Aβ(1-42) thermolysin truncated globulomer of example 5j

### Materials for Dot Blot:

### Aβ Standards:

Serial dilution of Aβ antigens in columns 1-20 in 20 mM NaH₂PO₄, 140 mM NaCl, pH 7.4+0.2 mg/ml BSA
   1) 10 pmol/μl
   2) 1 pmol/μl
   3) 0.1 pmol/μl
   4) 0.01 pmol/μl
   5) 0.001 pmol/μl

### Nitrocellulose:

Trans-Blot Transfer medium, Pure Nitrocellulose Membrane (0.45 μm); BioRad

### Anti-Mouse-AP:

AP326A (Chemicon)

### Anti-Rabbit-AP:

AP304A (Chemicon)

### Detection Reagent:

NBT/BCIP Tablets (Roche)

### Bovine Serum Albumin, (BSA):

11926 Serva

### Blocking Reagent:

5% low fat milk in TBS

### Buffer Solutions:

TBS
25 mM Tris/HCl-buffer pH 7.5
+150 mM NaCl
TTBS
25 mM Tris/HCl-buffer pH 7.5
+150 mM NaCl
+0.05% Tween 20
PBS+0.2 mg/ml BSA
20 mM NaH₂PO₄ buffer pH 7.4
+140 mM NaCl
+0.2 mg/ml BSA

### Antibody Solution I:

Either Rabbit sera samples or mouse monoclonal antibodies were used as Antibody solution I and prepared as in the following:
Rabbit sera samples (1:200 diluted in 20 ml 1% low fat milk in TBS)
Mouse monoclonal antibodies (diluted to 0.2 μg/ml in 20 ml 1% low fat milk in TBS)

### Antibody Solution II:

In the case Antibody solution I was Rabbit sera samples then Antibody solution II was Anti-Rabbit-AP. In the case Antibody solution I was mouse monoclonal antibodies then Antibody solution II was Anti-Mouse-AP.
Antibody solution II was prepared as followed:
Anti-Mouse-AP: 1:5000 dilution in 1% low fat milk in TBS
Anti-Rabbit-AP: 1:5000 dilution in 1% low fat milk in TBS

### Dot Blot Procedure:

1) 1 μl each of the different Aβ standards (in their 5 serial dilutions) were dotted onto the nitrocellulose membrane in a distance of approximately 1 cm from each other.
2) The Aβ standards dots were allowed to dry on the nitrocellulose membrane on air for at least 10 min at room temperature (RT) (=dot blot)
3) Blocking:
   The dot blot was incubated with 30 ml 5% low fat milk in TBS for 1.5 h at RT.
4) Washing:
   The blocking solution was discarded and the dot blot was incubated under shaking with 20 ml TTBS for 10 min at RT.
5) Antibody solution I:
   The washing buffer was discarded and the dot blot was incubated with antibody solution I for 2 h at RT.
6) Washing:
   The antibody solution I was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
7) Antibody solution II:
   The washing buffer was discarded and the dot blot was incubated with antibody solution II for 1 h at RT
8) Washing:
   The antibody solution II was discarded and the dot blot was incubated under shaking with 20 ml TTBS for 10 min at RT. The washing solution was discarded and the dot blot was incubated under shaking with 20 ml TTBS for 10 min at RT. The washing solution was discarded and the dot blot was incubated under shaking with 20 ml TBS for 10 min at RT.
9) Development:
   The washing solution was discarded. 1 tablet NBT/BCIP was dissolved in 20 ml H₂O and the dot blot was incubated for 4 min with this solution. The development was stopped by intensive washing with H₂O.

The results are shown in FIG. 8.

The thermolysin truncated (xC, yC) N-Met-Aβ(1-42) oligomers show a comparable recognition by the Aβ globulomer-specific antibody 7C6 and the polyclonal rabbit serum 5599 as the Aβ(1-42) thermolysin truncated globulomer. An exception is thermolysin truncated (15C, 36C) N-Met-Aβ(1-42) oligomer which is only recognized by the polyclonal rabbit serum 5599. This may be due to the cysteine mutation at position 36 in the thermolysin truncated (15C, 36C) N-Met-Aβ(1-42) oligomer which may interfere with the Aβ globulomer epitope recognition of 7C6. Furthermore, the antibody 7C6 and the polyclonal rabbit serum 5599 do not detect the (xC, yC) N-Met-Aβ(1-42) oligomers without thermolysin treatment which is comparable to Aβ(1-42) globulomer. Note, that polyclonal rabbit serum 5599 exhibits in general a high background reaction for all peptides dotted which can not be attributed to a specific recognition of the Aβ globulomer epitope (FIG. 8).

### Example 13

### Iodoacetamide Alkylation of (17C, 34C) Aβ(16-35) Oligomer with SELDI-MS Analysis

### A: (17C, 34C) Aβ(16-35) Oligomer Used for Alkylation:

The (17C, 34C) Aβ(16-35) oligomer (4p) of example 4p was used.

### B: Iodoacetamide Alkylation

B1: Control Samples without Iodoacetamide Alkylation
2.5 μl of the oligomer were diluted with 50 μl 100 mM Tris/HCl, 1 mM EDTA, pH 8.6 (5 min Helium aerated)
20 min incubation at 37° C.
20 μl H₂O were added
6 h incubation at room temperature
1 μl of sample was diluted in 49 μl 50% CH₃CN, 0.5% TFA

### B2: Iodoacetamide Alkylation Samples

2.5 μl of the oligomer were diluted with 50 μl 100 mM Tris/HCl, 1 mM EDTA, pH 8.6 (5 min Helium aerated)
20 min incubation at 37° C.
20 μl 100 mM iodoactamide (Sigma; Cat. no.: I1149) solution in H₂O were added
6 h incubation at room temperature
1 μl of sample was diluted in 49 μl 50% CH₃CN, 0.5% TFA

### B3: DTT Reduction Followed by Iodoacetamide Alkylation Samples

2.5 μl of the oligomer were diluted with 50 μl 100 mM Tris/HCl, 1 mM EDTA, 2 mM DTT, pH 8.6 (Helium aerated)
20 min incubation at 37° C.
20 μl 100 mM Iodoactamide (Sigma; Cat. no.: I1149) solution in H₂O were added
6 h incubation at room temperature
1 μl of sample was diluted in 49 μl 50% CH₃CN, 0.5% TFA

### C: Surface-Enhanced Laser Desorption Ionization-Mass Spectrometry (SELDI-MS) Quantification of the Oligomer

2 μL sample of samples B1 and B2 and B3 were individually spotted onto a H4 Protein Chip Array (BioRad; Cat. no. C573-0028).
The spots were allowed to dry on a warm incubator plate.
CHCA-solution:
   5 mg CHCA were dissolved in 150 μL acetonitrile+150 μL 1% TFA=stock solution; stored at −20° C.
   Of the stock solution 10 μL were diluted with 20 μL acetonitrile and 20 μL 1% TFA=working CHCA-solution.
   2 μL of the working CHCA-solution was applied onto the spots
The spots were allowed to dry on a warm incubator plate and analyzed by SELDI-MS (Surface-Enhanced Laser Desorption Ionization-Mass Spectrometry; BioRad, Protein chip SELDI system enterprise edition).
   Conditions: mass range: 800 to 10000 Da; focus mass: 2220 Da; matrix attenuation: 500 Da; sampling rate: 400 MHz; warming shots: 2 with energy: 1100 nj; data shots: 10 with energy 1000 nJoule; Partition 1 of 2.
   Analysis: the peak intensity of the respective double alkylated (2×CM), alkylated (1×CM) or non-alkylated (0×CM) oligomer mass peaks was detected.

The results are shown in FIG. 9.

In the absence of DTT as reducing agent iodoacetamide can not alkylate the —SH group of cysteine if this has formed a covalent S—S bridge, the cystine, with another cysteine. Hence, absence of iodoacetamide alkylation reaction of (17C, 34C) Aβ(16-35) oligomer as analyzed by SELDI shows S—S formation has occurred in (17C, 34C) Aβ(16-35) oligomer. That iodoacetamide alkylation reaction can in principle occur was verified under reducing conditions using DTT to destroy S—S and generate free —SH groups which then were found to be amenable for iodoacetamide alkylation (FIG. 9).

### Example 14

### Recognition of (17C, 34C) Aβ(16-35) Oligomer by Aβ Globulomer-Specific Anti-Bodies Using Immunoprecipitation with SELDI-MS Detection

A: Activation of Dynabeads with Monoclonal Mouse Antibodies
The stock-suspension of dynabeads (Dynabeads M-280 Sheep anti-Mouse IgG, Invitrogen; Cat. no.: 112.02) was shaken carefully to prevent foaming.
1 mL was aseptically removed and transferred to a 1.5 mL reaction vial.
The dynabeads were washed 3 times 5 min with 1 mL immunoprecipitation (IP)-wash buffer (IP-wash-buffer: PBS (20 mM NaH₂PO₄, 140 mM NaCl, pH 7.4), 0.1% BSA). During the washing procedure the supernatant was carefully removed while the dynabeads were immobilized at the side of the reaction vial with a magnetic separator stand (MSS).
The washed dynabeads were incubated with 40 μg Aβ antibody in 1 mL PBS, 0.1% BSA
The activation was carried out by overnight incubation under shaking at 4° C.
The activated dynabeads were washed 4 times 30 min (again using the MSS) with 1 mL IP-wash buffer (PBS (20 mM NaH₂PO₄, 140 mM NaCl, pH 7.4), 0.1% BSA).
The activated dynabeads were resuspended with 1 mL PBS, 0.1% BSA, 0.02% NaAzide; vortexed and centrifuged briefly.
The antibody activated dynabeads were stored at 4° C. until further use.

### B: Preparation of Samples Used for Immunoprecipitation:

1: (17C, 34C) Aβ(16-35) Oligomer without Iodoacetamide Alkylation
2.5 μl of (17C, 34C) Aβ(16-35) oligomer (4p) of example 4p were diluted with 50 μl 100 mM Tris/HCl, 1 mM EDTA, pH 8.6 (5 min Helium aerated)
20 min incubation at 37° C.
20 μl H₂O were added
6 h incubation at room temperature
dialysis overnight against 5 mM NaH₂PO₄, 35 mM NaCl, pH 7.4 in Slide-A-Lyzer Mini Dialysis Units Plus Float, 3500 MWCO (Thermo Scientific, #69558)
   2: (17C, 34C) Aβ(16-35) Oligomer with DTT and Iodoacetamide Alkylation
2.5 μl of (17C, 34C) Aβ(16-35) oligomer (4p) of example 4p were diluted with 50 μl 100 mM Tris/HCl, 1 mM EDTA, 2 mM DTT, pH 8.6 (Helium aerated)
20 min incubation at 37° C.
20 μl 100 mM iodoactamide (Sigma; Cat. no.: I1149) solution in H₂O were added
6 h incubation at room temperature
dialysis overnight against 5 mM NaH₂PO₄, 35 mM NaCl, pH 7.4 in Slide-A-Lyzer Mini Dialysis Units Plus Float, 3500 MWCO (Thermo Scientific, #69558)

### C: Immunoprecipitation (IP)

(17C, 34C) Aβ(16-35) oligomer sample B1 and B2 were diluted with 20 mM NaH₂PO₄, 140 mM NaCl; 0.05% Tween 20, pH 7.4+0.1% BSA to a final concentration of 1 μg/ml.
25 μL of each antibody activated dynabeads of the following list were incubated with 0.1 mL of the diluted samples:
   10F11-Dynabead
   7C6-Dynabeads
   IgG2a-Dynabeads (used as an isotype control for IP background)
   IgG2b-Dynabeads (used as an isotype control for IP background)
The immunoprecipitation was carried out by 18 h incubation under shaking at 6° C.
The dynabeads were immobilized with the MSS.
The supernatant was carefully removed and discarded.
The dynabeads were washed as follows:
   2 times 5 minutes with 500 μL 20 mM NaH₂PO₄, 140 mM NaCl, pH 7.4+0.1% BSA;
   1 time 3 minutes with 500 μL 2 mM NaH₂PO₄, 14 mM NaCl, pH 7.4;
   important: after the last removal of the washing buffer the reaction vials were centrifuged, placed back in the MSS and the remaining drops of fluid carefully removed;
   10 μL 50% CH₃CN, 0.5% TFA in H₂O were added to the reaction vial and vortexed;
   the reaction vials were incubated 10 minutes at RT under shaking;
   the dynabeads were immobilized with the MSS;
   the supernatant comprising the immunoprecipitated eluted Aβ species was carefully withdrawn (═IP-eluate).

### D: Surface-Enhanced Laser Desorption Ionization-Mass Spectrometry (SELDI-MS) Quantification of the Immunoprecipitated (17C, 34C) Aβ(16-35) Oligomer

2 μL IP-eluate was spotted onto a H4 Protein Chip Array (BioRad; Cat. no. C573-0028).
The spots were allowed to dry on a warm incubator plate.
CHCA-solution:
   5 mg CHCA were dissolved in 150 μL acetonitrile+150 μL 1% TFA=stock solution; stored at −20° C.
   Of the stock solution 10 μL were diluted with 20 μL acetonitrile and 20 μL 1% TFA=working CHCA-solution.
   2 μL of the working CHCA-solution was applied onto the spots
The spots were allowed to dry on a warm incubator plate and analyzed by SELDI-MS (Surface-Enhanced Laser Desorption Ionization-Mass Spectrometry; BioRad, Protein chip SELDI system enterprise edition).
   Conditions: mass range: 800 to 10000 Da; focus mass: 2220 Da; matrix attenuation: 500 Da; sampling rate: 400 MHz; warming shots: 2 with energy: 1350 nj; data shots: 10 with energy 1300 nJoule; Partition 1 of 2.
   Analysis: the peak intensity of the respective Aβ(16-35) peptide mass peaks were quantified.

The results are shown in FIGS. 10A and 10B.

The (17C, 34C) Aβ(16-35) oligomer is recognized by the Aβ globulomer epitope specific antibodies 7C6 and 10F11. The unspecific background level during the immunoprecipitation was controlled by the isotype control antibodies IgG2a and IgG2b which show a significantly lower signal intensity in SELDI-MS analysis (FIG. 10A). If the Cysteine S—S covalent reduced by DTT to free —SH and a subsequent iodoacetamide reaction is carried out at the free —SH, the subsequent IP revealed that (17C, 34C) Aβ(16-35) oligomer conformation has been destroyed as the (17C, 34C) Aβ(16-35) peptide with one or two alkylated —SH groups (1× or 2×CM) is no longer recognized by 7C6 or 10F11. Only due to unsufficient DTT reduction and/or iodoacetamide reaction remaining non-alkylated (17C, 34C) Aβ(16-35) oligomer with intact cysteine S—S covalent bond (0×CM) was immunoprecipitated. In conclusion, the (17C, 34C) Aβ(16-35) oligomer contained the Aβ globulomer epitope while a linearized (17C, 34C) Aβ(16-35) peptide did not.

### Reference Examples

### Recombinant N-Met Aβ (1-42) Peptide

Cloning and Expression: The sequence encoding amyloid-beta peptide was cloned and expressed in E. coli using a pET29 vector. The expressed peptide completely retained its N-terminal methionine residue and represents the native sequence of amyloid beta from positions 0 to position 42 (from within the Amyloid Precursor Protein, APP). Peptide was expressed using E. coli BL21 (DE3) cells. Cells were grown at 30° C. until the culture OD₆₀₀ nm reached 0.45, then expression of Aβ was induced by addition of 1 mM IPTG, and the flasks were transferred to an orbital shaker at 41° C. Cells were harvested 3 hour post-induction. The insoluble fraction yielded a product of the expected size, as visualized on a Comassie-stained SDS protein gel.

Purification of Recombinantly Expressed Aβ. The starting material for all isolated samples was cell paste frozen at −80° C. obtained from harvests of E. coli cell cultures. Frozen cell paste was added to 5-10 volumes of lysis buffer (100 mM Tris final pH 7.5) at 4° C. Benzonase, (EMD Biosciences, Madison, Wis.) was added to a concentration of 0.1 μl/ml of cell lysate and stirred until the pellet was uniformly resuspended (45-60 min). Cell lysis was performed using a M-110L microfluidizer (Microfluidics, Newton, Pa.). The lysed material at 15° C. was spun at 23000×G in a JLA 16.25 rotor (Beckman Instruments, Palo Alto, Calif.) for 30 min at 4° C. The material was washed three times by adding cold 50 mM Tris buffer at pH 7.5 to 7.8 and then the resuspended pellets were homogenized and spun at 23000×G in a JLA 16.25 rotor. This was followed by a water wash to remove the Tris buffer. The pellet was resuspended in water containing 0.1% trifluoroacetic acid. The resuspended sample was shell frozen and put on a lyophilizer.

10-20 g of lyophilized material was added to DMSO at 37° C. and homogenized using a tissue homogenizer and stirred for 1 h then allowed to sit overnight. The sample was then spun in two 250 mL nalgene bottles at 25° C. for 30 minutes, 23000×G in a JLA16.25 rotor. The DMSO supernate was decanted and another 50 mL DMSO was added to each bottle, homogenized and spun and then this DMSO was decanted and also saved. The pellets were discarded.

The DMSO extract was carefully poured into a 6000-8000 cut-off dialysis membrane, (Spectrum Laboratories, Rancho Dominguez, Calif.), sufficient to hold about 1.5 L. It was dialyzed against 10 L of 15% acetonitrile to which 10 mL of concentrated ammonia (0.1% v/v) had been added. This was allowed to dialyze for four hours on the bench. Periodically, the dialysis membrane was taken out so as to redistribute the dense DMSO and accelerate the dialysis process. At the end of four hours, the membrane was placed into a fresh change of 10 L of buffer and the process continued for another two hours. At the end of the dialysis, the sample was removed and centrifuged at 23000×G for 30 min at 25° C. The sample was transferred to a 2 L cylinder and diluted two fold with 0.1% ammonia in water to a total volume as large as 2000 ml.

A 2.2×25 cm, (95 mL) stainless column was hand packed with 15-20 micron, 300 A, PLPR-S reversed phase resin from Polymer Labs (Amherst, Mass.). It was taken through a cycle from 75% acetonitrile+0.1% ammonia, (75% B) and equilibrated to 10% B. The column was connected to a Pharmacia P500 pump (Amersham Biosciences, Piscataway, N.J.), and 1400 mL of the peptide solution was pumped through the column overnight on the bench at room temperature over ˜16.7 hr. The next morning, the column was washed with the P500 pump with about 250 mL 10% B and then connected to a Beckman HPLC (Palo Alto, Calif.). Washing was continued with 10% B until the absorbance at 280 nm came to baseline and then a gradient was initiated from 10° AB to 30% B over 200 minutes (0.1% gradient). The full-scale absorbance was kept at 1 absorbance unit and the flow at 5 mL/min. The material was hand collected in about 50 fractions of about 10 mL. 100 uL of each of the fractions were put on a speed vac, dried and 100 uL of 1× sample buffer added to the tubes. The material was concentrated on a 3500 cut-off membrane on an Amicon (Millipore Inc, Billerica, Calif.) stirred cell and concentrated from ˜350 to 50 mL it was then lyophilized to dryness overnight.

Purified Aβ peptide produced exhibited an observed mass of 4645 to 4648 Da. This was consistent with the presence of N-terminal methionine as expected from the DNA expression sequence employed.

### Reference Examples

### Reference Example 1

### Aβ(1-42) Globulomer

The Aβ(1-42) synthetic peptide (H-1368, Bachem, Bubendorf, Switzerland) was suspended in 100% 1,1,1,3,3,3-hexafluoro-2-propanol (HFIP) at 6 mg/mL and incubated for complete solubilization under shaking at 37° C. for 1.5 h. The HFIP acts as a hydrogen-bond breaker and is used to eliminate pre-existing structural inhomogeneities in the Aβ peptide. HFIP was removed by evaporation in a SpeedVac and Aβ(1-42) resuspended at a concentration of 5 mM in dimethylsulfoxide and sonicated for 20 s. The HFIP-pre-treated Aβ(1-42) was diluted in phosphate-buffered saline (PBS) (20 mM NaH₂PO₄, 140 mM NaCl, pH 7.4) to 400 μM and 1/10 volume 2% sodium dodecyl sulfate (SDS) (in H₂O) added (final concentration of 0.2% SDS). An incubation for 6 h at 37° C. resulted in the 16/20-kDa Aβ(1-42) globulomer intermediate. The 38/48-kDa Aβ(1-42) globulomer was generated by a further dilution with three volumes of H₂O and incubation for 18 h at 37° C. After centrifugation at 3000 g for 20 min the sample was concentrated by ultrafiltration (30-kDa cut-off), dialysed against 5 mM NaH₂PO₄, 35 mM NaCl, pH 7.4, centrifuged at 10000 g for 10 min and the supernatant comprising the 38/48-kDa Aβ(1-42) globulomer withdrawn.

### Reference Example 2

### N-Met Aβ(1-42) Globulomer

6 mg human amyloid β(1-42) peptide (Bachem Biosciences, King of Prussia, Pa.) or recombinant N-Met Aβ (1-42) peptide+2×500 μL HFIP (hexafluoroisopropanol) (6 mg/mL suspension) in two 1.6 mL Eppitubes (2×500 μL portions) (Eppendorf Northamerica, Westbury, N.Y.) were shaken for 1.5 h at 37° C., dried in speedvac for 1.5 h and then resuspended in 2 aliquots of 132 μL each of DMSO, sonicated in a water bath for 20 seconds, shaken gently for 10 min on a plate agitator; and stored at −20° C. 690 μL 20 mM NaPO₄; 140 mM NaCl; pH 7.4 buffer were filled in a 15 mL Falcon tube, 60 μL 5 mM amyloid stock suspension in DMSO (400 μM amyloid) and then 75 μl 2% SDS in water (0.2% SDS) were added. The mixture was incubated for 6-8 h at 37° C. and 2475 μL water (4 fold dilution with water) were added for a final volume of 3.3 mL. The mixture was incubate for 18-20 h at 37° C., centrifuged 10 min at 3000×G and finally the supernatant was concentrated to 1 mL by 30 kDa Centriprep (Millipore Inc, Billerica, Calif.). The sample was dialyzed overnight in PBS/4 (PBS=phosphate buffered saline, 1 mM KCl, 154 mM NaCl, 4 mM phosphate, pH 7.4; PBS/432 PBS diluted 1 to 4 with distilled water, pH 7.4 final) at 4° C. in a 12-15 kDa cut-off dialysis tubing. The concentrate was centrifuged at 10,000×G for 10 min in an Eppendorff tube (clear pellet), aliquoted in 250 μL and freezed at −20° C.

### Reference Example 3

### Aβ(20-42) Globulomer

1.59 ml of the Aβ(1-42) globulomer preparation of reference example 3 were admixed with 38 ml of buffer (50 mM MES/NaOH, pH 7.4) and 200 μl of a 1 mg/ml thermolysin solution (Roche) in water. The reaction mixture was stirred at RT for 20 h. Then 80 μl of a 100 mM EDTA solution, pH 7.4, in water were added and the mixture was furthermore adjusted to an SDS content of 0.01% with 400 μl of a 1% strength SDS solution. The reaction mixture was concentrated to approx. 1 ml via a 15 ml 30 kDa Centriprep tube. The concentrate was admixed with 9 ml of buffer (50 mM MES/NaOH, 0.02% SDS, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 2% strength SDS solution in water. The sample was centrifuged at 10000 g for 10 min and the Aβ(20-42) globulomer supernatant was withdrawn.

### Reference Example 4

### Aβ(12-42) Globulomer

2 ml of the Aβ(1-42) globulomer preparation of reference example 3 were admixed with 38 ml buffer (5 mM sodium phosphate, 35 mM sodium chloride, pH 7.4) and 150 μl of a 1 mg/ml GIuC endoproteinase (Roche) in water. The reaction mixture was stirred for 6 h at RT, and a further 150 μl of a 1 mg/ml GIuC endoproteinase (Roche) in water were subsequently added. The reaction mixture was stirred at RT for another 16 h, followed by addition of 8 μl of a 5 M DIFP solution. The reaction mixture was concentrated to approx. 1 ml via a 15 ml 30 kDa Centriprep tube. The concentrate was admixed with 9 ml of buffer (5 mM sodium phosphate, 35 mM sodium chloride, pH 7.4) and again concentrated to 1 ml. The concentrate was dialyzed at 6° C. against 1 l of buffer (5 mM sodium phosphate, 35 mM NaCl) in a dialysis tube for 16 h. The dialysate was adjusted to an SDS content of 0.1% with a 1% strength SDS solution in water. The sample was centrifuged at 10000 g for 10 min and the Aβ(12-42) globulomer supernatant was withdrawn.

### Reference Example 5

### Aβ(1-40) Monomer (0.1% NaOH)

1 mg Aβ(1-40) (Bachem Inc., cat. no. H-1194) was dissolved in 232.6 μl 0.1% NaOH in H₂O (freshly prepared) (=4.3 mg/ml=1 nmol/1 μl) and immediately shaken for 30 sec. at room temperature to get a clear solution. The sample was stored at −20° C. for further use.

### Reference Example 6

### Aβ(1-42) Monomer (0.1% NaOH)

1 mg Aβ(1-42) (Bachem Inc., cat. no. H-1368) were dissolved in 222.2 μl 0.1% NaOH in H₂O (freshly prepared) (=4.5 mg/ml=1 nmol/1 μl) and immediately shaken for 30 sec. at room temperature to get a clear solution. The sample was stored at −20° C. for further use.

### Reference Example 7

### Aβ Fibrils

1 mg Aβ(1-42) (Bachem Inc. Catalog Nr.: H-1368) were dissolved in 500 μl aqueous 0.1% NH₄OH (Eppendorff tube) and the sample was stirred for 1 min at room temperature. The sample was centrifuged for 5 min at 10000×g and the supernatant was withdrawn. 100 μl of this freshly prepared Aβ(1-42) solution were neutralized with 300 μl 20 mM NaH₂PO₄; 140 mM NaCl, pH 7.4. The pH was adjusted to pH 7.4 with 1% HCl. The sample was incubated for 24 h at 37° C. and centrifuged (10 min at 10000×g). The supernatant was discarded and the fibril pellet washed twice with 400 μl 20 mM NaH₂PO₄, 140 mM NaCl, pH 7.4 and then finally resuspended with 400 μl of 20 mM NaH₂PO₄; 140 mM NaCl, pH 7.4 by vortexing for 1 min.

### Reference Example 8

### sAPPα

Supplied from Sigma (cat. no. S9564; 25 μg in 20 mM NaH₂PO₄; 140 mM NaCl; pH 7.4). The sAPPα was diluted with 20 mM NaH₂PO₄, 140 mM NaCl, pH 7.4, 0.2 mg/ml BSA to 0.1 mg/ml (=1 pmol/μl).

### Reference Example 9

### Polyclonal Antiserum 5599

The polyclonal antiserum 5599 was obtained in the manner as polyclonal antiserum 5600 described in WO 2004/067561, Example 25a, Serum a1 5600, with the exception that Aβ(20-42) globulomer was used instead of 5600 LPH conjugated Aβ(20-42) globulomer.

We claim:

1. An amyloid β peptide analogue comprising an amino acid sequence, wherein the sequence (i) forms a loop, (ii) has at least 66% identity to native human Aβ peptide or a portion thereof, (iii) comprises at least 6 contiguous amino acid residues and (iv) has at least 2 non-contiguous amino acid residues which are covalently linked with each other; or a derivative thereof.

2. The amyloid β peptide analogue of claim 1, wherein the amino acid sequence is a peptidomimetic.

3. The amyloid β peptide analogue of claim 1, wherein the loop is a β-hairpin loop.

4. The amyloid β peptide analogue of claim 1, wherein native Aβ human peptide or the portion thereof is Aβ(X . . . Y), X being selected from the group consisting of the numbers 1 . . . 23, 15 . . . 23, or 18 . . . 22 and Y being selected from the group consisting of the numbers 28 . . . 43, or 28 . . . 43.

5. The amyloid β peptide analogue of claim 1, wherein the 6 contiguous amino acid residues comprise the sequence VGSN or DVGSNK or AED.

6. The amyloid β peptide analogue of claim 1, wherein the amino acid sequence comprises the sequence F₁₉X₂₀A₂₁-Q-A₃₀I₃₁I₃₂, with X₂₀ representing an amino acid and Q being an amino acid sequence comprising the sequence VGSN.

7. The amyloid β peptide analogue of claim 6, wherein at least part of the amino acid sequence Q forms the loop and the amino acid sequences F₁₉X₂₀A₂₁ and A₃₀I₃₁I₃₂ are in anti-parallel orientation.

8. The amyloid β peptide analogue of claim 1, wherein the amino acid sequence comprises a sequence selected from the group consisting of SEQ ID NO:1-368, at least two amino acid residues of said sequence being modified so as to form an intra-sequence covalent linkage.

9. The amyloid β peptide analogue of claim 1, wherein the amino acid sequence of the amyloid β peptide analogue is a sequence selected from the group consisting of SEQ ID NO:369-698, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence;
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₁ is alanine, valine, glycine, serine, or an amino acid residue which is covalently linked to another amino acid residue of the sequence;
X₂₂ is glutamic acid, aspartic acid, or an amino acid residue which is covalently linked to another amino acid residue of the sequence;
X₂₉ is glycine, alanine, serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
at least one amino acid residue selected from the group consisting of X₁₂, X₁₃, X₁₄, X₁₅, X₁₆, X₁₇, X₁₈, X₁₉, X₂₀, X₂₁ and X₂₂ and at least one amino acid residue selected from the group consisting of X₂₉, X₃₀, X₃₁, X₃₂, X₃₃, X₃₄, X₃₅, X₃₆, X₃₇, X₃₈, X₃₉ being covalently linked with each other.

10. The amyloid β peptide analogue of claim 1, wherein the amino acid sequence of the amyloid β peptide analogue comprises the sequence X₂₀-Q-X₂₄X₂₅X₂₆X₂₇X₂₈X₂₉A₃₀I₃₁, with each of X₂₀, X₂₄ X₂₅, X₂₆, X₂₇, X₂₈, X₂₉ independently representing an amino acid and Q being an amino acid sequence comprising the sequence AED.

11. The amyloid β peptide analogue of claim 10, wherein at least part of the amino acid sequence X₂₄X₂₅X₂₆X₂₇ forms the loop and the amino acid sequences X₂₀A₂₁E₂₂D₂₃ and X₂₈X₂₉A₃₀I₃₁ are in anti-parallel orientation.

12. The amyloid β peptide analogue of claim 1, wherein the amino acid sequence of the amyloid β peptide analogue is a sequence selected from the group consisting of SEQ ID NO:699-960, wherein
X₁₂ is valine, leucine, isoleucine, alanine, methionine, or an amino acid residue which is covalently linked to another amino acid residue of the sequence;
X₁₃ is histidine, tyrosine, serine, methionine, or an amino acid residue which is covalently linked to another amino acid residue of the sequence;
X₁₄ is histidine, tyrosine, serine, methionine, or an amino acid residue which is covalently linked to another amino acid residue of the sequence;
X₁₅ is glutamine, asparagine, methionine, serine, or an amino acid which is covalently linked to another amino acid residue of the sequence;
X₁₆ is lysine, arginine, or an amino acid residue which is covalently linked to another amino acid residue of the sequence;
X₁₇ is leucine, isoleucine, valine, phenylalanine, methionine, or an amino acid residue which is covalently linked to another amino acid residue of the sequence;
X₁₈ is valine, leucine, isoleucine, alanine, methionine, or an amino acid residue which is covalently linked to another amino acid residue of the sequence;
X₁₉ is phenylalanine, tyrosine, valine, leucine, isoleucine, methionine, or an amino acid residue which is covalently linked to another amino acid residue of the sequence;
X₂₀ is phenylalanine, tyrosine, valine, leucine, isoleucine, methionine, or an amino acid residue which is covalently linked to another amino acid residue of the sequence;
X₂₄ is valine, leucine, isoleucine, alanine, methionine, or an amino acid residue which is covalently linked to another amino acid residue of the sequence;
X₂₅ is glycine, alanine, serine, or an amino acid residue which is covalently linked to another amino acid residue of the sequence;
X₂₆ is serine, glycine, alanine, threonine, or an amino acid residue which is covalently linked to another amino acid residue of the sequence;
X₂₇ is asparagine, glutamine, methionine, or an amino acid residue which is covalently linked to another amino acid residue of the sequence;
X₂₈ is lysine, arginine, or an amino acid residue which is covalently linked to another amino acid residue of the sequence;
X₂₉ is glycine, alanine, serine, or an amino acid residue which is covalently linked to another amino acid residue of the sequence;
X₃₀ is alanine, valine, glycine, serine, or an amino acid residue which is covalently linked to another amino acid residue of the sequence;
X₃₁ is isoleucine, leucine, valine, phenylalanine, methionine, or an amino acid residue which is covalently linked to another amino acid residue of the sequence;
X₃₂ is isoleucine, leucine, valine, phenylalanine, methionine, or an amino acid residue which is covalently linked to another amino acid residue of the sequence;
X₃₃ is glycine, alanine, serine, or an amino acid residue which is covalently linked to another amino acid residue of the sequence;
X₃₄ is leucine, isoleucine, valine, phenylalanine, methionine, or an amino acid residue which is covalently linked to another amino acid residue of the sequence;
X₃₅ is methionine, valine, leucine, isoleucine, alanine, or an amino acid residue which is covalently linked to another amino acid residue of the sequence;
X₃₆ is valine, leucine, isoleucine, alanine, methionine, or an amino acid residue which is covalently linked to another amino acid residue of the sequence;
X₃₇ is glycine, alanine, serine, or an amino acid residue which is covalently linked to another amino acid residue of the sequence;
X₃₈ is glycine, alanine, serine, or an amino acid residue which is covalently linked to another amino acid residue of the sequence; and
X₃₉ is valine, leucine, isoleucine, alanine, methionine, or an amino acid residue which is covalently linked to another amino acid residue of the sequence,
at least one amino acid residue selected from the group consisting of X₁₂, X₁₃, X₁₄, X₁₅, X₁₆, X₁₇, X₁₈, X₁₉, X₂₀, and at least one amino acid residue selected from the group consisting of X₂₉, X₃₀, X₃₁, X₃₂, X₃₃, X₃₄, X₃₅, X₃₆, X₃₇, X₃₈, X₃₉ being covalently linked with each other.

13. The derivative of claim 1 wherein the derivative is an oligomer comprising a plurality of amyloid β peptide analogues.

14. A process for preparing the amyloid β peptide analogue of claim 1, which process comprises
(i) providing a peptide or peptidomimetic thereof;
(ii) subjecting the peptide or peptidomimetic to conditions sufficient for the formation of the linkage.

15. A process for preparing the oligomer of claim 13, which process comprises
(i) providing a peptide or peptidomimetic thereof;
(ii) subjecting the peptide or peptidomimetic to conditions sufficient for the formation of the oligomer and the linkage.

16. A composition comprising the amyloid β peptide analogue as in claim 1 and optionally a pharmaceutical acceptable carrier.

17. Use of an amyloid β peptide analogue or derivative as in claim 1 for preparing a pharmaceutical composition for active immunization in treating or preventing amyloidosis.

18. Use of an amyloid β peptide analogue or derivative as in claim 1 for preparing a composition for diagnosing amyloidosis.

19. A method of identifying an agent capable of binding to an amyloid β peptide analogue or derivative as in claim 1, which method comprises the steps of: a) exposing one or more agents of interest to the amyloid β peptide analogue or derivative for a time and under conditions sufficient for the one or more agents to bind to the amyloid β peptide analogue or derivative; and b) identifying those agents which bind to the amyloid β peptide analogue or derivative.

20. A method of providing an antibody capable of binding to an amyloid β peptide analogue or derivative as in claim 1, which comprises
i) providing an antigen comprising the amyloid β peptide analogue or derivative;
ii) exposing an antibody repertoire to said antigen; and
iii) selecting from said repertoire an antibody which binds to the amyloid β peptide analogue or derivative.
